(19) United States
(12) Patent Application Publication
(10) Pub. No.: US 2006/0004195 A1
(43) Pub. Date: Jan. 5, 2006

(54) Alpha,beta-unsaturated esters and acids by stereoselective dehydration

(76) Inventors: Xiaohu Deng, San Diego, CA (US); Neelakandha Mani, San Diego, CA (US); Christopher M. Mapes, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/168,938

(22) Filed: Jun. 28, 2005

Related U.S. Application Data
(60) Provisional application No. 60/584,227, filed on Jun. 30, 2004.

(51) Int. Cl. C07D 43/02; C07D 413/02; C07D 417/02

(52) U.S. Cl. 540/603; 544/140; 544/284; 546/118; 546/275.4; 544/353; 548/131; 548/152; 548/217; 548/240; 544/238; 546/146; 548/364.7

(57) ABSTRACT

There are provided by the present invention certain pyrazole based CCK-1 receptor modulators which have the general formula: 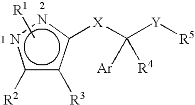
wherein Ar is an aromatic or heteroaromatic group, X is a hydrocarbon linker, Y is a bond or hydrocarbon linker and R¹, R², R³, R⁴ and R⁵ are certain organic substituents, methods for making the same, and stereoselective dehydration methods for generally making α,β-unsaturated esters, acids and their derivatives.

This application claims the benefit of U.S. provisional patent application Ser. No. 60/584,227, filed on Jun. 30, 2004, the contents of which is incorporated herein by reference.

### FIELD OF THE INVENTION

This invention relates to CCK-1 receptor modulators for the treatment of gastrointestinal and CNS disorders. More particularly, this invention relates to certain pyrazole compounds useful as selective agonists or antagonists of the CCK-1 receptor as well as methods for making such compounds.

### BACKGROUND OF THE INVENTION

Cholecystokinin (CCK) is a brain-gut peptide hormone located both in the gastrointestinal system and in the central nervous system. The actions of CCK are mediated by two G-protein coupled receptors: CCK-1 (formerly CCK-A) and CCK-2 (formerly CCK-B/gastrin). These CCK receptors are expressed throughout the gastrointestinal system and in different parts of the central nervous system including the cortex, the striatum, the hypothalamus, the hippocampus, the olfactory bulb, the vagal afferent neurones, in different enteric nerves and in the genital tract.

CCK has a number of biological actions. CCK is the primary hormonal regulator of gall bladder contraction in response to a meal. CCK stimulates pancreatic and biliary secretions and regulates GI motility and specifically gut and colonic motility. CCK promotes protein synthesis and cell growth, especially in the GI system and in the pancreas. CCK is involved in mediating satiety after a meal. CCK is an important neuromodulator and neurotransmitter involved in anxiety and panic disorder. CCK modulates the release of dopamine. CCK is also known to antagonize morphine and beta-endorphin induced analgesia and the action on nociception. A review of CCK receptors, ligands and the activities thereof may be found in Tullio, P. et al. Exp. Opin. Invest. Drugs 2000, 9(1), 129-146.

A number of CCK-1 receptor antagonists are presently in clinical trials including, tarazepide, devazepide, and lintitript. Phase III equivalent trials are in progress by Rotta Research Group and Forest Laboratories on dexioxiglumide, a CCK-1 antagonist for the treatment of constipation, irritable bowel syndrome and non-ulcer dyspepsia.

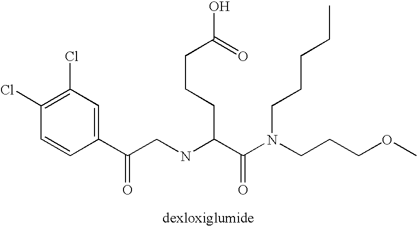

Also, Kaken Pharmaceuticals and Mitsubishi-Tokyo Pharmaceuticals are awaiting registration in Japan on loxiglumide, a CCK-1 receptor antagonist for the treatment of GI cancers and pancreatitis. Loxiglumide is the racemate of dexloxiglumide.

A number of CCK-1 receptor agonists are under preclinical investigation. Glaxo SmithKline, Inc. is investigating GW 5823, GW 7854, GW 7178, and GW 8573, 1,5-benzodiaepines for the treatment of gallstones, gastrointestinal disease, and obesity.

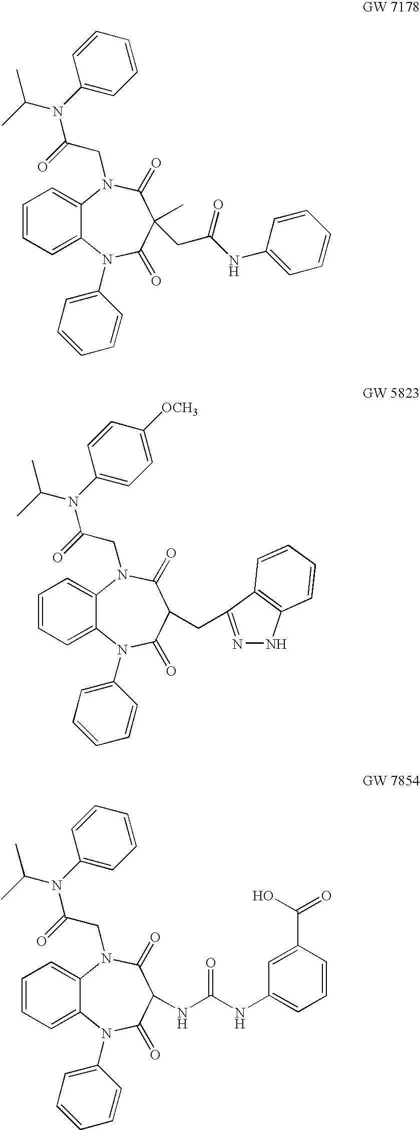

Also, Pfizer is investigating the CCK-1 receptor agonist, PD 170292, for obesity.

In U.S. Pat. Nos. 4,826,868 and 5,164,381 there are disclosed certain pyrazoles for alleviating inflammation and treating cardiovascular disorders in mammals having the general formula: 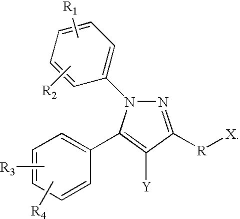
These compounds are not taught to be CCK-1 receptor modulators nor suggested to be useful in the treatment of disease states mediated by CCK-1 receptor activity.

In U.S. Pat. No. 5,051,518 there are disclosed certain pyrazoles for alleviating inflammation and treating cardiovascular disorders in mammals having the general formula: 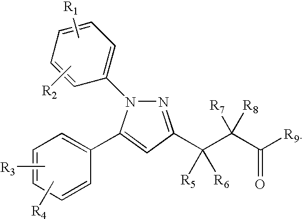
These compounds are not taught to be CCK-1 receptor modulators nor suggested to be useful in the treatment of disease states mediated by CCK-1 receptor activity.

Applicants have now discovered that certain pyrazoles as described below are useful CCK-1 receptor modulators, agonists and antagonists, and most particularly antagonists. As such, these compounds are useful to treat a number of disease states mediated by CCK. Applicants have also discovered methods for making such compounds, including methods for large scale synthesis of such compounds.

### SUMMARY OF THE INVENTION

There are provided by the present invention CCK-1 receptor antagonists, and methods of making the same, which have the general formula: 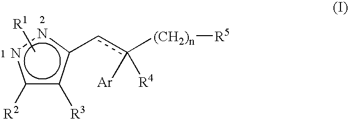
wherein,
R¹ is a 1- or 2-position substituent selected from the group consisting of —H,
   a) phenyl, optionally mono-, di-, or tri-substituted with R^{p} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)—, or —(CH₂)₁₋₂N(C₁₋₄alkyl)(CH₂)—;
      R^{p} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y})R^{z} (wherein R^{y} and R^{z} are independently selected from —H, —C₁₋₆alkyl, and —C₁₋₆alkenyl, or R^{y} and R^{z} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y})R^{z}, —(N—R^{t})COR^{t}, —(N—R^{t})SO₂C₁₋₆alkyl (wherein R^{t} is —H or —C₁₋₆alkyl or two R^{t} in the same substituent may be taken together with the amide of attachment to form an otherwise aliphatic hydrocarbon ring, said ring having 4 to 6 members), —(C═O)C₁₋₆alkyl, —(S═(O)ₘ)—C₁₋₆alkyl (wherein m is selected from 0, 1, and 2), —SO₂N(R^{y})R^{z}, —SCF₃, halo, —CF₃, —OCF₃, —COOH, and —COOC₁₋₆alkyl;
   b) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{p};
   c) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{p};
   d) naphthyl, optionally mono-, di-, or tri-substituted with R^{p};
   e) a monocyclic aromatic hydrocarbon group having five ring atoms, having a carbon atom which is the point of attachment, having one carbon atom replaced by >O, >S, >NH, or >N(C₁₋₄alkyl), having up to two additional carbon atoms optionally replaced by N, optionally mono- or di-substituted with R^{p} and optionally benzo fused on the condition that two or fewer of said carbon ring atoms are replaced by a heteroatom, where the benzo fused moiety is optionally mono-, di-, or tri-substituted with R^{p};
   f) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{p} and optionally benzo fused, where the benzo fused moiety is optionally mono- or di-substituted with R^{p};
   g) adamantanyl or monocyclic C₅₋₇cycloalkyl, optionally having one or two carbon members optionally replaced with >O, >NH, or >N(C₁₋₄alkyl), optionally having one or two unsaturated bonds in the ring, and optionally having one of the ring atoms substituted with —OH, ═O, or —CH₃;
   h) a —C₁₋₈alkyl; and
   i) —C₁₋₄alkyl, mono-substituted by a substituent selected from the group consisting of any one of a) to g);
R² is selected from the group consisting of:
   i) phenyl, optionally mono-, di-, or tri-substituted with R^{q} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{q} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y})R^{z} (wherein R^{y} and R^{z} are independently selected from —H, —C₁₋₆alkyl, and —C₁₋₆alkenyl, or R^{y} and R^{z} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y})R^{z}, —(N—R^{t})COR^{t}, —(N—R^{t})SO₂C₁₋₆alkyl (wherein R^{t} is —H or —C₁₋₆alkyl or two R^{t} in the same substituent may be taken together with the amide of attachment to form an otherwise aliphatic hydrocarbon ring, said ring having 4 to 6 members), —(C═O)C₁₋₆alkyl, —(S═(O)ₘ)—C₁₋₆alkyl (wherein m is selected from 0, 1, and 2), —SO₂N(R^{y})R^{z}, —SCF₃, halo, —CF₃, —OCF₃, —COOH, and —COOC₁₋₆alkyl;
   ii) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{q};
   iii) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{q};
   iv) naphthyl, optionally mono-, di-, or tri-substituted with R^{q};
   v) a monocyclic aromatic hydrocarbon group having five ring atoms, having a carbon atom which is the point of attachment, having one carbon atom replaced by >O, >S, >NH, or >N(C₁₋₆alkyl), having up to one additional carbon atoms optionally replaced by N, optionally mono- or di-substituted with R^{q} and optionally benzo fused on the condition that two or fewer of said carbon ring atoms are replaced by a heteroatom, where the benzo fused moiety is optionally mono-, di-, or tri-substituted with R^{q}; and
   vi) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{p} and optionally benzo fused, where the benzo fused moiety is optionally mono- or di-substituted with R^{q};
R³ is selected from the group consisting of —H, halo, and —C₁₋₆alkyl;
n is selected from 0, 1, and 2, with the proviso that where R⁵ is attached through —S—, n is 1 or 2;
R⁴ is selected from the group consisting of —H, halo, and —C₁₋₆alkyl or is absent in the case where the double bond is present in the above structure;
Ar is selected from the group consisting of:
   A) phenyl, optionally mono-, di-, or tri-substituted with R^{r} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{r} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y})R^{z} (wherein R^{y} and R^{z} are independently selected from —H, —C₁₋₆alkyl, and —C₁₋₆alkenyl, or R^{y} and R^{z} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y})R^{z}, —(N—R^{t})COR^{t}, —(N—R^{t})SO₂C₁₋₆alkyl (wherein R^{t} is —H or —C₁₋₆alkyl or two R^{t} in the same substituent may be taken together with the amide of attachment to form an otherwise aliphatic hydrocarbon ring, said ring having 4 to 6 members), —(C═O)C₁₋₆alkyl, —(S═(O)ₘ)—C₁₋₆alkyl (wherein m is selected from 0, 1, and 2), —SO₂N(R^{y})R^{z}, —SCF₃, halo, —CF₃, —OCF₃, —COOH, and —COOC₁₋₆alkyl;
   B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r};
   C) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r};
   D) naphthyl, optionally mono-, di-, or tri-substituted with R^{r};
   E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r} and optionally benzo fused on the condition that two or fewer of said carbon ring atoms are replaced by a heteroatom, where the benzo fused moiety is optionally mono-, di-, or tri-substituted with R^{r}; and
   F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r} and optionally benzo fused, where the benzo fused moiety is optionally mono- or di-substituted with R^{r};
R⁵ is selected from the group consisting of:
   I) —COOR⁶, where R⁶ is selected from the group consisting of —H and —C₁₋₄alkyl,
   II) —CONR⁷R⁸, where R⁷ and R⁸ are independently selected from the group consisting of —H, —C₁₋₆alkyl, and —C₃₋₆cycloalkyl, optionally hydroxy substituted, or R⁷ and R⁸ may be taken together with the nitrogen of attachment to form an otherwise aliphatic hydrocarbon ring, said ring having 5 to 7 members, optionally having one carbon replaced with >O, ═N—, >NH, or >N(C₁₋₄alkyl), and optionally having one or two unsaturated bonds in the ring; and
III) tetrazolyl, [1,2,4]triazol-3-ylsulfanyl, [1,2,4]triazol-3-ylsulfonyl, [1,2,4]triazole-3-sulfinyl and [1,2,3]triazol-4-ylsulfanyl, [1,2,3]triazol-4-ylsulfonyl, [1,2,3]triazol-4-sulfinyl;
   and enantiomers, diastereomers, and pharmaceutically acceptable salts and esters thereof.

Regarding methods of making compounds according to this invention that comprise a stereoselective dehydration of an α-hydroxyester, there are provided by the present invention synthetic methods of making compounds which have the general formula: 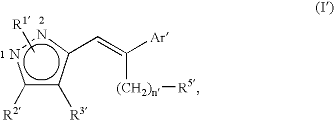
esters, enantiomers, diastereomers, racemics, and pharmaceutically acceptable salts thereof,
wherein,
R¹ is a 1- or 2-position substituent selected from the group consisting of —H,
   a) phenyl, optionally mono-, di-, or tri-substituted with R^{p′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{p′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t″})COR^{t}, —(N—R^{t′})SO₂C₁₋₆alkyl (wherein R^{t′} is —H or —C₁₋₆alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH, and —COOC₁₋₆alkyl;
   b) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{p′};
   c) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{p′};
   d) naphthyl, optionally mono-, di-, or tri-substituted with R^{p′};
   e) a monocyclic aromatic hydrocarbon group having five ring atoms, having a carbon atom which is the point of attachment, having one carbon atom replaced by >O, >S, >NH, or >N(C₁₋₄alkyl), having up to two additional carbon atoms optionally replaced by N, optionally mono- or di-substituted with R^{p′} and optionally benzo or pyrido fused on the condition that two or fewer of said carbon ring atoms are replaced by a heteroatom, where the benzo or pyrido fused moiety is optionally mono-, di-, or tri-substituted with R^{p′};
   f) a monocyclic aromatic hydrocarbon group having six ring atoms, having a carbon atom which is the point of attachment, having one or two carbon atoms replaced by N, optionally mono- or di-substituted with R^{p′} and optionally benzo or pyrido fused, where the benzo or pyrido fused moiety is optionally mono- or di-substituted with R^{p′};
   g) adamantanyl or monocyclic C₅₋₇cycloalkyl, optionally having one or two carbon members optionally replaced with >O, >NH, or >N(C₁₋₄alkyl), optionally having one or two unsaturated bonds in the ring, and optionally having one of the ring atoms substituted with —OH, =0 or —CH₃;
   h) a —C₁₋₈alkyl; and
   i) —C₁₋₄alkyl, mono-substituted by a substituent selected from the group consisting of any one of a) to g);
R²′ is selected from the group consisting of:
   i) phenyl, optionally mono-, di-, or tri-substituted with R^{q′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{q′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t″})COR^{t}, —(N—R^{t′})SO₂C₁₋₆alkyl (wherein R^{t′} is H or C₁₋₆alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH and —COOC₁₋₆alkyl;
   ii) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{q′};
   iii) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{q′};
   iv) naphthyl, optionally mono-, di-, or tri-substituted with R^{q′};
   v) a monocyclic aromatic hydrocarbon group having five ring atoms, having a carbon atom which is the point of attachment, having one carbon atom replaced by >O, >S, >NH, or >N(C₁₋₆alkyl), having up to one additional carbon atoms optionally replaced by N, optionally mono- or di-substituted with R^{q′} and optionally benzo or pyrido fused on the condition that two or fewer of said carbon ring atoms are replaced by a heteroatom, where the benzo or pyrido fused moiety is optionally mono-, di-, or tri-substituted with R^{q′}; and
   vi) a monocyclic aromatic hydrocarbon group having six ring atoms, having a carbon atom which is the point of attachment, having one or two carbon atoms replaced by N, optionally mono- or di-substituted with R^{q′} and optionally benzo or pyrido fused, where the benzo or pyrido fused moiety is optionally mono- or di-substituted with R R^{q′};
R^{3′} is selected from the group consisting of —H, halo, and —C₁₋₆alkyl;
n′ is 0;
Ar′ is selected from the group consisting of:
   A) phenyl, optionally mono-, di-, or tri-substituted with R^{r′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl) or —(CH₂)₁₋₂N(C₁₋₄alkyl)(CH₂)—;
      R^{r′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆-alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t″})COR^{t′}, —(N—R^{t′})SO₂C₁₋₆alkyl (wherein R^{t′} is —H or —C₁₋₆alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH, and —COOC₁₋₆alkyl;
   B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r′};
   C) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r′};
   D) naphthyl, optionally mono-, di-, or tri-substituted with R^{r′};
   E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r′} and optionally benzo or pyrido fused on the condition that two or fewer of said carbon ring atoms are replaced by a heteroatom, where the benzo or pyrido fused moiety is optionally mono-, di-, or tri-substituted with R^{r′};
   F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r′} and optionally benzo or pyrido fused, where the benzo or pyrido fused moiety is optionally mono- or di-substituted with R^{r′};
   G) adamantanyl or monocyclic C₅₋₇cycloalkyl, optionally having one or two carbon members optionally replaced with >O, >S, >NH, or >N(C₁₋₄alkyl), optionally having one or two unsaturated bonds in the ring, and optionally having one of the ring atoms substituted with —OH, =0 or —CH₃;
   H) a —C₁₋₈alkyl wherein the carbon of attachment bears no hydrogen substituents, optionally mono-substituted by a substituent selected from the group consisting of any one of a) to g), and optionally mono-, di-, or tri-substituted by a R^{4′};
   I) —C₂alkenyl or —C₂alkynyl, optionally mono-substituted by a substituent selected from the group consisting of any one of a) to h); and
R^{5′} is COOR^{6′}, where R^{6′} is selected from the group consisting of —H and —C₁₋₄alkyl.

Regarding methods of making compounds according to this invention that comprise a stereoselective dehydration of an α-hydroxyester, there are provided by the present invention synthetic methods of making compounds which have the general formulae: 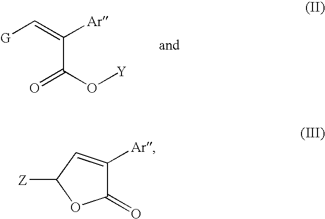
esters, enantiomers, diastereomers, racemics, and pharmaceutically acceptable salts thereof,
wherein,
G is selected from the group consisting of
   a) phenyl, optionally mono-, di-, or tri-substituted with R^{p″} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{p″} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y″} and R^{z″} are independently selected from —H, —C₁₋₆alkyl, and —C₁₋₆alkenyl, or R^{y′} and R^{z″} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y″})R^{z″}, —(N—R^{t″})COR^{t″}, —(N—R^{t″})SO₂C₁₋₆alkyl (wherein R^{t″} is —H or —C₁₋₆alkyl, or two R^{t} in the same substituent may be taken together with the amide of attachment to form an otherwise aliphatic hydrocarbon ring, said ring having 4 to 6 members), —(C═O)C₁₋₆alkyl, —(S═(O)_{m″})—C₁₋₆alkyl (wherein m″ is selected from 0 and 2), —SO₂N(R^{y″})R^{z″}, —SCF₃, halo, —CF₃, —OCF₃, —COOH, and —COOC₁₋₆alkyl;
   b) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{p″};
   c) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{p″};
   d) naphthyl, optionally mono-, di-, or tri-substituted with R^{p″};
   e) a monocyclic aromatic hydrocarbon group having five ring atoms, having a carbon atom which is the point of attachment, having one carbon atom replaced by >O, >S, >NH, or >N(C₁₋₄alkyl), having up to two additional carbon atoms optionally replaced by N, optionally mono- or di-substituted with R^{p″} and optionally benzo or pyrido fused on the condition that two or fewer of said carbon ring atoms are replaced by a heteroatom, where the benzo or pyrido fused moiety is optionally mono-, di-, or tri-substituted with R^{p″};
   f) a monocyclic aromatic hydrocarbon group having six ring atoms, having a carbon atom which is the point of attachment, having one or two carbon atoms replaced by N, optionally mono- or di-substituted with R^{p″} and optionally benzo or pyrido fused, where the benzo or pyrido fused moiety is optionally mono- or di-substituted with R^{p″};
   g) adamantanyl or monocyclic C₅₋₇cycloalkyl, optionally having one or two carbon members optionally replaced with >O, >S, >NH, or >N(C₁₋₄alkyl), and optionally having one or two unsaturated bonds in the ring and optionally having one of the ring atoms substituted with —OH, ═O, or —CH₃;
   h) a —C₁₋₈alkyl, optionally mono-, di-, or tri-substituted with R^{p″} or a substituent selected from the group consisting of any one of a) to g);
   i) —C₂alkenyl or —C₂alkynyl, optionally substituted with a substituent selected from the group consisting of any one of a) to h); and
   j) —COOR⁷″ where R⁷″ is —C₁₋₈alkyl, aryl, heteroaryl, or C₄₋₈cycloalkyl;
Ar″ is selected from the group consisting of:
   A) phenyl, optionally mono-, di-, or tri-substituted with R^{r″} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
      R^{r″} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y″})R^{z″} (wherein R^{y″} and R^{z″} are independently selected from —H, —C₁₋₆alkyl, and —C₁₋₆alkenyl, or R^{y′} and R^{Z″} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y″})R^{z″}, —(N—R^{t″})COR^{t″}, —(N—R^{t″})SO₂C₁₋₆alkyl (wherein R^{t″} is —H or —C₁₋₆alkyl, or two R^{t″} in the same substituent may be taken together with the amide of attachment to form an otherwise aliphatic hydrocarbon ring, said ring having 4 to 6 members), —(C═O)C₁₋₆alkyl, —(S═(O)_{m″})—C₁₋₆alkyl (wherein m″ is selected from 0 or 2), —SO₂N(R^{y″})R^{z″}, —SCF₃, halo, —CF₃, —OCF₃, —COOH, and —COOC₁₋₆alkyl;
   B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r″};
   C) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r″};
   D) naphthyl, optionally mono-, di-, or tri-substituted with R^{r″};
   E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r″} and optionally benzo or pyrido fused on the condition that two or fewer of said carbon ring atoms are replaced by a heteroatom, where the benzo or pyrido fused moiety is optionally mono-, di-, or tri-substituted with R^{r″}; and
   F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r″} and optionally benzo or pyrido fused, where the benzo or pyrido fused moiety is optionally mono- or di-substituted with R^{r″};
   G) adamantanyl or monocyclic C₅₋₇cycloalkyl, optionally having one or two carbon members optionally replaced with >O, >S, >NH or >N(C₁₋₄alkyl) and optionally having one or two unsaturated bonds in the ring and optionally having one of the ring atoms substituted with —OH, ═O, or —CH₃;
   H) a —C₁₋₈alkyl wherein the carbon of attachment bears no hydrogen substituents, optionally mono-, di-, or tri-substituted by a R^{r″} or a substituent selected from the group consisting of any one of a) to g); and
   I) —C₂alkenyl or —C₂alkynyl, optionally substituted with a substituent selected from the group consisting of any one of a) to h);
Y is —H or —C₁₋₄alkyl; and
Z is —C₁₋₈alkyl or —OC₁₋₈alkyl.

### DETAILED DESCRIPTION OF THE INVENTION

Considering the above referenced U.S. Pat. No. 5,051,518, columns 20 and 21, Applicant's invention does not include compounds of the following formula, and/or racemic mixtures of such compounds and/or pharmaceutical compositions containing such compounds or racemic mixtures thereof:

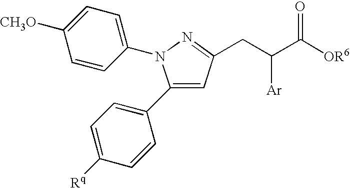

where R^{q}, Ar and R⁶ are selected concurrently from the groups consisting of:

| CP# | R^{q} | Ar | R⁶ |
|---|---|---|---|
| R1 | —Cl | phenyl- | —CH₂CH₃ |
| R2 | —Cl | 3,4-diMeO-phenyl- | —CH₂CH₃ |
| R3 | —Cl | 4-MeO-phenyl- | —CH₂CH₃ |
| R4 | —CH₃ | 2-naphthyl- | —CH₂CH₃ |
| R5 | —CH₃ | 1-naphthyl- | —CH₂CH₃ |
| R6 | —CH₃ | 2-MeO-phenyl- | —CH₂CH₃ |
| R7 | —CH₃ | 2-pyridyl- | —CH₂CH₃ |
| R8 | —CH₃ | 2-carboxymethyl-phenyl- | —CH₂CH₃ |
| R9 | —CH₃ | 3-pyridyl- | —CH₂CH₃ |
| R10 | —Cl | 4-MeO-phenyl- | —H |
| R11 | —Cl | 3,4-diMeO-phenyl- | —H |
| R12 | —CH₃ | 2-naphthyl- | —H |
| R13 | —CH₃ | 1-naphthyl- | —H |
| R14 | —CH₃ | 2-MeO-phenyl- | —H |
| R15 | —CH₃ | 2-carboxy-phenyl- | —H |
| R16 | —CH₃ | 4-biphenyl | —CH₂CH₃ |
| R17 | —CH₃ | 4-biphenyl | —H |

The instant invention does include the use of such compounds and/or racemic mixtures thereof and/or pharmaceutical compositions containing such compounds or racemic mixtures thereof to treat patients (humans and other mammals) with disorders related to the modulation of the CCK-1 receptor. The instant invention includes methods of making such compounds and/or racemic mixtures thereof.

Preferably R¹, optionally substituted with R^{p} as described above, is selected from the group consisting of hydrogen,
a) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
b) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
c) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
d) naphthyl,
e) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl,
f) pyridinyl, pyridinyl-N-oxide, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, 1-oxy-pyridin-2, 3, or 4-yl,
g) cyclopentyl, cyclohexyl, cycloheptyl, piperidin-2, 3 or 4-yl, 2-pyrrolin-2, 3, 4 or 5-yl, 3-pyrrolin-2 or 3-yl, 2-pyrazolin-3, 4 or 5-yl, morpholin-2, 3, 5 or 6-yl, thiomorpholin-2, 3, 5 or 6-yl, piperazin-2, 3, 5 or 6-yl, pyrrolidin-2 or 3-yl, homopiperidinyl, adamantanyl,
h) methyl, ethyl, n-propyl, i-propyl, n-butyl, i-butyl, t-butyl, n-pentyl, pent-2-yl, hexyl, hex-2-yl, and
   i) —C₁₋₂alkyl mono-substituted with any one of the preferred substituents of a) to g).

Most preferably R¹, optionally substituted with R^{p} as described above, is selected from the group consisting of —H, methyl, phenyl, benzyl, cyclohexyl, cyclohexylmethyl, pyridinyl, pyridinylmethyl and pyridinyl-N-oxide. Specific R¹ are selected from the group consisting of phenyl, 2-methoxy-phenyl, 3-methoxy-phenyl, 4-methoxy-phenyl, 2,3-dimethoxy-phenyl, 3,4-dimethoxy-phenyl, 2-chloro-phenyl, 3-chloro-phenyl, 4-chloro-phenyl, 2,4-dichloro-phenyl, 3,4-dichlorophenyl, 2,4-dichlorophenyl, 2,5-dichlorophenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 4-trifluoromethyl-phenyl, 3-trifluoromethoxy-phenyl, 4-trifluoromethoxy-phenyl, 4-t-butyl-phenyl, benzyl, cyclohexyl, pyridin-2-yl, pyridin-3-yl, pyridin-4-yl, 4-trifluoromethyl-2-pyridyl, 2-pyridyl-N-oxide, 4-methanesulfonyl-phenyl, 4-phenoxy-phenyl, 4-isopropyl-phenyl, 4-ethoxy-phenyl, 4-hydroxy-phenyl, 4-pyridinyl-methyl, benzo[1,3]diox-5-yl, 2,3-dihydro benzo[1,4]dioxin-6-yl, and cyclohexylmethyl.

Preferably, R^{p} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SOCH₃, —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

Most preferably, R^{p} is selected from the group consisting of methyl, methoxy, ethoxy, chloro, fluoro, trifluoromethyl, trifluoromethoxy, t-butyl, methanesulfonyl, phenoxy, isopropyl, and hydroxy.

Preferably, R², optionally substituted with R^{q} as described above, is selected from the group consisting of:
i) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
ii) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
iii) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
iv) naphthyl,
v) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, and
vi) pyridinyl, pyridinyl-N-oxide, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl.

Most preferably, R², optionally substituted with R^{q} as described above, is selected from the group consisting of phenyl, naphthalenyl, pyridinyl, thiophenyl, benzothiophenyl, furanyl, benzofuranyl, indolyl, indolinyl, isoquinolinyl, and quinolinyl. Specific R² are selected from the group consisting of 4-methyl-phenyl, 2-chloro-phenyl, 3-chloro-phenyl, 4-chloro-phenyl, 3,4-dichloro-phenyl, benzo[1,3]dioxol-5-yl, 2,3-dihydro benzo[1,4]dioxin-6-yl, 4-methoxy-phenyl, phenyl, 4-phenoxy-phenyl, naphthalen-2-yl, pyridin-3-yl, 2 -chloro-pyridin-3-yl, pyridin-4-ylmethyl, 4-benzyloxy-phenyl, 4-dimethylamino-phenyl, 4-bromo-3-methyl-phenyl, 3-methoxy-4-methyl-phenyl, 3-cyclopentyloxy-4-methoxy-phenyl, 4-bromo-2-chloro-phenyl, 4-bromo-phenyl, 3-dimethylamino-phenyl, 4-morpholin-1-yl-phenyl, 4-pyrrolidin-1-yl-phenyl, 4-(N-propylamino)-phenyl, 4-(N-isobutylamino)-phenyl, 4-diethylamino-phenyl, 4-(N-allylamino)-phenyl, 4-(N-isopropylamino)-phenyl, 4-(N-methyl-N-propylamino)-phenyl, 4-(N-methyl-N-isopropylamino)-phenyl, 4-(N-methyl-N-ethylamino)-phenyl, 4-amino-phenyl, 4-(N-methyl-N-propylamino)-2-chloro-phenyl, 4-(N-ethyl-N-methylamino)-2-chloro-phenyl, 4-(pyrrolidin-1-yl)-2-chloro-phenyl, 4-azetidinyl-phenyl, 4-(pyrrolidin-2-one-1-yl)-phenyl, 4-bromo-3-methyl-phenyl, 4-chloro-3-methyl-phenyl, 1-methyl-5-indolinyl, 5-indolinyl, 5-isoquinolinyl, 6-quinolinyl, benzo[1,3]diox-5-yl, and 7-methoxy-benzofuran-2-yl.

Preferably, R^{q} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SOCH₃, —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

Most preferably, R^{q} is selected from the group consisting of methyl, bromo, chloro, methoxy, cyclopentyloxy, phenoxy, benzyloxy, pyrrolidinyl, N-methyl-N-ethylamino and dimethylamino. Preferably, there are 0, 1, or 2 R^{q} substituents.

Preferably, R³ is selected from the group consisting of —H, —F, —Cl, —Br, and —CH₃.

Most preferably, R³ is —H.

Preferably, n is 0 or 1.

Preferably, R⁴ is selected from the group consisting of —H, —F, and —CH₃.

Most preferably, R⁴ is —H.

In one preferred embodiment of the invention, the Ar attached carbon is saturated and has the configuration

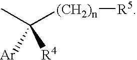

In another preferred embodiment of the present invention, the Ar attached carbon is unsaturated and has the configuration

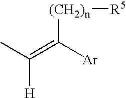

Preferably Ar, optionally substituted with R^{r} as described above, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, and
F) pyridinyl, pyridinyl-N-oxide, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl.

Most preferably, Ar, optionally substituted with R^{r} as described above, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl and pyridinyl. Specific Ar are selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 3-iodo-phenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, 3-trifluoromethylsulfanyl-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, and 2-methyl-3-trifluoromethyl-phenyl. Preferably, there are 0, 1, or 2 R^{r} substituents.

Preferably, R^{r} is selected from the group consisting of —OH, —CH₃, —CH₂CH₃, -propyl, -t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SOCH₃, —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

Most preferably, R^{r} is selected from the group consisting of methyl, methoxy, ethoxy, isopropoxy, dimethylamino, fluoro, chloro, iodo, trifluoromethyl, trifluoromethoxy, nitro, phenyl, and trifluoromethylsulfanyl.

Preferably, R⁵ is selected from the group consisting of:
I) —COOH, —COOCH₃, —COOCH₂CH₃,
II)—CONH(CH₃), —CONH(CH₂CH₃), —CONH(CH₂CH₂CH₃), —CONH(CH(CH₃)₂), —CONH(CH₂CH₂CH₂CH₃), —CONH(CH(CH₃)CH₂CH₃), —CONH(C(CH₃)₃), —CON H(cyclohexyl), —CON H(2-hydroxy-cyclohexyl), —CON(CH₃)₂, —CONCH₃(CH₂CH₃), —CONCH₃(CH₂CH₂CH₃), —CONCH₃(CH(CH₃)₂), —CONCH₃(CH₂CH₂CH₂CH₃), —CONCH₃(CH(CH₃)CH₂CH₃), —CONCH₃(C(CH₃)₃), —CON(CH₂CH₃)₂, —CO-piperidin-1-yl, —CO-morpholin-4-yl, —CO-piperazin-1-yl, —CO-imidazolidin-1-yl, —CO-pyrrolidin-1-yl, —CO-2-pyrrolin-1-yl, —CO-3-pyrrolin-1-yl, —CO-2-imidazolin-1-yl, —CO-piperidin-1-yl, and
III) -tetrazolyl, 1H-[1,2,4)triazol-5-ylsulfinyl, 1H-[1,2,4]triazol-5-ylsulfonyl, 1H-[1,2,4]triazol-5-ylsulfanyl,

Most preferably, R⁵ is selected from the group consisting of —COOH and tetrazol-5-yl.

It is understood that when any substituent generic symbol is used herein in a plurality of substitution positions, the assignment of specific substituents in each of such substitution positions is made independently of any other assignment in any other of such substitution positions. Analogously, when any index is used herein in a plurality of positions, the assignment of specific index values in each of such positions is made independently of any other assignment in any other of such positions.

Preferred compounds of formula (I′) are the preferred, more preferred, and most preferred compounds of formula (I) with the substituents described as in their non-prime designated counterparts in the foregoing substituent assignments, with the following differences:
Most preferably, R^{p′} is selected from the group consisting of methyl, methoxy, ethoxy, chloro, fluoro, trifluoromethyl, trifluoromethoxy, t-butyl, methanesulfonyl, phenoxy, isopropyl, and hydroxy;

Preferably, R^{q′} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SO(CH₃), —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl;
Preferably, R^{3′} is selected from the group consisting of —H, —F, —Cl, —Br, and —CH₃;
Most preferably, R³ is —H;
Preferably, Ar′, optionally substituted with R^{r′} as described above, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl;
   Most preferably, Ar′, optionally substituted with R^{r′} as described above, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl. Specific Ar′ are selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃. Preferably, there are 0, 1, or 2 R^{r′} substituents;
   Preferably, R^{r′} is selected from the group consisting of —OH, —CH₃, —CH₂CH₃, -propyl, -t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —Br, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl;
   Most preferably, R^{r′} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl;
   Preferably, R^{5′} is —COOR^{6′}, where —COOR^{6′} is —COOH or a hydrolysable group;
   Most preferably, R^{5′} is selected from the group consisting of —COOCH₃, —COOCH₂CH₃, and —COOCH(CH₃)₂.

Preferred compounds of formula (II) are those in which preferred substituents are described as follows:
Preferably, G, optionally substituted with R^{p″} as described above, is selected from the group consisting of
a) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
b) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
c) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
d) naphthyl,
e) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl,
f) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, 1-oxy-pyridin-2, 3, or 4-yl,
g) cyclopentyl, cyclohexyl, cycloheptyl, piperidin-2, 3 or 4-yl, 2-pyrrolin-2, 3, 4 or 5-yl, 3-pyrrolin-2 or 3-yl, 2-pyrazolin-3, 4 or 5-yl, morpholin-2, 3, 5 or 6-yl, thiomorpholin-2, 3, 5 or 6-yl, piperazin-2, 3, 5 or 6-yl, pyrrolidin-2 or 3-yl, homopiperidinyl, adamantanyl,
h) methyl, isopropyl, t-butyl, t-hexyl, —CF₃, —CF₂C₁₋₄alkyl,
i) ethenyl, ethynyl, cinnamyl, and
j) —COOmethyl, —COOphenyl, —COObenzyl, —COOcyclohexyl, —COOi-pentyl;
   Most preferably, G, optionally substituted with R^{p″} as described above, is selected from the group consisting of phenyl, cyclohexyl, pyridinyl, and pyrazolyl. Specific G are selected from the group consisting of phenyl, 2 -methoxy-phenyl, 3-methoxy-phenyl, 4-methoxy-phenyl, 2,3-dimethoxy-phenyl, 3,4-dimethyoxy-phenyl, 2-chloro-phenyl, 3-chloro-phenyl, 4-chloro-phenyl, 2,4-dichloro-phenyl, 3,4-dichlorophenyl, 2,4-dichlorophenyl, 2,5-dichlorophenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 4-trifluoromethyl-phenyl, 3-trifluoromethoxy-phenyl, 4-trifluoromethoxy-phenyl, 4-t-butyl-phenyl, 4-trifluoromethyl-2-pyridyl, 4-methanesulfonyl-phenyl, 4-phenoxy-phenyl, 4-isopropyl-phenyl, 4-ethoxy-phenyl, 4-hydroxy-phenyl, benzo[1,3]diox-5-yl, 2,3-dihydro benzo[1,4]dioxin-6-yl, 3-pyrazolyl, pyridin-2-yl, pyridin-3-yl, pyridin-4-yl, cyclohexyl, morpholinyl, t-butyl, —CF₃, methyl, isopropyl, ethenyl, cinnamyl, —COOmethyl, —COOphenyl, and —COOcyclohexyl;
   Preferably, R^{p″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl;
   Most preferably, R^{p″} is selected from the group consisting of methyl, methoxy, ethoxy, chloro, fluoro, trifluoromethyl, trifluoromethoxy, t-butyl, methanesulfonyl, phenoxy, isopropyl, and hydroxy;
   Preferably, Ar″, optionally substituted with R^{r″} as described above, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl;
   Most preferably, Ar″, optionally substituted with R^{r″} as described above, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl. Specific Ar″ are selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃. Preferably, there are 0, 1, or 2 R^{r″} substituents;
   Preferably, R^{r″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl;
   Most preferably, R^{r″} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl;
   Preferably, Y is —H, methyl, ethyl, isopropyl, or propyl.

Preferred compounds of formula (III) are those in which preferred substituents are described as follows:
Preferably, Ar″, optionally substituted with R^{r″} as described above, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl;
   Most preferably, Ar″, optionally substituted with R^{r″} as described above, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl. Specific Ar″ are selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃. Preferably, there are 0, 1, or 2 R^{r″} substituents;
   Preferably, R^{r″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl]thiomorpholin-4-yl, piperazin-1-yl, pyrrolidin-1-yl, and homopiperidin-1-yl;
   Most preferably, R^{r″} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl;
   Preferably, Z is selected from the group consisting of methyl, ethyl, propyl, isopropyl, butyl, t-butyl, isobutyl, hexyl, methoxy, ethoxy, propoxy, isopropoxy, butoxy, and hexyloxy;
   More preferably, Z is methyl or methoxy.

The “pharmaceutically acceptable salts and esters thereof” refer to those salt and ester forms of the compounds of the present invention which would be apparent to the pharmaceutical chemist, i.e., those which are non-toxic and which would favorably affect the pharmacokinetic properties of said compounds of the present invention. Those compounds having favorable pharmacokinetic properties would be apparent to the pharmaceutical chemist, i.e., those which are non-toxic and which possess such pharmacokinetic properties to provide sufficient palatability, absorption, distribution, metabolism and excretion. Other factors, more practical in nature, which are also important in the selection, are cost of raw materials, ease of crystallization, yield, stability, hygroscopicity and flowability of the resulting bulk drug. In addition, acceptable salts of carboxylates include sodium, potassium, calcium and magnesium. Examples of suitable cationic salts include hydrobromic, hydroiodic, hydrochloric, perchloric, sulfuric, maleic, fumaric, malic, tartatic, citric, benzoic, mandelic, methanesulfonic, hydroethanesulfonic, benzenesulfonic, oxalic, palmoic, 2-naphthalenesulfonic, p-toluenesulfonic, cyclohexanesulfamic and saccharic. Examples of suitable esters include such esters where one or more carboxyl substituents is replaced with p-methoxybenzyloxycarbonyl, 2,4,6-trimethylbenzyloxycarbonyl, 9-anthryloxycarbonyl, CH₃SCH₂COO—, tetrahydrofur-2-yloxycarbonyl, tetrahydropyran-2-yloxycarbonyl, fur-2-uloxycarbonyl, benzoylmethoxycarbonyl, p-nitrobenzyloxycarbonyl, 4-pyridylmethoxycarbonyl, 2,2,2-trichloroethoxycarbonyl, 2,2,2-tribromoethoxycarbonyl, t-butyloxycarbonyl, t-amyloxycarbonyl, diphenylmethoxycarbonyl, triphenylmethoxycarbonyl, adamantyloxycarbonyl, 2-benzyloxyphenyloxycarbonyl, 4-methylthiophenyloxycarbonyl, or tetrahydropyran-2-yloxycarbonyl.

Preferred compounds of Table 1a, which were made in some embodiments of this invention according to the synthetic methods outlined in Scheme A and as described in Method 2, are given by the formula:

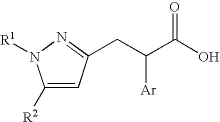

where R², R¹ and Ar are selected concurrently from the groups consisting of:

TABLE 1a

| EX | R² | R¹ | Ar | [M + H]⁺ |
|---|---|---|---|---|
| 1 | (3,4-Dichloro- | (4-Methoxy- | (3-Methyl-phenyl)- | 481.1 |
| | phenyl)- | phenyl)- | [(S) enantiomer, Na⁺ | |
| | | | salt] | |
| 2 | (3,4-Dichloro- | (4-Methoxy- | (3-Methyl-phenyl)- | 481.1 |
| | phenyl)- | phenyl)- | | |
| 3 | (3,4-Dichloro- | (4-Methoxy- | (3-Methyl-phenyl)- | 481.1 |
| | phenyl)- | phenyl)- | [(R) enantiomer] | |
| 4 | (3,4-Dichloro- | (4-Methoxy- | (3-Methyl-phenyl)- | 481.1 |
| | phenyl)- | phenyl)- | [(S) enantiomer, TFA | |
| | | | salt] | |
| 5 | (4-Methyl-phenyl)- | (4-Methoxy- | (4-Methoxy-phenyl)- | 443.2 |
| | | phenyl)- | | |
| 6 | (4-Methyl-phenyl)- | (4-Methoxy- | (3-Methoxy-phenyl)- | 443.2 |
| | | phenyl)- | | |
| 7 | (4-Methyl-phenyl)- | (4-Methoxy- | (3-Chloro-phenyl)- | 447.2 |
| | | phenyl)- | | |
| 8 | (4-Methyl-phenyl)- | (4-Methoxy- | (4-Methyl-phenyl)- | 427.2 |
| | | phenyl)- | | |
| 9 | (4-Methyl-phenyl)- | (4-Methoxy- | (4-Chloro-phenyl)- | 447.2 |
| | | phenyl)- | | |
| 10 | (2-Chloro-phenyl)- | (4-Methoxy- | Naphthalen-1-yl- | 483.1 |
| | | phenyl)- | | |
| 11 | (2-Chloro-phenyl)- | (4-Methoxy- | (3-Chloro-phenyl)- | 467.1 |
| | | phenyl)- | | |
| 12 | (3,4-Dichloro- | (4-Methoxy- | Phenyl- | 467.1 |
| | phenyl)- | phenyl)- | | |
| 13 | Benzo[1,3]dioxol- | (4-Methoxy- | (3-Methoxy-phenyl)- | 473.2 |
| | 5-yl- | phenyl)- | | |
| 15 | Phenyl- | (4-Methoxy- | Naphthalen-2-yl- | 449.2 |
| | | phenyl)- | | |
| 16 | (4-Phenoxy- | (4-Methoxy- | (3-Nitro-phenyl)- | 536.2 |
| | phenyl)- | phenyl)- | | |
| 17 | Benzo[1,3]dioxol- | (4-Methoxy- | Benzo[1,3]dioxol-5-yl- | 487.2 |
| | 5-yl- | phenyl)- | | |
| 18 | (3,4-Dichloro- | (4-Methoxy- | (2,3-Difluoro-phenyl)- | 503.1 |
| | phenyl)- | phenyl)- | | |
| 19 | (3,4-Dichloro- | (4-Methoxy- | (2-Trifluoromethyl- | 535.1 |
| | phenyl)- | phenyl)- | phenyl)- | |
| 20 | (3,4-Dichloro- | (4-Methoxy- | (3-Ethoxy-phenyl)- | 511.1 |
| | phenyl)- | phenyl)- | | |
| 21 | (4-Methyl-phenyl)- | (3,4-Dichloro- | (2-Fluoro-3- | 537.1 |
| | | phenyl)- | trifluoromethyl- | |
| | | | phenyl)- | |
| 22 | (4-Phenoxy- | (4-Methoxy- | (4-Trifluoromethoxy- | 575.2 |
| | phenyl)- | phenyl)- | phenyl)- | |
| 23 | Benzo[1,3]dioxol- | (4-Methoxy- | (3-Trifluoromethoxy- | 527.1 |
| | 5-yl- | phenyl)- | phenyl)- | |
| 24 | (4-Methyl-phenyl)- | (3,4-Dichloro- | (3-Iodo-phenyl)- | 577.0 |
| | | phenyl)- | | |
| 25 | (4-Methyl-phenyl)- | (3,4-Dichloro- | (3,5-Dimethyl-phenyl)- | 479.1 |
| | | phenyl)- | | |
| 26 | (4-Methyl-phenyl)- | (3,4-Dichloro- | (3-Trifluoromethyl- | 551.0 |
| | | phenyl)- | sulfanyl-phenyl)- | |
| 27 | Benzo[1,3]dioxol- | (4-Methoxy- | Naphthalen-1-yl- | 493.2 |
| | 5-yl- | phenyl)- | | |
| 28 | Benzo[1,3]dioxol- | (4-Methoxy- | Naphthalen-1-yl- | 493.2 |
| | 5-yl- | phenyl)- | [(R) enantiomer] | |
| 29 | Benzo[1,3]dioxol- | (4-Methoxy- | Naphthalen-1-yl- | 493.2 |
| | 5-yl- | phenyl)- | [(S) enantiomer] | |
| 30 | (4-Methoxy- | (4-Methoxy- | (3-Methoxy-phenyl)- | 459.2 |
| | phenyl)- | phenyl)- | | |
| 31 | (4-Methoxy- | (4-Methoxy- | (3-Methoxy-phenyl)- | 459.2 |
| | phenyl)- | phenyl)- | [(R) enantiomer] | |
| 32 | (4-Methoxy- | (4-Methoxy- | (3-Methoxy-phenyl)- | 459.2 |
| | phenyl)- | phenyl)- | [(S) enantiomer] | |
| 33 | (4-Chloro-phenyl)- | (4-Methoxy- | Biphenyl-4-yl- | 509.2 |
| | | phenyl)- | | |
| 34 | (4-Chloro-phenyl)- | (4-Methoxy- | (4-Methyl-phenyl)- | 447.2 |
| | | phenyl)- | | |
| 35 | (4-Chloro-phenyl)- | (4-Methoxy- | (3-Methyl-phenyl)- | 447.1 |
| | | phenyl)- | | |
| 36 | (4-Chloro-phenyl)- | (4-Methoxy- | (3-Methoxy-phenyl)- | 463.1 |
| | | phenyl)- | | |
| 37 | (4-Chloro-phenyl)- | (4-Methoxy- | (3-Chloro-phenyl)- | 467.2 |
| | | phenyl)- | | |
| 38 | (4-Methyl-phenyl)- | (4-Chloro-phenyl)- | Naphthalen-1-yl- | 467.1 |
| 39 | (4-Methyl-phenyl)- | (3-Chloro-phenyl)- | (3-Chloro-phenyl)- | 451.0 |
| 40 | (4-Methyl-phenyl)- | (4-Methyl-phenyl)- | (3-Methyl-phenyl)- | 411.1 |
| 41 | (4-Methyl-phenyl)- | (4-Trifluoromethyl- | Phenyl- | 451.0 |
| | | phenyl)- | | |
| 42 | (4-Methyl-phenyl)- | (3,4-Dichloro- | (3-Methoxy-phenyl)- | 481.0 |
| | | phenyl)- | | |
| 43 | (4-Methyl-phenyl)- | Benzyl- | (2-Chloro-phenyl)- | 431.0 |
| 44 | (4-Methyl-phenyl)- | Benzyl- | (3-Trifluoromethyl- | 465.0 |
| | | | phenyl)- | |
| 45 | (4-Methyl-phenyl)- | Benzyl- | Naphthalen-2-yl- | 447.1 |
| 46 | (4-Methyl-phenyl)- | (3,4-Dichloro- | (2,3-Dichloro-phenyl)- | 519.0 |
| | | phenyl)- | | |
| 142 | (4-Methyl-phenyl)- | (4-Methoxy- | (2-Methyl-phenyl)- | 427.5 |
| | | phenyl)- | | |
| 143 | (4-Methyl-phenyl)- | (4-Methoxy- | (2-Fluoro-phenyl)- | 431.2 |
| | | phenyl)- | | |
| 144 | (4-Methyl-phenyl)- | (4-Methoxy- | (2,6-Dichloro-phenyl)- | 481.1 |
| | | phenyl)- | | |
| 145 | (4-Methyl-phenyl)- | (4-Methoxy- | (3-Methoxy-phenyl)- | 443.2 |
| | | phenyl)- | | |
| 146 | (4-Methyl-phenyl)- | (4-Methoxy- | (2,3-Dimethoxy- | 473.2 |
| | | phenyl)- | phenyl)- | |
| 147 | (4-Methyl-phenyl)- | (4-Methoxy- | (2-Chloro-phenyl)- | 447.1 |
| | | phenyl)- | | |
| 148 | (4-Methyl-phenyl)- | (4-Methoxy- | (3-Methyl-phenyl)- | 427.2 |
| | | phenyl)- | | |
| 149 | (4-Methyl-phenyl)- | (4-Methoxy- | (3,4-Dichloro-phenyl)- | 481.1 |
| | | phenyl)- | | |
| 150 | (4-Methyl-phenyl)- | (4-Methoxy- | Phenyl- | 413.2 |
| | | phenyl)- | | |
| 151 | (4-Methyl-phenyl)- | (4-Methoxy- | Naphthalen-1-yl- | 463.2 |
| | | phenyl)- | [(R) enantiomer] | |
| 152 | (4-Methyl-phenyl)- | (4-Methoxy- | Naphthalen-1-yl- | 463.2 |
| | | phenyl)- | [(S) enantiomer] | |
| 153 | (4-Methyl-phenyl)- | (4-Methoxy- | Benzo[b]thiophen-4-yl- | 469.1 |
| | | phenyl)- | | |
| 154 | (4-Methyl-phenyl)- | (4-Chloro-phenyl)- | (3-Chloro-phenyl)- | 451.0 |
| 155 | (4-Methyl-phenyl)- | (4-Chloro-phenyl)- | (3-Methyl-phenyl)- | 431.0 |
| 156 | (4-Methyl-phenyl)- | (4-Chloro-phenyl)- | Phenyl- | 417.1 |
| 157 | (4-Methyl-phenyl)- | (4-Chloro-phenyl)- | (3-Methoxy-phenyl)- | 447.1 |
| 158 | (4-Methyl-phenyl)- | (4-Chloro-phenyl)- | (2-Chloro-phenyl)- | 451.0 |
| 159 | (4-Methyl-phenyl)- | (4-Chloro-phenyl)- | (3-Trifluoromethyl- | 485.0 |
| | | | phenyl)- | |
| 160 | (4-Methyl-phenyl)- | (4-Chloro-phenyl)- | Naphthalen-2-yl- | 467.1 |
| 161 | (4-Methyl-phenyl)- | (3-Chloro-phenyl)- | Naphthalen-1-yl- | 467.1 |
| 162 | (4-Methyl-phenyl)- | (3-Chloro-phenyl)- | Phenyl- | 417.1 |
| 163 | (4-Methyl-phenyl)- | (3-Chloro-phenyl)- | (3-Methoxy-phenyl)- | 447.1 |
| 164 | (4-Methyl-phenyl)- | (3-Chloro-phenyl)- | (2-Chloro-phenyl)- | 451.0 |
| 165 | (4-Methyl-phenyl)- | (3-Chloro-phenyl)- | (3-Trifluoromethyl- | 485.0 |
| | | | phenyl)- | |
| 166 | (4-Methyl-phenyl)- | (3-Chloro-phenyl)- | Naphthalen-2-yl- | 467.1 |
| 167 | (4-Methyl-phenyl)- | (4-Methyl-phenyl)- | Naphthalen-1-yl- | 447.1 |
| 168 | (4-Methyl-phenyl)- | (4-Methyl-phenyl)- | (3-Chloro-phenyl)- | 431.0 |
| 169 | (4-Methyl-phenyl)- | (4-Methyl-phenyl)- | Phenyl- | 397.1 |
| 170 | (4-Methyl-phenyl)- | (4-Methyl-phenyl)- | (3-Methoxy-phenyl)- | 427.1 |
| 171 | (4-Methyl-phenyl)- | (4-Methyl-phenyl)- | (2-Chloro-phenyl)- | 431.0 |
| 172 | (4-Methyl-phenyl)- | (4-Methyl-phenyl)- | (3-Trifluoromethyl- | 466.1 |
| | | | phenyl)- | |
| 173 | (4-Methyl-phenyl)- | (4-Methyl-phenyl)- | Naphthalen-2-yl- | 447.1 |
| 174 | (4-Methyl-phenyl)- | (4-Trifluoromethyl- | Naphthalen-1-yl- | 501.1 |
| | | phenyl)- | | |
| 175 | (4-Methyl-phenyl)- | (4-Trifluoromethyl- | (3-Chloro-phenyl)- | 485.0 |
| | | phenyl)- | | |
| 176 | (4-Methyl-phenyl)- | (4-Trifluoromethyl- | (3-Methyl-phenyl)- | 465.1 |
| | | phenyl)- | | |
| 177 | (4-Methyl-phenyl)- | (4-Trifluoromethyl- | (3-Methoxy-phenyl)- | 481.1 |
| | | phenyl)- | | |
| 178 | (4-Methyl-phenyl)- | (4-Trifluoromethyl- | (2-Chloro-phenyl)- | 485.0 |
| | | phenyl)- | | |
| 179 | (4-Methyl-phenyl)- | (4-Trifluoromethyl- | (3-Trifluoromethyl- | 519.1 |
| | | phenyl)- | phenyl)- | |
| 180 | (4-Methyl-phenyl)- | (4-Trifluoromethyl- | Naphthalen-2-yl- | 501.1 |
| | | phenyl)- | | |
| 181 | (4-Methyl-phenyl)- | (3,4-Dichloro- | Naphthalen-1-yl- | 501.0 |
| | | phenyl)- | | |
| 182 | (4-Methyl-phenyl)- | (3,4-Dichloro- | (3-Chloro-phenyl)- | 485.0 |
| | | phenyl)- | | |
| 183 | (4-Methyl-phenyl)- | (3,4-Dichloro- | (3-Methyl-phenyl)- | 465.1 |
| | | phenyl)- | | |
| 184 | (4-Methyl-phenyl)- | (3,4-Dichloro- | Phenyl- | 451.0 |
| | | phenyl)- | | |
| 185 | (4-Methyl-phenyl)- | (3,4-Dichloro- | (2-Chloro-phenyl)- | 485.0 |
| | | phenyl)- | | |
| 186 | (4-Methyl-phenyl)- | (3,4-Dichloro- | (3-Trifluoromethyl- | 519.0 |
| | | phenyl)- | phenyl)- | |
| 187 | (4-Methyl-phenyl)- | (3,4-Dichloro- | Naphthalen-2-yl- | 501.0 |
| | | phenyl)- | | |
| 188 | (4-Methyl-phenyl)- | (3,4-Dichloro- | (3-Nitro-phenyl)- | 496.1 |
| | | phenyl)- | | |
| 189 | (4-Methyl-phenyl)- | (3,4-Dichloro- | Benzo[1,3]dioxol-5-yl- | 495.1 |
| | | phenyl)- | | |
| 190 | (4-Methyl-phenyl)- | (3,4-Dichloro- | Benzo[b]thiophen-4-yl- | 507.0 |
| | | phenyl)- | | |
| 191 | (4-Methyl-phenyl)- | (3,4-Dichloro- | (2,3-Difluoro-phenyl)- | 487.1 |
| | | phenyl)- | | |
| 192 | (4-Methyl-phenyl)- | (3,4-Dichloro- | (2-Trifluoromethyl- | 519.1 |
| | | phenyl)- | phenyl)- | |
| 193 | (4-Methyl-phenyl)- | (3,4-Dichloro- | (4-Trifluoromethoxy- | 535.0 |
| | | phenyl)- | phenyl)- | |
| 194 | (4-Methyl-phenyl)- | (3,4-Dichloro- | (3-Trifluoromethoxy- | 535.1 |
| | | phenyl)- | phenyl)- | |
| 195 | (4-Methyl-phenyl)- | Benzyl- | Naphthalen-1-yl- | 447.1 |
| 196 | (4-Methyl-phenyl)- | Benzyl- | (3-Chloro-phenyl)- | 431.0 |
| 197 | (4-Methyl-phenyl)- | Benzyl- | (3-Methyl-phenyl)- | 411.1 |
| 198 | (4-Methyl-phenyl)- | Benzyl- | Phenyl- | 398.1 |
| 199 | (4-Methyl-phenyl)- | Benzyl- | (3-Methoxy-phenyl)- | 427.1 |
| 200 | (4-Chloro-phenyl)- | (4-Methoxy- | (2-Chloro-4-fluoro- | 485.1 |
| | | phenyl)- | phenyl)- | |
| 201 | (4-Chloro-phenyl)- | (4-Methoxy- | (2-Chloro-phenyl)- | 467.1 |
| | | phenyl)- | | |
| 202 | (4-Chloro-phenyl)- | (4-Methoxy- | (2,6-Dichloro-phenyl)- | 501.1 |
| | | phenyl)- | | |
| 203 | (4-Chloro-phenyl)- | (4-Methoxy- | (2-Methoxy-phenyl)- | 463.1 |
| | | phenyl)- | | |
| 204 | (4-Chloro-phenyl)- | (4-Methoxy- | Phenyl- | 433.1 |
| | | phenyl)- | | |
| 205 | (4-Chloro-phenyl)- | (4-Methoxy- | (2-Methyl-phenyl)- | 447.1 |
| | | phenyl)- | | |
| 206 | (4-Chloro-phenyl)- | (4-Methoxy- | (2-Fluoro-phenyl)- | 451.1 |
| | | phenyl)- | | |
| 207 | (4-Chloro-phenyl)- | (4-Methoxy- | Naphthalen-1-yl- | 483.1 |
| | | phenyl)- | | |
| 208 | (4-Chloro-phenyl)- | (4-Methoxy- | Pyridin-3-yl- | 434.1 |
| | | phenyl)- | | |
| 209 | (3,4-Dichloro- | (4-Methoxy- | (3-Chloro-phenyl)- | 501.0 |
| | phenyl)- | phenyl)- | | |
| 210 | (3,4-Dichloro- | (4-Methoxy- | Naphthalen-1-yl- | 517.1 |
| | phenyl)- | phenyl)- | | |
| 211 | (3,4-Dichloro- | (4-Methoxy- | (3-Methoxy-phenyl)- | 497.1 |
| | phenyl)- | phenyl)- | | |
| 212 | (3,4-Dichloro- | (4-Methoxy- | Naphthalen-2-yl- | 517.1 |
| | phenyl)- | phenyl)- | | |
| 213 | (3,4-Dichloro- | (4-Methoxy- | (3-Nitro-phenyl)- | 512.1 |
| | phenyl)- | phenyl)- | | |
| 214 | (3,4-Dichloro- | (4-Methoxy- | Benzo[1,3]dioxol-5-yl- | 511.1 |
| | phenyl)- | phenyl)- | | |
| 215 | (3,4-Dichloro- | (4-Methoxy- | (2-Fluoro-3- | 553.1 |
| | phenyl)- | phenyl)- | trifluoromethyl- | |
| | | | phenyl)- | |
| 216 | (3,4-Dichloro- | (4-Methoxy- | (4-Trifluoromethoxy- | 551.1 |
| | phenyl)- | phenyl)- | phenyl)- | |
| 217 | (3,4-Dichloro- | (4-Methoxy- | (3-Iodo-phenyl)- | 593.0 |
| | phenyl)- | phenyl)- | | |
| 218 | (3,4-Dichloro- | (4-Methoxy- | (3,5-Dimethyl-phenyl)- | 495.1 |
| | phenyl)- | phenyl)- | | |
| 219 | (3,4-Dichloro- | (4-Methoxy- | (2,3-Dichloro-phenyl)- | 535.0 |
| | phenyl)- | phenyl)- | | |
| 220 | Benzo[1,3]dioxol- | (4-Methoxy- | (3-Methyl-phenyl)- | 457.1 |
| | 5-yl- | phenyl)- | | |
| 221 | Benzo[1,3]dioxol- | (4-Methoxy- | (3-Chloro-phenyl)- | 477.1 |
| | 5-yl- | phenyl)- | | |
| 222 | Benzo[1,3]dioxol- | (4-Methoxy- | Phenyl- | 443.1 |
| | 5-yl- | phenyl)- | | |
| 223 | Benzo[1,3]dioxol- | (4-Methoxy- | Naphthalen-2-yl- | 493.1 |
| | 5-yl- | phenyl)- | | |
| 224 | Benzo[1,3]dioxol- | (4-Methoxy- | (3-Nitro-phenyl)- | 488.1 |
| | 5-yl- | phenyl)- | | |
| 225 | Benzo[1,3]dioxol- | (4-Methoxy- | (2,3-Difluoro-phenyl)- | 479.1 |
| | 5-yl- | phenyl)- | | |
| 226 | Benzo[1,3]dioxol- | (4-Methoxy- | (2-Trifluoromethyl- | 511.1 |
| | 5-yl- | phenyl)- | phenyl)- | |
| 227 | Benzo[1,3]dioxol- | (4-Methoxy- | (3-Ethoxy-phenyl)- | 487.2 |
| | 5-yl- | phenyl)- | | |
| 228 | Benzo[1,3]dioxol- | (4-Methoxy- | (2-Fluoro-3- | 529.1 |
| | 5-yl- | phenyl)- | trifluoromethyl- | |
| | | | phenyl)- | |
| 229 | Benzo[1,3]dioxol- | (4-Methoxy- | (4-Trifluoromethoxy- | 527.1 |
| | 5-yl- | phenyl)- | phenyl)- | |
| 230 | Benzo[1,3]dioxol- | (4-Methoxy- | (3-Trifluoromethyl- | 543.1 |
| | 5-yl- | phenyl)- | sulfanyl-phenyl)- | |
| 231 | Benzo[1,3]dioxol- | (4-Methoxy- | (3-Iodo-phenyl)- | 569.1 |
| | 5-yl- | phenyl)- | | |
| 232 | Benzo[1,3]dioxol- | (4-Methoxy- | (3,5-Dimethyl-phenyl)- | 471.2 |
| | 5-yl- | phenyl)- | | |
| 233 | Benzo[1,3]dioxol- | (4-Methoxy- | (2,3-Dichloro-phenyl)- | 511.1 |
| | 5-yl- | phenyl)- | | |
| 234 | (4-Methoxy- | (4-Methoxy- | (3-Methyl-phenyl)- | 443.2 |
| | phenyl)- | phenyl)- | | |
| 235 | (4-Methoxy- | (4-Methoxy- | (3-Chloro-phenyl)- | 463.1 |
| | phenyl)- | phenyl)- | | |
| 236 | (4-Methoxy- | (4-Methoxy- | Naphthalen-1-yl- | 479.2 |
| | phenyl)- | phenyl)- | | |
| 237 | (4-Methoxy- | (4-Methoxy- | Naphthalen-2-yl- | 479.2 |
| | phenyl)- | phenyl)- | | |
| 238 | Phenyl- | (4-Methoxy- | (3-Chloro-phenyl)- | 433.1 |
| | | phenyl)- | | |
| 239 | Phenyl- | (4-Methoxy- | Naphthalen-1-yl- | 449.2 |
| | | phenyl)- | | |
| 240 | Phenyl- | (4-Methoxy- | (3-Methoxy-phenyl)- | 429.2 |
| | | phenyl)- | | |
| 241 | Phenyl- | (4-Methoxy- | Phenyl- | 399.2 |
| | | phenyl)- | | |
| 242 | (2-Chloro-phenyl)- | (4-Methoxy- | (3-Methoxy-phenyl)- | 463.1 |
| | | phenyl)- | | |
| 243 | (2-Chloro-phenyl)- | (4-Methoxy- | Phenyl- | 433.1 |
| | | phenyl)- | | |
| 244 | (2-Chloro-phenyl)- | (4-Methoxy- | Naphthalen-2-yl- | 483.1 |
| | | phenyl)- | | |
| 245 | (4-Phenoxy- | (4-Methoxy- | (3-Methyl-phenyl)- | 505.2 |
| | phenyl)- | phenyl)- | | |
| 246 | (4-Phenoxy- | (4-Methoxy- | (3-Chloro-phenyl)- | 525.2 |
| | phenyl)- | phenyl)- | | |
| 247 | (4-Phenoxy- | (4-Methoxy- | Naphthalen-1-yl- | 541.2 |
| | phenyl)- | phenyl)- | | |
| 248 | (4-Phenoxy- | (4-Methoxy- | (3-Methoxy-phenyl)- | 521.2 |
| | phenyl)- | phenyl)- | | |
| 249 | (4-Phenoxy- | (4-Methoxy- | Phenyl- | 491.2 |
| | phenyl)- | phenyl)- | | |
| 250 | (4-Phenoxy- | (4-Methoxy- | Naphthalen-2-yl- | 541.2 |
| | phenyl)- | phenyl)- | | |
| 251 | (4-Phenoxy- | (4-Methoxy- | Benzo[1,3]dioxol-5-yl- | 535.2 |
| | phenyl)- | phenyl)- | | |
| 252 | (4-Phenoxy- | (4-Methoxy- | (2,3-Difluoro-phenyl)- | 527.2 |
| | phenyl)- | phenyl)- | | |
| 253 | (4-Phenoxy- | (4-Methoxy- | (2-Trifluoromethyl- | 559.2 |
| | phenyl)- | phenyl)- | phenyl)- | |
| 254 | (4-Phenoxy- | (4-Methoxy- | (3-Ethoxy-phenyl)- | 535.2 |
| | phenyl)- | phenyl)- | | |
| 255 | (4-Phenoxy- | (4-Methoxy- | (2-Fluoro-3- | 577.2 |
| | phenyl)- | phenyl)- | trifluoromethyl- | |
| | | | phenyl)- | |
| 256 | (4-Phenoxy- | (4-Methoxy- | (3-Trifluoromethoxy- | 575.2 |
| | phenyl)- | phenyl)- | phenyl)- | |
| 257 | (4-Phenoxy- | (4-Methoxy- | (3-Trifluoromethyl- | 591.2 |
| | phenyl)- | phenyl)- | sulfanyl-phenyl)- | |
| 258 | (4-Phenoxy- | (4-Methoxy- | (3-lodo-phenyl)- | 617.1 |
| | phenyl)- | phenyl)- | | |
| 259 | (4-Phenoxy- | (4-Methoxy- | (3,5-Dimethyl-phenyl)- | 519.2 |
| | phenyl)- | phenyl)- | | |
| 260 | (4-Phenoxy- | (4-Methoxy- | (2,3-Dichloro-phenyl)- | 559.1 |
| | phenyl)- | phenyl)- | | |

Preferred compounds of Table 1b, which were made in some embodiments of this invention according to the synthetic methods outlined in Schemes A, H, J and L, are given by the formula:

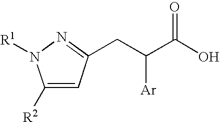

where R², R¹ and Ar are selected concurrently from the groups consisting of:

TABLE 1b

| | | | | [M + H]⁺ |
|---|---|---|---|---|
| EX | R² | R¹ | Ar | *[M − H]⁻ |
| 77 | (4-Bromo- | (4-Methyl-phenyl)- | (3-Methyl-phenyl)- | 475/477 |
| | phenyl)- | | | |
| 85 | (4-Bromo-2- | (4-Methyl-phenyl)- | (3-Methyl-phenyl)- | 509/511 |
| | chloro-phenyl)- | | | |
| 106 | Quinolin-6-yl- | (4-Methyl-phenyl)- | (3-Methyl-phenyl)- | 448.2 |
| 126 | (3,4-Dichloro- | (4-Ethoxy-phenyl)- | (3-Chloro-phenyl)- | *513 |
| | phenyl)- | | | |
| 127 | Naphthalen-2-yl- | (2,5-Dichloro- | (3-Chloro-phenyl)- | 521/523 |
| | | phenyl)- | | |
| 128 | Naphthalen-2-yl- | (4-Ethoxy-phenyl)- | (3-Chloro-phenyl)- | 497.1 |
| 319 | Benzo[1,3]dioxol-5-yl- | (4-Methyl-phenyl)- | (3-Methyl-phenyl)- | |
| 320 | (4-Chloro- | (4-Methoxy- | 3-Isopropoxy- | |
| | phenyl)- | phenyl)- | | |
| 321 | Naphthalen-2-yl- | Benzyl- | (3-Methyl-phenyl)- | |
| 322 | Benzo[1,3]dioxol- | Benzyl | (3-Methyl-phenyl)- | |
| | 5-yl- | | | |
| 323 | (3,4-Dichloro- | (2,4-Dichloro- | (2,5-Dimethyl- | |
| | phenyl)- | phenyl)- | phenyl)- | |
| 324 | (3,4-Dichloro- | (2,4-Dichloro- | (3-Chloro-phenyl)- | |
| | phenyl)- | phenyl)- | | |
| 325 | (3,4-Dichloro- | (2,4-Dichloro- | (3-Isoproxy-phenyl)- | |
| | phenyl)- | phenyl)- | | |
| 326 | (3,4-Dichloro- | (2,4-Dichloro- | (2-Fluoro-5-methyl- | |
| | phenyl)- | phenyl)- | phenyl)- | |
| 327 | (3,4-Dichloro- | (2,4-Dichloro- | (2-Methyl-3-trifluoro- | |
| | phenyl)- | phenyl)- | methyl-phenyl)- | |
| 328 | (3,4-Dichloro- | (4-Hydroxy- | (3-Methyl-phenyl)- | |
| | phenyl)- | phenyl)- | [(S) enantiomer] | |
| 329 | (3,4-Dichloro- | (4-Ethoxy-phenyl)- | (3-Methyl-phenyl)- | |
| | phenyl)- | | | |
| 330 | Naphthalen-2-yl- | (4-Ethoxy-phenyl)- | (3-Chloro-phenyl)- | |
| 331 | (3,4-Dichloro- | (4-Ethoxy-phenyl)- | (3-Chloro-phenyl)- | |
| | phenyl)- | | | |
| 332 | (3,4-Dichloro- | (2,5-Dichloro- | (3-Chloro-phenyl)- | |
| | phenyl)- | phenyl)- | | |
| 333 | (4-Chloro- | (4-Methoxy- | (4-Chloro-phenyl)- | |
| | phenyl)- | phenyl)- | | |
| 334 | (3,4-Dichloro- | (4-Methoxy- | (3-Trifluoromethyl- | |
| | phenyl)- | phenyl)- | sulfanyl-phenyl)- | |

Compound 328 was made by demethylation of Compound 1.

Preferred compounds of Table 2, which were made in some embodiments of this invention according to the synthetic methods outlined in Scheme A and as described in Method 2 or Example 71, are given by the formula:

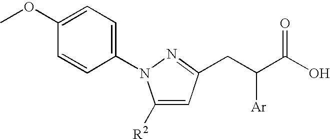

where R² and Ar are selected concurrently from the groups consisting of:

TABLE 2

| EX | R² | Ar | [M + H]⁺ |
|---|---|---|---|
| 14 | (4-Methoxy-phenyl)- | Benzofuran-3-yl- | 469.2 |
| 71 | (4-Methyl-phenyl)- | (1H-indol-3-yl)- | 452.2 |
| 72 | (4-Methyl-phenyl)- | (1-Methyl-1H-indol-3-yl)- | 466.2 |
| 261 | (3,4-Dichloro-phenyl)- | Benzofuran-3-yl- | 507.1 |
| 262 | Benzo[1,3]dioxol-5-yl- | Benzofuran-3-yl- | 483.2 |
| 263 | Phenyl- | Benzofuran-3-yl- | 439.1 |
| 264 | (2-Chloro-phenyl)- | Benzofuran-3-yl- | 473.1 |
| 265 | (4-Phenoxy-phenyl)- | Benzofuran-3-yl- | 531.2 |

Preferred compounds of Table 3a, which were made in some embodiments of this invention according to the synthetic methods outlined in Schemes A, B, C, D and H, and as described in Examples 64-68, 73 and 74, are given by the formula:

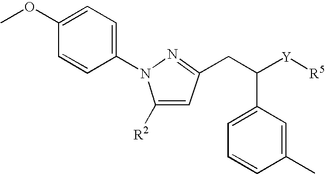

where R² and R⁵—Y— are selected concurrently from the groups consisting of:

TABLE 3a

| EX | R² | R⁵—Y— | [M + H]⁺ |
|---|---|---|---|
| 64 | (4-Methyl-phenyl)- | (2-Hydroxy-cyclohexyl- | 524.2 |
| | | carbamoyl)- | |
| 65 | (4-Methyl-phenyl)- | Carbamoyl- | 426.2 |
| 66 | (4-Methyl-phenyl)- | (Dimethyl-carbamoyl)- | 454.2 |
| 67 | (4-Methyl-phenyl)- | (Methyl-carbamoyl)- | 440.2 |
| 68 | (4-Methyl-phenyl)- | (4-Methyl-piperazine-1- | 509.2 |
| | | carbonyl)- | |

Preferred compounds of Table 3b, which were made in some embodiments of this invention according to the synthetic methods outlined in Schemes D and I, are given by the formula:

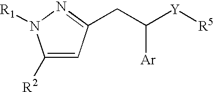

where R² and R⁵-Y— are selected concurrently from the groups consisting of:

TABLE 3b

| EX | R² | R¹ | Ar | R⁵-Y- | [M + H]⁺ |
|---|---|---|---|---|---|
| 74 | (4-Methyl- | (4-Methoxy- | (3-Methyl- | (1H-Tetrazol-5-yl)- | 451.2 |
| | phenyl)- | phenyl)- | phenyl)- | | |
| 129 | (3,4-Dichloro- | (4-Methoxy- | (3-Methyl- | (1H-Tetrazol-5-yl)- | 505.3 |
| | phenyl)- | phenyl)- | phenyl)- | [(S) enantiomer] | |
| 130 | (3,4-Dichloro- | (4-Methoxy- | (3-Methyl- | (1H-Tetrazol-5-yl)- | 505.1 |
| | phenyl)- | phenyl)- | phenyl)- | [racemic] | |
| 131 | (3,4-Dichloro- | (4-Methoxy- | (3-Methyl- | (1H-Tetrazol-5-yl)- | 505.3 |
| | phenyl)- | phenyl)- | phenyl)- | [(R) enantiomer] | |
| 132 | Benzo[1,3]dioxol- | (2,5-Dichloro- | (3-chloro- | (1H-Tetrazol-5-yl)- | 539.0 |
| | 5-yl- | phenyl)- | phenyl)- | | |
| 135 | 3,4-Dichloro- | (4-Methoxy- | (3-Methyl- | (2H-[1,2,4]Triazol- | 550.1 |
| | phenyl- | phenyl)- | phenyl)- | 3-ylsulfanylmethyl)- | |
| 136 | (4-Methyl- | (4-Methyl- | (3-Methyl- | (2H-[1,2,4]Triazole- | 496.2 |
| | phenyl)- | phenyl)- | phenyl)- | 3-sulfinylmethyl)- | |
| 137 | (4-Methyl- | (4-Methyl- | (3-Methyl- | (2H-[1,2,4]Triazole- | 512.2 |
| | phenyl)- | phenyl)- | phenyl)- | 3-sulfonylmethyl)- | |
| 138 | 3,4-Dichloro- | (4-Methoxy- | (3-Methyl- | (2H-[1,2,4]Triazole- | 582.3 |
| | phenyl- | phenyl)- | phenyl)- | 3-sulfonylmethyl)- | |
| | | | | [(S) enantiomer] | |
| 335 | (4-Methyl- | (4-Methyl- | (3-Methyl- | (2H-[1,2,4]Triazol- | |
| | phenyl)- | phenyl)- | phenyl)- | 3-ylsulfanylmethyl)- | |

Preferred compounds of Table 4, which were made in some embodiments of this invention according to the synthetic methods outlined in Schemes E and F, and as described in Methods 4 and 6, are given by the formula:

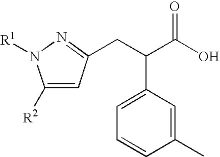

where R² and R¹ are selected concurrently from the groups consisting of:

TABLE 4

| | | | [M + |
|---|---|---|---|
| EX | R² | R¹ | H]⁺ |
| 53 | (4-Phenoxy-phenyl)- | (4-tert-Butyl-phenyl)- | 531.2 |
| 54 | (3,4-Dichloro-phenyl)- | (4-Methanesulfonyl-phenyl)- | 529.1 |
| 55 | Benzo[1,3]dioxol-5-yl- | (2-Chloro-phenyl)- | 461.0 |
| 57 | (3-Chloro-phenyl)- | (2,4-Dichloro-phenyl)- | 485.1 |
| 58 | (4-Benzyloxy-phenyl)- | (4-Trifluoromethoxy-phenyl)- | 573.5 |
| 59 | (4-Dimethylamino-phenyl)- | (4-Methyl-phenyl)- | 440.3 |
| 60 | (3-Methoxy-4-methyl- | (4-Methyl-phenyl)- | 441.3 |
| | phenyl)- | | |
| 61 | (3-Cyclopentyloxy-4- | (4-Methyl-phenyl)- | 511.4 |
| | methoxy-phenyl)- | | |
| 62 | (4-Bromo-3-methyl- | (4-Phenoxy-phenyl)- | 567.4 |
| | phenyl)- | | |
| 266 | Naphthalen-2-yl- | (2,4-Dichloro-phenyl)- | 501.0 |
| 267 | Naphthalen-2-yl- | (2-Chloro-phenyl)- | 467.1 |
| 268 | Naphthalen-2-yl- | (4-Methanesulfonyl-phenyl)- | 511.1 |
| 269 | Naphthalen-2-yl- | (4-tert-Butyl-phenyl)- | 489.2 |
| 270 | Naphthalen-2-yl- | (4-Trifluoromethoxy-phenyl)- | 517.5 |
| 271 | Naphthalen-2-yl- | (4-Methyl-phenyl)- | 447.3 |
| 272 | Naphthalen-2-yl- | (4-Phenoxy-phenyl)- | 525.4 |
| 273 | (3,4-Dichloro-phenyl)- | (2,4-Dichloro-phenyl)- | 519.0 |
| 274 | (3,4-Dichloro-phenyl)- | (2-Chloro-phenyl)- | 485.0 |
| 275 | (3,4-Dichloro-phenyl)- | (4-tert-Butyl-phenyl)- | 507.1 |
| 276 | Benzo[1,3]dioxol-5-yl- | (2,4-Dichloro-phenyl)- | 495.0 |
| 277 | Benzo[1,3]dioxol-5-yl- | (4-Methanesulfonyl-phenyl)- | 505.1 |
| 278 | Benzo[1,3]dioxol-5-yl- | (4-tert-Butyl-phenyl)- | 483.2 |
| 279 | (3-Chloro-phenyl)- | (2-Chloro-phenyl)- | 451.0 |
| 280 | (3-Chloro-phenyl)- | (4-Methanesulfonyl-phenyl)- | 495.1 |
| 281 | (3-Chloro-phenyl)- | (4-tert-Butyl-phenyl)- | 473.2 |
| 282 | (4-Phenoxy-phenyl)- | (2,4-Dichloro-phenyl)- | 543.1 |
| 283 | (4-Phenoxy-phenyl)- | (2-Chloro-phenyl)- | 509.1 |
| 284 | (4-Phenoxy-phenyl)- | (4-Methanesulfonyl-phenyl)- | 553.1 |
| 285 | (4-Benzyloxy-phenyl)- | (4-Methyl-phenyl)- | 503.4 |
| 286 | (4-Benzyloxy-phenyl)- | (4-Phenoxy-phenyl)- | 581.5 |
| 287 | (4-Dimethylamino-phenyl)- | (4-Trifluoromethoxy-phenyl)- | 510.1 |
| 288 | (4-Dimethylamino-phenyl)- | (4-Phenoxy-phenyl)- | 518.4 |
| 289 | (4-Bromo-3-methyl- | (4-Methyl-phenyl)- | 489.3 |
| | phenyl)- | | |
| 290 | (3-Methoxy-4-methyl- | (4-Trifluoromethoxy-phenyl)- | 511.1 |
| | phenyl)- | | |
| 291 | (3-Methoxy-4-methyl- | (4-Phenoxy-phenyl)- | 519.4 |
| | phenyl)- | | |
| 292 | (3-Cyclopentyloxy-4- | (4-Trifluoromethoxy-phenyl)- | 581.4 |
| | methoxy-phenyl)- | | |
| 293 | (3-Cyclopentyloxy-4- | (4-Phenoxy-phenyl)- | 589.5 |
| | methoxy-phenyl)- | | |
| 294 | (4-Chloro-3-methyl- | (4-Isopropyl-phenyl)- | 473.2 |
| | phenyl)- | | |

Preferred compounds of Table 5a, which were made in some embodiments of this invention according to the synthetic methods outlined in Schemes E and F, and as described in Methods 4 and 6, are given by the formula:

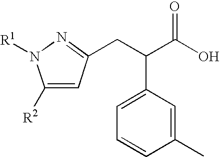

where R² and R¹ are selected concurrently from the groups consisting of:

TABLE 5a

| EX | R² | R¹ | [M + H]⁺ |
|---|---|---|---|
| 52 | Naphthalen-2-yl- | Pyridin-2-yl- | 434.2 |
| 56 | Pyridin-3-yl- | (2,4-Dichloro-phenyl)- | 452.0 |
| 295 | (3,4-Dichloro-phenyl)- | Pyridin-2-yl- | 452.1 |
| 296 | Benzo[1,3]dioxol-5-yl- | Pyridin-2-yl- | 428.1 |
| 297 | (3-Chloro-phenyl)- | Pyridin-2-yl- | 418.1 |
| 298 | (4-Phenoxy-phenyl)- | Pyridin-2-yl- | 476.2 |
| 299 | Pyridin-3-yl- | (4-tert-Butyl-phenyl)- | 440.2 |

Preferred compounds of Table 5b, which were made in some embodiments of this invention according to the synthetic methods outlined in Scheme L, and as described in Example 105, are given by the formula:

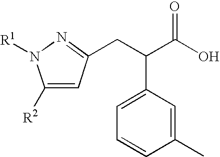

where R² and R¹ are selected concurrently from the groups consisting of:

TABLE 5b

| EX | R² | R¹ | [M + H]⁺ |
|---|---|---|---|
| 78 | (4-Dimethylamino-phenyl)- | Pyridin-2-yl- | 427.2 |
| 80 | Naphthalen-2-yl- | (5-Trifluoromethyl- | |
| | | pyridin-2-yl)- | |
| 81 | (2-Chloro-pyridin-3-yl)- | (2,4-Dichloro-phenyl)- | 486/488 |
| 89 | Naphthalen-2-yl- | Pyridin-4-ylmethyl- | 448.3 |
| 92 | Naphthalen-2-yl- | Pyridin-2-yl- [(S) | 434.1 |
| | | enantiomer] | |
| 93 | Naphthalen-2-yl- | Pyridin-2-yl- [(R) | 434.1 |
| | | enantiomer] | |
| 105 | Naphthalen-2-yl- | (1-Oxy-pyridin-2-yl)- | 450.1 |
| 337 | (3,4-Dichloro-phenyl)- | (5-Trifluoromethyl- | |
| | | pyridin-2-yl)- | |

Preferred compounds of Table 6, which were made in some embodiments of this invention according to the synthetic methods outlined in Schemes E, F and L, and as described in Methods 4 and 6, are given by the formula:

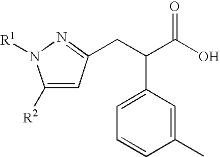

where R² and R¹ are selected concurrently from the groups consisting of:

TABLE 6

| EX | R² | R¹ | [M + H]⁺ |
|---|---|---|---|
| 47 | Naphthalen-2-yl- | H— | 357.2 |
| 49 | (3,4-Dichloro-phenyl)- | Methyl | 388.9 |
| 51 | Naphthalen-2-yl- | Cyclohexyl- | 439.2 |
| 300 | (3,4-Dichloro-phenyl)- | Cyclohexyl- | 457.0 |
| 301 | Benzo[1,3]dioxol-5-yl- | Cyclohexyl- | 433.3 |
| 302 | (3-Chloro-phenyl)- | H— | 341.1 |
| 303 | (3-Chloro-phenyl)- | Methyl | 355.0 |
| 304 | (3-Chloro-phenyl)- | Cyclohexyl- | 423.2 |
| 305 | (4-Phenoxy-phenyl)- | H— | 399.1 |
| 306 | (4-Phenoxy-phenyl)- | Cyclohexyl- | 481.1 |
| 307 | (4-Dimethylamino-phenyl)- | Cyclohexyl- | 432.4 |
| 308 | (4-Bromo-3-methyl-phenyl)- | Cyclohexyl- | 481.4 |
| 309 | (3-Cyclopentyloxy-4- | Cyclohexyl- | 503.5 |
| | methoxy-phenyl)- | | |
| 338 | (3,4-Dichloro-phenyl)- | H— | |

Preferred compounds of Table 7, which were made in some embodiments of this invention according to the synthetic methods outlined in Schemes E and F, and as described in Methods 4 and 6, are given by the formula:

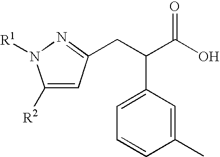

R² and R¹ are selected concurrently from the groups consisting of:

TABLE 7

| EX | R² | R¹ | [M + H]⁺ |
|---|---|---|---|
| 63 | (7-Methoxy-benzofuran-2-yl)- | (4-Phenoxy-phenyl)- | 545.4 |
| 310 | (7-Methoxy-benzofuran-2-yl)- | (4-Trifluoromethoxy- | 537.3 |
| | | phenyl)- | |
| 311 | (7-Methoxy-benzofuran-2-yl)- | (4-Methyl-phenyl)- | 467.4 |
| 312 | (7-Methoxy-benzofuran-2-yl)- | Cyclohexyl- | 459.4 |

Preferred compounds of Table 8a, which were made in some embodiments of this invention according to the synthetic methods outlined in Schemes E and F, and as described in Methods 4 and 6, are given by the formula:

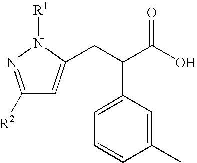

R² and R¹ are selected concurrently from the groups consisting of:

TABLE 8a

| EX | R² | R¹ | [M + H]⁺ |
|---|---|---|---|
| 48 | (3,4-Dichloro-phenyl)- | Methyl | 388.9 |
| 50 | Naphthalen-2-yl- | Cyclohexyl- | 439.2 |
| 313 | (4-Bromo-3-methyl-phenyl)- | Cyclohexyl- | 481.4 |
| 314 | (3,4-Dichloro-phenyl)- | Cyclohexyl- | 457.0 |
| 315 | Benzo[1,3]dioxol-5-yl- | Cyclohexyl- | 433.2 |
| 316 | (3-Chloro-phenyl)- | Methyl | 355.0 |
| 317 | (3-Chloro-phenyl)- | Cyclohexyl- | 423.1 |
| 318 | (4-Phenoxy-phenyl)- | Cyclohexyl- | 481.1 |

Preferred compounds of Table 8b, which were made in some embodiments of this invention according to the synthetic methods outlined in Scheme L, are given by the formula:

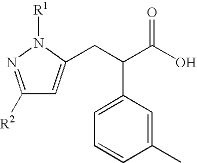

R² and R¹ are selected concurrently from the groups consisting of:

TABLE 8b

| EX | R² | R¹ | [M + H]⁺ |
|---|---|---|---|
| 79 | Naphthalen-1-yl | Pyridin-2-yl | 434.2 |
| 82 | Benzo[1,3]dioxol-5-yl- | Cyclohexylmethyl- | 447.2 |
| 83 | Naphthalen-2-yl- | Benzyl- | |
| 84 | (4-Dimethylamino-phenyl)- | Benzyl- | |
| 88 | Naphthalen-2-yl- | Pyridin-4-ylmethyl- | 448.3 |
| 90 | (3-Dimethylamino-phenyl)- | (4-Methyl-phenyl)- | 440.3 |
| 339 | (4-Dimethylamino-phenyl)- | (4-Methanesulfonyl- | |
| | | phenyl)- | |
| 340 | Benzo[1,3]dioxol-5-yl- | Benzyl- | |
| 341 | (3-Dimethylamino-phenyl)- | (2,5-Dimethyl-phenyl)- | |
| 342 | (3-Dimethylamino-phenyl)- | (4-Methoxy-phenyl)- | |

Preferred compounds of Table 9, which were made in some embodiments of this invention according to the synthetic methods outlined in Scheme L, are given by the formula:

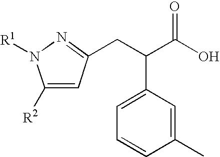

where R² and R¹ are selected concurrently from the groups consisting of:

TABLE 9

| EX | R² | R¹ | [M + H]⁺ |
|---|---|---|---|
| 86 | (4-Dimethylamino-phenyl)- | (4-Methyl-phenyl)- | 440.2 |
| 87 | (1-Methyl-2,3-dihydro-1H-indol-5-yL)- | (4-Methyl-phenyl)- | 452.3 |
| 91 | (3-Dimethylamino-phenyl)- | (4-Methyl-phenyl)- | 440.4 |
| 94 | (4-Allylamino-phenyl)- | (4-Methyl-phenyl)- | 452.6 |
| 95 | (2-Chloro-4-pyrrolidin-1-yl-phenyl)- | (4-Methyl-phenyl)- | 500.1 |
| 96 | (4-Diethylamino-phenyl)- | (4-Methyl-phenyl)- | 468.3 |
| 97 | (4-Isobutylamino-phenyl)- | (4-Methyl-phenyl)- | 468.3 |
| 98 | (4-Morpholin-4-yl-phenyl)- | (4-Methyl-phenyl)- | 482.2 |
| 99 | [2-Chloro-4-(ethyl-methyl-amino)-phenyl]- | (4-Methyl-phenyl)- | 488.1 |
| 100 | [4-(Ethyl-methyl-amino)-phenyl]- | (4-Methyl-phenyl)- | 454.3 |
| 101 | [4-(Isopropyl-methyl-amino)-phenyl]- | (4-Methyl-phenyl)- | 468.3 |
| 102 | (4-Acetylamino-phenyl)- | (4-Methyl-phenyl)- | 454.3 |
| 103 | [4-(Formyl-methyl-amino)-phenyl]- | (4-Methyl-phenyl)- | 454.3 |
| 104 | [4-(2-Oxo-pyrrolidin-1-yl)-phenyl]- | (4-Methyl-phenyl)- | 480.3 |
| 107 | (4-Amino-phenyl)- | (4-Methyl-phenyl)- | 412.2 |
| 344 | (4-Dimethylamino-phenyl)- | Cyclohexylmethyl- | |
| 345 | (4-Dimethylamino-phenyl)- | Pyridin-4-ylmethyl- | |
| 346 | (4-Dimethylamino-phenyl)- | Benzyl- | |
| 347 | (3-Dimethylamino-phenyl)- | (2,5-Dimethyl-phenyl)- | |
| 348 | (3-Dimethylamino-phenyl)- | (4-Methoxy-phenyl)- | |
| 349 | (4-Piperidin-1-yl-phenyl)- | (4-Methyl-phenyl)- | |
| 350 | [4-(Methyl-propyl-amino)-phenyl]- | (4-Methyl-phenyl)- | |
| 351 | (4-Isopropylamino-phenyl)- | (4-Methyl-phenyl)- | |
| 352 | (4-Pyrrolidin-1-yl-phenyl)- | (4-Methyl-phenyl)- | |
| 353 | (4-Propylamino-phenyl)- | (4-Methyl-phenyl)- | |
| 354 | [2-Chloro-4-(methyl-propyl-amino)-phenyl]- | (4-Methyl-phenyl)- | |
| 355 | (4-Azetidin-1-yl-phenyl)- | (4-Methyl-phenyl)- | |
| 356 | [4-(Acetyl-methyl-amino)-phenyl]- | (4-Methyl-phenyl)- | |

Preferred compounds of Table 10, which were made in some embodiments of this invention according to the synthetic methods outlined in Scheme H, are given by the formula:

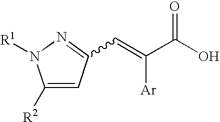

where R², R¹ and Ar are selected concurrently from the groups consisting of:

TABLE 10

| | | | | [M + H]⁺ |
|---|---|---|---|---|
| EX | R² | R¹ | Ar | *[M − H]⁻ |
| 75 | (3,4-Dichloro- | (4-Methoxy- | (3-Methyl-phenyl)- | 479.0 |
| | phenyl)- | phenyl)- | [(E) stereoisomer] | |
| 108 | (3,4-Dichloro- | (4-Ethoxy-phenyl)- | (3-Chloro-phenyl)- | *511/ |
| | phenyl)- | | [(Z) stereoisomer] | 513 |
| 109 | (3,4-Dichloro- | (4-Ethoxy-phenyl)- | (3-Chloro-phenyl)- | 513 |
| | phenyl)- | | [(E) stereoisomer] | |
| 110 | (3,4-Dichloro- | Pyridin-2-yl- | (3-Chloro-phenyl)- | *468 |
| | phenyl)- | | [(Z) stereoisomer] | |
| 111 | (3,4-Dichloro- | (2,5-Dichloro- | (3-Chloro-phenyl)- | *535/ |
| | phenyl)- | phenyl)- | [(Z) stereoisomer] | 537 |
| 112 | Naphthalen-2-yl- | (2,5-Dichloro- | (3-Chloro-phenyl)- | 519/ |
| | | phenyl)- | [(Z) stereoisomer] | 521 |
| 113 | Naphthalen-2-yl- | (4-ethoxy-phenyl)- | (3-Chloro-phenyl)- | 495.1 |
| | | | [(Z) stereoisomer] | |
| 114 | (3,4-Dichloro- | (4-Methoxy- | Phenyl- | 465.1 |
| | phenyl)- | phenyl)- | [(Z) stereoisomer] | |
| 115 | (3,4-Dichloro- | (4-Methoxy- | (3-Chloro-phenyl)- | 499.0 |
| | phenyl)- | phenyl)- | [(Z) stereoisomer] | |
| 116 | (3,4-Dichloro- | (4-Methoxy- | (4-Chloro-phenyl)- | 499.0 |
| | phenyl)- | phenyl)- | [(Z) stereoisomer] | |
| 117 | (3,4-Dichloro- | (4-Methoxy- | (4-Methoxy-phenyl)- | 495.0 |
| | phenyl)- | phenyl)- | [(Z) stereoisomer] | |
| 118 | (3,4-Dichloro- | (4-Methoxy- | (3,4-Dichloro-phenyl)- | 533.0 |
| | phenyl)- | phenyl)- | [(Z) stereoisomer] | |
| 119 | (3,4-Dichloro- | (4-Methoxy- | (4-Methyl-phenyl)- | 479.1 |
| | phenyl)- | phenyl)- | [(Z) stereoisomer] | |
| 120 | (3,4-Dichloro- | (4-Methoxy- | (3-Methyl-phenyl)- | 479.1 |
| | phenyl)- | phenyl)- | [(Z) stereoisomer] | |
| 121 | Benzo[1,3]dioxol-5-yl- | (4-Ethoxy-phenyl)- | (3-Chloro-phenyl)- | 489.1 |
| | | | [(Z) stereoisomer] | |
| 122 | Benzo[1,3]dioxol-5-yl- | (2,5-Dichloro- | (3-Chloro-phenyl)- | 513.0 |
| | | phenyl)- | [(Z) stereoisomer] | |
| 123 | Benzo[1,3]dioxol-5-yl- | (2,5-Dichloro- | (3-Chloro-phenyl)- | 513 |
| | | phenyl)- | [(E) stereoisomer] | |
| 124 | (3,4-Dichloro- | (4-Methoxy- | (3,4-Dichloro-phenyl)- | 532.9 |
| | phenyl)- | phenyl)- | [(E) stereoisomer] | |
| 125 | Benzo[1,3]dioxol-5-yl- | (4-Ethoxy-phenyl)- | (3-Chloro-phenyl)- | 489.1 |
| | | | [(E) stereoisomer] | |
| 357 | (3,4-Dichloro- | (4-Methoxy- | Phenyl- | |
| | phenyl)- | phenyl)- | [(E) stereoisomer] | |
| 358 | (3,4-Dichloro- | (4-Methoxy- | (3-Chloro-phenyl)- | |
| | phenyl)- | phenyl)- | [(E) stereoisomer] | |
| 359 | (3,4-Dichloro- | (4-Methoxy- | (4-Chloro-phenyl)- | |
| | phenyl)- | phenyl)- | [(E) stereoisomer] | |
| 360 | (3,4-Dichloro- | (4-Methoxy- | (4-Methoxy-phenyl)- | |
| | phenyl)- | phenyl)- | [(E) stereoisomer] | |
| 361 | (3,4-Dichloro- | (4-Methoxy- | (3,4-Dichloro-phenyl)- | |
| | phenyl)- | phenyl)- | [(E) stereoisomer] | |
| 362 | (3,4-Dichloro- | (4-Methoxy- | (3-Methyl-phenyl)- | |
| | phenyl)- | phenyl)- | [(E) stereoisomer] | |
| 363 | (3,4-Dichloro- | (4-Methoxy- | (4-Methyl-phenyl)- | |
| | phenyl)- | phenyl)- | [(E) stereoisomer] | |
| 364 | Benzo[1,3]dioxol-5-yl- | (4-Ethoxy-phenyl)- | (3-Chloro-phenyl)- | |
| | | | [(E) stereoisomer] | |

The preferred compounds that follow were made in some embodiments of this invention according to the synthetic methods outlined in Schemes A, B, C, D and J and as described in Examples 76, 139, 133, 134, 140, 141, 336 and 343:
3-[5-(3,4-Dichloro-phenyl)-1-(4-methoxy-phenyl)-1H-pyrazol-3-yl]-2-methyl-2-m-tolyl-propionic acid (Example 76);
3-[5-(3,4-Dichloro-phenyl)-1-(4-methoxy-phenyl-1H-pyrazol-3-yl]-2-fluoro-2-m-tolyl-propionic acid (Example 139);
3-[5-(3,4-Dichloro-phenyl)-1-(2,4-dichloro-phenyl)-1H-pyrazol-3-yl]-2-(3-dimethylamino-phenyl)-propionic acid (Example 133);
3-[5-(3,4-Dichloro-phenyl)-1-(2,4-dichloro-phenyl)-1H-pyrazol-3-yl]-2-quinolin-8-yl-propionic acid (Example 134);
4-(1,5-Di-p-tolyl-1H-pyrazol-3-yl)-3-m-tolyl-butyric acid (Example 140);
5-[5-(3,4-Dichloro-phenyl)-1-(4-methoxy-phenyl)-1H-pyrazol-3-yl]4-m-tolyl-pentanoic acid (Example 141);
5-{2-[5-(3,4-Dichloro-phenyl)-2-(4-methoxy-phenyl)-2H-pyrazol-3-yl]-1-m-tolyl-ethyl}-1H-tetrazole (Example 336); and
3-[2-(4-Methoxy-phenyl)-5-p-tolyl-2H-pyrazol-3-yl]-2-naphthalen-1-yl-propionic acid (Example 343).

Preferred compounds of Table 11, which are made in some embodiments of this invention according to the synthetic methods outlined in Schemes A, E and F, are given by the formula:

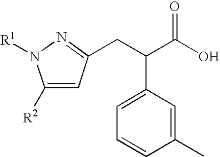

R² and R¹ are selected concurrently from the groups consisting of:

TABLE 11

| EX | R² | R¹ |
|---|---|---|
| 365 | Naphthalen-2-yl- | Pyridin-3-yl- |
| 366 | Naphthalen-2-yl- | Pyridin-4-yl- |
| 367 | Naphthalen-2-yl- | (6-Methyl-pyridin-2-yl)- |
| 368 | Naphthalen-2-yl- | (3-Methoxy-pyridin-2-yl)- |
| 369 | Naphthalen-2-yl- | (5-Methoxy-pyridin-2-yl)- |
| 370 | Naphthalen-2-yl- | (6-Methoxy-pyridin-3-yl)- |
| 371 | Naphthalen-2-yl- | (4-Ethoxy-pyridin-2-yl)- |
| 372 | Naphthalen-2-yl- | (4-Dimethylamino-phenyl)- |
| 373 | Naphthalen-2-yl- | (5-Dimethylamino-2-methoxy-phenyl)- |
| 374 | (3,5-Bis-dimethylamino- | (4-Methyl-phenyl)- |
| | phenyl)- | |
| 375 | (3-Dimethylamino-4- | (4-Methyl-phenyl)- |
| | methoxy-phenyl)- | |

Preferred compounds of Table 12, which may be made in some embodiments of this invention according to the synthetic methods outlined in Schemes A, B, C, D, H and J, are given by the formula:

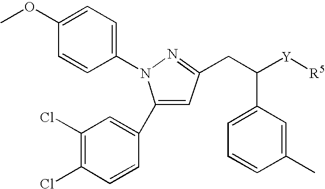

where R⁵—Y— is selected from the groups consisting of:

TABLE 12

| EX | R⁵—Y— |
|---|---|
| 376 | (5-Oxo-4,5-dihydro-1H-[1,2,4]triazol-3-ylsulfanyl)-methyl- |
| 377 | (3H-[1,2,3]Triazol-4-ylsulfanyl)-methyl- |
| 378 | (2H-[1,2,4]Triazole-3-sulfinyl)-methyl- |

Preferred compounds of Table 13, which may be made in some embodiments of this invention according to the synthetic methods outlined in Scheme H, are given by the formula:

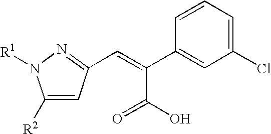

R² and R¹ of such (Z) stereoisomeric compounds are selected concurrently from the groups consisting of:

TABLE 13

| EX | R² | R¹ |
|---|---|---|
| 379 | (4-Dimethylamino-phenyl)- | (4-Dimethylamino-phenyl)- |
| 380 | (4-Dimethylamino-phenyl)- | Naphthalen-2-yl- |
| 381 | (4-Dimethylamino-phenyl)- | (4-Chloro-phenyl)- |
| 382 | (4-Dimethylamino-phenyl)- | Phenyl- |
| 383 | (4-Dimethylamino-phenyl)- | Benzo[1,3]dioxol-5-yl- |
| 384 | Naphthalen-2-yl- | (4-Dimethylamino-phenyl)- |
| 385 | Naphthalen-2-yl- | Naphthalen-2-yl- |
| 386 | Naphthalen-2-yl- | (4-Chloro-phenyl)- |
| 387 | Naphthalen-2-yl- | Phenyl- |
| 388 | Naphthalen-2-yl- | Benzo[1,3]dioxol-5-yl- |
| 389 | (4-Chloro-phenyl)- | (4-Dimethylamino-phenyl)- |
| 390 | (4-Chloro-phenyl)- | Naphthalen-2-yl- |
| 391 | (4-Chloro-phenyl)- | (4-Chloro-phenyl)- |
| 392 | (4-Chloro-phenyl)- | Phenyl- |
| 393 | (4-Chloro-phenyl)- | Benzo[1,3]dioxol-5-yl- |
| 394 | Phenyl- | (4-Dimethylamino-phenyl)- |
| 395 | Phenyl- | Naphthalen-2-yl- |
| 396 | Phenyl- | (4-Chloro-phenyl)- |
| 397 | Phenyl- | Phenyl- |
| 398 | Phenyl- | Benzo[1,3]dioxol-5-yl- |
| 399 | Benzo[1,3]dioxol-5-yl- | (4-Dimethylamino-phenyl)- |
| 400 | Benzo[1,3]dioxol-5-yl- | Naphthalen-2-yl- |
| 401 | Benzo[1,3]dioxol-5-yl- | (4-Chloro-phenyl)- |
| 402 | Benzo[1,3]dioxol-5-yl- | Phenyl- |
| 403 | Benzo[1,3]dioxol-5-yl- | Benzo[1,3]dioxol-5-yl- |

The preferred compounds that follow are made in some embodiments of this invention according to Scheme A and as described in Method 2:
2-Benzofuran-3-yl-3-[1-(4-methoxy-phenyl)-5-p-tolyl-1H-pyrazol-3-yl]-propionic acid; and
2-Benzofuran-3-yl-3-[5-(4-chloro-phenyl)-1-(4-methoxy-phenyl)-1H-pyrazol-3-yl]-propionic acid.

The compounds as described above may be made according to processes within the skill of the art and/or which are described in the schemes and examples that follow. To obtain the various compounds herein, starting materials may be employed which carry the ultimately desired substituents through the reaction scheme with or without protection as appropriate. Starting materials may be obtained from commercial sources or synthesized by methods known to one skilled in the art. Alternatively, it may be necessary to employ, in the place of the ultimately desired substituent, a suitable group, which may be carried through the reaction scheme and replaced as appropriate with the desired substituent. In the Schemes, the pyrazole is depicted with broken lines indicating that the conventional position of the unsaturation is dependent upon the position of the R¹ substituent. Any product containing a chiral center may be separated into its enantiomers by HPLC using a chiral stationary phase.

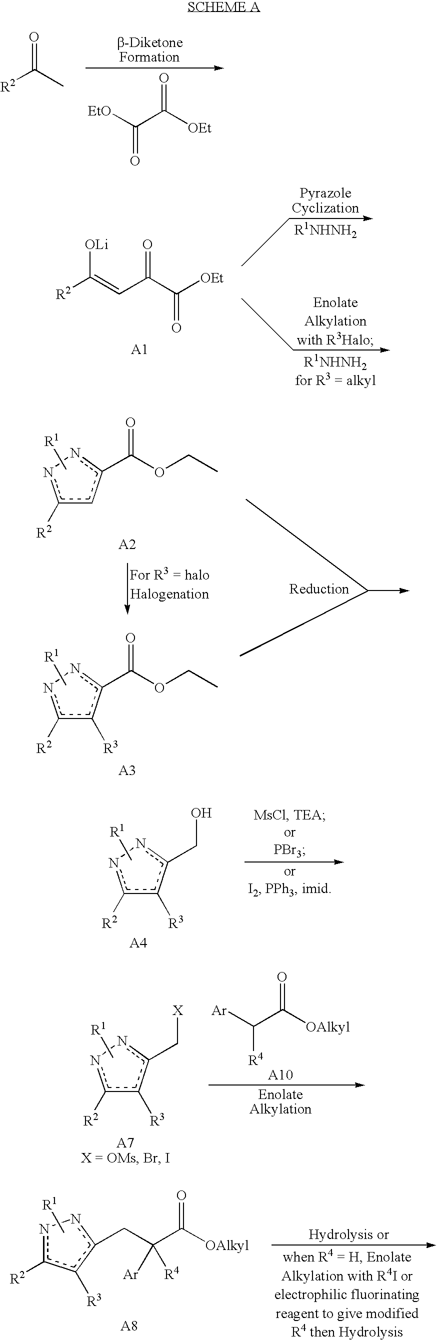

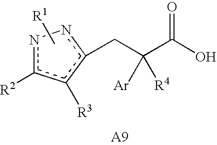

Referring to Scheme A, there are disclosed the following notes and additions. A1 is preferably isolated as an enol salt. In addition to the lithium, the sodium and potassium salts may also be used. A2 is formed as a mixture of regioisomers with either the 1,5- or 1,3-isomer predominating. A2 regioisomers may be separated and carried forward individually. The reduction to A4 may be effected with a number of reducing agents including DIBAL-H and LiAlH₄. The conversion of alcohol A4 to bromide, iodide or mesylate A7 may be carried out with various agents including PBr₃, CBr₄/PPh₃, I₂/imidazole, or MsCl/TEA. The enolate alkylation to A8 may be carried out with R⁴ as hydrogen or alkyl. When R⁴ is hydrogen in A8, R⁴ as alkyl or halogen may be obtained in A9 by enolate alkylation or electrophilic fluorination. Various starting materials A10 may be purchased or certain such starting materials may be synthesized by homologation of aryl aldehydes using chemistry described by Wang, Z. et al. (Synth. Commun. 1999, 29(13), 2321), or Saito, T. et al. (J. Am. Chem. Soc. 1998, 120(45), 11633-11644).

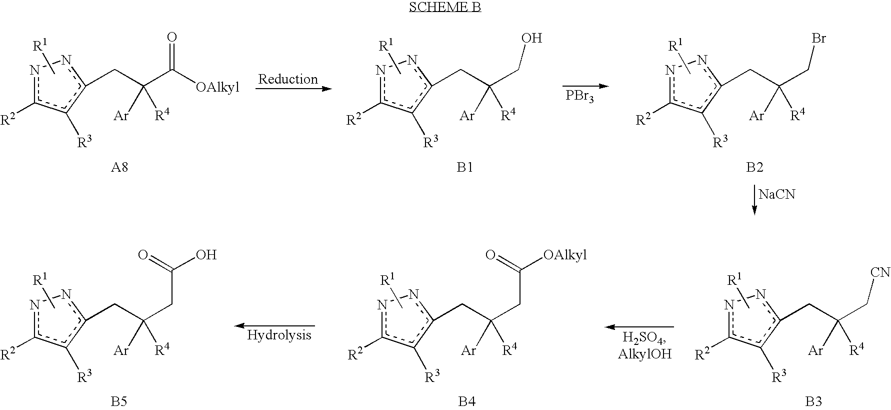

Referring to Scheme B, there are disclosed the following notes and additions. The reduction to B1 may be effected with a number of reducing agents including DIBAL-H and LiAlH₄. Displacement of the hydroxy to form bromide B2 can be carried out using a variety of reagents including PBr₃, or CBr₄/PPh₃. Hydrolysis of the nitrile B3 to the ester B4 can be carried out with a variety of acids including HCl, TsOH, or H₂SO₄. Hydrolysis of the ester B4 to the acid B5 can be performed under basic conditions generally using LiOH. As with the reduction of ester A8 to B1, ester B4 may be reduced to a n+1 analogue of B1, which will produce according to the teachings in Scheme B, a n=2 analogue of B5. Thus, according to Scheme B, both a n=1 and n=2 acid B5 is produced.

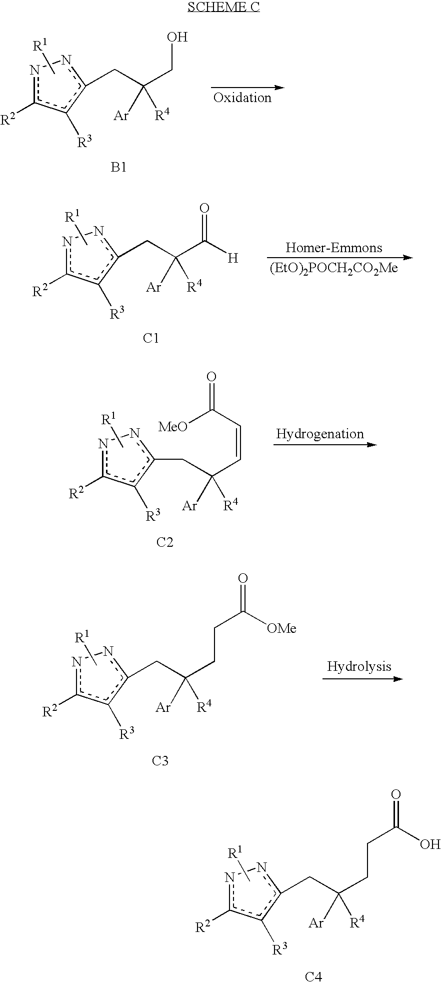

Referring to Scheme C, there are disclosed the following notes and additions. Oxidation of B1 to Cl can be performed using procedures such as the Dess-Martin or Swern oxidations. Hydrogenation to form C3 can be done with a variety of catalytic hydrogenation conditions such as Raney Nickel, Pd/C, CoCl₂/NaBH₄, RhCl(PPh₃)₃. Hydrolysis of ester C3 is generally done under basic conditions with LiOH, but other bases could be used.

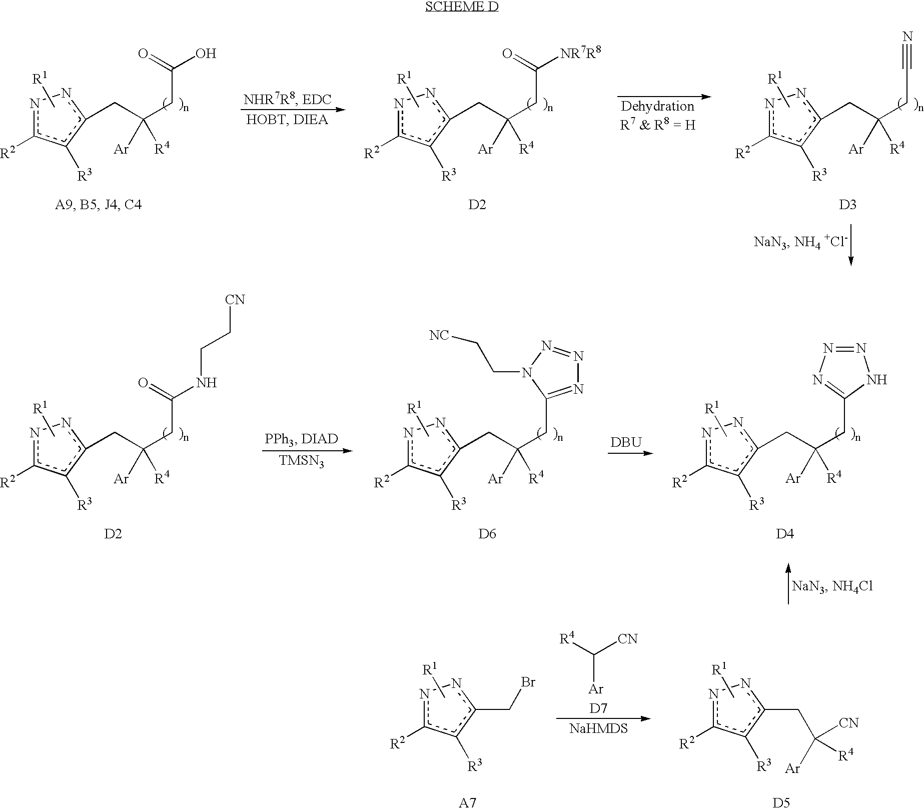

Referring to Scheme D, there are disclosed the following notes and additions. As shown, any of the acids, A9, B5, J4, or C4 can be employed as a starting material. Formation of amide D2 can be performed using a variety of amide bond forming conditions (see: Klausner, Y. S., Bodzansky, M. Synthesis 1974, 8, 549-559). Dehydration with TFAA followed by cyclization of the cyano with NaN₃ gave the desired tetrazole D4. Additionally D5 can be synthesized by addition of bromide A7 to the anion of nitrile D7. Compound D5 can then be converted to the tetrazole D4 using NaN₃. Alternatively the specific amide D2 can be converted to the protected tetrazole D6 using TMSN₃ under Mitsunobu conditions, deprotection with DBU then provides D4.

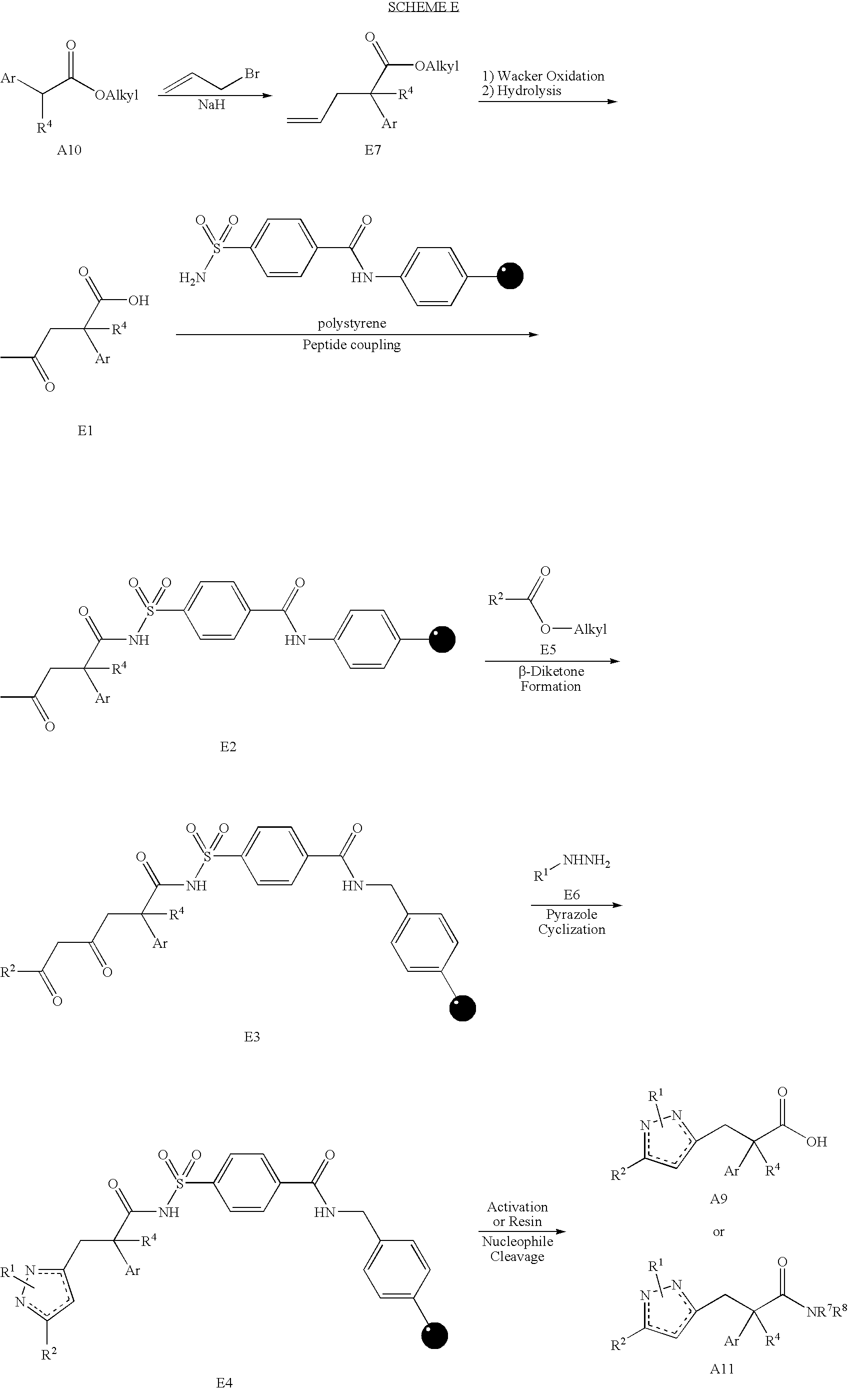

Referring to Scheme E, there are disclosed the following notes and additions. In the manufacture of starting material E1, an aryl acetic acid ester such as A10 is condensed with appropriate terminal olefinic alkyl halide followed by Wacker oxidation to give the ester E7. Hydrolysis of the ester will give the methyl ketone E1. Coupling of acid E1 is to Kenner's safety-catch resin can be accomplished with a variety of peptide coupling reagent including CDI, PyBOP, HOBt. Condensation with E5 gives E3, which is then cyclized with the appropriate hydrazine to give the desired pyrazole E4 as a mixture of regioisomers. Activation of the resin with TMSCH₂N₂ followed by cleavage with hydroxide gives acids A9 as a mixture of regioisomers, which can be separated by HPLC. Alternatively, the activated sulfonamide resin can be cleaved with amine nucleophiles to provide amides A11. Scheme E follows a process similar to that disclosed by Shen, D.-M., et al. (Org. Lett. 2000, 2(18), 2789-2792).

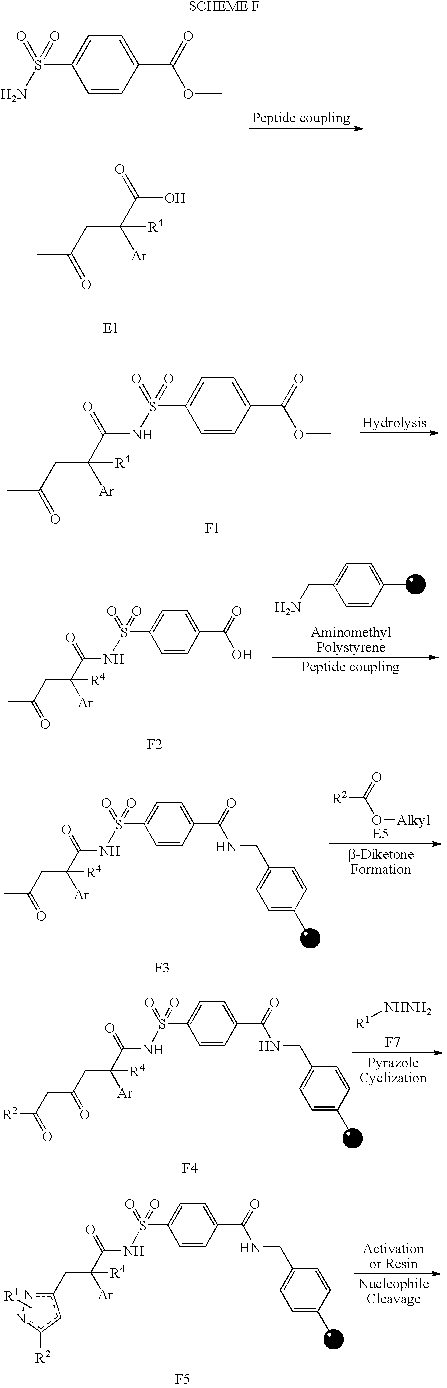

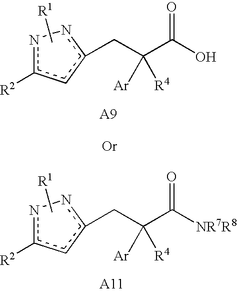

Referring to Scheme F, there are disclosed the following notes and additions. Compounds of type A9 and A11 can be synthesized in a manner similar to scheme E, this approach is outlined in scheme F. In this case a sulfonamide linker is coupled to E1 prior to attachment to resin, to facilitate quantitation of resin loading. Acid F2 is then coupled to macroporous aminomethyl polystyrene support to provide F3, which is similar to E2. Scheme F proceeds from F3 to A9 or A11 in an analogous fashion to Scheme E. Use of macroporous resin provides higher yields of product and easier handling of reactions than the resin used in scheme E.

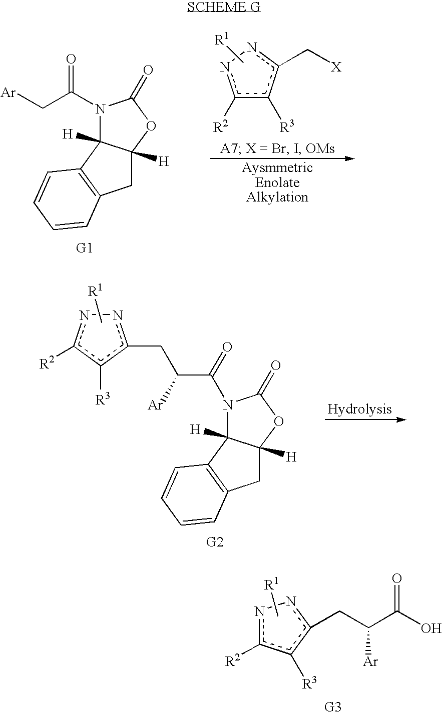

Referring to Scheme G, there are disclosed the following notes and additions. Using the appropriate chiral auxiliary attached to the Ar-acetic acid derivative G1, enolate alkylation by pyrrole A7 affords the desired stereochemistry about the new stereocenter in G2. In addition, other chiral auxiliaries such as the valine and phenylalanine derived oxazolidinones of Evans can also be used. Alternatively, the opposite enantiomer of the chiral auxiliary depicted can be used to synthesize the opposite absolute stereochemistry of G3. As depicted, G3 is the (S) configuration when R⁴ is H and the depicted chiral auxiliary is used. For R⁴ other than H and for other chiral auxiliaries, the absolute configuration G3 may be either the one shown or the opposite configuration depending upon the conditions used.

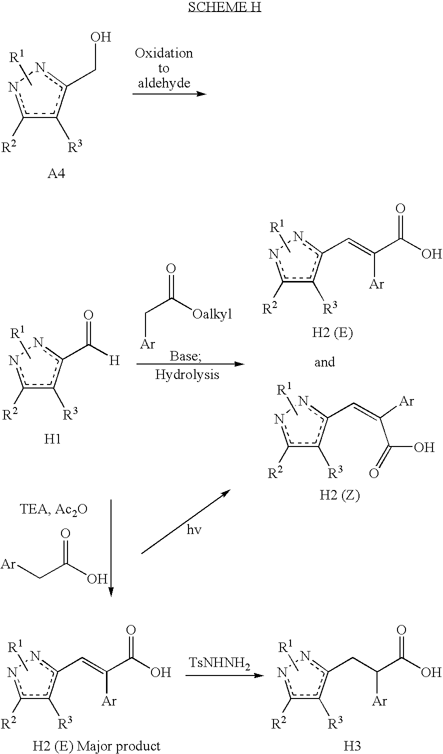

Referring to Scheme H, there are disclosed the following notes and additions. Oxidation of the alcohol A4 can be performed using Dess-Martin or Swern oxidation conditions to provide aldehyde H1. H1 can be condensed with an Ar-acetic acid ester using standard aldol condensation conditions to give the olefin-ester as a mixture of the E- and Z-isomers, which upon hydrolysis affords acids H2 (E) and H2 (Z). The E- and Z-isomers may be separated by chromatography. Alternatively the acid H2 (E) can be obtained directly via a Perkin condensation using an arylacetic acid and Ac₂O. In this case, only acid H2 (E) is formed. Furthermore, photoisomerization of the isolated E- or Z-isomer results in the creation of a mixture of E- and Z-isomers. Additionally reduction of the olefin with TsNHNH₂, or other reducing agent can provide the saturated analogs H3.

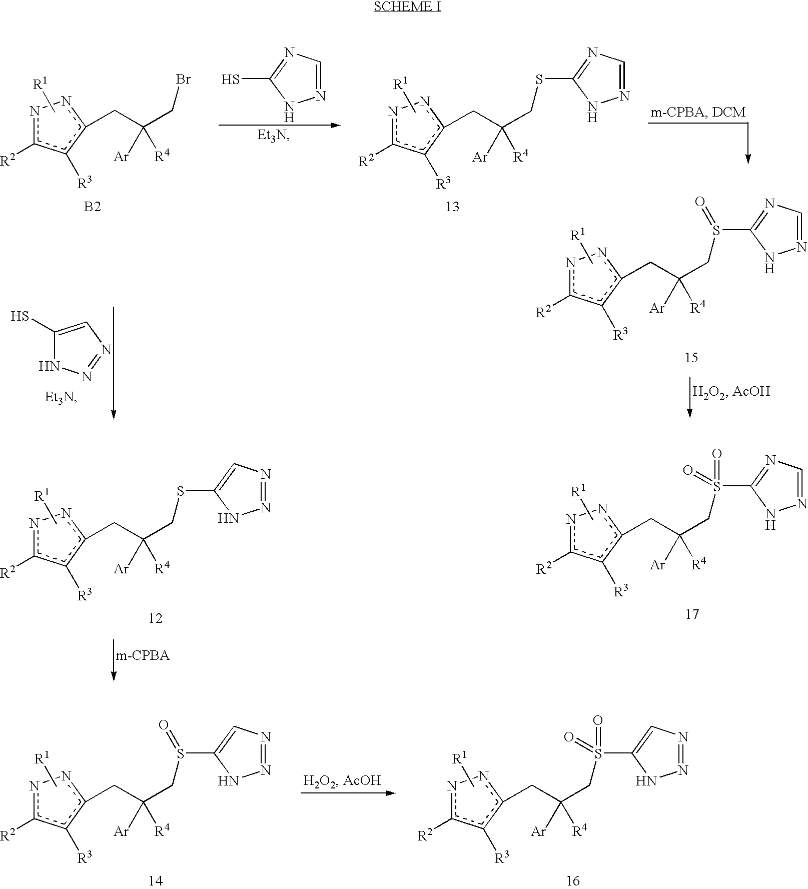

Referring to Scheme I, there are disclosed the following notes and additions. The alkyl bromide B2 can be displaced with several thiol linked heterocycles to give compounds such as I2 or I3. Additionally, the sulfur can be selectively oxidized to the sulfinyl compounds with an oxidant such as mCPBA to afford I4 and I5. Additionally these compounds can be further oxidized to the sulfonyl linked heterocycles by oxidation with such agents as H₂O₂. To obtain analogues of I2 through I7 in which n=2, an n+1 bromide B2 may be used as the starting material. The n+1 bromide B2 may be obtained as described in the paragraph following Scheme B.

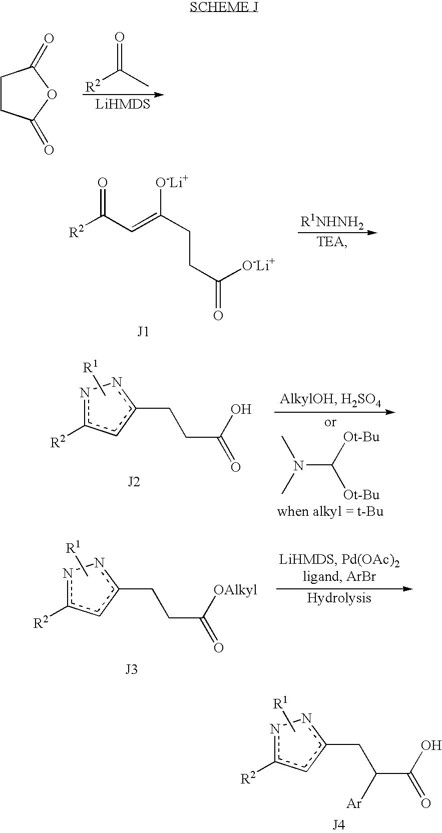

Referring to Scheme J, there are disclosed the following notes and additions. Succinic anhydride can be reacted with the enolate of a methyl ketone to provide enolates of type J1. Additions of hydrazines provide pyrazoles J2 as a mixture of 1,3- and 1,5 regioisomers, these isomers can be readily separated by standard chromatographic methods. Esterification can be performed with a variety of alkyl groups to form esters J3, the preferred Alkyl group being t-Butyl. Coupling of an aryl bromide with the enolate of J3 using the conditions described by Moradi, M. A. and Buchwald, S. L. (J. Am. Chem. Soc. 2001, 123(33), 7996-8002) then provide the ester of J4, which can be hydrolyzed to J4.

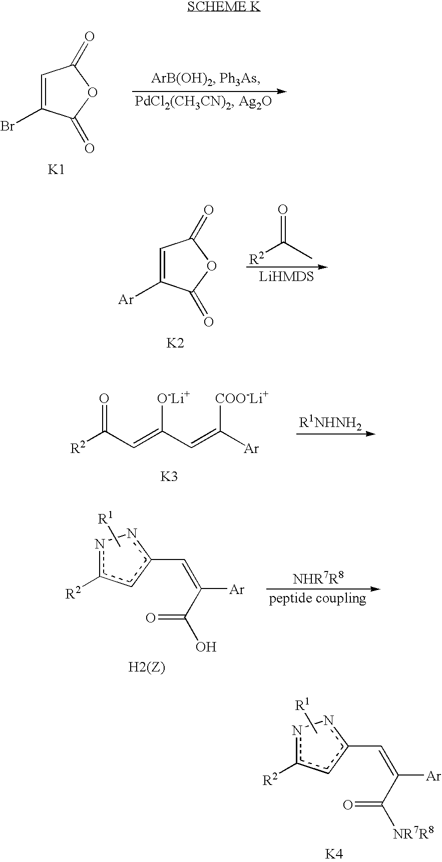

Referring to Scheme K, there are disclosed the following notes and additions. Bromomaleic anhydride can be coupled with aryl boronic acids using Suzuki coupling conditions to provide compounds of type K2. Addition of the enolate of a methyl ketone affords enolates of type K2, which can then be treated with a hydrazine to afford a mixture of 1,3- and 1,5-substituted pyrazoles H2 with exclusively to (Z) olefin geometry shown. These pyrazole regioisomers can be readily separated by chromatography. Pyrazoles H2 may be converted to amides K4 through peptide coupling. Pyrazole H2 may be esterified to produce an alkene equivalent compound A8, which can be used, as disclosed in Scheme B, to produce the n=1 and n=2 analogues.

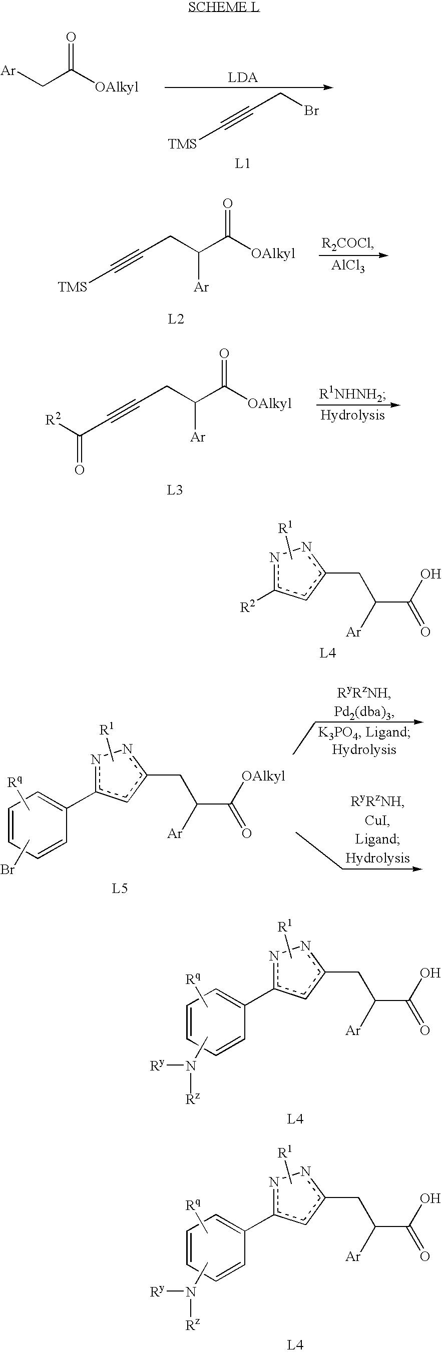

Referring to Scheme L, there are disclosed the following notes and additions. Arylacetic acid esters can be alkylated with propargyl bromides of type L1 to form alkynes of type L2. If the alkyl group is a chiral auxiliary such as depicted in scheme G this transformation can be performed to produce enatiomerically pure compounds of type L2. Friedel-Crafts type coupling of the alkyne L2 with and acid chloride then provides alkynyl ketone L3. Addition of a hydrazine followed by hydrolysis of the ester provides pyrazoles of type L4 as a mixture of 1,3- and 1,5-regioisomers. In addition if the esters L5 contain a halogen on any of the aromatic rings (chemistry is specifically indicated for R² in the scheme) the compound can be coupled with an amine or amide using either the copper or palladium coupling conditions described by Klapars, A. et al. (J. Am. Chem. Soc. 2001, 123(31), 7727-7729) and Wolfe, J. P. et al. (J. Org. Chem. 2000, 65(4), 1158-1174) to obtain nitrogen substituted compounds L4 upon hydrolysis. Additionally if any of the aromatic rings in L4 are a pyridine they can be oxidized to the N-oxide using mCPBA. The racemic mixtures of compounds L4 and L5 can optionally be separated into their individual pure enantiomers through chiral chromatography.

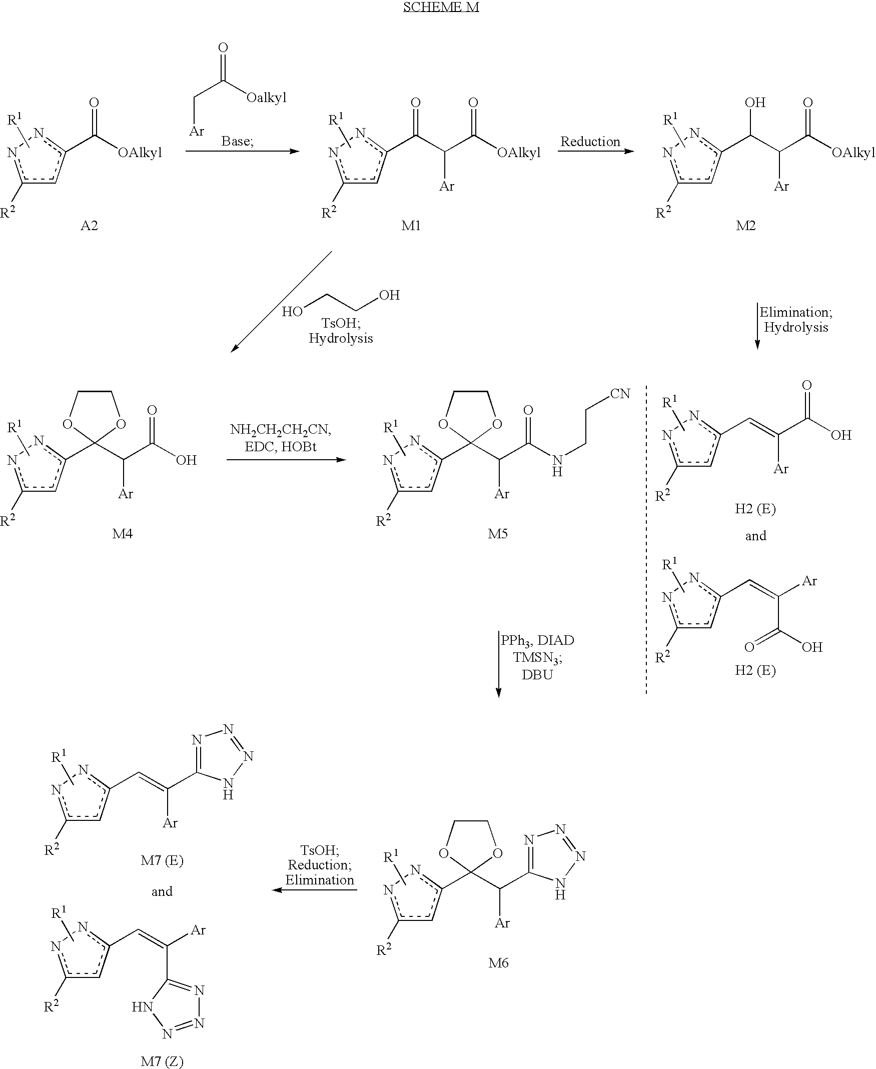

Referring to Scheme M, there are disclosed the following notes and additions. Pyrazole esters of type A2 of either regioisomeric form can be condensed with the enolate of a phenylacetic acid ester to form ketoester M1. Reduction of M1 to the alcohol followed by elimination of the β-hydroxy ester in the presence of base results in the ester of H2, which can then be hydrolyzed to form acid H2 as a mixture of (E) and (Z) isomers. These isomers can be separated by chromatographic methods. Alternatively the ketone M1 can be protected as the ketal, and the ester hydrolyzed to form M4. Amide coupling and tetrazole formation can then be performed using the procedures outlined in scheme D to provide M6. Deprotection, reduction, and elimination as previously described then afford olefinic tetrazoles of the type M7.

In addition to the teachings provided by foregoing Schemes, there are disclosed the following notes and additions regarding the making compounds of formula (I) by processes that are stereoselective and/or regioselective.

It is understood that the teachings provided by foregoing Schemes are not meant to be mutually exclusive with the teachings provided by the following Schemes in their application to chemically meaningful combinations of process steps.

Furthermore, scheme labeling is provided herein only for the convenience of scheme designation, but it is not meant to imply any limitation to the schemes themselves. In addition, scheme labeling provided herein is not meant to imply any limitation to and/or exclusion of any chemically meaningful combination made in light of the ordinary skill in the art, and/or in light of the present disclosure, of the teachings in one or several of the schemes provided herein.

Terms such as “stereoselective”, “stereoselectivity”, and morphologic variations thereof refer to the production of stereoisomeric products in unequal amounts. As conventionally used, enantiomeric excess (often abbreviated as “ee”) means herein |F₍₊₎−F₍₋₎|, where F₍₊₎ denotes mole fraction (or mass fraction) of enantiomer (+), F₍₋₎ denotes mole fraction (or mass fraction) of enantiomer (−), and F₍₊₎+F₍₋₎=1. When given as a percentage, enantiomeric excess is 100·|F₍₊₎−F₍₋₎|. Terms such as “enantiomerically pure”, “optically pure”, and morphologic variations thereof refer to products that satisfy ee >99%.

Terms such as “racemic”, “racemate”, and morphologic variations thereof apply as used herein to mixtures in which the enantiomers are present in equimolar amounts (ee=0) and such mixtures do not exhibit optical activity.

Terms such as “regioselectivity”, “regioselective”, and morphologic variations thereof refer to the existence of a preferential direction of bond making or breaking over other possible directions. Regioselectivity extent is given in terms of a percentage (which is also referred to as regioisomeric excess) of a desired product with certain bonding pattern that is formed in excess of other product or products with some other bonding pattern.

Embodiments of processes illustrated herein include, when chemically meaningful, one or more steps such as hydrolysis, halogenation, protection, and deprotection. These steps can be implemented in light of the teachings provided herein and the ordinary skill in the art.

Embodiments of this invention provide compounds with a desired bonding pattern and/or with a desired chirality by processes that have a small number of synthetic steps. Such small number of steps makes embodiments of this invention particularly suitable for synthetic processes where significant quantities of the desired compound are to be obtained. Scale-up processes are examples of such embodiments.

According to embodiments of this invention, compounds with a desired chirality are synthesized with no need to resort to column chromatographic separation. Furthermore, the compounds with a desired chirality are synthesized in embodiments of this invention with no need to resort to process steps that involve expensive chiral auxiliary compounds.

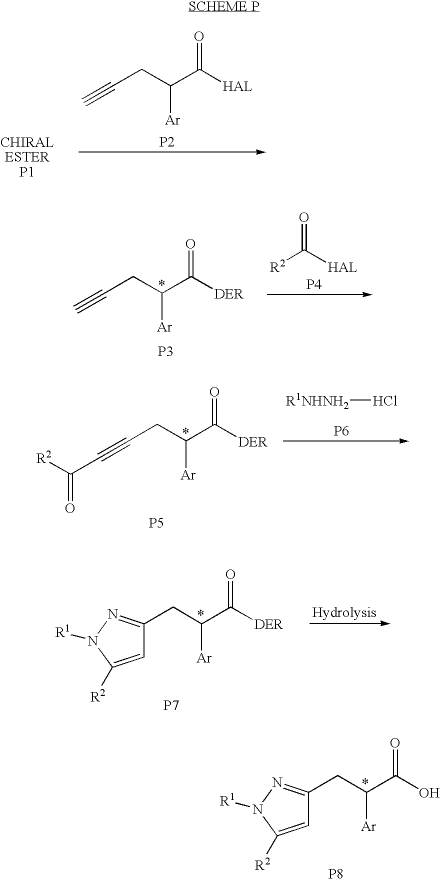

Referring to Scheme P, there are disclosed the following notes and additions. Stereoselectivity is introduced through an acetylenic ketone, such as P5, obtained from a coupling of chiral acetylenic addition product P3 and an acid halide P4. Product P3 is obtained by a stereoselective addition of a chiral ester, such as P1, with an acetylenic acid halide, such as P2. Substituent HAL in P2 and P4 is an appropriate leaving group.

The addition reaction with a chiral ester and an acetylenic acid halide was developed in the context of this invention. It was found in the context of this invention that compounds P3 can be produced by this reaction with high enantiomeric excess regarding the stereogenic center shown in Scheme P with an asterisk. This enantiomeric excess was in embodiments of this invention at least 80%. Referring to diastereomeric excess (de), embodiments of this invention yield P3 with a high diastereomeric excess. Embodiments of this invention produced P3 with de of at least about 80%. Diastereomeric excess with respect to the chirality of a stereogenic center for any pair of diaestereomers is defined analogously as enantiomeric excess is defined above.

The chiral ester was added to a cooled medium. The medium was obtained by mixing an organic base with an acid halide in an organic solvent. Acid chlorides are examples of such acid halides, tertiary amines are examples of such bases, and low polarity solvents are examples of such solvents. Trialkyl amines are preferred tertiary amines, and dimethylethyl amine is a more preferred embodiment. Other amines such as triethyl amine, diethylmethyl amine, and mixtures thereof can be used in embodiments of this invention, preferably tertiary amines whose molecular volume is comparable to that of dimethylethyl amine. An estimate of molecular volumes for such comparison can be performed by resorting to consultation of standard tables of atomic and molecular parameters, including radii, bond lengths, volumes, and molecular properties that lead to an indirect estimate of molecular volumes.

Toluene is a preferred organic solvent. Other solvents such as hexane and mixtures thereof can be used in embodiments of this invention. Preferred solvents are those that are not significantly more polar than toluene, so that the solvent medium preferably has a dielectric constant not greater than about 6, and more preferably not greater than about 3. Organic solvents whose dielectric constant is not greater than about 6 are referred herein as “low polarity organic solvents”. The cooled medium is preferably at a temperature in the range from about −70° C. to about −85° C.

Compound P2 is more preferably an acid halide, in which case the substituent HAL is a halo group, more preferably Cl or Br, and most preferably Cl. Substituent Ar is defined above. Substituent DER is determined by the choice of ester P1. In some embodiments of this invention, ester P1 is ethyl lactate, in which case -DER is 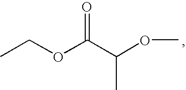
where “O-” denotes the attachment member. In general, -DER is -O-DER′ where DER′ is the moiety of the chiral ester that attaches through the O member to form a compound P3.

Compound P2 is either available or it can be prepared by an acid halide formation reaction. In embodiments of this invention in which HAL is Cl, and Ar is m-tolyl, compound P2 was obtained from 2-m-tolyl-pent-4-ynoic acid and oxalyl chloride under suitable acid chloride formation conditions.

The acid that is used in the formation of the acetylenic compound from which an acetylenic acid halide is subsequently formed is either available or it can be obtained by an alkylation reaction. In some embodiments, 2-m-tolyl-pent-4-ynoic acid was obtained by alkylating m-tolyl acetic acid with propargyl bromide under suitable alkylation conditions.

The alkylation and acid halide formation steps are not displayed in Scheme P for brevity, but they can be implemented in light of the teachings provided herein. Starting reagents for the alkylation and acid halide formation reactions are readily available or can be prepared according to methodology within the ordinary skill in the art.

An asterisk (*) next to a C center in the schemes provided herein denotes a single stereogenic center. The chirality of the stereogenic center of compound P3 is determined by the chirality in chiral ester P1. In some embodiments, P1 was chosen to be (S)-(−)-ethyl lactate, so that each stereogenic center denoted by an asterisk in scheme P was in such case an S-center. Accordingly, the local stereospecific environment of the center 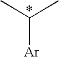
in Scheme P was the S-center 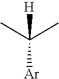
in such embodiments. This choice is illustrative, and another election is possible. For example, the stereogenic center can be R, in which case a chiral ester with R chirality is suitably chosen. A desired chirality can also be introduced by using a hydroxy ester, such as an α-hydroxycarboxylic ester 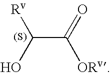
When such α-hydroxycarboxylic ester is used, DER is 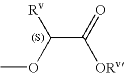
and DER′ is 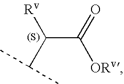
so that the α-hydroxycarboxylic ester is DER′-OH. R^{v} and R^{v′} are groups such that compound P7 can be hydrolyzed to P8. R^{v} and R^{v′} are independently chosen preferably from the group of linear and branched C₁₋₄alkyl.

In some embodiments, compound P3 is a chiral 2-arylpentynoic acid derivative. An example of such P3 is 2-m-tolyl-pent-4-ynoic acid 1-ethoxycarbonyl-ethyl ester.

Chiral acetylenic ketone P5 is obtained by coupling suitably substituted acid halide P4 with the addition product P3. HAL in compound P4 is defined as with respect to P2. This coupling is performed in some embodiments of this invention by a Sonogashira reaction.

Sonogashira reaction conditions include the presence of a palladium-containing catalyst, such as palladium on carbon, Pd(PPh₃)₂Cl₂, Pd₂(dba)₃, Pd₂(dba)₃.CHCl₃, Pd(P^{t}Bu₃)₂, Pd₂(dba)₃.CHCl₃/Pd(P^{t}Bu₃)₂, Pd(OAc)₂, Pd(PhCN)₂Cl₂, and PdCl₂, and a base, such as N-methylmorpholine (NMM), triethyl amine, 1,4-dimethylpiperazine, diisopropylethyl amine, and mixtures thereof in a solvent such as THF, DME, dioxane, DCE, DCM, toluene, acetonitrile, and mixtures thereof at a temperature from 0° C. to 100° C. Preferred bases are not significantly stronger than NMM and they are compatible with the presence of Cu(1) species in the medium.

A copper compound is used as a catalyst in this reaction, such as Cu(I) compound. Such Cu(I) catalyst is preferably incorporated in the reaction medium as substoichiometric quantities of a copper salt, such as CuI or CuBrMe₂S. The use of phosphine ligands, such as PPh₃ or P(^{t}Bu)₃, is part of the methodology of some embodiments of the present invention.

As in other process steps in the context of embodiments of this invention, the use of a high polarity solvent may increase the rate and reduce by-product formation in these reactions. Such high polarity solvent is provided in some embodiments as a mixture of a first solvent with a cosolvent that increases the dielectric constant of the mixture with respect to the dielectric constant of such first solvent. For example, one of ordinary skill in the art will recognize in light of this disclosure that the use of water as such cosolvent may increase the rate and reduce by-product formation in these reactions.

In a preferred embodiment, the palladium source is Pd₂(dba)₃.CHCl₃/Pd(P^{t}Bu₃)₂, Pd(PPh₃)₂Cl₂, or palladium on carbon, the base is NMM, the solvent is THF, toluene, THF with toluene, or a mixture of 1,2-dimethoxyethane (DME) and water, and the temperature is between room temperature and 80° C. In a particularly preferred embodiment, the palladium source is Pd(PPh₃)₂Cl₂, the base is NMM, the solvent is THF with toluene, a catalytic quantity of CuI or CuBrMe₂S is used, and the reaction temperature is room temperature to reflux temperature, most preferably room temperature.

R² and HAL are defined above. In some embodiments, compound P5 is 6-(3,4-dichloro-phenyl)-6-oxo2-m-tolyl-hex-4-ynoic acid 1-ethoxycarbonyl-ethyl ester.

Regioselectivity with respect to the pyrazole framework in P7 is achieved by a condensation reaction involving compound P5 and a suitably substituted hydrazine P6. In some embodiments P6 is a suitably substituted hydrazine in other than free base form, referred to herein as non-free base form, in which the hydrazine is in the presence of an acid, thus forming the combinations that these two components form when they are present in the same medium. An example of such embodiments is a suitably substituted hydrazine hydrochloride. In other embodiments, P6 is a suitably substituted hydrazine in free base form. P6 is preferably a suitably substituted hydrazine in non-free base form in embodiments of the process shown in Scheme P. Substituent R¹ in P6 is defined above, and it is chosen according to the type of substitution desired in product P8.

Compound P7 is a pyrazole derivative wherein n=1 and R³ is H. Other embodiments of this pyrazole derivative, and also of P8 and other pyrazole derivatives referred to herein, such as Q3, Q8, R5.1, R5-R8, and S8 in the following Schemes, can have other assignments of n and R³ in light of the definitions of n and R³ given above, and they can be prepared according to teachings given herein, such as the teachings provided in the context of Scheme A.

The term “substituted” as applied to the hydrazines referred to in condensations described herein is to be read in light of the generic form of compounds P6, where R¹ is defined herein, and it can be, inter alia, H. Therefore, “substituted hydrazine” in this context includes “substituted” (wherein R¹ is a substituent other than H) and “unsubstituted” (wherein R¹ is H) hydrazine as exemplified by P6 together with the definition of R¹ given herein.

The regioselective condensation reaction with an acetylenic ketone and a suitably substituted hydrazine to produce a preferred bonding pattern in compound P7 was developed in the context of this invention. It was found that compounds with a nitrogen substitution pattern in the pyrazole framework as shown in P7 in the surrounding chemical environment of compounds of this invention can be produced by this reaction with high regioselectivity, which reached in embodiments of this invention at least about 80%, or a molar ratio of 1:4, with the isomer in excess being the isomer with the pyrazole framework substituted as shown in Scheme P.

An inorganic base and a suitably substituted hydrazine were added in embodiments of this invention to a solution of acetylenic ketone P5 and later quenched with an acidic solution to obtain a medium with an acidic pH.

Examples of acidic solutions are aqueous acidic solutions, such that their acidity is suitable to bring the medium pH to a sufficiently low pH value. Quenching to an acidic pH was performed in some embodiments with HCl(aq) until the medium pH was in the range from about 2 to about 3. The hydrazine in embodiments of this invention is preferably incorporated as a hydrochloride, and one example of suitably substituted hydrazines used in the context of this invention is 4-methoxyphenyl hydrazine.HCl.

Compound P7 in Scheme P shows a pyrazole framework 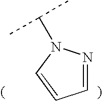
with one of the nitrogen members in the pyrazole framework substituted. This substitution is illustrated in P7 by substituent R¹. It is understood that the other regioisomer is also produced in the same step of formation of P7; and that such other regioisomer has substituent R¹ in the nitrogen member of the pyrazole framework that is shown unsubstituted in Scheme P, whereas the substituted nitrogen member in the same framework is unsubstituted in such other regioisomer.

The solvent in the solution of P5 is preferably an organic solvent, such as benzene, DCM, DCE, THF, DMF, acetonitrile, hexamethylphosphoramide (HMPA), hexane, pentane, alcohol, and mixtures thereof. It was found in the context of this invention that the regioselectivity for the nitrogen substitution pattern in the pyrazole framework can be controlled by selecting the protic or non-protic character of the solvent. Regioselectivity for the nitrogen substitution pattern in the pyrazole framework shown in Scheme P (1-(R¹)-1H-pyrazol substitution) was achieved in embodiments of this invention with a non-protic solvent (a solvent that does not readily release a proton, i.e., a solvent that does not have acidic hydrogens; these non-protic solvents do not have hydrogen atoms attached to highly electronegative atoms, such as N and O), such as THF, TMF, and combinations thereof, preferably THF. Other illustrative non-protic solvents include ether, toluene, and dichloromethane. The other nitrogen substitution pattern, 2-(R¹)-2H-pyrazol substitution, was preferentially obtained with a protic solvent (a solvent that more readily releases a proton, i.e., a solvent that has relatively acidic hydrogens; these protic solvents have hydrogen atoms attached to highly electronegative atoms, such as N and O), such as a carboxylic acid, water, an alcohol and alcohol mixtures, mixtures thereof, and preferably methanol, ethanol, and mixtures thereof.

Examples of inorganic bases that can be used in this condensation are alkali metal hydroxides, such as KOH, NaOH, and mixtures thereof, and alkali metal carbonates, such as Na₂CO₃, K₂CO₃, Cs₂CO₃, and mixtures thereof. Other bases that would perform in this reaction medium as the bases exemplified herein can also be used. A carbonate is preferred, such as Cs₂CO₃.

Embodiments of this invention achieved regioselectivity referred to the nitrogen substitution in the pyrazole framework of at least 1:4, wherein the more abundant isomer conforms to the nitrogen substitution pattern exhibited by compound P7 where the condensation is performed under suitable conditions described herein. In some embodiments, P5 was 6-(3,4-dichloro-phenyl)-6-oxo-2-m-tolyl-hex-4-ynoic acid 1-ethoxycarbonyl-ethyl ester, and P6 was 4-methoxyphenyl hydrazine.HCl, in which case P7 was embodied by 3-[5-(3,4-dichloro-phenyl)-1-(4-methoxy-phenyl)-1H-pyrazol-3-yl]-2-m-tolyl-propionic acid 1-ethoxycarbonyl-ethyl ester. A smaller amount of isomer 3-[5-(3,4-dichloro-phenyl)-2-(4-methoxy-phenyl)-2H-pyrazol-3-yl]-2-m-tolyl-propionic acid 1-ethoxycarbonyl-ethyl ester (P7′) was also formed (nitrogen substitution pattern “2-( . . . )-2H-pyrazol”, a pattern that is not shown in Scheme P), and the molar ratio of this two products was 1:4 referred to relative amounts of P7′ and P7, or 20% and 80%, respectively.

Removal of substituent DER by a suitable process leads to the formation of the final product. Scheme P illustrates an embodiment of P7 wherein DER is such that P7 is an ester, such as a lactate ester. In such embodiments, substituent DER is preferably removed by hydrolysis. Removal of DER leads to product P8. Acetic and hydrochloric acids were used in some embodiments of this invention in the ester hydrolysis.

In some embodiments, compound P7 was 3-[5-(3,4-dichloro-phenyl)-1-(4-methoxy-phenyl)-1H-pyrazol-3-yl]-2-m-tolyl-propionic acid 1-ethoxycarbonyl-ethyl ester, in which case P8 was (S)-3-[5-(3,4-dichloro-phenyl)-1-(4-methoxy-phenyl)-1H-pyrazol-3-yl]-2-m-tolyl-propionic acid. This embodiment of P8 was obtained with an S-enantiomeric excess ee(S) of at least about 80%, which corresponds to a molar enantiomeric ratio R/S of at least about 1:9.

The enantiomeric excess of a product obtained according to the present invention can be increased by crystallization, whether the product is obtained by a synthesis as in Scheme P or by resolution of a racemate. An enantiomeric excess of 80% may be acceptable for some applications of compounds P8. Embodiments of P8 that are to be eventually obtained in enantiomerically pure form are further purified by crystallization.

Embodiments of acids include herein any one of the acid forms such as the acid itself and derivatives thereof such as salts, whether any such salt is isolated or in solution. For example, embodiments of P8 accordingly include P8 salts.

Enantiomeric purification of compounds P8 (not displayed in Scheme P as an additional step) was developed in the context of this invention. It was found in the context of this invention that compounds P8 crystallize under suitable conditions. A salt of P8 is formed to this effect. Such salt is preferably an inorganic salt, such as an alkali metal salt. Other salts are amine salts.

For example an aqueous solution of an inorganic base, preferably a hydroxide, was added to a solution of P8 in an organic solvent, such as THF. Examples of such hydroxides are sodium and potassium hydroxides, but other bases can also be used. Evaporation in a rarefied environment of some of the mixture components is performed until a small amount of water is left in the medium. This residue with a small amount of water is dissolved in a suitable solvent and subsequently crystallized out of a suitable crystallization medium.

It was found in the context of this invention that a suitable crystallization medium is provided by a medium with at least one solvent component, “first component”, and at least another component, “second component”. The first component is such that the residue is soluble therein, and the second component is such that the residue is less soluble than in the first component. For example the residue can be insoluble in the second component; in other embodiments the residue is relatively less soluble in such second component. THF is a preferred embodiment of the first component, and CH₃CN is a preferred embodiment of the second component.

In a preferred crystallization process, the residue with a small amount of water is dissolved in the first component, and then the second component is added, from which medium the P8 salt separates. The term “crystallization” is generically used herein for this process, but it is understood that the salt separates in some embodiments as a crystalline product, in other embodiments it separates as a semicrystalline product, and it can separate in other embodiments as an amorphous product.

In addition to the preferred THF —CH₃CN medium as first-second component medium, other illustrative first-second component media include MeOH—CH₃CN, CH₂Cl₂-toluene, CH₂Cl₂-hexane, and CH₂Cl₂-(toluene-hexane) media, wherein “(toluene-hexane)” refers to mixtures of toluene and hexane. THF, MeOH and CH₂Cl₂ are examples of first component, and CH₃CN, toluene, hexane, and (toluene-hexane) are examples of second component.

In preferred embodiments, this amount of water left in the medium does not differ by more than about 20% from an equimolar amount of water with respect to the amount of P8 salt. For example, in some embodiments this amount of water did not exceed about 1.2 times the amount of water that would be equimolar to the amount of P8 salt. In other embodiments, this amount of water was not less than about 0.8 times the amount of water that would be equimolar to the amount of P8 salt. In these embodiments, the amount of water left in the medium is within about 20% of the water amount that would be equimolar with the amount of P8 salt. In more preferred embodiments, this amount of water left in the medium does not differ by more than about 10% from an equimolar amount of water with respect to the amount of P8 salt, in still more preferred embodiments, this amount of water left in the medium does not differ by more than about 5% from an equimolar amount of water with respect to the amount of P8 salt, and in most preferred embodiments this amount of water left in the medium is about equimolar with respect to the amount of P8 salt.

Crystallization in the context of this invention permits not only enantiomeric enrichment, but also the enrichment of a desired regioisomer. Products with a desired enantiomeric excess and/or a desired degree of regioisomeric enrichment are obtained by crystallization as described herein.

It was found in the context of this invention that inorganic and organic salts are obtained by this crystallization method. Examples of inorganic salts are sodium and potassium salts. Examples of organic salts are amine salts, such as meglumine, tromethamine, tributylamine, and ethylene diamine salts.

The terms “compound (I)” in the context of this invention refer to any of the forms of compound (I), such as the solvent free compound, a solvate thereof, including a hydrate thereof, the compound as in solution, and any crystalline, semicrystalline (semicrystalline referring to a mixture of crystalline and amorphous material), or amorphous form thereof, and mixtures thereof. For example, the terms “a salt of P8” include any one of the forms of such salt, whether anhydrous, or in the form of a solvate, such as any form of hydrate. The same illustration applies to Q8, R8, and S8. Furthermore, the crystallization described herein also applies to the final products obtained according to this invention, such as the final products referred to in Schemes Q, R, and S.

Enantiomeric excess achieved by crystallization according to this invention can readily reach and exceed 90%, and also enantiomeric purity. Regioisomeric enrichment achieved by crystallization according to this invention converts a product with about 80% (regioisomeric excess of at least 80%) of one regioisomer to a product with at least 90% (regioisomeric excess of at least 90%) of the same regioisomer, and embodiments of this invention achieved a regioisomeric enrichment such that the crystallization product was at least 99% (regioisomeric excess of at least 99%) in one of the regioisomers.

When P8 was embodied by (S)-3-[5-(3,4-dichloro-phenyl)-1-(4-methoxy-phenyl)-1H-pyrazol-3-yl]-2-m-tolyl-propionic acid, purification by crystallization led to the isolation of an enantiomerically pure salt, such as (S)-sodium 3-[5-(3,4-dichloro-phenyl)-1-(4-methoxy-phenyl)-1H-pyrazol-3-yl]-2-m-tolyl-propionate, with embodiments of this invention reaching ee(S)>99.9%.

Embodiments of processes schematically illustrated in Scheme P comprise a 6-step synthesis (these steps referring in some embodiments to alkylation, acid halide formation, stereoselective addition, regioselective condensation, and hydrolysis) in which a chosen chirality at a specific stereogenic center is generated at an early synthetic stage by a stereoselective addition between a chiral ester, such as P1, and an acid halide, such as P2. Chiral acetylenic ketone P3 is thus generated. Such embodiments also comprise regioselective condensation and recrystallization enantioenrichment to an optically pure final product. A stereoselective addition in some embodiments of this invention was implemented by using an inexpensive chiral reagent such as (S)-(−)-ethyl lactate.

In contrast with embodiments of the present invention, synthetic processes that rely on other approaches, such as processes that require column chromatographic separation, comprise at least eight steps. Also in contrast with embodiments of the present invention, other processes rely on expensive chiral auxiliary reagents.

Some embodiments include methods of making a compound of formula (I), enantiomers, diastereomers, racemics, pharmaceutically acceptable salts, esters, and amides thereof, comprising: an addition reaction of a chiral ester and an acetylenic acid halide to form a chiral acetylenic addition product.

Some embodiments include methods of making a compound of formula (I), enantiomers, diastereomers, racemics, pharmaceutically acceptable salts, esters, and amides thereof, comprising a condensation in a solvent of a substituted hydrazine and an acetylenic ketone to form a pyrazole derivative, said pyrazole derivative having a pyrazole framework with one of the two nitrogen members in said pyrazole framework substituted according to a regioselectivity pattern of at least 65% yield in one of the two regioisomers, wherein said regioselectivity pattern is determined by selecting said solvent as one of a protic solvent and a non-protic solvent. In some embodiments, said condensation is a regioselective condensation.

Some embodiments include methods of making a compound of formula (I), enantiomers, diastereomers, racemics, pharmaceutically acceptable salts, esters, and amides thereof, comprising: crystallizing a salt of the pyrazole acid derivative of formula (I-A) 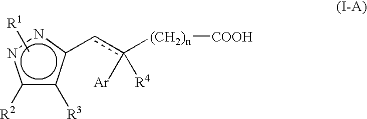
out of a medium to form a crystallization product, wherein said medium before said crystallizing contains an amount of said salt of said pyrazole acid derivative, said medium contains a water amount, and wherein said water amount is within about 20% of the water amount equimolar with said amount of said salt.

Some embodiments include products, enantiomers, diastereomers, racemics, pharmaceutically acceptable salts, esters, and amides thereof, obtained by a method comprising: crystallizing a salt of the pyrazole acid derivative of formula (I-A) 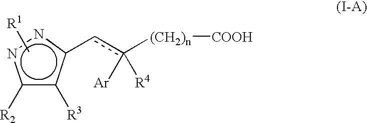
out of a medium, wherein said medium contains an amount of said salt of said pyrazole acid derivative, said medium contains a water amount, and wherein said water amount is within about 20% of the water amount equimolar with said amount of said salt.

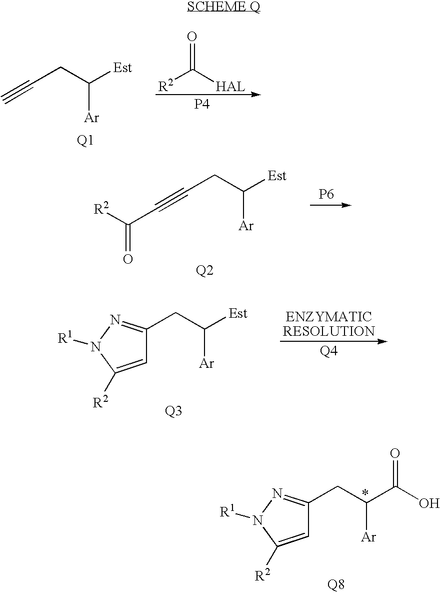

Referring to Scheme Q, there are disclosed the following notes and additions. Acetylenic ketone Q2 is obtained by coupling suitably substituted acid halide P4 with Q1 as described in Scheme Q. This coupling is performed in some embodiments of this invention by a Sonogashira reaction as described in Scheme P.

“Est” is an ester group, such as C(O)(Rox), where Rox is preferably a C₁₋₄alkoxy, wherein “C₁₋₄” denotes herein a linear or branched chain for said alkoxy, such as ethoxy. Compound Q1 is either available or it can be prepared by alkylation as described in Scheme P.

Condensation with a suitably substituted hydrazine P6 is performed as indicated in Scheme P to obtain racemic product Q3. As indicated in the context of Scheme P, compounds with a nitrogen substitution pattern in the pyrazole frameowrk as shown in Q3 in the surrounding chemical environment of compounds of this invention can be produced by this reaction with high regioselectivity, which reached in embodiments of this invention at least about 80%, or a molar ratio of 1:4, with the isomer in excess being the isomer with the pyrazole framework substituted as shown in Scheme Q. Chiral product Q8 is obtained from Q3, preferably by enzymatic resolution Q4.

Enzymatic resolution of compounds Q3 was developed in the context of this invention. It was found in the context of this invention that compounds Q3 could be enzymatically resolved to achieve an enantiomeric excess of at least 90% with an enzyme suitable for hydrolyzing one enantiomer (for example enantiomer (S)) while leaving the other enantiomer (for example enantiomer (R)) esterified. Embodiments of this enzymatic resolution utilized an enzyme comprising a lipase. Examples of lipases include Mucor miehei, lyo; Rhizomucor miehei; and Candida cyclindracea, of which Mucor miehei, lyo, is the preferred lipase. Commercial lipase products used in embodiments of this invention are known as Altus catalyst #8. The enzyme was used in a buffered medium mixed with solutions of compound Q3 in a suitable solvent, such as isopropyl alcohol/toluene. Enzymatic resolution quenching and separation of resolution products lead to product Q8.

When one enantiomer in a mixture of enantiomers is to be enriched, for example when the S-enantiomer is the desired stereospecific form of Q8, the other enantiomer-rich fraction, for example the R-enantiomer enriched fraction, is preferably racemized and incorporated into the process as product Q3 that is subject to enzymatic resolution Q4. Racemization is accomplished, for example, by adding a base, such as KHMDS (potassium bis(trimethylsilyl)amide, also known as potassium hexamethyldisilazide), to a solution of the ester to be racemized (the R-enantiomer enriched ester in some embodiments of this invention).

Preferred bases include bases whose pKₐ is greater than about 23, and more preferably greater than about 25. One of ordinary skill in the art will recognize in light of this disclosure that the use of a base whose pKₐ is chosen according to the direction provided herein will cause the removal of a proton from the stereogenic center and that subsequent reprotonation at the same center will result in racemization of the ester.

Racemization quenching and product separation lead to racemates that can be incorporated in the enzymatic resolution through a recycling process. This recycling process comprises at least one cycle of racemization and enzymatic resolution. The implementation of this recycling step (not displayed in Scheme Q) leads to a quantitatively improved recovery of the desired enantiomer.

As indicated in Scheme P with respect to P8, product Q8 can be further purified by crystallization. Embodiments of this invention lead to the production of the a salt form of Q8 with ee(S)≧99.9%. In some embodiments of this invention, Q1 was 2-m-tolyl-pent-4-ynoic acid ethyl ester, Q2 was 6-(3,4-dichloro-phenyl)-6-oxo-2-m-tolyl-hex-4-ynoic acid ethyl ester, Q3 was 3-[5-(3,4-dichloro-phenyl)-1-(4-methoxy-phenyl)-1H-pyrazol-3-yl]-2-m-tolyl-propionic acid ethyl ester, and Q8 was (S)-3-[5-(3,4-dichloro-phenyl)-1-(4-methoxy-phenyl)-1H-pyrazol-3-yl]-2-m-tolyl-propionic acid, or a salt thereof, such as (S)-sodium 3-[5-(3,4-dichloro-phenyl)-1-(4-methoxy-phenyl)-1H-pyrazol-3-yl]-2-m-tolyl-propionate.

Embodiments of processes schematically illustrated in Scheme Q comprise a 3-step convergent synthesis of a pyrazole framework from acetylenic ketone Q2 by a regioselective condensation. An additional step of enzymatic resolution Q4 comprises kinetic resolution through enzyme-catalyzed hydrolysis of a racemic ester with the pyrazole framework incorporated therein. Optical purity following enzymatic resolution Q4 in embodiments of this invention was at least 92% (ee >92%). Embodiments of such 4-step synthesis according to the present invention contrast with other synthetic approaches that rely on at least eight synthetic steps.

Some embodiments include methods of making a compound of formula (I), enantiomers, diastereomers, racemics, pharmaceutically acceptable salts, esters, and amides thereof, comprising: enzymatically resolving with a lipase a esterified pyrazole derivative of formula (Q3′) 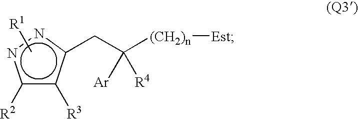
wherein the Ar attached carbon forms a stereogenic center, Est is a substituent chosen from the definition of R⁵ such that Est is a carboxylic acid ester group.

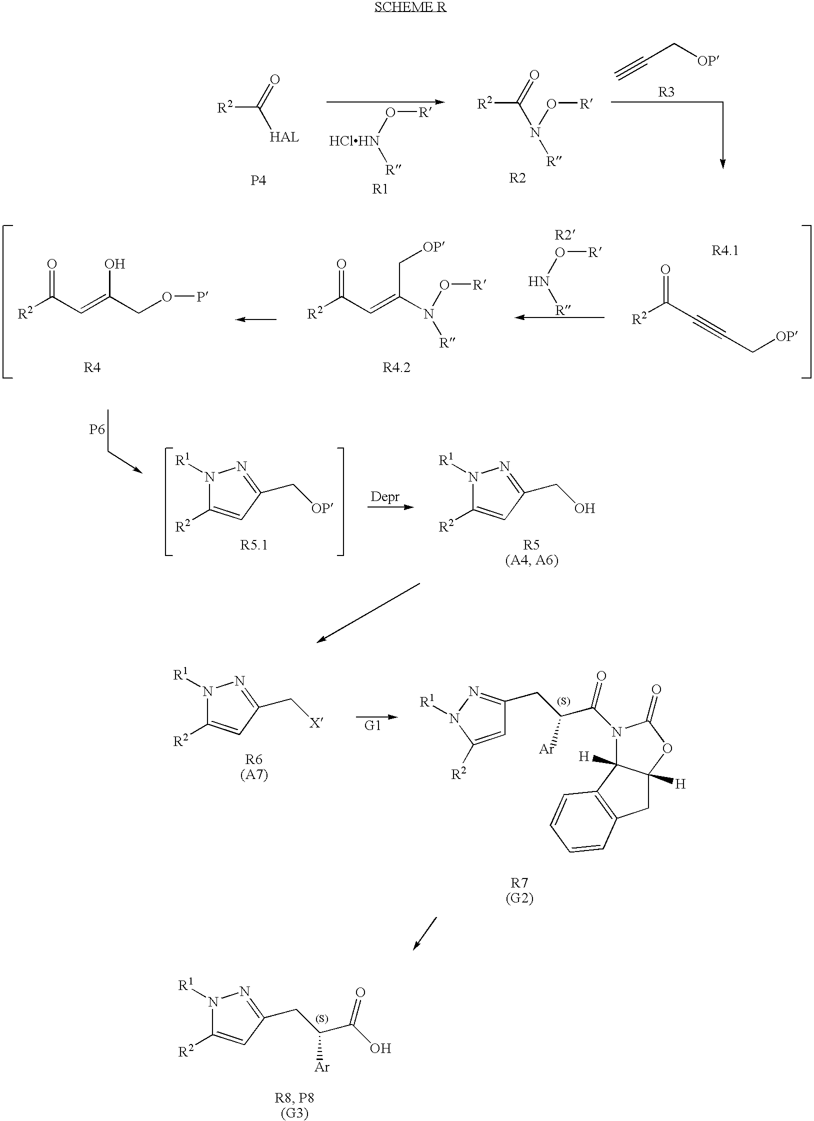

Referring to Scheme R, there are disclosed the following notes and additions. In some embodiments of this invention, a specific stereoisomer was obtained by stereoselective enolate alkylation of a product of condensation with a substituted hydrazine. Regioselective condensation was performed in some embodiments between a substituted hydrazine and a β-diketone, such as R4 that shows a β-diketone in its enol form. Reference herein to one tautomer of any compound that can exist in more than one tautomeric form includes a reference to any other tautomeric form that is not explicitly referred to. For example, reference to structure R4 in an enol form (as shown in Scheme R) also refers to the same structure in its keto form.

Amide R2 is obtained from acid halide P4 and amine R1. Substituents R′ and R″ are independently chosen, preferably as C₁₋₄alkyl, and most preferably R′ is CH₃ and R″ is CH₃.

Amide R2 reacts with acetylenic ether R3 to form acetylenic ketone R4.1, which reacts with amine R2′ to form β-enaminoketone R4.2 which, under acidic conditions hydrolyzes in situ to β-diketone R4, shown in Scheme R in its enol form. Regioselective condensation produces R5.1 which can be deprotected as in Depr in Scheme R, to form pyrazole alcohol R5.

Amide R2 is preferably prepared through a controlled temperature quench that generates, in addition to R2, amine R2′. Acetylenic ketone R4.1 is preferably obtained by propargylating R2 and subsequently quenching the raction mixture with an acidic substance at about 0° C. The acidic substance is chosen so that it preferably comprises a chemically compatible acid capable of regulating the medium pH to a moderately acidic value, such as to an aqueous layer pH of about 5.

In other embodiments of this invention, quenching is performed with a saturated aqueous solution of ammonium chloride. In these embodiments, R2 converts to an amine, such as β-aminoketone R4.3: 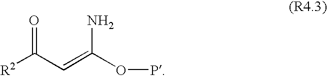
This amine, and also β-enaminoketone R4.2, also participate in the condensation reaction with suitably substituted hydrazine P6 as described herein to form R5.1 in a high regioselectivity process.

Substituent P′ in R3 is preferably a heterocyclic ring attached by a C that is next to a heteroatom, more preferably the heterocyclic ring has only one heteroatom, most preferably this heteroatom is O and P′ is tetrahydropyranyl (THP). Any other suitable protecting group that can subsequently be removed in a deprotection step can be used as P′. Groups P′ that form ethers OP′ are preferred groups.

β-Enaminoketone R4.2 is formed in situ in the addition of amine R2′ to acetylenic ketone R4.1. The enamino group in R4.2 undergoes in situ hydrolysis under aqueous acidic conditions to form 1-diketone R4, shown in Scheme R in its enol form. Analysis of the reaction layer (organic layer) reveals that R4 predominates over R4.1. In embodiments of this invention the molar ratio of the amount of R4.1 to the amount of R4 in the mixture was about 5:95, respectively. The species in this mixture do not need isolation for further processing. Suitably substituted hydrazine P6 in other than a free base form and an inorganic base are added to this mixture to form pyrazole derivative R5.1. An example of P6 in non-free base form is a suitably substituted hydrazine hydrochloride. As indicated herein for this condensation, a carbonate is a preferred inorganic base. It was found in the context of this invention that this pyrazole derivative formation achieves high regioselectivity of, in some embodiments, at least 90%, and in some embodiments at least 95%, with R5.1 (one regioisomer, with nitrogen substitution pattern 1-(R¹)-1H-pyrazol) being formed preferentially with respect to the pyrazole derivative that has R¹ as a substituent in the nitrogen member of the pyrazole framework shown unsusbstituted in Scheme R (the other regioisomer, with nitrogen substitution pattern 2-(R¹)-2H-pyrazol). The molar ratio in embodiments of this invention referring to the ratio of the amount of R5.1 to the amount of the other regioisomer (not shown in Scheme R) was about 98:2. The condensation reaction with hydrazine P6 is thought to take place with R4 and also with R4.2, and furthermore with R4.3 when this substance is present.

Suitably substituted hydrazine P6 is used in some embodiments of this invention in a free base form. When the suitably substituted hydrazine P6 is in free base form, the isomer with the nitrogen substitution pattern in the pyrazole framework that corresponds to the 2-(R¹)-2H-pyrazol substitution (not shown in Scheme R) is preferentially formed. No inorganic base is preferably used in such embodiments with a hydrazine in free base form.

Pyrazole derivative R5.1 undergoes deprotection to generate pyrazole alcohol R5. When P′ is THP, this deprotection is preferably performed by using tosic acid in an alcoholic medium, such as methanol.

Pyrazole alcohol R5 can be isolated or it can be maintained in solution and converted to R6, where substituent X′ is a suitable substituent for the stereoselective alkylation with G1 to form R7 as described in Scheme G. X′ is preferably halo, more preferably Br or I, and most preferably I, in which case R5 is halogenated to R6.

In embodiments in which pyrazole alcohol R5 is isolated, such isolation is preferentially performed by precipitation from a low polarity medium, such as heptane. Halogenation of R5 can be achieved by converting the hydroxyl group with a suitable reagent to a leaving group in a halogenation step, such as by mesylation of the alcohol and subsequent reaction with iodide or bromide.

Halogenated pyrazole derivative R6 can be isolated as shown in Scheme R. Such isolation is not needed in some embodiments, in which R6 is kept in the organic medium for stereoselective alkylation. Halogenated pyrazole derivative R6 is the alkylating agent that reacts with derivative G1 to form chiral R7. This chiral compound R7 does not require its isolation for further processing, and it is subject in embodiments of this invention to an oxidative hydrolysis and acidification to yield pyrazole acid R8.

G1 is obtained in embodiments of this invention from an acid, such as 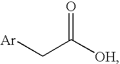
and a chiral tetrahydro-indeno-oxazole in the presence of an organic base, such as triethylamine, and an activating agent. A preferred activating agent is pivaloyl chloride. A preferred organic solvent for this reaction is a low polarity solvent, such as toluene.

As indicated in Scheme R by the symbols within parenthesis, R7 is converted to R8 analogously as G2 is converted to G3 according to Scheme G. Product R8 can further be purified as described above. Also as indicated in Scheme R by the symbols within parenthesis, R6 is in some embodiments obtained from R5 by halogenation, and A7 is obtained from A4 or A6 by halogenation as shown in Scheme A.

As described herein, R8 salts can be prepared (not shown in Scheme R). Inorganic and organic salts of R8, such as alkali metal salts and amine salts, were prepared in embodiments of this invention. Also as described herein, it was found in the context of this invention that these salts can be isolated by crystallization, and that embodiments of such crystallization are crystalline material, and other embodiments comprise a mixture of crystalline and amorphous material, the latter embodiments being referred to as being semicrystalline.

Some embodiments include methods of making a compound of formula (I), enantiomers, diastereomers, racemics, pharmaceutically acceptable salts, esters, and amides thereof, comprising: a condensation of a substituted hydrazine and at least one of a β-diketone, a β-enaminoketone, and a β-aminoketone to form a pyrazole derivative, said pyrazole derivative having a pyrazole framework with one of the nitrogen members in said pyrazole framework substituted. In some embodiments said condensation is a regioselective condensation.

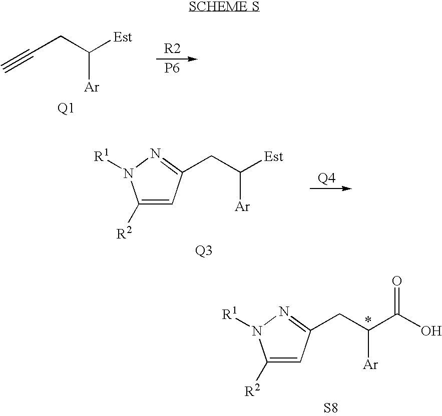

Referring to Scheme S, there are disclosed the following notes and additions. A product of the addition of acetylenic ester Q1 to amide R2 is regioselectively condensed with suitably substituted hydrazine P6 to form racemic Q3.

Q1 can be obtained by propargylation of the corresponding ester Ar—CH₂-Est. In some embodiments, the reaction of Q1 with R2 is quenched with a saturated aqueous solution of ammonium chloride and then the organic layer is treated with P6 to regioselectively form racemic Q3.

Scheme S shows another strategy for forming species that will condense with a suitably substituted hydrazine in a high regioselective process. The nitrogen substitution in the pyrazole framework as shown in Q3 in Scheme S was in embodiments of this invention in a molar ratio of about 98:2 referring to the amount of the isomer shown in Q3 with respect to the isomer that would have the substituent R¹ in the nitrogen member that is shown unsubstituted in Q3.

Substituent Est is defined above. Regioselective condensation with suitably substituted hydrazine P6 according to Schemes R and S is performed under conditions similar to those described in Schemes P and Q. Compound S8 is obtained by enzymatic resolution Q4 as described in Scheme Q.

Some embodiments include methods of making a compound of formula (I), enantiomers, diastereomers, racemics, pharmaceutically acceptable salts, esters, and amides thereof, comprising: an addition of an acetylenic ester to an amide to form an addition product, and a condensation of said addition product with a substituted hydrazine to form a pyrazole ester derivative of formula Q3′ 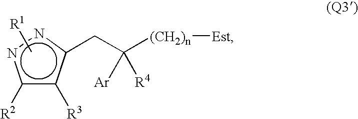
wherein the group Est in Q3′ is a substituent chosen from the definition of R⁵ such that Est is a carboxylic acid ester group. In some embodiments said condensation is a regioselective condensation.

The assignments R³=H and n=1 in the structures displayed in Schemes P-S are used as illustrations and they are not meant as limitations of the processes illustrated in Schemes P-S. As indicated above, it is understood that the teachings provided herein can be used together to apply the processes illustrated in Schemes P-S to the general range of assignments for R³ and n as defined herein. Accordingly to this description, P7 is one embodiment of P7′ and P8 is an embodiment of P8′, wherein P7′ and P8′ are also within the scope of the present invention, and they are represented by the following structures: 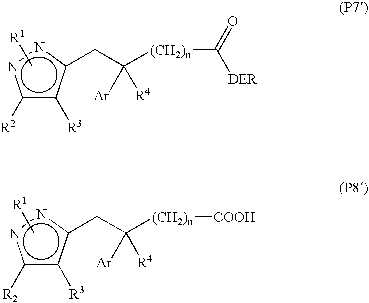
Furthermore, Q3 is one embodiment of Q3′, Q8 is one embodiment of Q8′ (with the same structural representation as P8′), and S8 is an embodiment of S8′ (with the same structural representation as P8′), wherein Q3′, Q8′ and S8′ are also within the scope of the present invention, and they are represented by the following structures (structures for Q8′ and S8′ not given because they have the same structural representation as P8′): 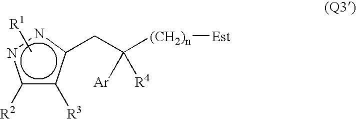
In addition, R5 is an embodiment of R5′, R6 is an embodiment of R6′, and R8 is an embodiment of R8′, wherein R5′, R6′, and R8′ are also within the scope of the present invention, and they are represented by the following structures:

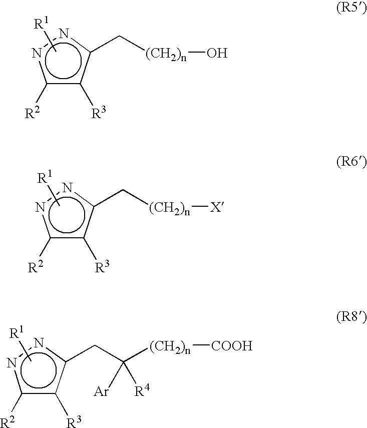

This invention encompasses methods for making a compound of formula (I′), (II), or (III), esters, enantiomers, diastereomers, racemics and pharmaceutically acceptable salts thereof, comprising: providing an α-hydroxyester compound having
(a) an α-carbon member that is alkylated through an intervening methylene with a group that does not have a dehydration-removable hydrogen bonded to said methylene,
(b) an ester moiety with its carboxy group attached directly to said α-carbon member, and
(c) a substituent attached to said α-carbon member, wherein the volume of said substituent is greater than the volume of said ester moiety; and treating said α-hydroxyester compound with a dehydrating agent.

Examples of such α-hydroxyester compounds are compounds S5, S13, T6, and 604, shown in the Schemes below, where the α-carbon member is the carbon member that has a hydroxy group attached to it. Examples of such alkylation groups are G in compound S3, and the pyrazole moiety in S13, T6, and 604. Examples of such alkylation groups together with an intervening methylene group are the moieties G-CH₂— in compound S3 and (substituted)pyrazole-CH₂— in S13, T6, and 604. Examples of such ester moiety are the groups 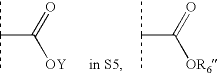
in S13, respectively, and the group —COOCH₃ in T6 and 604, in all of which the carboxy group is directly attached to the α-carbon member of the α-hydroxyester compound. The groups Y and R⁶ are a —C₁₋₃alkyl. Examples of such substituent are Ar″ in S5, and Ar′ in S13 and in T6. A particular embodiment of Ar′ is shown by the 3-chlorophenyl group in 604.

This invention also encompasses methods for making a compound of formula (II), esters, enantiomers, diastereomers, racemics and pharmaceutically acceptable salts thereof, comprising: providing an α-hydroxyester compound of formula S5; and treating said compound of formula S5 with a dehydrating agent.

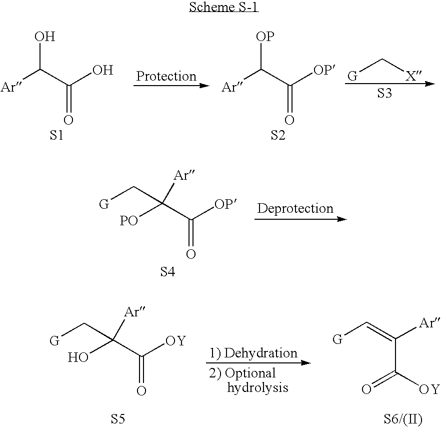

Referring to Scheme S-1, there are disclosed the following notes and additions. The protection, alkylation, and deprotection steps shown in Scheme S-1 are illustrative examples of generating an α-hydroxyester, such as α-hydroxyester S5. Formation of the cis olefin product S6 is accomplished through a selective dehydration of hydroxy esters S5. In some embodiments of this invention, these hydroxy esters are obtained by alkylation of a mandelic acid analog. Such mandelic acid analog refers to any one of the forms S1 (unprotected), S2 (protected with groups P and P′), or partially protected with only one of the OH groups being protected (not shown). It is understood that the choice of any one of these forms, whether referred to explicitly as S1 or S2, is within the ordinary skill in this synthetic methodology in light of the teachings provided herein. Any sensitive functional groups present as substituents in these intermediates may be orthogonally protected, as appropriate. Suitable protecting group schemes will be apparent to one skilled in the art. Mandelic acid analogs may be commercially available or they may be prepared using known procedures. The mandelic acid analogs may be in racemic or enantiopure form. The hydroxyl and carboxylic acid functionalities in mandelic acid derivatives S1 are suitably protected with P and P′, either separately, or in one step, for example, where P and P′ are taken together to form a ring, as in a [1,3]-dioxolan-4-one.

Alkylation of the protected mandelic acid derivative S2 involves treatment with a base such as n-BuLi, LDA, LiHMDS, or NaH, followed by reaction with an appropriate alkylating agent S3, where X″ is a leaving group in an alkylation reaction. The nature of such leaving group can be determined in light of the skills in this synthetic methodology and the teachings provided herein. Examples of such leaving group are provided by the groups: iodide, bromide, chloride, tosylate, and the like. G and Y are defined as above for compound of formula (II).

The protecting group(s), such as a dioxolanone protective group, in S4 is (are) then removed to form hydroxy ester S5. Preferably the base is sodium methoxide, revealing a methyl ester. The reaction may be performed using basic alcoholysis conditions. Alcoholysis is performed with alcohols such as C₁₋₃alkylOH, and mixtures thereof, and preferably methanol. Alternatively, the hydrolysis may be perfomed using acidic conditions, such as refluxing methanol with catalytic sulfuric acid. Additional embodiments for the saponification are known to one skilled in the art.

Development of a selective elimination to introduce the double bond in S5 to generate products S6 is within the scope of the invention. Treatment of hydroxy ester S5 with a dehydrating agent such as triflic anhydride (Tf₂O), fluorosulfonic anhydride, and methanesulfonyl chloride, with or without the addition of base, will effect dehydration. Preferably, triflic anhydride is used. In some embodiments of this invention, Tf₂O is preferably used in the presence of pyridine. Preferred solvents include dichloromethane. It is envisaged that other embodiments of this invention include the use of non-polar solvents such as toluene, pentane, benzene, mixtures thereof, and mixtures with dichloromethane. Preferred temperature ranges are between 0° C. and about 35° C.

In some embodiments of this invention, selectivity for the Z olefin isomer (substituent G and carboxyl group on same side of olefin) was at least about 95%, in some embodiments such selectivity was above 95%, and in other embodiments such selectivity was at least about 99%. In some embodiments of this invention, selectivity for the Z olefin as shown in Schemes S-1A, S-2, T, and T-1 below was at least about 95%, in some embodiments such selectivity was above 95%, and in other embodiments such selectivity was at least about 99%.

The resulting α,β-unsaturated ester of formula (II) where Y is —C₁₋₃alkyl can be optionally and subsequently hydrolyzed to form acids S6 (where Y is H), using conditions known to one skilled in the art. Preferred conditions include aqueous LiOH in dioxane at reflux temperature. In some embodiments, the E isomer is formed to an extent not significantly greater than about 10%. As it can be appreciated by anyone of ordinary skill in the art, this isomer can be easily separated, for example, by crystallization.

It was found in the context of this invention that when Ar′ is bigger than R^{5′} in formula (I′), or when Ar″ is bigger than G in formula (II), the dehydration is stereoselective and the Z stereoisomer is generated. This result is in contrast with conventional methodology that can readily produce the E stereoisomer (with a bonding structure as depicted in S6, but with the moieties G and Ar″ on the same side of olefin). In particular, conventional synthetic methods for constructing a double bond as shown in compund of formula (II) are not stereselective or generally favor the E-stereochemistry. Analogously, the Z stereoisomer is preferentially produced for formula (I′) as well.

The present invention addresses the synthesis, including large-scale synthesis, of compounds of formulae (I′), (II) and (III). In contrast with the present invention, conventional methodologies rely on the synthesis of the E isomer, which is subsequently isomerized photochemically to the Z isomer. Large scale synthesis, however, needs a more efficient synthetic method for making the Z isomer.

Some conventional methods are reportedly stereoselective, such as the Horner-Wadsworth-Emmons reaction and its Still-Gennari and Ando variations. Such methods, however, gave mixtures of E and Z isomers when applied to the synthesis of compounds according to the present invention.

Although the present invention is not limited by specific explanations or theories on the mechanisms for the stereoselective dehydration, it is conceived that dehydration in a strictly E2 (trans-elimination) fashion should provide the desired Z-double bond geometry. Advantages of embodiments of this invention include the use of starting materials and reagents that are readily available and inexpensive. Addional advantages in embodiments of this invention include high yields in the synthetic steps. Furthermore, other advantages include the generation of intermediates that are isolated in high purity or are purified by crystallization, thus avoiding the need for chromatographic purifications.

It is understood in light of the present disclosure, that the teachings provided herein can be generally implemented by one of ordinary skill in the art, for resources on how to estimate volumes of atoms, molecules and moieties thereof are available as part of the standard literature. See, for example, Chem3D Pro software package providing elements for computing properties including surface areas and molecular volumes (http://www.hallogram.com/science/chem3dpro/); compiled reference materials and related student handouts are available at http://bmbiris.bmb.uga.edu/wampler/8200/size/.

This invention also encompasses methods for making a compound of formula (III), esters, enantiomers, diastereomers, racemics, and pharmaceutically acceptable salts thereof, comprising: providing an α-hydroxyester compound of formula S23; and treating said compound of formula S23 with a dehydrating agent.

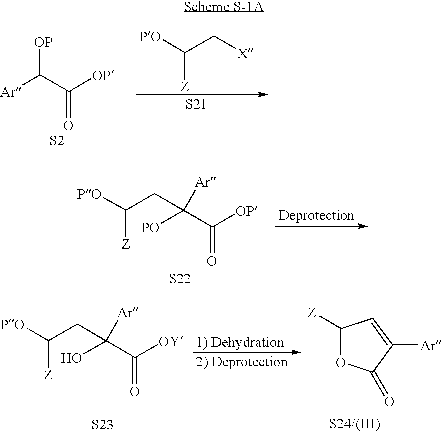

Referring to Scheme S-1A, compounds of formula (III) may also be prepared using methods of the present invention. The alkylation and deprotection steps shown in Scheme S-1A are additional illustrative examples of generating an α-hydroxyester, such as α-hydroxyester S23. Reference to compounds of formula S23 imply reference to any one of structures as shown in Scheme S-1A where the hydroxyl group explicitly shown therein is protected and structures such as the one shown in Scheme S-1A where such hydroxyl group is not protected. The protection/deprotection involved in the choice of any of such structures S23 can be done with the ordinary skill in this synthetic technology and in light of the teachings provided herein. Alkylation of mandelic acid analogs (such as any of structures S1 and S2 in light of the remarks concerning these structures given in the context of Scheme S-1) with an alkylating agent of formula S21, where Z is as defined for formula (III) or may be taken together with OP″ to form a cyclic acetal, generates compounds of formula S22, and P″ is preferably an acid-labile protecting group. Preferred conditions for the alkylation, deprotection of protecting group P, and dehydration steps are as described for Scheme S-1. Y′ is —C₁₋₄alkyl. Y′ in S23 is introduced through the deprotection step or, where the deprotection step yields the corresponding acid of S23, an ester of S23 is formed in a subsequent step. Examples of embodiments of groups P, P′ and P″ in Schemes S-1, S-1A, and S-2 include the hydroxyl and acid proecting groups given herein. Following the dehydration step, resulting intermediates may be treated in situ or during workup with acid, such as 1 N HCl, if necessary, to liberate an aldehyde or free alcohol, and promote cyclization with ester —CO₂Y to generate butenolides of formula S24/(III). Illustrative examples, although not limiting, of such teachings are provided by Scheme S-1A, and by Examples 619 and 622.

This invention also encompasses methods for making a compound of formula (I′), esters, enantiomers, diastereomers, racemics, and pharmaceutically acceptable salts thereof, comprising: providing an α-hydroxyester compound of formula S13; and treating said compound of formula S13 with a dehydrating agent.

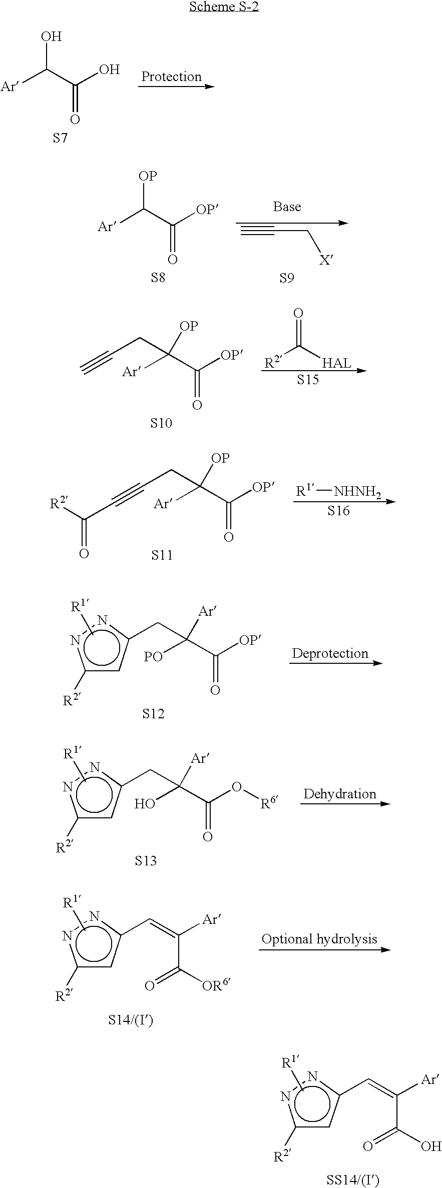

Referring to Scheme S-2, there are the following notes and additions. The protection, acetylenic derivative formation, coupling, condensation and deprotection steps shown in Scheme S-2 are additional illustrative examples of generating an α-hydroxyester, such as α-hydroxyester S13. It is understood that structures given throughout this specification with explicit reference to at least one protection group refer to any one of such structures with all the protecting groups present, with some of the protecting groups present, and with none of the protecting groups present. For example, reference to structure S8 refers to any of: the structre as shown expliclitly in Scheme S-2, such structure with the group P present and the group P′ not present (unprotected OH), such structure with the group P′ present and the group P not present (unprotected OH), and such structure with neither P nor P′ present (both OH groups unprotected). Therefore, although structures S7 and S8 are given explicitly in Scheme S-2, reference to S7 (or to S8) is understood to refer to whichever form S7 or S8 is relevant in the specific context. Protection/deprotection with such groups and the choice of whichever form of protection might be given for a specific reaction step are matters within the ordinary skill in this synthetic technology in light of the teachings provided herein. The chemistry described in Scheme S-1 may also be used to form compounds of formula (I′), wherein additional reactions shown above are used to form the desired pyrazole ring. It is understood that one of ordinary skill in the art will be able to implement the teachings contained herein to supplement Schemes S-1, S-2 and T to make compounds of formula (I′) or (I). Illustrative examples, although not limiting, of such teachings are the teachings provided in the context of the Schemes disclosed above.

Alkylation of the protected mandelic acid derivative S8 is obtained using the methods shown in Scheme S-1. Alkylation of S8 involves treatment with a base such as n-BuLi, LDA, LiHMDS, or NaH, followed by reaction with an appropriate propargyl derivative S9, where X′ is bromide, chloride, iodide, tosylate, or the like (or TMS-protected propargyl bromide as shown in Scheme L) to form product S10. R^{6′} in S13 is introduced through the deprotection step or, where the deprotection step yields the corresponding acid of S13, an ester of S13 is formed in a subsequent step.

Protected hydroxy ester S11 is obtained by coupling suitably substituted activated acid derivative S15 with the addition product S10. HAL in compound S15 is a halo group, more preferably Cl or Br, and most preferably Cl. Alternatively, HAL may be chosen to form a suitably activated amide, such as a Weinreb amide, where HAL is —N(OMe)Me. This coupling is performed in some embodiments of this invention by a Sonogashira reaction. In other embodiments the alkynyl anion may be formed by treatment with a suitable base such as n-BuLi or NaH, and the resulting anion reacted with the corresponding Weinreb amide of S15, for example, where HAL is —N(OMe)Me, to form S11. Starting reagents for the alkylation and acid halide formation reactions are readily available or can be prepared according to methodologies within the ordinary skill in the art.

Sonogashira reaction conditions and catalysts are described herein in, for example, the context of Scheme P.

Regioselectivity with respect to the pyrazole framework in S12 is achieved by a condensation reaction involving compound S11 and a suitably substituted hydrazine S16, where R^{1′} is defined as described herein. See, for example, description of regioselective control in the context of Scheme P. In some embodiments S16 is a suitably substituted hydrazine in other than free base form, referred to herein as non-free base form, in which the hydrazine is in the presence of an acid, thus forming the combinations that these two components form when they are present in the same medium. An example of such embodiments is a suitably substituted hydrazine hydrochloride. In other embodiments, S16 is a suitably substituted hydrazine in free base form. S16 is preferably a suitably substituted hydrazine in non-free base form in embodiments of the process shown in Scheme S-2. Substituent R¹ in S16 is defined above, and it is chosen according to the type of substitution desired in product S12. Alternatively, if hydrazine is used (where R¹ is hydrogen), a subsequent alkylation step following the pyrazole formation allows access to either N-1 or N-2 alkylated pyrazoles using methods known to one skilled in the art. Dehydration of S13 and optional hydrolysis are performed as described in the context of Schemes S-1, S-1A and S2, and the substuent symbols are defined as for compound (I′).

The structure label “S14/(I′)” indicates that S14 represents as shown a structure that falls within the class of structures given by formula (I′), and analogously for the label “SS14/(I′)”.

In a particular embodiment, this invention also encompasses methods for making a compound of formula (I′), esters, enantiomers, diastereomers, racemics, and pharmaceutically acceptable salts thereof, comprising: providing an α-hydroxyester compound of formula T6; and treating said compound of formula T6 with a dehydrating agent.

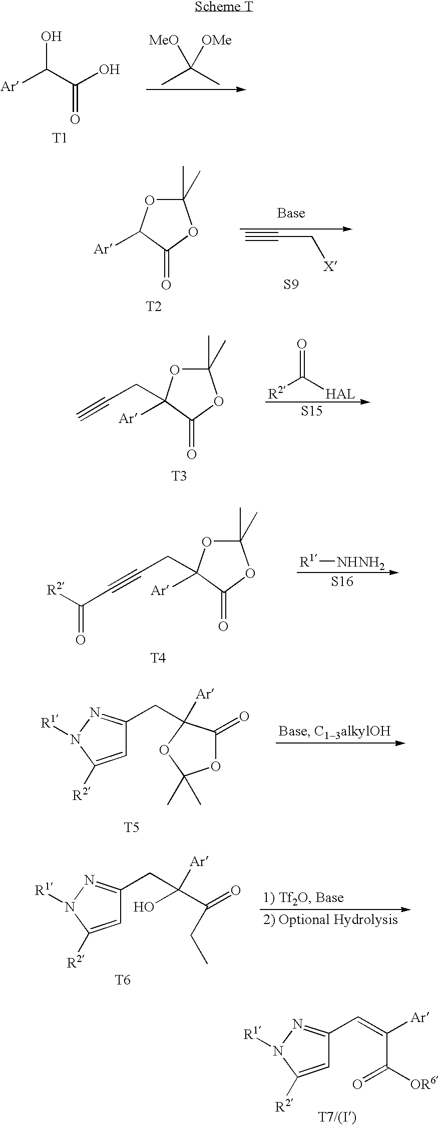

A more specific embodiment of a reaction sequence that includes stereospecific dehydration of an α-hydroxyester is shown in Scheme T, where the substituents and reaction conditions are defined as above for Scheme S-2. The steps that are shown in Scheme T that lead to the formation of T6 are additional illustrative examples of generating an α-hydroxyester, such as α-hydroxyester T6. As indicated above, acids T7, or the corresponding α,β-unsaturated esters, can be processed into compounds of formula (I′) or formula (I) using the procedures taught herein. Substituent symbols in this Scheme are defined as in Schemes above where the same symbols are also used. The structure label “T7/(I′)” indicates that T7 represents as shown a structure that falls within the class of structures given by formula (I′).

In a more particular embodiment, this invention also encompasses methods for making a compound of formula 606, esters, enantiomers, diastereomers, racemics, and pharmaceutically acceptable salts thereof, comprising: providing an α-hydroxyester compound of formula 604; and treating said compound of formula 604 with a dehydrating agent.

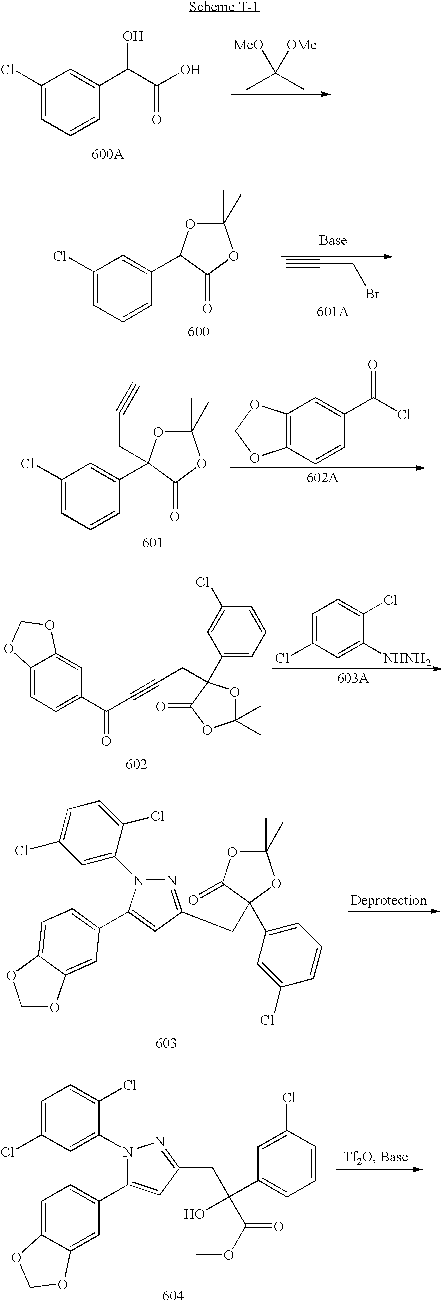

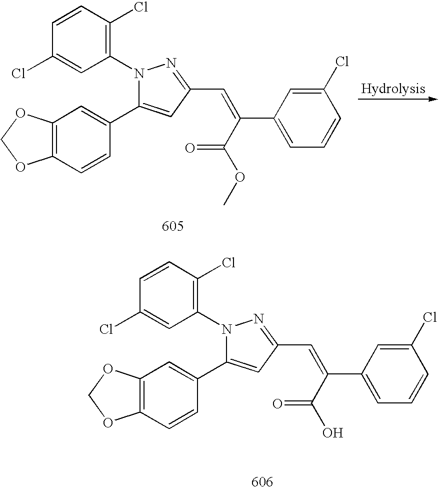

According to Scheme T-1, compound 606 may be prepared using the methods of the invention, as described in the previous schemes, and in Examples 600-606.

Further embodiments of this invention include methods of making compounds of formula (II), esters, enantiomers, diastereomers, racemics and pharmaceutically acceptable salts thereof as described above, further comprising at least any one of the following features:
said dehydrating agent is one of triflic anhydride, fluorosulfonic anhydride, methanesulfonyl chloride, and chemically compatible mixtures thereof;
said dehydrating agent is triflic anhydride in the presence of pyridine;
further comprising a hydrolysis subsequent to said treating with said dehydrating agent;
further comprising obtaining said α-hydroxyester compound of formula S5 by alkylating a mandelic acid analog of formula S2; in more specific embodiments further comprising protecting said mandelic acid analog prior to said alkylating to form a protected alkylated product, and deprotecting said protected alkylated product to form a compound of formula S5; in even more specific embodiments said alkylating comprises treating said mandelic acid analog of formula S2 with one of n-BuLi, LDA, LiHMDS, NaH, and chemically compatible mixtures thereof, and reacting with an alkylating agent S3;
wherein the substituent groups are defined by any one of the definitions of the substituent groups for compounds of formula (II) given herein.

Further embodiments of this invention include methods of making compounds of formula (III), esters, enantiomers, diastereomers, racemics and pharmaceutically acceptable salts thereof as described above, further comprising at least any one of the following features:
said dehydrating agent is one of triflic anhydride, fluorosulfonic anhydride, methanesulfonyl chloride, and chemically compatible mixtures thereof;
said dehydrating agent is triflic anhydride in the presence of pyridine;
further comprising obtaining said α-hydroxyester compound of formula S23 by alkylating a mandelic acid analog of formula S2; in more specific embodiments further comprising protecting said mandelic acid analog prior to said alkylating to form a protected alkylated product, and deprotecting said protected alkylated product to form a compound of formula S23; in even more specific embodiments said alkylating comprises treating said mandelic acid analog of formula S2 with one of n-BuLi, LDA, LiHMDS, NaH, and chemically compatible mixtures thereof, and reacting with a alkylating agent of formula S21;
wherein the substituent groups are defined by any one of the definitions of the substituent groups for compounds of formula (III) given herein.

Further embodiments of this invention include methods of making compounds of formula (I′), esters, enantiomers, diastereomers, racemics and pharmaceutically acceptable salts thereof as described above, further comprising at least any one of the following features:
said dehydrating agent is one of triflic anhydride, fluorosulfonic anhydride, methanesulfonyl chloride, and chemically compatible mixtures thereof;
said dehydrating agent is triflic anhydride in the presence of pyridine;
further comprising a hydrolysis subsequent to said treating with said dehydrating agent;
further comprising obtaining said α-hydroxyester compound of formula S8 by alkylating a mandelic acid analog of formula S7 with an acetylenic halide S9 to form an acetylenic addition compound S10; in more specific embodiments, further comprising coupling said addition compound with a compound of formula S15 to form an addition compound of formula S11; in even more specific embodiments, further comprising condensing said compound of formula S11 with a suitably substituted hydrazine S16 to form a pyrazole derivative;
in even more specific embodiments, further comprising dehydrating said pyrazole derivative to form an ester of compound of formula S14; and in even more specific embodiments, further comprising hydrolyzing said ester of compound of formula S14 to obtain said compound of formula S14;
wherein the substituent groups are defined by any one of the definitions of the substituent groups for compounds of formula (I′) given herein.

Further embodiments of this invention include methods of making compounds of formula (I′), esters, enantiomers, diastereomers, racemics and pharmaceutically acceptable salts thereof as described above, further comprising at least any one of the following features:
further comprising obtaining said α-hydroxyester compound of formula T6 by alkylating a mandelic acid analog of formula T2 with an acetylenic halide S9 to form an acetylenic addition compound T3; in more specific embodiments, further comprising coupling said addition compound T3 with a compound of formula S15 to form an addition compound of formula T4; in even more specific embodiments, further comprising condensing said compound of formula T4 with a suitably substituted hydrazine S16 to form a pyrazole derivative of formula T5; further comprising deprotecting said pyrazole derivative of formula T5;
in even more specific embodiments, further comprising dehydrating said pyrazole derivative of formula T6 to form an ester of compound of formula T7;
and in even more specific embodiments, further comprising hydrolyzing said ester of compound of formula T7 to obtain said compound of formula T7;
wherein the substituent groups are defined by any one of the definitions of the substituent groups for compounds of formula (I′) given herein.

Further embodiments of this invention include methods of making a compound of formula 606, esters, enantiomers, diastereomers, racemics and pharmaceutically acceptable salts thereof as described above, further comprising at least any one of the following features:
further comprising obtaining said α-hydroxyester compound of formula 606 by alkylating a mandelic acid analog of formula 600 with propargyl bromide to form an acetylenic addition compound of formula 601; in more specific embodiments, further comprising coupling said addition compound 601 with a compound of formula 602A to form an addition compound of formula 602; in even more specific embodiments, further comprising condensing said compound of formula 602 with hydrazine 603A to form a pyrazole derivative of formula 603; further comprising deprotecting said pyrazole derivative of formula 603;
in even more specific embodiments, further comprising dehydrating said pyrazole derivative of formula 604 to form an ester of compound of formula 605; and in even more specific embodiments, further comprising hydrolyzing said ester of compound of formula 605 to obtain said compound of formula 606.

One skilled in the art will recognize that compounds of formula (I′) obtained according to any of the Schemes S-2, T, and T-1 are a subset of compounds of formula (I), where n is 0 and R⁵ is —COOH or —COOC₁₋₃alkyl. Additionally, compounds of formula (I′) can further be transformed to obtain the corresponding compounds of formula (I) by saturating the α,β-unsaturated bond in compounds of formula (I′). For example, such saturation can be accomplished by hydrogenation. In other illustrative embodiments, such saturation can be accomplished with a reducing agent, as shown in Scheme H. Alternatively, compounds of formula (I′) or their saturated counterparts may be homologated or otherwise transformed according to the methods shown in Schemes B, C, D, I, K (peptide coupling step), L, P (ester formation and selective hydrolysis), or Q (enzymatic resolution) to obtain compounds of formula (I).

Choice of the more suitable of the Schemes disclosed herein, or of any combination thereof, can be made in light of the teachings provided herein and the form of the desired final product (I), (I′), (II), or (III). For example, embodiments of Scheme P are preferred for a compound with Ar and H substituents at the stereogenic center, such as the title compound in Example 4. As an additional illustration, embodiments of Scheme Q are more suitable for compounds with Ar and another substituent other than H at the stereogenic center, such as the title compound in Example 76. Embodiments of Schemes S-1, S-2, and T are more preferred when the final product is an α,β-unsaturated compound of formula (II) or (I′).

Processes according to the present invention include embodiments in which the regioselective and/or the stereoselective constraints are removed. For example, regioselective reactions involving an inorganic base, a substituted hydrazine, and an acetylenic ketone in a reaction medium that are referred to above as involving a chiral acetylenic ketone to form a chiral pyrazole derivative can also be performed in some embodiments with an acetylenic ketone that has no chirality to form a pyrazole derivative that has no chirality. For example, the title compound in Example 75 illustrates an embodiment of compound (I) in which chirality concerning a single stereogenic center is not relevant because it has no single stereogenic center. Furthermore, when a final chiral compound is desired with no regioselectivity concerns, stereoselective synthetic steps taught herein can be combined with non- or low-regioselective synthetic steps, also taught herein.

During any of the processes for preparation of the compounds of the present invention, it may be necessary and/or desirable to protect sensitive or reactive groups on any of the molecules concerned. In addition, compounds of the invention may be modified by using protecting groups; such compounds, precursors, or prodrugs are also within the scope of the invention. This may be achieved by means of conventional protecting groups, such as those described in “Protective Groups in Organic Chemistry”, ed. J. F. W. McOmie, Plenum Press, 1973; and T. W. Greene & P. G. M. Wuts, “Protective Groups in Organic Synthesis”, 3^{rd} ed., John Wiley & Sons, 1999. The protecting groups may be removed at a convenient subsequent stage using methods known from the art.

Hydroxyl Protecting Groups

Protection for the hydroxyl group includes methyl ethers, substituted methyl ethers, substituted ethyl ethers, substituted benzyl ethers, and silyl ethers.

Substituted Methyl Ethers. Examples of substituted methyl ethers include methyoxymethyl, methylthiomethyl, t-butylthiomethyl, (phenyldimethylsilyl)-methoxymethyl, benzyloxymethyl, p-methoxybenzyl-oxymethyl, (4-methoxy-phenoxy)methyl, guaiacolmethyl, t-butoxymethyl, 4-pentenyloxymethyl, siloxymethyl, 2-methoxyethoxymethyl, 2,2,2-trichloro-ethoxymethyl, bis(2-chloroethoxy)methyl, 2-(trimethylsilyl)-ethoxymethyl, tetrahydropyranyl, 3-bromotetrahydropyranyl, tetrahydrothio-pyranyl, 1-methoxycyclohexyl, 4-methoxytetrahydropyranyl, 4-methoxytetrahydrothio-pyranyl, 4-methoxytetrahydrothiopyranyl S,S-dioxido, 1-[(2-chloro-4-methyl)phenyl]-4-methoxypiperidin-4-yl, 1,4-dioxan-2-yl, tetrahydrofuranyl, tetrahydrothiofuranyl and 2,3,3a,4,5,6,7,7a-octahydro-7,8,8-trimethyl-4,7-methanobenzofuran-2-yl.

Substituted Ethyl Ethers. Examples of substituted ethyl ethers include 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, and benzyl.

Substituted Benzyl Ethers. Examples of substituted benzyl ethers include p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyl-diphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthal-imidophenyl)methyl, 4,4′,4″-tris(levulinoyloxyphenyl)-methyl, 4,4′,4″-tris(benzoyloxyphenyl)methyl, 3-(Imidazol-1-ylmethyl)bis(4′,4″-dimethoxy-phenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, and benzisothiazolyl S,S-dioxido.

Silyl Ethers. Examples of silyl ethers include trimethylsilyl, triethylsilyl, triisopropylsilyl, dimethylisopropylsilyl, diethylisopropylsilyl, dimethylthexylsilyl, t-butyidimethylsilyl, t-butyldiphenylsilyl, tribenzylsilyl, tri-p-xylylsilyl, triphenylsilyl, diphenylmethylsilyl, and t-butylmethoxyphenylsilyl.

Esters. In addition to ethers, a hydroxyl group may be protected as an ester. Examples of esters include formate, benzoylformate, acetate, chloro-acetate, dichloroacetate, trichloroacetate, trifluoroacetate, methoxyacetate, triphenylmethoxyacetate, phenoxyacetate, p-chlorophenoxyacetate, p-P-phenylacetate, 3-phenylpropionate, 4-oxopentanoate(levulinate), 4,4-(ethylene-dithio)pentanoate, pivaloate, adamantoate, crotonate, 4-methoxycrotonate, benzoate, p-phenylbenzoate, 2,4,6-trimethylbenzoate(mesitoate).

Carbonates. Examples of carbonates include methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, and methyl dithiocarbonate.

Assisted Cleavage. Examples of assisted cleavage include 2-iodobenzoate, 4-azidobutyrate, 4-nitro-4-methylpentanoate, o-(dibromo-methyl)benzoate, 2-formylbenzenesulfonate, 2-(methylthiomethoxy)ethyl carbonate, 4-(methylthiomethoxy)butyrate, and 2-(methylthiomethoxy-methyl)benzoate.

Miscellaneous Esters. Examples of miscellaneous esters include 2,6-dichloro-4-methylphenoxyacetate, 2,6-dichloro-4-(1,1,3,3-tetramethylbutyl)-phenoxyacetate, 2,4-bis(1,1-dimethylpropyl)phenoxyacetate, chlorodiphenyl-acetate, isobutyrate, monosuccinoate, (E)-2-methyl-2-butenoate(tigloate), o-(methoxycarbonyl)benzoate, p-P-benzoate, α-naphthoate, nitrate, alkyl N,N,N′,N′-tetramethylphosphorodiamidate, N-phenylcarbamate, borate, dimethylphosphinothioyl, and 2,4-dinitrophenylsulfenate.

Sulfonates. Examples of sulfonates include sulfate, methanesulfonate-(mesylate), benzylsulfonate, and tosylate.

Protection for 1,2- and 1,3-DIOLS

Cyclic Acetals and Ketals. Examples of cyclic acetals and ketals include methylene, ethylidene, 1-t-butylethylidene, 1-phenylethylidene, (4-methoxy-phenyl)ethylidene, 2,2,2-trichloroethylidene, acetonide (isopropylidene), cyclopentylidene, cyclohexylidene, cycloheptylidene, benzylidene, p-methoxybenzylidene, 2,4-dimethoxybenzylidene, 3,4-dimethoxybenzylidene, and 2-nitrobenzylidene.

Cyclic Ortho Esters. Examples of cyclic ortho esters include methoxymethylene, ethoxymethylene, dimethoxymethylene, 1-methoxy-ethylidene, 1-ethoxyethylidine, 1,2-dimethoxyethylidene, α-methoxy-benzylidene, 1-(N,N-dimethylamino)ethylidene derivative, α-(N,N-dimethylamino)benzylidene derivative, and 2-oxacyclopentylidene.

Silyl Derivatives. Examples of silyl derivatives include di-t-butylsilylene group, and 1,3-(1,1,3,3-tetraisopropyldisiloxanylidene) derivative.

Amino Protecting Groups

Protection for the amino group includes carbamates, amides, and special —NH protective groups.

Carbamates. Examples of carbamates include methyl and ethyl carbamates, substituted ethyl carbamates, assisted cleavage carbamates, photolytic cleavage carbamates, urea-type derivatives, and miscellaneous carbamates. Examples of methyl and ethyl carbamates include methyl and ethyl, 9-fluorenylmethyl, 9-(2-sulfo)fluorenylmethyl, 9-(2,7-dibromo)fluorenyl-methyl, 2,7-di-t-butyl-[9-(10,10-dioxo-10,10,10,10-tetrahydrothioxanthyl)]-methyl, and 4-methoxyphenacyl.

Substituted Ethyl. Examples of substituted ethyl carbamates include 2,2,2-trichloroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridyl)ethyl, 2-(N,N-dicyclohexyl-carboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxy-benzyl, p-nitrobenzyl, p-bromobenzyl, p-chlorobenzyl, 2,4-dichlorobenzyl, 4-methylsulfinylbenzyl, 9-anthrylmethyl and diphenylmethyl.

Assisted Cleavage. Examples of assisted cleavage include 2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-chloro-p-acyloxybenzyl, p-(dihydroxyboryl)benzyl, 5-benzisoxazolylmethyl, and 2-(trifluoromethyl)-6-chromonylmethyl.

Photolytic Cleavage. Examples of photolytic cleavage include m-nitrophenyl, 3,5-dimethoxybenzyl, o-nitrobenzyl, 3,4-dimethoxy-6-nitrobenzyl, and phenyl(o-nitrophenyl)methyl.

Urea-Type Derivatives. Examples of urea-type derivatives include phenothiazinyl-(10)-carbonyl derivative, N′-p-toluenesulfonylaminocarbonyl, and N′-phenylaminothiocarbonyl.

Miscellaneous Carbamates. Examples of miscellaneous carbamates include t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethylcarboxamido)benzyl, 1,1-dimethyl-3-(N,N-dimethylcarboxamido)propyl, 1,1-dimethylpropynyl, di(2-pyridyl)methyl, 2-furanylmethyl, 2-iodoethyl, isobornyl, isobutyl, isonicotinyl, p-(p′-methoxy-phenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, and 2,4,6-trimethylbenzyl.

Amides. Examples of amides include N-formyl, N-acetyl, N-chloroacetyl, N-trichloroacetyl, N-trifluoroacetyl, N-phenylacetyl, N-3-phenylpropionyl, N-picolinoyl, N-3-pyridylcarboxamide, N-benzoylphenylalanyl derivative, N-benzoyl, N-p-phenylbenzoyl.

Assisted Cleavage. N-o-nitrophenylacetyl, N-o-nitrophenoxyacetyl, N-acetoacetyl, (N′-dithiobenzyloxycarbonylamino)acetyl, N-3-(p-hydroxyphenyl)-propionyl, N-3-(o-nitrophenyl)propionyl, N-2-methyl-2-(o-nitrophenoxy)-propionyl, N-2-methyl-2-(o-phenylazophenoxy)propionyl, N-4-chlorobutyryl, N-3-methyl-3-nitrobutyryl, N-o-nitrocinnamoyl, N-acetylmethionine derivative, N-o-nitrobenzoyl, N-o-(benzoyloxymethyl)benzoyl, and 4,5-diphenyl-3-oxazolin-2-one.

Cyclic Imide Derivatives. N-phthalimide, N-dithiasuccinoyl, N-2,3-diphenylmaleoyl, N-2,5-dimethylpyrrolyl, N-1,1,4,4-tetramethyldisilylaza-cyclopentane adduct, 5-substituted 1,3-dimethyl-1,3,5-triazacyclohexan-2-one, 5-substituted 1,3-dibenzyl-1,3,5-triazacyclohexan-2-one, and 1-substituted 3,5-dinitro-4-pyridonyl.

Special—NH Protective Groups

Examples of special NH protective groups include:

N-Alkyl and N-Aryl Amines. N-methyl, N-allyl, N-[2-(trimethylsilyl)ethoxy]methyl, N-3-acetoxypropyl, N-(1-isopropyl-4-nitro-2-oxo-3-pyrrolin-3-yl), quaternary ammonium salts, N-benzyl, N-4-methoxybenzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, and N-2-picolylamine N′-oxide.

Imine Derivatives. N-1,1-dimethylthiomethylene, N-benzylidene, N-p-methoxybenzylidene, N-diphenylmethylene, N-[(2-pyridyl)mesityl]methylene, and N—(N′,N′-dimethylaminomethylene).

Protection for the Carbonyl Group

Acyclic Acetals and Ketals. Examples of acyclic acetals and ketals include dimethyl, bis(2,2,2-trichloroethyl), dibenzyl, bis(2-nitrobenzyl) and diacetyl.

Cyclic Acetals and Ketals. Examples of cyclic acetals and ketals include 1,3-dioxanes, 5-methylene-1,3-dioxane, 5,5-dibromo-1,3-dioxane, 5-(2-pyridyl)-1,3-dioxane, 1,3-dioxolanes, 4-bromomethyl-1,3-dioxolane, 4-(3-butenyl)-1,3-dioxolane, 4-phenyl-1,3-dioxolane, 4-(2-nitrophenyl)-1,3-dioxolane, 4,5-dimethoxymethyl-1,3-dioxolane, O, O′-phenylenedioxy and 1,5-dihydro-3H-2,4-benzodioxepin.

Acyclic Dithio Acetals and Ketals. Examples of acyclic dithio acetals and ketals include S,S′-dimethyl, S,S′-diethyl, S,S′-dipropyl, S,S′-dibutyl, S,S′-dipentyl, S,S′-diphenyl, S,S′-dibenzyl and S,S′-diacetyl.

Cyclic Dithio Acetals and Ketals. Examples of cyclic dithio acetals and ketals include 1,3-dithiane, 1,3-dithiolane and 1,5-dihydro-3H-2,4-benzo-dithiepin.

Acyclic Monothio Acetals and Ketals. Examples of acyclic monothio acetals and ketals include O-trimethylsilyl-5-alkyl, O-methyl-5-alkyl or —S-phenyl and O-methyl-S-2-(methylthio)ethyl.

Cyclic Monothio Acetals and Ketals. Examples of cyclic monothio acetals and ketals include 1,3-oxathiolanes.

Miscellaneous Derivatives

O-Substituted Cyanohydrins. Examples of O-substituted cyanohydrins include O-acetyl, O-trimethylsilyl, O-1-ethoxyethyl and O-tetrahydropyranyl.

Substituted Hydrazones. Examples of substituted hydrazones include N,N-dimethyl and 2,4-dinitrophenyl.

Oxime Derivatives. Examples of oxime derivatives include O-methyl, O-benzyl and O-phenylthiomethyl.

Imines

Substituted Methylene Derivatives, Cyclic Derivatives. Examples of substituted methylene and cyclic derivatives include oxazolidines, 1-methyl-2-(1′-hydroxyalkyl)imidazoles, N,N′-dimethylimidazolidines, 2,3-dihydro-1,3-benzothiazoles, diethylamine adducts, and methylaluminum bis(2,6-di-t-butyl-4-methylphenoxide)(MAD) complex.

Monoprotection of Dicarbonyl Compounds

Selective Protection of α- and β-Diketones. Examples of selective protection of α- and β-diketones include enamines, enol acetates, enol ethers, methyl, ethyl, i-butyl, piperidinyl, morpholinyl, 4-methyl-1,3-dioxolanyl, pyrrolidinyl, benzyl, S-butyl, and trimethylsilyl.

Cyclic Ketals, Monothio and Dithio Ketals. Examples of cyclic ketals, monothio and dithio ketals include bismethylenedioxy derivatives and tetramethylbismethylenedioxy derivatives.

Protection for the Carboxyl Group

Esters

Substituted Methyl Esters. Examples of substituted methyl esters include 9-fluorenylmethyl, methoxymethyl, methylthiomethyl, tetrahydropyranyl, tetrahydrofuranyl, methoxyethoxymethyl, 2-(trimethylsilyl)ethoxymethyl, benzyl-oxymethyl, phenacyl, p-bromophenacyl, α-methylphenacyl, p-methoxyphen-acyl, carboxamidomethyl, and N-phthalimidomethyl.

2-Substituted Ethyl Esters. Examples of 2-substituted ethyl esters include 2,2,2-trichloroethyl, 2-haloethyl, Ω-chloroalkyl, 2-(trimethylsilyl)ethyl, 2-methylthioethyl, 1,3-dithianyl-2-methyl, 2-(p-nitrophenylsulfenyl)ethyl, 2-(p-toluenesulfonyl)ethyl, 2-(2′-pyridyl)ethyl, 2-(diphenylphosphino)ethyl, 1-methyl-1-phenylethyl, t-butyl, cyclopentyl, cyclohexyl, allyl, 3-buten-1-yl, 4-(trimethylsilyl)-2-buten-1-yl, cinnamyl, α-methylcinnamyl, phenyl, p-(methylmercapto)phenyl and benzyl.

Substituted Benzyl Esters. Examples of substituted benzyl esters include triphenylmethyl, diphenylmethyl, bis(o-nitrophenyl)methyl, 9-anthrylmethyl, 2-(9,10-dioxo)anthrylmethyl, 5-dibenzosuberyl, 1-pyrenylmethyl, 2-(trifluoromethyl)-6-chromylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-sulfobenzyl, piperonyl, 4-picolyl and p-P-benzyl.

Silyl Esters. Examples of silyl esters include trimethylsilyl, triethylsilyl, t-butyldimethylsilyl, i-propyldimethylsilyl, phenyldimethylsilyl and di-t-butylmethylsilyl.

Activated Esters. Examples of activated esters include thiols.

Miscellaneous Derivatives. Examples of miscellaneous derivatives include oxazoles, 2-alkyl-1,3-oxazolines, 4-alkyl-5-oxo-1,3-oxazolidines, 5-alkyl-4-oxo-1,3-dioxolanes, ortho esters, phenyl group and pentaminocobalt(III) complex.

Stannyl Esters. Examples of stannyl esters include triethylstannyl and tri-n-butylstannyl.

Amides and Hydrazides

Amides. Examples of amides include N,N-dimethyl, pyrrolidinyl, piperidinyl, 5,6-dihydrophenanthridinyl, o-nitroanilides, N-7-nitroindolyl, N-8-Nitro-1,2,3,4-tetrahydroquinolyl, and p-P-benzenesulfonamides.

Hydrazides. Examples of hydrazides include N-phenyl and N,N′-diisopropyl.

Compounds of the present invention may be used in pharmaceutical compositions to treat patients (humans and other mammals) with disorders involving the action of the CCK-1 receptor. As CCK-1 receptor modulators the compounds may be divided into compounds, which are pure or partial agonists and compounds that are antagonists. Where the compound is a CCK-1 receptor antagonist, it may be used in the treatment of pain, drug dependence, anxiety, panic attack, schizophrenia, pancreatic disorder, secretory disorder, motility disorders, functional bowel disease, biliary colic, anorexia and cancer. Where the compound is a CCK-1 receptor agonist, it may be used in the treatment of obesity, hypervigilance and gallstones.

The preferred route is oral administration, however compounds may be administered by intravenous infusion or topical administration. Oral doses range from about 0.05 to 100 mg/kg, daily, taken in 1-4 separate doses. Some compounds of the invention may be orally dosed in the range of about 0.05 to about 50 mg/kg daily, while others may be dosed at 0.05 to about 20 mg/kg daily. Infusion doses can range from about 1.0 to 1.0×10⁴ μg/kg/min of inhibitor, admixed with a pharmaceutical carrier over a period ranging from several minutes to several days. For topical administration compounds of the present invention I may be mixed with a pharmaceutical carrier at a concentration of about 0.1 to about 10% of drug to vehicle.

The pharmaceutical compositions can be prepared using conventional pharmaceutical excipients and compounding techniques. Oral dosage forms may be elixers, syrups, capsules tablets and the like. Where the typical solid carrier is an inert substance such as lactose, starch, glucose, methylcellulose, magnesium sterate, dicalcium phosphate, mannitol and the like; and typical liquid oral excipients include ethanol, glycerol, water and the like. All excipients may be mixed as needed with disintegrants, diluents, granulating agents, lubricants, binders and the like using conventional techniques known to those skilled in the art of preparing dosage forms. Parenteral dosage forms may be prepared using water or another sterile carrier.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions.

### EXAMPLES

NMR spectra were obtained on either a Bruker model DPX400 (400 MHz) or DPX500 (500 MHz) spectrometer. The format of the ¹H NMR data below is: chemical shift in ppm down field of the tetramethylsilane reference (multiplicity, coupling constant J in Hz, integration).

Mass spectra were obtained on an Agilent series 1100 MSD using electrospray ionization (ESI) in either positive or negative mode as indicated. The “mass calculated” for a molecular formula is the monoisotopic mass of the compound.

Reported HPLC retention times (Rₜ) are in minutes with the following methods:

Protocol for Reversed-Phase HPLC (Method A):

Instrument: Agilent HPLC 1100;
Column: Zorbax Eclipse XDB-C8, 5 μm, 4.6×150 mm;
Flow rate: 0.75 mL/min; x=220 & 254 nm;

Gradient (Acetonitrile/Water):

| | | |
|---|---|---|
| 1) | 0.0 min | 1% Acetonitrile |
| 2) | 8.0 min | 99% Acetonitrile |
| 3) | 12.0 min | 99% Acetonitrile |

Protocol for Reversed-Phase HPLC (Method B):
Instrument: Agilent HPLC 1100;
Column: Xterra™, RP18, 3.5 μm, 4.6×50 mm;
Flow rate: 1.5 mL/min; λ=220 & 254 nm;

Gradient (Acetonitrile/Water):

| | | |
|---|---|---|
| 1) | 0.0 min | 85% Acetonitrile |
| 2) | 3.5 min | 1.0% Acetonitrile |
| 3) | 5 min | 1.0% Acetonitrile |

Protocol for Chiral HPLC (Method C):
Instrument: Agilent HPLC 1100;
Chiral Column: Chiralpak AD, 4.6×250 mm;
Column Manufacturer: Chiral Technologies Inc.;
Mobile Phase: 85:15 Ethanol/Hexane with 0.1% TFA;
Flow Rate: 0.75 mL/min; λ=220 & 254 nm
   Protocol for Semi-Preparation. Chiral HPLC (Method D):
Instrument: Agilent HPLC 1100;
Chiral Column: Chiralpak AD, 20×250 mm;
Column Manufacturer: Chiral Technologies Inc.;
Mobile Phase: 85:15 Ethanol/Hexane with 0.1% TFA;
Flow Rate: 7 mL/min; λ=220 & 254 nm
   Reversed-Phase HPLC (Method E):
Column: Zorbax Eclipse XDB-C8, 5 μm, 4.6×150 mm;
Flow rate: 0.75 mL/min; λ=220 & 254 nm;

Gradient (Acetonitrile/Water):

| | | |
|---|---|---|
| 1) | 8.0 min | 1%-99% Acetonitrile |
| 2) | 10.0 min | 99% Acetonitrile |

Chiral HPLC (Method F):
Column: Chiralcel AD 0.46×25 cm;
Mobile Phase: 85:15 Ethanol/Hexane with 0.07% TFA;
Flow rate: 1 mL/min; λ=220 & 254 nm
   Reversed-Phase HPLC (Method G):
Column: XTerra Prep MS C18, 5 μm, 19×50 mm;
Mobile Phase: Acetonitrile/Water with 0.1% TFA;
Flow rate: 25 mL/min; λ=220 & 254 nm;

Gradient:

| | | |
|---|---|---|
| 1) | 0.0 min | 15% Acetonitrile |
| 2) | 13.0 min | 99% Acetonitrile |
| 3) | 15.0 min | 99% Acetonitrile |

Protocol for Reversed-Phase HPLC (Method H):
Manufactured by Agilent HPLC 1100;
Column: Chromolith SpeedROD, 4.6×50 mm;
Mobile Phase: Acetonitrile/Water with 0.1% TFA;
Flow rate: 5 mL/min; λ=220 & 254 nm;

Gradient (Acetonitrile/Water):

| | | |
|---|---|---|
| 1) | 0.0 min | 85% Acetonitrile |
| 2) | 2.0 min | 1.0% Acetonitrile |
| 3) | 2.5 min | 1.0% Acetonitrile |

Protocol for Reversed-Phase HPLC (Method I):
Manufactured by Agilent HPLC 1100;
Column: Xterra™, RP18, 3.5 μm, 4.6×50 mm;
Mobile Phase: Acetonitrile/Water with 10 mM NH₄OH;
Flow rate: 1 mL/min; λ=220 & 254 nm;

Gradient (Acetonitrile/Water):

| | | |
|---|---|---|
| 1) | 0.0 min | 1% Acetonitrile |
| 2) | 7.0 min | 99% Acetonitrile |
| 3) | 10.0 min | 99% Acetonitrile |

HPLC Method J; (Chiral)
Chiralcel AD 0.46 cm×25 cm column
Flow rate: 1 mL/min; λ=220 nm & 254 nm
Solvent: 60/40 EtOH/Hexane
Gradient conditions: Isocratic

### Example 1

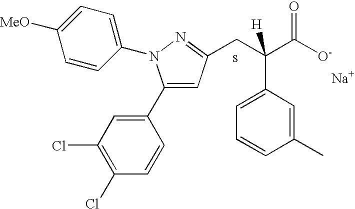

### (S)-Sodium; 3-[5-(3,4-dichloro-phenyl)-1-(4-methoxy-phenyl)-1H-pyrazol-3-yl]-2-m-tolyl-propionate

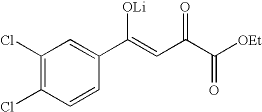

A. Lithium 4-(3,4-dichlorophenyl)-4-hydroxy-2-oxo-but-3-enoic acid ethyl ester. In a dried 1-L round-bottomed flask, lithium bis(trimethylsilyl)amide in tetrahydrofuran (THF) (265 mL, 0.265 mol) was concentrated under reduced pressure to a solid using a rotary evaporator at 25-30° C. Anhydrous diethyl ether (200 mL) was added and this stirred suspension of LHMDS in diethyl ether was cooled to −78° C. under N₂. 3,4-Dichloracetophenone (50.0 g, 0.265 mol) in diethyl ether (200 mL) was slowly added to the reaction mixture over 15 min. The mixture was allowed to stir for 60 min, and diethyl oxalate (36.0 mL, 0.265 mol) in diethyl ether (75 mL) was then added over 20 min. After 90 min, the mixture was allowed to warm to room temperature (rt) and stirred overnight. The light yellow solids were filtered, washed with diethyl ether and dried in vacuum to afford 78.4 g of lithium 4-(3,4-dichlorophenyl)-4-hydroxy-2-oxo-but-3-enoic acid ethyl ester as a white solid. This material was used in the next step without further purification.

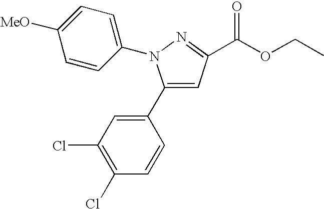

B. 5-(3,4-Dichloro-phenyl)-1-(4-methoxy-Phenyl)-1H-pyrazole-3-carboxylic acid ethyl ester. A stirred suspension of lithium 4-(3,4-dichlorophenyl)-4-hydroxy-2-oxo-but-3-enoic acid ethyl ester (90.7 g, 0.307 mol) and 4-methoxyphenyl hydrazine hydrochloride (54.0 g, 0.309 mol) in EtOH (600 mL) was heated to 55° C. for 5 h then stirred at rt overnight. HPLC analysis showed a 4:1 mixture of 5-(3,4-dichloro-phenyl)-1-(4-methoxy-phenyl)-1H-pyrazole-3-carboxylic acid ethyl ester and 5-(3,4-dichloro-phenyl)-2-(4-methoxy-phenyl)-2H-pyrazole-3-carboxylic acid ethyl ester. The precipitated solids were filtered and washed with EtOH. The solids were recrystallized with 1:1 CH₃CN/MeOH to recover 9.0 g of minor product 5-(3,4-dichloro-phenyl)-2-(4-methoxy-phenyl)-2H-pyrazole-3-carboxylic acid ethyl ester. Crystallization was repeated several times to recover 71.0 g of major product 5-(3,4-dichloro-phenyl)-1-(4-methoxy-phenyl)-1H-pyrazole-3-carboxylic acid ethyl ester. The crude filtrate was purified by column chromatography (silica gel, 4:1 hexane/ethyl acetate (EtOAc)) to recover another 17.6 g of 5-(3,4-dichloro-phenyl)-1-(4-methoxy-phenyl)-1H-pyrazole-3-carboxylic acid ethyl ester for a total combined yield of 74%. HPLC: Rₜ=10.57 (Method E). MS (ES+): mass calculated for C₁₉H₁₆Cl₂N₂O₃, 391.25; m/z found 392.3 [M+H]⁺. ¹H NMR (400 MHz, CDCl₃): 7.37 (d, J=2.0 Hz, 1H), 7.35 (d, J=8.4 Hz, 1H), 7.26-7.22 (m, 2H), 7.04 (s, 1H), 6.97 (dd, J=8.0, 1.0 Hz, 1H), 6.95-6.88 (m, 2H), 4.45 (q, J=7.1 Hz, 2H), 3.84 (s, 3H), 1.42 (q, J=7.1 Hz, 3H).

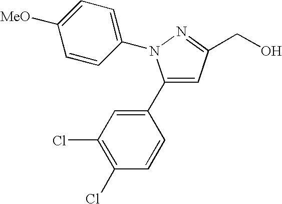

C. [5-(3,4-Dichlorophenyl)-1-(4-methoxyphenyl)-1H-pyrazol-3-yl]-methanol. To a stirred solution of 5-(3,4-dichloro-phenyl)-1-(4-methoxy-phenyl)-1H-pyrazole-3-carboxylic acid ethyl ester (55.7 g, 0.140 mol) in THF (150 mL) at −78° C. under N₂ was slowly added a 1.0 M solution of diisobutylaluminum hydride (DIBAL-H) (350 mL, 0.35 mol) over 45 min. The solution was allowed to stir for 20 min then warmed to rt over 90 min. The mixture was then cooled to 0° C., and a saturated solution of potassium sodium tartrate (300 mL) and EtOAc (400 mL) was added. The slurry mixture was stirred overnight whereupon both layers became clear. The organic layer was extracted with EtOAc (2×75 mL), dried with Na₂SO₄, filtered and concentrated. The crude product was dried under vacuum to recover 46.8 g (96%) of the title compound. This was used in the next step without further purification. HPLC: Rₜ=9.16 (Method E). MS (ES+): mass calculated for C₁₇H₁₄Cl₂N₂O₂, 349.21; m/z found 371.1 [M+Na]⁺. ¹H NMR (400 MHz, CDCl₃): 7.39 (d, J=2.1 Hz, 1H), 7.34 (d, J=3.6 Hz, 1H), 7.20-7.09 (m, 2H), 6.97 (dd, J=8.36, 2.1 Hz, 1H), 6.91-6.79 (m, 2H), 6.43 (s, 1H), 4.69 (s, 2H), 3.74 (s, 3H).

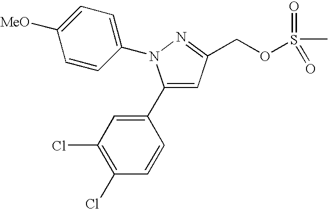

D. Methanesulfonic acid 5-(3,4-dichlorophenyl)-1-(4-methoxyphenyl)-1H-pyrazol-3-ylmethyl ester. To a stirred solution of [5-(3,4-dichlorophenyl)-1-(4-methoxyphenyl)-1H-pyrazol-3-yl]-methanol (7.2 g, 0.021 mol) in THF (125 mL) and triethylamine (TEA) (4.6 mL, 0.033 mol) was added methanesulfonyl chloride (2.5 mL, 0.031 mol). The reaction mixture was stirred at 45° C. for 4 h. The reaction mixture was cooled to rt, quenched with H₂O (75 mL) then washed with EtOAc (3×50 mL). The organic layer was dried over Na₂SO₄, filtered and concentrated to oil. This crude pyrazole mesylate was used in the next step without further purification. HPLC: Rₜ=10.03 (Method E). MS (ES+): mass calculated for C₁₈H₁₈Cl₂N₂O₄S, 427.30; m/z found 428.1 [M+H]⁺.

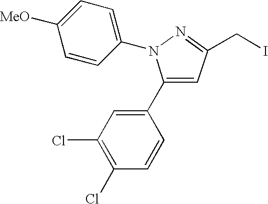

E. 5-(3,4-Dichloro-phenyl)-3-iodomethyl-1-(4-methoxy-phenyl)-1H-pyrazole. A stirred solution of methanesulfonic acid 5-(3,4-dichlorophenyl)-1-(4-methoxyphenyl)-1H-pyrazol-3-ylmethyl ester (8.80 g, 0.0206 mol) and NaI (4.64 g, 0.0309 mol) in acetone (175 mL) was refluxed for 90 min. The thick reaction slurry was cooled to rt, quenched with H₂O (200 mL) and extracted with EtOAc (3×75 mL). The organic layer was dried with Na₂SO₄, filtered and concentrated to a dark oil. The crude oil was purified by column chromatography (silica gel, 85:15 hexane/EtOAc) to obtain 9.15 g (97%) of the title compound after two steps. HPLC: Rₜ=11.03 (Method E). MS (ES+): mass calculated for C₁₇H₁₃Cl₂IN₂O, 459.10; m/z found 460.9 [M+H]⁺. ¹H NMR (400 MHz, CDCl₃): 7.37 (d, J=2.0 Hz, 1H), 7.34 (d, J=8.3 Hz, 1H), 7.18 (d, J=8.8 Hz, 2H), 6.95 (dd, J=6.3, 2.0 Hz, 1H), 6.88 (d, J=9.1 Hz, 2H), 6.55 (s, 1H), 4.47 (s, 2H), 3.83 (s, 3H).

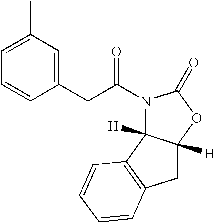

F. (3a S,8aR)-3-(2-m-Tolyl-acetyl)-3,3a,8,8a-tetrahydro-indeno[1,2-d]oxazol-2-one. To a stirred solution of m-tolylacetic acid (8.57 g, 0.0571 mol), 2-chloro-1-methylpyridinium iodide (19.0 g, 0.0744 mol) and (3aS-cis)-(−)-3,3a,8,8a-tetrahydro-2H-indeno[1,2-d]-oxazol-2-one (10.0 g, 0.0571 mol) in CH₂Cl₂ (130 mL) were added TEA (18.0 mL, 0.129 mol) and 4-dimethylaminopyridine (DMAP, 1.39 g, 0.0114 mol) at 0° C. The reaction mixture was stirred at rt for 3 h then treated with hexane (130 mL). The resulting slurry was passed through a pad of silica gel, eluting with 3:2 EtOAc/hexane. The filtrate was concentrated to an oil and recrystallized in hot hexane to recover 13 g (74%) of the title compound as a white solid. HPLC: Rₜ=9.85 (Method E). MS (ES+): mass calculated for C₁₉H₁₇NO₃, 307.36; m/z found 330.2 [M+Na]⁺. ¹H NMR (400 MHz, CDCl₃): 7.65 (d, J=7.6 Hz, 1H), 7.08-7.37 (m, 7H), 5.95 (d, J=6.8 Hz, 1H), 5.27-5.31 (m, 1H), 4.26 (dd, J=15.9, 39.1 Hz, 2H), 3.40 (d, J=3.5 Hz, 2H), 2.34 (s, 3H).

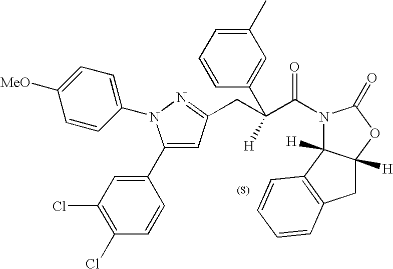

G. (2S,3aS,8aR)-3-{3-[5-(3,4-Dichlorophenyl)-1-(4-methoxyphenyl)-1H-pyrazol-3-yl]-2-m-tolyl-propionyl}-3,3a,8,8a-tetrahydro-indeno[1,2-d]oxazol-2-one. To a stirred solution of (3aS,8aR)-3-(2-m-tolyl-acetyl)-3,3a,8,8a-tetrahydro-indeno[1,2-d]oxazol-2-one (product of Step F, 12 g, 0.039 mol) in THF (100 mL) was added 1.0 M sodium 1,1,1,3,3,3-hexamethyldisilazane (NaHMDS) (41 mL, 0.041 mol) in THF at −78° C. The mixture was stirred for 45 min at −78° C. then treated with 5-(3,4-dichloro-phenyl)-3-iodomethyl-1-(4-methoxy-phenyl)-1-H-pyrazole (product of Step E, 18.4 g, 0.0405 mol) in THF (100 mL). The reaction mixture was allowed to warm to rt overnight and then was quenched with H₂O (100 mL) and concentrated to half the volume. The aqueous layer was washed with EtOAc (3×75 mL). The extracted organic layer was washed with saturated NaCl, dried over Na₂SO₄, filtered and concentrated to an oil. The crude oil was purified by flash column chromatography (silica gel, 7:3 hexane/EtOAc) to obtain 20.7 g of the title compound (83%) as white foam. HPLC: Rₜ=11.38 (Method E). MS (ES+): mass calculated for C₃₆H₂₉Cl₂N₃O₄, 638.55; m/z found 660.3 [M+Na]⁺. ¹H NMR (400 MHz, CDCl₃): 7.52 (d, J=7.6 Hz, 1H), 7.11-7.35 (m, 8H), 6.93-6.99 (m, 3H), 6.74-6.82 (m, 3H), 6.20 (s, 1H), 5.89 (d, J=6.8 Hz, 1H), 5.58 (q, J=6.1, 4.5 Hz, 1H), 5.11-5.15 (m, 1H), 3.8 (s, 3H), 3.72 (dd, J=10.6, 4.1 Hz, 1H), 3.33 (br, s, 2H), 3.07 (dd, J=9.8, 4.8 Hz, 1H), 2.37 (s, 3H).

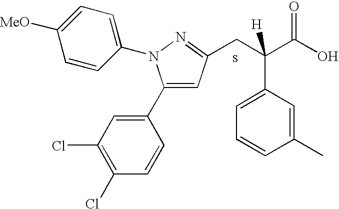

H. (S)-3-[5-(3,4-Dichloro-phenyl)-1-(4-methoxy-phenyl)-1H-pyrazol-3-yl]-2-m-tolyl-propionic acid. To a stirred solution of (2S,3aS,8aR)-3-{3-[5-(3,4-dichlorophenyl)-1-(4-methoxyphenyl)-1H-pyrazol-3-yl]-2-m-tolyl-propionyl}-3,3a,8,8a-tetrahydro-indeno[1,2-d]oxazol-2-one (20.7 g, 0.0323 mol) in THF (230 mL) and H₂O (45 mL) at 0° C. was added 30% H₂O₂ (15.0 mL, 0.147 mol) followed by LiOH hydrate (2.75 g, 0.0655 mol) in H₂O (15 mL). The reaction mixture was allowed to warm to rt and was stirred for 90 min. The mixture was cooled to 0° C. and then quenched with 1.5 N Na₂SO₃ (20 mL) maintaining pH 9-10. The mixture was concentrated to ¹/4 volume, then treated with H₂O (200 mL) and acidified to pH 1-2 using 3 N HCl. The aqueous layer was washed with EtOAc (3×100 mL). The combined organic layers were dried with Na₂SO₄, filtered and concentrated to ¼ volume. Solid crystals that developed overnight were filtered and washed with cold 1:1 hexane/EtOAc. The chiral auxiliary was recovered in 66% yield (3.72 g). The filtrate was purified by flash chromatography (7:3 hexane/EtOAc with 0.3% MeOH) to afford 12.7 g (81.5%) of (S)-3-[5-(3,4-dichloro-phenyl)-1-(4-methoxy-phenyl)-1H-pyrazol-3-yl]-2-m-tolyl-propionic acid as orange oil. HPLC: Rₜ=10.44 (Method E). MS (ES+): mass calculated for C₂₆H₂₂Cl₂N₃, 481.37; m/z found 503.2 [M+Na]⁺. ¹H NMR (400 MHz, CDCl₃): 7.12-7.31 (m, 9H), 6.90 (dd, J=6.3, 2.0 Hz, 1H), 6.86 (d, J=9.1 Hz, 2H), 6.21 (s, 1H), 4.07-4.15 (m, 1H), 3.82 (s, 3H), 3.53 (dd, J=9.3, 5.3 Hz, 1H), 3.10 (dd, J=9.1, 5.8 Hz, 1H), 2.35 (s, 3H).

I. (S)-Sodium; 3-[5-(3,4-dichloro-phenyl)-1-(4-methoxy-phenyl)-1H-pyrazol-3-yl]-2-m-tolyl-propionate. To a stirred solution of (S)-3-[5-(3,4-dichloro-phenyl)-1-(4-methoxy-phenyl)-1H-pyrazol-3-yl]-2-m-tolyl-propionic acid (12.7 g, 0.0264 mol) in THF (125 mL) was added aqueous NaOH (1.05 g, 0.0264 mol in H₂O, 10 mL) at 0° C. The mixture was stirred for 30 min at 0° C. then concentrated to an oil under reduced pressure using a rotary evaporator (25-30° C.). The oil was diluted in THF (150 mL), chilled in an ice bath and CH₃CN (50 mL) was added where upon a precipitate developed. The suspension was stirred for 2 h, filtered and then washed with CH₃CN to afford 10.9 g (67%) of the title compound as a white solid. HPLC: Rₜ=7.10 (Method F). HRMS: exact mass of [M+H]+calculated for C₂₆H₂₂Cl₂N₂O₃, 481.1086; m/z found, 481.1079. M.P. 295.5-297.5° C. Anal. Calcd for C₂₅H₁₈Cl₂N₂NaO₃: C, 61.49; H, 3.72; N, 5.74. Found: C, 61.98; H, 4.14; N, 5.43. Optical rotation [α]²⁰₅₈₉+58.8°(c=0.1, EtOH). ¹H NMR (400 MHz, D₂O); 6.90-6.93 (m, 2H), 6.77 (t, J=7.3 Hz, 1H), 6.61 (d, J=9.1 Hz, 2H), 6.53 (d, J=7.3 Hz, 1H), 6.38 (t, J=8.6 Hz, 4H), 6.12 (d, J=8.1 Hz, 1H), 5.46 (s, 1H), 3.55-3.63 (m, 1H), 3.22 (s, 3H), 3.06-3.18 (m, 2H), 1.81 (s, 3H). ¹³C NMR (100 MHz. DMSO-d₆): 175.3, 157.9, 152.5, 143.6, 139.2, 135.7, 132.1, 130.7, 130.5, 130.1, 130.0, 129.2, 128.0, 127.7, 126.9, 126.1, 125.4, 124.5, 113.7,107.0, 54.9, 54.5, 32.6, 20.6 ppm.

Method 1

### Synthesis of 3-Bromomethyl-1,5-diaryl-1H-pyrazoles (Pyrazole Bromides)

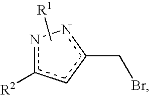 such as:

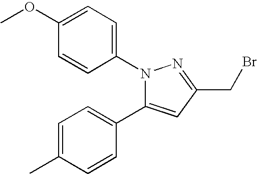

### 3-Bromomethyl-1-(4-methoxy-phenyl)-5-p-tolyl-1H-pyrazole

A solution of phosphorus tribromide (9.31 g, 34.5 mmol) in CH₂Cl₂ (186 mL) was added drop-wise to a stirred solution of [1-(4-methoxy-phenyl)-5-p-tolyl-1H-pyrazol-3-yl]-methanol (7.80 g, 26.5 mmol; prepared analogously to the procedure described in Step C of Example 1) in 50 mL CH₂Cl₂ at 0° C. The reaction mixture was stirred for an additional 18 h at rt, and then the mixture was neutralized by addition of 40% NaOH with cooling in an ice bath. The organic layer was separated and dried over Na₂SO₄, and solvent was removed under reduced pressure. The residue was purified by silica gel chromatography (CH₂Cl₂) yielding 8.09 g (86%) of 3-bromomethyl-1-(4-methoxy-phenyl)-5-p-tolyl-1H-pyrazole. HPLC: Rₜ=10.38. (Method A). MS (ES+): mass calculated for C₁₈H₁₇BrN₂O, 356.05; m/z found 357.5 [M+H]⁺. ¹H NMR (400 MHz, CDCl₃): 7.42 (s, 4H), 7.39-7.34 (m, 2H), 7.02-6.98 (m, 2H), 6.69 (s, 2H), 4.73 (s, 2H), 3.97 (s, 1H), 2.49 (s, 3H).

Method 2

### General Method for the Synthesis of 3-(1,5-Diaryl-1H-pyrazol-3-yl)-2-aryl-propionic Acids (A9)

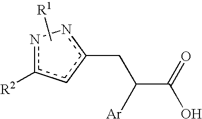

Scheme A. In each of eight 10-mL test tubes, 60% NaH in mineral oil (18 mg, 0.45 mmol) was suspended in 5 mL of N,N-dimethylformamide (DMF) at 0° C. under N₂. Then, to each test tube, a unique phenyl-acetic acid ester (A10) was added, and the reaction mixtures were stirred for 1 h. Equal portions of the first such mixture were then loaded into the six wells of the first row of a 48-well Robbins block under N₂, and equal portions of the next mixture were loaded into the six wells of the second row, and so on, until all eight reaction mixtures had been apportioned, and all forty-eight wells had been loaded. Then, 0.15 mmol of one of six different pyrazole bromides (A7, prepared analogously to the procedure described in Method 1) in 0.5 mL DMF was loaded into each of eight wells of the first of six orthogonal columns of the block, and 0.15 mmol of a second pyrazole bromide in 0.5 mL DMF was loaded into each of eight wells of the second column of the block, and so on, yielding a matrix of forty-eight unique reaction mixtures. After the block was shaken for 18 h at rt, 0.3 mL of 2 M aqueous LiOH was added to each well, and the block was shaken an additional 18 h at rt. The solutions were drained into the 48 wells of a Beckman microtiter collection plate, and the solvent was removed under reduced pressure. Each residue was dissolved in 1.5 mL of DMF and purified on a Gilson 215 prep-HPLC system (Method G; recoveries of 12-34 mg for the products, 16-44% yield, isolated as TFA salts).

### Example 2

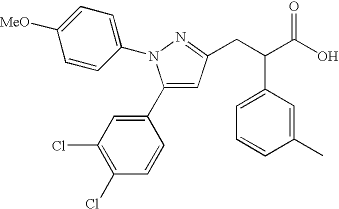

### 3-[5-(3,4-Dichloro-phenyl)-1-(4-methoxy-phenyl)-1H-pyrazol-3-yl]-2-m-tolyl-propionic acid

The title compound was prepared by Method 2: HPLC: Rₜ=10.46 (Method A), Rₜ=4.81, 7.95 (Method C). MS (ES+): mass calculated for C₂₆H₂₂Cl₂N₂O₃, 480.10; m/z found 481.1 [M+H]⁺. ¹H NMR (400 MHz, CDCl₃): 7.31-7.28 (m, 2H), 7.22 (d, J=7.6 Hz, 1H), 7.21-7.18 (m, 2H), 7.14-7.08 (m, 3H), 6.89 (dd, J=5.3, 2.0 Hz, 1H), 6.85 (d, J=8.5 Hz, 2H), 6.22 (s, 1H), 4.13-4.07 (m, 1H), 3.82 (s, 3H), 3.52 (dd, J=14.4, 9.1, Hz, 1H), 3.12 (dd, J=10.1, 5.3 Hz, 1H), 2.01 (s, 3H).

### Example 3

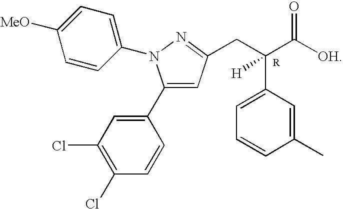

### (R)-3-[5-(3,4-Dichloro-phenyl)-1-(4-methoxy-phenyl)-1H-pyrazol-3-yl]-2-m-tolyl-propionic acid

The racemate (Example 2) was prepared by Method 2, and the title compound was separated by semi-preparative HPLC (Method D). HPLC: Rₜ=10.44 (Method A), Rₜ=4.81 (Method C). MS (ES+): mass calculated for C₂₆H₂₂Cl₂N₂O₃, 480.10; m/z found 481.1 [M+H]⁺. Optical rotation [α]²⁰₅₈₉-91.0 (c=0.1, EtOH). ¹H NMR (400 MHz, CDCl₃): 7.31 (t, J=2.2 1H), 7.29 (s, 1H), 7.22 (d, J=7.4 Hz, 1H), 7.20-7.16 (m, 2H), 7.16-7.09 (m, 3H), 6.89 (dd, J=8.4, 2.1 Hz, 1H), 6.87-6.84 (m, 2H), 6.22 (s, 1H), 4.10 (dd, J=9.2, 6.1 Hz, 1H), 3.83 (s, 3H), 3.51 (dd, J=15.0, 9.7 Hz, 1H), 3.11 (dd, J=15.2, 5.2 Hz, 1H), 2.34 (s, 3H).

### Example 4

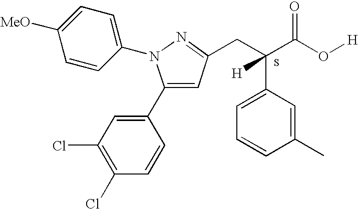

### (S)-3-[5-(3,4-dichloro-phenyl)-1-(4-methoxy-phenyl)-1H-pyrazol-3-yl]-2-m-tolyl-propionic acid

The racemate (Example 2) was prepared by Method 2, and the title compound was separated by semi-preparative HPLC (Method D). HPLC: Rₜ=10.44 (Method A), Rₜ=7.95 (Method C). MS (ES+): mass calculated for C₂₆H₂₂Cl₂N₂O₃, 480.10; m/z found 481.1 [M+H]⁺. ¹H NMR (400 MHz, CDCl₃): 7.31 (t, J=2.2 1H), 7.29 (s, 1H), 7.22 (d, J=7.4 Hz, 1H), 7.20-7.16 (m, 2H), 7.16-7.09 (m, 3H), 6.89 (dd, J=8.4, 2.1 Hz, 1H), 6.87-6.84 (m, 2H), 6.22 (s, 1H), 4.10 (dd, J=9.2, 6.1 Hz, 1H), 3.83 (s, 3H), 3.51 (dd, J=15.0, 9.7 Hz, 1H), 3.11 (dd, J=15.2, 5.2 Hz, 1H), 2.34 (s, 3H).

### Example 5

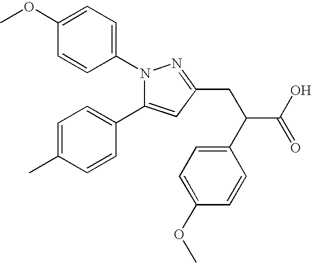

### 2-(4-Methoxy-phenyl)-3-[-1-(4-methoxyl-phenyl)-5-p-tolyl-1H-pyrazol-3-yl]-propionic acid

The title compound was prepared by Method 2: HPLC: Rₜ=9.51 (Method A). MS (ES+): mass calculated for C₂₇H₂₆N₂O₄, 442.21; m/z found 443.2 [M+H]⁺. ¹H NMR (400 MHz, CDCl₃): 7.30 (d, J=8.5 Hz, 2H), 7.14 (d, J=8.5 Hz, 2H), 7.07-7.04 (m, 4H), 6.86 (d, J=8.5 Hz, 2H), 6.81 (d, J=8.5 Hz, 2H), 6.17 (s, 1H), 4.01 (dd, J=9.4, 5.3 Hz, 1H), 3.79 (s, 6H), 3.50 (dd, J=15.0, 9.1 Hz, 1H), 3.10 (dd, J=15.0, 6.0 Hz, 1H), 2.32 (s, 3H).

### Example 6

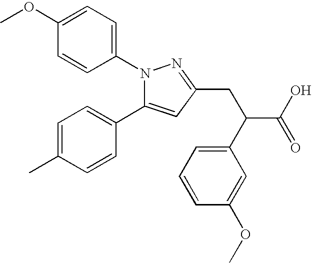

### 2-(3-Methoxy-phenyl)-3-[1-(4-methoxy-phenyl)-5-p-toly-1H-pyrazol-3-yl]-propionic acid

The title compound was prepared by Method 2: HPLC: Rₜ=9.58 (Method A). MS (ES+): mass calculated for C₂₇H₂₆N₂O₄, 442.19; m/z found 443.2 [M+H]⁺. ¹H NMR (400 MHz, CDCl₃): 7.27-7.22 (m, 2H), 7.17-7.12 (m, 2H), 7.08-7.02 (m, 3H), 6.99-6.92 (m, 2H), 6.84-6.79 (m, 2H), 6.18 (s, 1H), 4.01 (dd, J=9.4, 5.3 Hz, 1H), 3.80 (s, 6H), 3.50 (dd, J=15.0, 9.1 Hz, 1H), 3.10 (dd, J=15.0, 6.0 Hz, 1H), 2.32 (s, 3H).

### Example 7

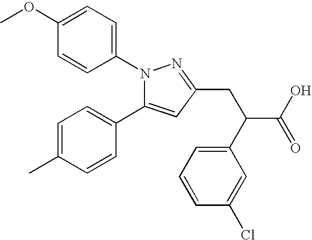

### 2-(3-Chloro-phenyl)-3-[1-(4-methoxy-phenyl)-5-p-toly-1H-pyrazol-3-yl]-propionic acid

The title compound was prepared by Method 2: HPLC: Rₜ=9.99 (Method A). MS (ES+): mass calculated for C₂₇H₂₅ClN₂O₃, 446.16; m/z found 447.2 [M+H]⁺. ¹H NMR (400 MHz, CDCl₃): 7.38-7.36 (m, 2H), 7.27-7.25 (m, 2H), 7.16-7.11 (m, 2H), 7.08-7.02 (m, 4H), 6.84-6.78 (m, 2H), 6.18 (s, 1H), 4.13-4.07 (m, 1H), 3.08 (s, 3H), 3.51 (dd, J=14.9, 9.0 Hz, 1H), 3.10 (dd, J=15.0, 6.0 Hz, 1H), 2.32 (s, 3H).

### Example 8

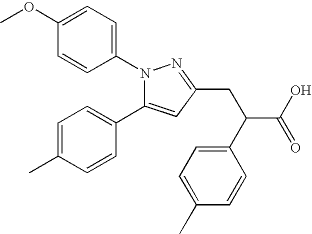

### 3-[1-(4-Methoxy-phenyl)-5-p-toly-1H-pyrazol-3-yl]-2-p-tolyl-propionic acid

The title compound was prepared by Method 2: HPLC: Rₜ=9.89 (Method A). MS (ES+): mass calculated for C₂₇H₂₆N₂O₃, 426.19; m/z found 427.2 [M+H]⁺. ¹H NMR (400 MHz, CDCl₃): 7.28-7.25 (m, 2H), 7.18-7.12 (m, 4H), 7.08-7.02 (m, 4H), 6.83-6.79 (m, 2H), 6.19 (s, 1H), 4.13-4.05 (m, 1H), 3.80 (s, 3H), 3.50 (dd, J=15.0, 9.1 Hz, 1H), 3.10 (dd, J=15.0, 6.0 Hz, 1H), 2.32 (s, 6H).

### Example 9

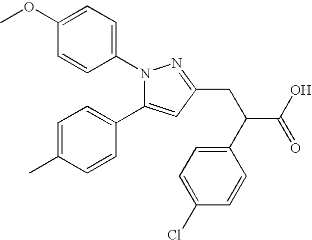

### 2-(4-Chloro-phenyl)-3-[1-(4-methoxy-phenyl)-5-p-toly-1H-pyrazol-3-yl]-propionic acid

The title compound was prepared by Method 2: HPLC: Rₜ=10.00 (Method A). MS (ES+): mass calculated for C₂₇H₂₃ClN₂O₃, 446.14; m/z found 447.2 [M+H]⁺. ¹H NMR (400 MHz, CDCl₃): 7.37 (br, s, 4H), 7.14-7.11 (m, 2H), 7.09-7.01 (m, 4H), 6.83-6.80 (m, 2H), 6.16 (s, 1H), 4.15-4.11 (m, 1H), 3.80 (s, 3H), 3.50 (dd, J=15.0, 9.1 Hz, 1H), 3.10 (dd, J=15.0, 6.0 Hz, 1H), 2.32 (s, 3H).

### Example 10

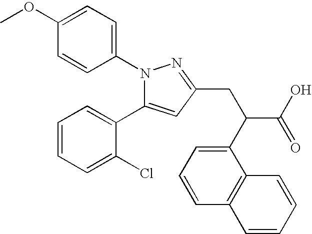

### 3-[5-(2-Chloro-phenyl)-1-(4-methoxy-phenyl)-1H-pyrazol-3-yl]-2-naphthalen-1-yl-propionic acid

The title compound was prepared by Method 2: HPLC: Rₜ=9.87 (Method A). MS (ES+): mass calculated for C₂₉H₂₃ClN₂O₃, 482.14; m/z found 483.1 [M+H]⁺. ¹H NMR (400 MHz, CDCl₃): 8.14 (d, J=8.3 Hz, 1H), 7.80 (d, J=7.8 Hz, 2H), 7.62-7.59 (m, 1H), 7.52-7.44 (m, 3H), 7.33-7.29 (m, 1H), 7.26-7.22 (m, 1H), 7.16-7.12 (m, 1H), 7.05-7.01 (m, 2H), 7.00-6.97 (m, 1H), 6.75-6.71 (m, 2H), 6.08 (s, 1H), 4.98 (dd, J=8.6, 6.6 Hz, 1H), 3.77 (dd, J=19.2, 4.2 Hz, 1H), 3.75 (s, 3H), 3.34 (dd, J=14.6, 6.57 Hz, 1H).

### Example 11

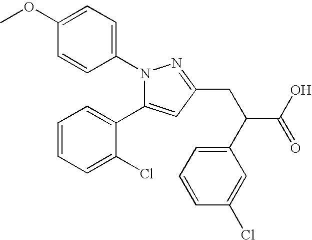

### 2-(3-Chloro-phenyl)-3-[5-(2-chloro-phenyl)-1-(4-methoxy-phenyl)-1H-pyrazol-3-yl]-propionic acid

The title compound was prepared by Method 2: HPLC: Rₜ=9.78 (Method A). MS (ES+): mass calculated for C₂₅H₂₀Cl₂N₂O₃, 466.09; m/z found 467.1 [M+H]⁺. ¹H NMR (400 MHz, CDCl₃): 7.37-7.34 (m, 2H), 7.29-7.24 (m, 4H), 7.19-7.07 (m, 2H), 7.14 (dd, J=8.0, 2.0 Hz, 2H), 6.77-6.73 (m, 2H), 6.16 (s, 1H), 4.14 (dd, J=8.3, 1.7 Hz, 1H), 3.76 (s, 3H), 3.53 (dd, J=14.7, 8.0 Hz, 1H), 3.17 (dd, J=15.2, 8.0 Hz, 1H).

### Example 12

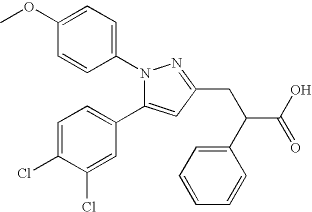

### 3-[5-(3,4-Dichloro-phenyl)-1-(4-methoxy-phenyl)-1H-pyrazol-3-yl]-2-phenyl-propionic acid

The title compound was prepared by Method 2: HPLC: Rₜ=9.78 (Method A). MS (ES+): mass calculated for C₂₅H₂₀Cl₂N₂O₃, 466.09; m/z found 467.1 [M+H]⁺. ¹H NMR (400 MHz, CDCl₃): 7.37-7.34 (m, 2H), 7.29-7.24 (m, 4H), 7.19-7.07 (m, 2H), 7.14 (dd, J=8.0, 2.0 Hz, 2H), 6.77-6.73 (m, 2H), 6.16 (s, 1H), 4.14 (dd, J=8.3, 1.7 Hz, 1H), 3.76 (s, 3H), 3.53 (dd, J=14.7, 8.0 Hz, 1H), 3.17 (dd, J=15.2, 8.0 Hz, 1H).

### Example 13

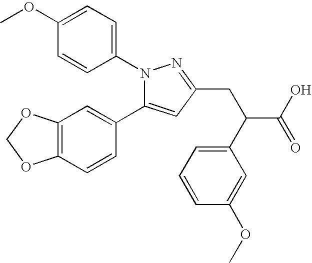

### 3-[5-Benzo[1,3]dioxol-5-yl-1-(4-methoxy-phenyl)-1H-pyrazol-3-yl]-2-(3-methoxy-phenyl)-propionic acid

The title compound was prepared by Method 2: HPLC: Rₜ=9.03 (Method A). MS (ES+): mass calculated for C₂₇H₂₄N₂O₆, 472.16; m/z found 473.2 [M+H]⁺. ¹H NMR (400 MHz, CDCl₃): 7.10-7.01 (m, 2H), 6.97-6.93 (m, 2H), 6.77 (d, J=8.3 Hz, 1H), 6.73 (t, J=2.2 Hz, 1H), 6.62 (d, J=8.5 Hz, 2H), 6.51 (d, J=8.8 Hz, 1H), 6.44 (dd, J=8.0 Hz, 1.7 Hz, 1H), 6.39 (d, J=1.2 Hz, 1H), 5.94 (s, 1H), 5.75 (s, 2H), 3.91 (dd, J=9.3, 5.8 Hz, 1H), 3.60 (s, 3H), 3.59 (s, 3H), 3.31 (dd, J=14.6, 9.3 Hz, 1H), 2.93 (dd, J=13.6, 6.5 Hz, 1H).

### Example 14

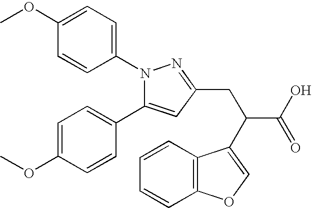

### 2-Benzofuran-3-yl-3-[1,5-bis-(4-methoxy-phenyl)-1H-pyrazol-3-yl]-propionic acid

The title compound was prepared by Method 2: HPLC: Rₜ=9.28 (Method A). MS (ES+): mass calculated for C₂₈H₂₄N₂O₅, 468.17; m/z found 469.2 [M+H]⁺. ¹H NMR (400 MHz, CDCl₃): 7.45 (d, J=2.0 Hz, 1H), 7.29-7.25 (m, 1H), 7.12-7.09 (m, 3H), 6.96-6.93 (m, 2H), 6.86-6.82 (m, 2H), 6.77-6.75 (m, 1H), 6.64-6.58 (m, 4H), 5.88 (s, 1H), 4.29 (dd, J=8.8, 6.0 Hz, 1H), 3.63 (s, 3H), 3.62 (s, 3H), 3.50 (dd, J=14.4, 9.3 Hz, 1H), 3.05 (dd, J=14.9, 6.2 Hz, 1H).

### Example 15

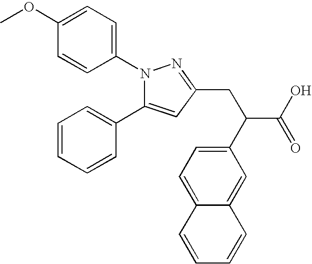

### 3-[1-(4-Methoxy-phenyl)-5-phenyl-1H-pyrazol-3-yl]-2-naphthalen-2-yl-propionic acid

The title compound was prepared by Method 2: HPLC: Rₜ=9.79 (Method A). MS (ES+): mass calculated for C₂₉H₂₄N₂O₃, 448.18; m/z found 449.2 [M+H]⁺. ¹H NMR (400 MHz, CDCl₃): 7.86-7.79 (m, 4H), 7.55-7.51 (m, 1H), 7.50-7.46 (m, 2H), 7.29-7.22 (m, 2H), 7.14-7.16 (m, 4H), 6.86-6.77 (m, 2H), 6.26 (s, 1H), 4.33 (dd, J=8.8, 6.3 Hz, 1H), 3.78 (s, 3H), 3.60 (dd, J=15.0, 8.8 Hz, 1H), 3.29 (dd, J=14.6, 6.0 Hz, 1H).

### Example 16

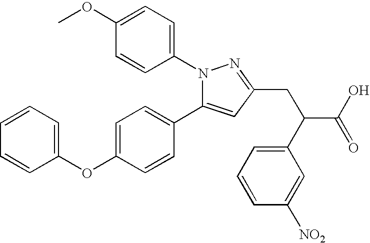

### 3-[1-(4-Methoxy-phenyl)-5-(4-phenoxy-phenyl)-1H-pyrazol-3-yl]-2-(3-nitro-phenyl)-propionic acid

The title compound was prepared by Method 2: HPLC: Rₜ=3.47 (Method B). MS (ES+): mass calculated for C₃₁H₂₅N₃O₆, 535.17; m/z found 536.2 [M+H]⁺. ¹H NMR (400 MHz, CDCl₃): 8.23 (t, J=1.5 Hz, 1H), 8.18-8.15 (m, 1H), 7.74 (d, J=7.5 Hz, 1H), 7.35 (t, J=7.5 Hz, 1H), 7.39-7.34 (m, 2H), 7.17-7.13 (m, 3H), 7.10-7.06 (m, 2H), 7.04-7.00 (m, 2H), 6.90-6.84 (m, 4H), 6.23 (s, 1H), 4.32 (dd, J=8.3, 6.5 Hz, 1H), 3.82 (s, 3H), 3.61 (dd, J=15.2, 8.6 Hz, 1H), 3.24 (dd, J=15.2, 6.3 Hz, 1H).

### Example 17

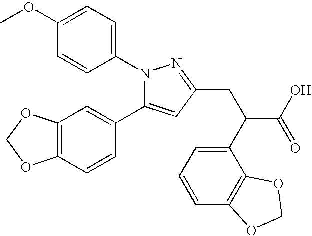

### 2-Benzo[1,3]dioxol-4-yl-3[5-benzo[1,3]dioxol-5-yl-1-(4-methoxy-phenyl)-1H-pyrazol-3-yl]-propionic acid

The title compound was prepared by Method 2: HPLC: Rₜ=2.91 (Method B). MS (ES+): mass calculated for C₂₇H₂₂N₂O₇, 486.14; m/z found 487.2 [M+H]⁺. ¹H NMR (400 MHz, CDCl₃): 7.18-7.14 (m, 2H), 6.89 (d, J=1.7 Hz, 1H), 6.86-6.83 (m, 2H), 6.81 (d, J=1.5 Hz, 1H), 6.74 (dd, J=19.2, 7.8 Hz, 2H), 6.65 (dd, J=7.83, 1.7 Hz, 1H), 6.59 (d, J=1.7 Hz, 1H), 6.17 (s, 1H), 5.95 (s, 4H), 4.06 (dd, J=9.1, 6.1 Hz, 1H), 3.81 (s, 3H), 3.48 (dd, J=15.2, 8.8 Hz, 1H), 3.10 (dd, J=15.9, 7.0 Hz, 1H).

### Example 18

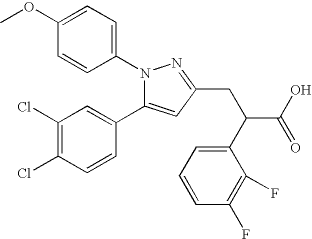

### 3-[5-(3,4-Dichloro-phenyl)-1-(4-methoxy-phenyl)-1H-pyrazol-3-yl]-2-(2,3-difluoro-phenyl)-propionic acid

The title compound was prepared by Method 2: HPLC: Rₜ=3.62 (Method B). MS (ES+): mass calculated for C₂₅H₁₈Cl₂F₂N₂O₃, 502.07; m/z found 503.1 [M+H]⁺. ¹H NMR (400 MHz, CDCl₃): 7.31 (d, J=8.3 Hz, 1H), 7.29 (d, J=2.0 Hz, 1H), 7.16-7.05 (m, 5H), 6.91-6.84 (m, 3H), 6.25 (s, 1H), 4.46 (dd, J=8.0, 7.0 Hz, 1H), 3.82 (s, 3H), 3.57 (dd, J=15.1, 8.3 Hz, 1H), 3.18 (dd, J=14.6, 7.0 Hz, 1H).

### Example 19

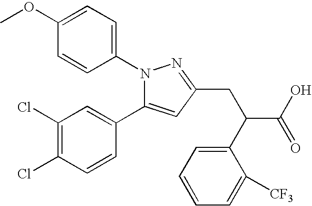

### 3-[5-(3,4-Dichloro-phenyl)-1-(4-methoxy-phenyl)-1H-pyrazol-3-yl]-2-(2-trifluoromethyl-phenyl)-propionic acid

The title compound was prepared by Method 2: HPLC: Rₜ=3.50 (Method B). MS (ES+): mass calculated for C₂₆H₁₉Cl₂F₃N₂O₃, 534.07; m/z found 535.1 [M+H]⁺. ¹H NMR (400 MHz, CDCl₃): 7.71-7.66 (m, 2H), 7.57 (t, J=8.3 Hz, 1H), 7.41 (t, J=7.3 Hz, 1H), 7.31 (s, 1H), 7.29 (d, J=1.5 Hz, 1H), 7.14-7.10 (m, 2H), 6.89 (dd, J=8.34, 2.2 Hz, 1H), 6.87-6.82 (m, 2H), 6.20 (s, 1H), 4.56 (dd, J=9.3, 5.5 Hz, 1H), 3.81 (s, 3H), 3.55 (dd, J=15.6, 8.5 Hz, 1H), 3.13 (dd, J=15.16, 6.0 Hz, 1H).

### Example 20

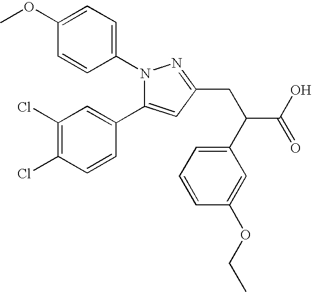

### 3-[5-(3,4-Dichloro-phenyl)-1-(4-methoxy-phenyl)-1H-pyrazol-3-yl]-2-(3-ethoxy-phenyl)-propionic acid

The title compound was prepared by Method 2: HPLC: Rₜ=5.34 (Method B). MS (ES+): mass calculated for C₂₇H₂₄Cl₂N₂O₄, 510.11; m/z found 511.1 [M+H]⁺. ¹H NMR (400 MHz, CDCl₃): 7.32 (s, 1H), 7.29 (d, J=2.2 Hz, 1H), 7.27-7.23 (m, 2H), 7.15-7.12 (m, 2H), 6.95-6.82 (m, 5H), 6.24 (s, 1H), 4.08 (dd, J=9.3, 5.5 Hz, 1H), 4.07 (q, J=13.8, 7.0 Hz, 2H), 3.82 (s, 3H), 3.52 (dd, J=15.6, 9.0 Hz, 1H), 3.14 (dd, J=15.4, 5.8 Hz, 1H), 1.40 (t, J=6.8 Hz, 3H).

### Example 21

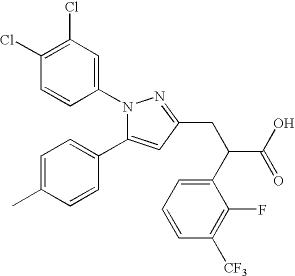

### 3-[1-(3,4-Dichloro-phenyl)-5-p-tolyl-1H-pyrazol-3-yl]-2-(2-fluoro-3-trifluoromethyl-phenyl)-propionic acid

The title compound was prepared by Method 2: HPLC: Rₜ=3.78 (Method B). MS (ES+): mass calculated for C₂₆H₁₈Cl₂F₄N₂O₃, 536.07; m/z found 537.1 [M+H]⁺. ¹H NMR (400 MHz, CDCl₃): 7.62 (t, J=6.0 Hz, 1H), 7.55 (t, J=6.8 Hz, 1H), 7.39 (d, J=2.2 Hz, 1H), 7.34 (d, J=8.5 Hz, 1H), 7.28-7.22 (m, 2H), 7.13 (d, J=8.0 Hz, 2H), 7.02 (d, J=8.0, 2H), 6.96 (dd, J=8.6, 2.5 Hz, 1H), 6.20 (s, 1H), 4.54 (t, J=7.8 Hz, 1H), 3.58 (dd, J=15.2, 7.8 Hz, 1H), 3.19 (dd, J=15.2, 7.5 Hz, 1H), 2.35 (s, 3H).

### Example 22

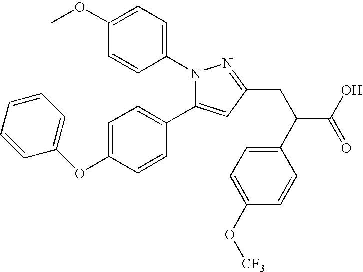

### 3-[1-(4-Methoxy-phenyl)-5-(4-phenoxy-phenyl)-1H-pyrazol-3-yl]-2-(4-trifluoromethoxyl-phenyl)-propionic acid

The title compound was prepared by Method 2: HPLC: Rₜ=3.60 (Method B). MS (ES+): mass calculated for C₃₂H₂₅F₃N₂O₅, 574.17; m/z found 575.2 [M+H]⁺. ¹H NMR (400 MHz, CDCl₃): 7.42-7.38 (m, 2H), 7.36-7.31 (m, 2H), 7.21-7.12 (m, 5H), 7.11-7.07 (m, 2H), 7.03-6.99 (m, 1H), 6.89-6.81 (m, 4H), 6.18 (s, 1H), 4.18 (dd, J=9.6, 5.3 Hz, 1H), 3.80 (s, 3H), 3.52 (dd, J=14.9, 9.4 Hz, 1H), 3.12 (dd, J=15.2, 5.6 Hz, 1H).

### Example 23

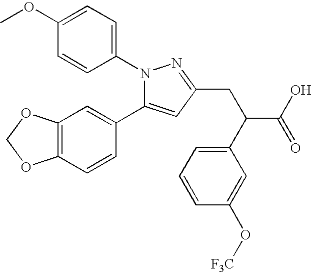

### 3-[5-Benzo[1,3]dioxo-5-yl-1-(4-methoxy-phenyl)-1H-pyrazol-3-yl]-2-(3-trifluoromethoxyl-phenyl)-propionic acid

The title compound was prepared by Method 2: HPLC: Rₜ=3.28 (Method B). MS (ES+): mass calculated for C₂₇H₂₁F₃N₂O₆, 526.14; m/z found 527.1 [M+H]⁺. ¹H NMR (400 MHz, CDCl₃): 7.38-7.29 (m, 2H), 7.22-7.20 (m, 1H), 7.15-7.11 (m, 3H), 6.86-6.82 (m, 2H), 6.70 (d, J=7.8 Hz, 1H), 6.60 (dd, J=8.34, 1.5 Hz, 1H), 6.54 (d, J=1.8 Hz, 1H), 6.13 (s, 1H), 5.94 (s, 2H), 4.13 (dd, J=8.6, 6.3 Hz, 1H), 3.81 (s, 3H), 3.52 (dd, J=14.9, 8.6 Hz, 1H), 3.16 (dd, J=15.2, 6.8 Hz, 1H).

### Example 24

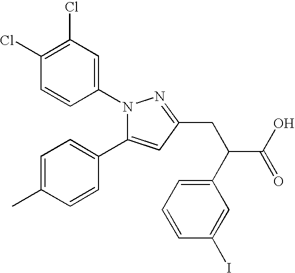

### 3-[1-(3,4-Dichloro-phenyl)-5-p-tolyl-1H-pyrazol-3-yl]-2-(3-iodo-phenyl)-propionic acid

The title compound was prepared by Method 2: HPLC: Rₜ=3.89 (Method B). MS (ES+): mass calculated for C₂₅H₁₉Cl₂1N₂O₂, 575.99; m/z found 577.0 [M+H]⁺. ¹H NMR (400 MHz, CDCl₃): 7.73 (t, J=2.0 Hz, 1H), 7.64-7.62 (m, 1H), 7.48 (d, J=2.5 Hz, 1H), 7.38-7.35 (m, 1H), 7.32 (d, J=8.6 Hz, 2H), 7.15-7.07 (m, 4H), 6.98 (dd, J=8.8, 2.3 Hz, 1H), 6.18 (s, 1H), 4.11 (dd, J=9.0, 6.3 Hz, 1H), 3.49 (dd, J=15.4, 8.8 Hz, 1H), 3.10 (dd, J=15.4, 6.3 Hz, 1H), 2.35 (s, 3H).

### Example 25

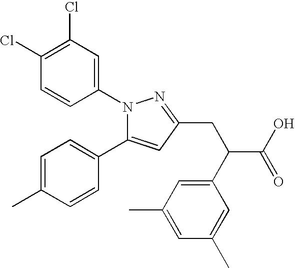

### 3-[1-(3,4-Dichloro-phenyl)-5-p-tolyl-1H-pyrazol-3-yl]-2-(3,5-dimethyl-phenyl)-propionic acid

The title compound was prepared by Method 2: HPLC: Rₜ=3.84 (Method B). MS (ES+): mass calculated for C₂₇H₂₄Cl₂N₂O₂, 478.12; m/z found 479.1 [M+H]⁺. ¹H NMR (400 MHz, CDCl₃): 7.45 (d, J=2.2 Hz, 1H), 7.35 (d, J=8.6 Hz, 1H), 7.12 (d, J=7.8 Hz, 2H), 7.06-7.03 (m, 2H), 7.00-6.98 (m, 2H), 6.97 (d, J=2.3 Hz, 1H), 6.93 (br, s, 1H), 6.22 (s, 1H), 4.05 (dd, J=6.0, 5.6 Hz, 1H), 3.51 (dd, J=15.2, 9.3 Hz, 1H), 3.09 (dd, J=15.2, 5.8 Hz, 1H), 2.36 (s, 3H), 2.31 (s, 6H).

### Example 26

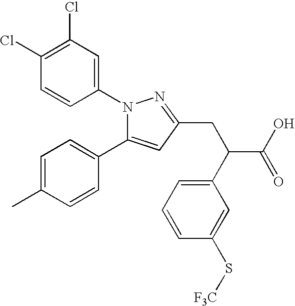

### 3-[1-(3,4-Dichloro-phenyl)-5-p-tolyl-1H-pyrazol-3-yl]-2-(3-trifluoromethylsulfanyl-phenyl)-propionic acid

The title compound was prepared by Method 2: HPLC: Rₜ=3.91 (Method B). MS (ES+): mass calculated for C₂₆H₁₉Cl₂F₃N₂O₂S, 550.05; m/z found 551.0 [M+H]⁺. ¹H NMR (400 MHz, CDCl₃): 7.67-7.65 (m, 1H), 7.61-7.57 (m, 1H), 7.55-7.51 (m, 1H), 7.45 (d, J=2.5 Hz, 1H), 7.41 (t, J=7.1 Hz, 1H), 7.32 (d, J=8.3 Hz, 2H), 7.12 (d, J=8.3 Hz, 2H), 7.04-7.01 (m, 2H), 6.95 (dd, J=8.6, 2.3 Hz, 1H), 6.15 (s, 1H), 4.19 (dd, J=8.6, 6.3 Hz, 1H), 3.53 (dd, J=15.4, 8.3 Hz, 1H), 3.16 (dd, J=14.9, 6.3 Hz, 1H), 2.37 (s, 3H).

### Example 27

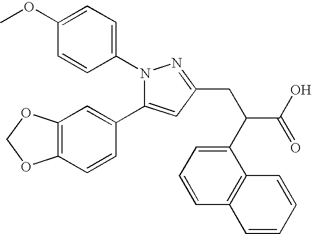

### 3-[5-Benzo[1,3]dioxol-5-yl-1-(4-methoxy-phenyl)-1H-pyrazol-3-yl]-2-naphthalen-1-yl-propionic acid

The title compound was prepared by Method 2: HPLC: Rₜ=9.47 (Method A). MS (ES+): mass calculated for C₃₀H₂₄N₂O₅, 492.17; m/z found 493.2 [M+H]⁺. ¹H NMR (400 MHz, CDCl₃): 8.13 (d, J=8.6 Hz, 1H), 7.88-7.84 (m, 2H), 7.79 (d, J=7.8 Hz, 1H), 7.58 (d, J=7.3 Hz, 1H), 7.51-7.43 (m, 3H), 7.08 (d, J=8.8 Hz, 1H), 6.80 (d, J=8.6 Hz, 2H), 6.6 (d, J=8.1 Hz, 1H), 6.53 (dd, J=8.1, 1.26 Hz, 1H), 6.46 (d, J=1.8 Hz, 1H), 6.09 (s, 1H), 5.93 (s, 2H), 4.95 (dd, J=8.6, 6.3 Hz, 1H), 3.79 (s, 3H), 3.73-3.65 (m, 1H), 3.25 (dd, J=14.6, 6.3 Hz, 1H).

### Example 28

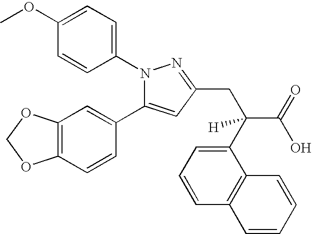

### (R)-3-[5-Benzo[1,3]dioxol-5-yl-1-(4-methoxy-phenyl)-1H-pyrazol-3-yl]-2-naphthalen-1-yl-propionic acid

The racemate (Example 27) was prepared by Method 2, and the title compound was isolated by semi-preparative chiral HPLC (Method D). HPLC: Rₜ=3.82 (Method C). MS (ES+): mass calculated for C₃₀H₂₄N₂O₅, 492.17; m/z found 493.2 [M+H]⁺. ¹H NMR (400 MHz, CDCl₃): 7.83-7.79 (m, 4H), 7.52 (dd, J=8.4, 1.6 Hz, 1H), 7.48-7.45 (m, 2H), 7.16-7.12 (m, 2H), 6.84-6.80 (m, 2H), 6.70-6.68 (m, 1H), 6.62 (dd, J=7.8, 2.0 Hz, 2H), 6.56 (d, J=1.8 Hz, 1H), 6.16 (s, 1H), 5.94 (s, 2H), 4.33 (dd, J=9.2, 5.6 Hz, 1H), 3.79 (s, 3H), 3.63 (dd, J=14.9, 9.0 Hz, 1H), 3.24 (dd, J=15.7, 5.1 Hz, 1H).

### Example 29

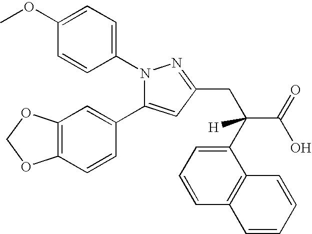

### (S)-3-[5-Benzo[1,3]dioxol-5-yl-1-(4-methoxy-phenyl)-1H-pyrazol-3-yl]-2-naphthalen-1-yl-propionic acid

The racemate (Example 27) was prepared by Method 2, and the title compound was isolated by semi-preparative chiral HPLC (Method D). HPLC: Rₜ=6.83 (Method C). MS (ES+): mass calculated for C₃₀H₂₄N₂O₅, 492.17; m/z found 493.2 [M+H]⁺. ¹H NMR (400 MHz, CDCl₃): 7.83-7.79 (m, 4H), 7.52 (dd, J=8.4, 1.6 Hz, 1H), 7.48-7.45 (m, 2H), 7.16-7.12 (m, 2H), 6.84-6.80 (m, 2H), 6.70-6.68 (m, 1H), 6.62 (dd, J=7.8, 2.0 Hz, 2H), 6.56 (d, J=1.8 Hz, 1H), 6.16 (s, 1H), 5.94 (s, 2H), 4.33 (dd, J=9.2, 5.6 Hz, 1H), 3.79 (s, 3H), 3.63 (dd, J=14.9, 9.0 Hz, 1H), 3.24 (dd, J=15.7, 5.1 Hz, 1H).

### Example 30

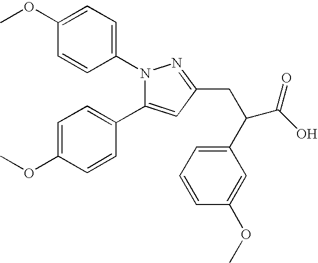

### 3-[1,5-Bis-(4-methoxy-phenyl)-1H-pyrazol-3-yl]-2-(3-methoxy-phenyl)-propionic acid

The title compound was prepared by Method 2: HPLC: Rₜ₌9.15 (Method A).

MS (ES+): mass calculated for C₂₇H₂₆N₂O₅, 458.18; m/z found 459.2 [M+H]⁺. ¹H NMR (400 MHz, CDCl₃): 7.26-7.22 (m, 2H), 7.16-7.13 (m, 2H), 7.08-7.05 (m, 2H), 6.97 (d, J=7.3 Hz, 1H), 6.93 (t, J=2.3 Hz, 1H), 6.83-6.77 (m, 5H), 6.16 (s, 1H), 4.12 (dd, J=9.9, 5.3 Hz, 1H), 3.80 (s, 3H), 3.79 (s, 3H), 3.78 (s, 3H), 3.52 (dd, J=14.2, 9.6 Hz, 1H), 3.12 (dd, J=15.2, 6.1 Hz, 1H).

### Example 31

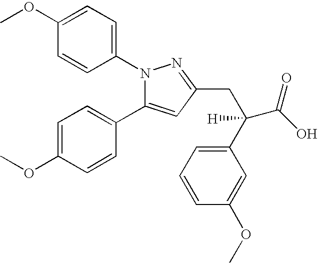

### (R)-3-[1,5-Bis-(4-methoxy-phenyl)-1H-pyrazol-3-yl]-2-(3-methoxy-phenyl)-propionic acid

The racemate (Example 30) was prepared by Method 2, and the title compound was isolated by semi-preparative chiral HPLC (Method D). HPLC: Rₜ=4.84 (Method C). MS (ES+): mass calculated for C₂₇H₂₆N₂O₅, 458.18; m/z found 459.2 [M+H]⁺. ¹H NMR (400 MHz, CDCl₃): 7.28-7.24 (m, 2H), 7.19-7.15 (m, 2H), 7.09-7.05 (m, 2H), 6.97 (d, J=7.8 Hz, 1H), 6.93 (t, J=2.0 Hz, 1H), 6.87-6.78 (m, 5H), 6.16 (s, 1H), 4.12 (dd, J=9.9, 6.2 Hz, 1H), 3.80 (s, 3H), 3.79 (s, 3H), 3.78 (s, 3H), 3.52 (dd, J=15.1, 9.5 Hz, 1H), 3.12 (dd, J=15.3, 5.5 Hz, 1H).

### Example 32

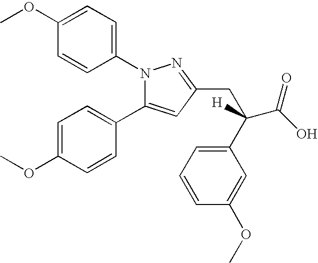

### (S)-3-[1,5-Bis-(4-methoxy-phenyl)-1H-pyrazol-3-yl]-2-(3-methoxy-phenyl)-propionic acid

The racemate (Example 30) was prepared by Method 2, and the title compound was isolated by semi-preparative chiral HPLC (Method D). HPLC: Rₜ=7.37 (Method C). MS (ES+): mass calculated for C₂₇H₂₆N₂O₅, 458.18; m/z found 459.2 [M+H]⁺. ¹H NMR (400 MHz, CDCl₃): 7.28-7.24 (m, 2H), 7.19-7.15 (m, 2H), 7.09-7.05 (m, 2H), 6.97 (d, J=7.8 Hz, 1H), 6.93 (t, J=2.0 Hz, 1H), 6.87-6.78 (m, 5H), 6.20 (s, 1H), 4.15 (dd, J=9.9, 6.2 Hz, 1H), 3.82 (s, 3H), 3.80 (s, 3H), 3.79 (s, 3H), 3.55 (dd, J=15.1, 9.5 Hz, 1H), 3.16 (dd, J=15.3, 5.5 Hz, 1H).

### Example 33

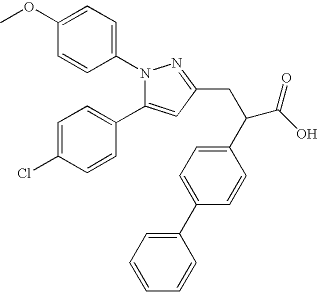

### 2-Biphenyl-4-yl-3-[5-(4-chloro-phenyl)-1-(4-methoxy-phenyl)-1H-pyrazol-3-yl]-propionic acid

The title compound was prepared by Method 2: HPLC: Rₜ=7.21 (Method A). MS (ES+): mass calculated for C₃₁H₂₅N₂O₃, 508.16; m/z found 509.2 [M+H]⁺. ¹H NMR (400 MHz, CDCl₃): 7.24-7.01 (m, 7H), 6.98-6.80 (m, 4H), 6.75-6.64 (m, 2H), 6.58-6.44 (m, 2H), 5.79 (s, 1H), 3.71 (m, 1H), 3.47 (s, 3H), 3.22-3.08 (m, 3H), 2.85-2.64 (m, 3H).

### Example 34

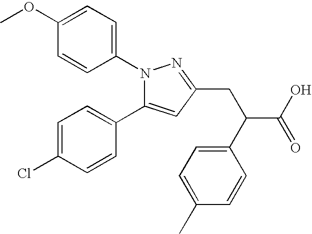

### 3-[5-(4-Chloro-phenyl)-1-(4-methoxy-phenyl)-1H-pyrazol-3-yl]-2-p-tolyl-propionic acid

The title compound was prepared by Method 2: HPLC: Rₜ=10.11 (Method A). MS (ES+): mass calculated for C₂₆H₂₃ClN₂O₃, 446.14; m/z found 447.2 [M+H]⁺. ¹H NMR (500 MHz, DMSO-d₆): 12.37 (br s, 1H), 7.40 (d, J=8.6 Hz, 2H), 7.26 (d, J=8.1 Hz, 2H), 7.18-7.11 (m, 6H), 6.95 (d, J=9.0 Hz, 2H), 6.40 (s, 1H), 3.98 (dd, J=6.3, 9.1 Hz, 1H), 3.77 (s, 3H), 3.34 (dd, J=9.1, 15.1 Hz, 1H), 2.92 (dd, J=6.2, 15.0 Hz, 1H), 2.27 (s, 3H).

### Example 35

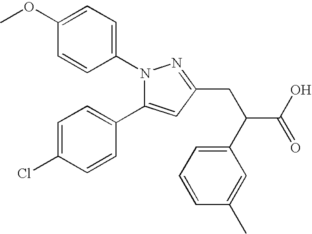

### 3-[5-(4-Chloro-phenyl)-1-(4-methoxy-phenyl)-1H-pyrazol-3-yl]-2-m-tolyl-propionic acid

The title compound was prepared by Method 2: HPLC: Rₜ=10.11 (Method A). MS (ES+): mass calculated for C₂₆H₂₃ClN₂O₃, 446.14; m/z found 447.1 [M+H]⁺. ¹H NMR (500 MHz, DMSO-d₆): 12.29 (br s, 1H), 7.40 (d, J=8.6 Hz, 2H), 7.22 (t, J=7.5 Hz, 1H), 7.19-7.15 (m, 3H), 7.13 (d, J=8.9 Hz, 2H), 7.08 (d, J=7.3 Hz, 1H), 6.95 (d, J=9.0 Hz, 2H), 6.40 (s, 1H), 3.98 (dd, J=6.0, 9.3 Hz, 1H), 3.77 (s, 3H), 2.92 (dd, J=6.0, 14.9 Hz, 1H), 2.30 (s, 3H).

### Example 36

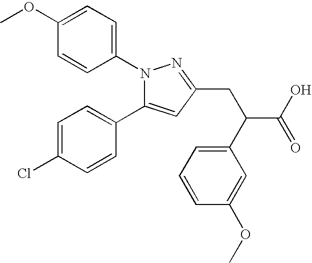

### 3-[5-(4-Chloro-phenyl)-1-(4-methoxy-phenyl)-1H-pyrazol-3-yl]-2-(3-methoxy-phenyl)-propionic acid

The title compound was prepared by Method 2: HPLC: Rₜ=9.79 (Method A). MS (ES+): mass calculated for C₂₆H₂₃ClN₂O₄, 462.13; m/z found 463.1 [M+H]⁺. ¹H NMR (500 MHz, DMSO-d₆): 12.29 (br s, 1H), 7.40 (d, J=8.5 Hz, 2H), 7.26 (t, J=7.9 Hz, 1H), 7.17 (d, J=8.5 Hz 2H), 7.13 (d, J=8.9 Hz, 2H), 6.96-6.92 (m, 4H), 6.84 (d, J=8.2 Hz, 1H), 6.42 (s, 1H), 4.01 (dd, J=6.1, 9.2 Hz, 1H), 3.78 (s, 3H), 3.74 (s, 3H), 2.93 (dd, J=6.1, 14.9 Hz, 1H).

### Example 37

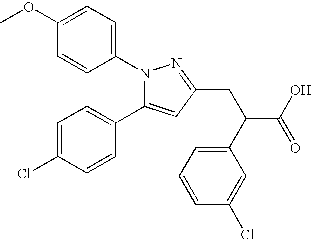

### 2-(3-Chloro-phenyl)-3-[5-(4-chloro-phenyl)-1-(4-methoxy-phenyl)-1H-pyrazol-3-yl]-propionic acid

The title compound was prepared by Method 2: HPLC: Rₜ=10.19 (Method A). MS (ES+): mass calculated for C₂₅H₂₀Cl₂N₂O₃, 466.09; m/z found 467.2 [M+H]⁺. ¹H NMR (400 MHz, DMSO-d₆): 7.45 (m, 1H), 7.43 (d, J=8.6 Hz, 2H), 7.39-7.34 (m, 3H), 7.18 (d, J=8.6 Hz, 2H), 7.13 (d, J=9.0 Hz, 2H), 6.97 (d, J=9.0 Hz, 2H), 4.11 (dd, J=6.8, 8.6 Hz, 1H), 3.79 (s, 3H), 3.38 (dd, J=8.4, 14.8 Hz, 1H), 3.01 (dd, J=6.8, 14.8 Hz, 1H).

### Example 38

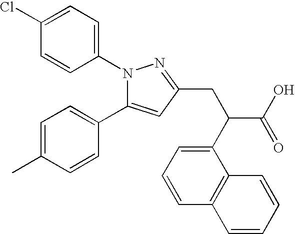

### 3-[1-(4-Chloro-phenyl)-5-p-tolyl-1H-pyrazol-3-yl]-2-naphthalen-1-yl-propionic acid

The title compound was prepared by Method 2: HPLC: Rₜ=10.66 (Method A). MS (ES+): mass calculated for C₂₉H₂₃ClN₂O₂, 466.14; m/z found 467.1 [M+H]⁺. ¹H NMR (500 MHz, DMSO-d₆): 12.52 (br s, 1H), 8.22 (d, J=8.3 Hz, 1H), 7.95 (d, J=8.0 Hz, 1H), 7.86 (d, J=8.1 Hz, 1H), 7.60-7.52 (m, 4H), 7.44 (d, J=8.9 Hz, 2H), 7.17-7.15 (m, 4H), 7.02 (d, J=8.1 Hz, 2H), 6.40 (s, 1H), 4.87 (dd, J=6.3, 8.6 Hz, 1H), 3.54 (dd, J=8.6, 14.9 Hz, 1H), 3.09 (dd, J=6.2, 14.9 Hz, 1H), 2.28 (s, 3H).

### Example 39

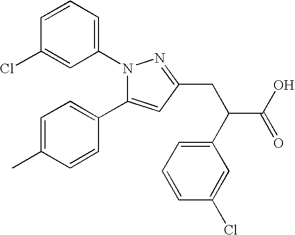

### 2-(3-Chloro-phenyl)-3-[1-(3-chloro-phenyl)-5-p-tolyl-1H-pyrazol-3-yl]-propionic acid

The title compound was prepared by Method 2: HPLC: Rₜ=10.56 (Method A). MS (ES+): mass calculated for C₂₅H₂₀Cl₂N₂O₂, 450.09; m/z found 451.0 [M+H]⁺. ¹H NMR (500 MHz, DMSO-d₆): 12.59 (br s, 1H), 7.44-7.31 (m, 7H), 7.18 (d, J=8.0 Hz, 2H), 7.08 (d, J=8.1 Hz, 2H), 7.05 (d, J=7.2 HZ, 1H), 6.38 (s, 1H), 4.10 (dd, J=6.8, 8.6 HZ, 1H), 3.00 (dd, J=6.7, 14.9 Hz, 1H), 2.30 (s, 3H).

### Example 40

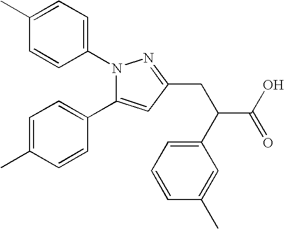

### 3-(1,5-Di-p-tolyl-1H-pyrazol-3-yl)-2-m-tolyl-propionic acid

The title compound was prepared by Method 2: HPLC: Rₜ=10.30 (Method A). MS (ES+): mass calculated for C₂₇H₂₆N₂O₂, 410.20; m/z found 411.1 [M+H]⁺. ¹H NMR (500 MHz, DMSO-d₆): 12.39 (br s, 1H), 7.24-7.17 (m, 5H), 7.13 (d, J=7.9 Hz, 2H), 7.09-7.02 (m, 5H), 6.32 (s, 1H), 3.98 (dd, J=6.0, 9.3 Hz, 1H), 2.92 (dd, J=6.0, 14.8 Hz, 1H), 2.31 (s, 3H), 2.30 (s, 3H), 2.28 (s, 3H).

### Example 41

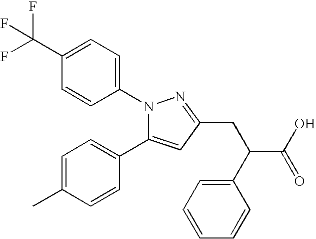

### 2-Phenyl-3-[5-p-tolyl-1-(4-trifluoromethyl-phenyl)-1H-pyrazol-3-yl]-propionic acid

The title compound was prepared by Method 2: HPLC: Rₜ=10.41 (Method A). MS (ES+): mass calculated for C₂₆H₂₁F₃N₂O₂, 450.16; m/z found 451.0 [M+H]⁺. ¹H NMR (500 MHz, DMSO-d₆): 12.40 (br s, 1H), 7.76 (d, J=8.5 Hz, 2H), 7.41-7.39 (m, 4H), 735 (t, J=7.7 Hz, 2H), 7.28 (m, 1H), 7.19 (d, 7.9 Hz, 2H), 7.09 (d, J=8.1 Hz, 2H), 6.40 (s, 1H), 4.06 (dd, J=6.3, 9.1 Hz, 1H), 3.40 (dd, J=9.0, 15 Hz, 1H), 2.98 (dd, J=6.3, 15 Hz, 1H), 2.31 (s, 3H).

### Example 42

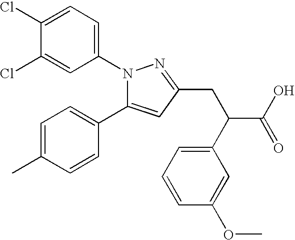

### 3-[1-(3,4-Dichloro-phenyl)-5-p-tolyl-1H-pyrazol-3-yl]-2-(3-methoxy-phenyl)-propionic acid

The title compound was prepared by Method 2: HPLC: Rₜ=10.61 (Method A). MS (ES+): mass calculated for C₂₆H₂₂Cl₂N₂O₃, 480.10; m/z found 481.0 [M+H]⁺. ¹H NMR (500 MHz, DMSO-d₆): 12.40 (br s, 1H), 7.62 (d J=8.7 Hz, 1H), 7.53 (d, J=2.5 Hz, 1H), 7.26 (d, J=7.9 Hz, 1H), 7.20 (d, J=7.9 Hz, 2H), 7.11 (d, J=8.1 Hz, 2H), 7.07 (dd, J=2.5, 8.6 Hz, 1H), 6.96 (d, J=7.7 Hz, 1H), 6.94 (s, 1H), 6.85 (dd, J=2.6, 8.3 Hz, 1H), 6.40 (s, 1H), 4.03 (dd, J=6.1, 9.2 Hz, 1H), 3.74 (s, 3H), 3.36 (dd, J=9.3, 15.1 Hz, 1H), 2.95 (dd, J=6.1, 15.0 Hz, 1H), 2.31 (s, 3H).

### Example 43

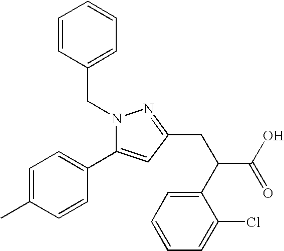

### 3-(1-Benzyl-5-p-tolyl-1H-pyrazol-3-yl)-2-(2-chloro-phenyl)-propionic acid

The title compound was prepared by Method 2: HPLC: Rₜ=9.95 (Method A). MS (ES+): mass calculated for C₂₆H₂₃ClN₂O₂, 430.14; m/z found 431.0 [M+H]⁺. ¹H NMR (500 MHz, DMSO-d₆): 12.60 (br s, 1H), 7.45-7.43 (m, 2H), 7.32-7.28 (m, 2H), 7.23-7.15 (m, 7H), 6.83 (d, J=9.0 Hz, 2H), 6.12 (s, 1H), 5.24 (s, 2H), 4.46 (t, J=7.8 Hz, 1H), 3.31 (dd, J=7.1, 14.6 Hz, 1H), 3.04 (dd, J=8.2, 14.6 Hz, 1H), 2.29 (s, 3H).

### Example 44

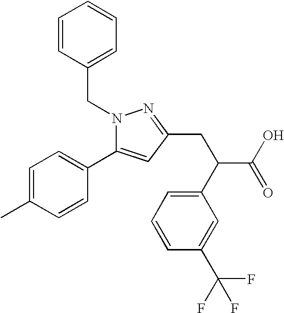

### 3-(1-Benzyl-5-p-tolyl-1H-pyrazol-3-yl)-2-(3-trifluoromethyl-phenyl)-propionic acid

The title compound was prepared by Method 2: HPLC: Rₜ=10.19 (Method A). MS (ES+): mass calculated for C₂₇H₂₃F₃N₂O₂, 464.17; m/z found 465.0 [M+H]⁺. ¹H NMR (500 MHz, DMSO-d₆): 12.60 (br s, 1H), 7.65-7.63 (m, 4H), 7.56 (t, J=7.9 Hz, 1H), 7.23-7.13 (m, 7H), 6.79 (m, 2H), 6.19 (s, 1H), 5.23 (s, 2H), 4.17 (t, J=7.9 Hz, 1H), 3.32 (dd, J=7.5, 14.7 Hz, 1H), 3.03 (dd, J=8.2, 14.7 Hz, 1H), 2.30 (s, 3H).

### Example 45

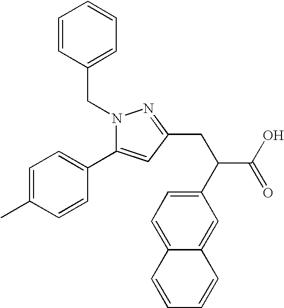

### 3-(1-Benzyl-5-p-tolyl-1H-pyrazol-3-yl)-2-naphthalen-2-yl-propionic acid

The title compound was prepared by Method 2: HPLC: Rₜ=10.13 (Method A). MS (ES+): mass calculated for C₃₀H₂₆N₂O₂, 446.20; m/z found 447.1 [M+H]⁺. ¹H NMR (500 MHz, DMSO-d₆): 12.42 (br s, 1H), 7.90-7.85 (m, 4H), 7.53-7.49 (m, 3H), 7.20-7.14 (m, 7H), 7.09 (t, J=7.6 Hz, 2H), 6.78 (d, J=7.3 Hz, 2H), 6.20 (s, 1H), 5.23 (s, 2H), 4.18 (t, J=7.8 Hz, 1H), 3.40 (dd, J=7.8, 14.8 Hz, 1H), 3.09 (dd, J=7.8, 14.7 Hz, 1H), 2.29 (s, 3H).

### Example 46

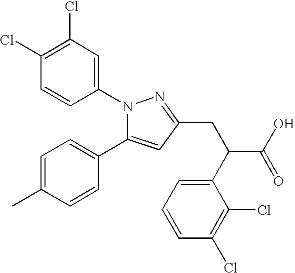

### 2-(2,3-Dichloro-phenyl)-3-[1-(3,4-dichloro-phenyl)-5-p-tolyl-1H-pyrazol-3-yl]-propionic acid

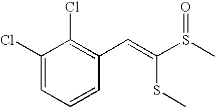

A. 1,2 Dichloro-3-(2-methanesulfinyl-2-methylsufanyl-vinyl)-benzene. To a stirred solution of methyl methylthiomethyl sulfoxide (4.97 g, 40.0 mmol) and 2,3-dichlorobenzaldehyde (5.00 g, 28.6 mmol) in 10 mL of THF was added 4 mL of triton-B (40% in MeOH). The resultant mixture was refluxed for 4 h. The solvent was removed under reduced pressure, and the residue was purified by silica gel chromatography (5:95 EtOAc/hexane) to afford 5.4 g (67.5%) of 1,2-dichloro-3-(2-methanesulfinyl-2-methylsufanyl-vinyl)-benzene. HPLC: Rₜ=8.99. (Method A). ¹H NMR (400 MHz, CDCl₃): 7.86 (s, 1H), 7.73 (dd, J=8.4, 0.9 Hz, 1H), 7.47 (dd, J=9.0, 0.6 Hz, 1H), 7.38-7.23 (m, 1H), 2.83 (s, 3H), 2.24 (s, 3H).

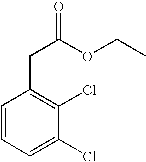

B. (2,3-Dichloro-phenyl)-acetic acid ethyl ester. A stirred solution of 1,2-dichloro-3-(2-methanesulfinyl-2-methylsufanyl-vinyl)-benzene (5.40 g, 19.3 mmol) in 30 mL of MeOH at 0° C. was bubbled with HCl gas for 10 min and then was allowed to warm to rt and stir for 0.5 h. The solvent was removed under reduced pressure, and the residue was purified by silica gel chromatography (5:95 EtOAc/hexane) to afford 3.08 g (73.4%) of (2,3-Dichloro-phenyl)-acetic acid ethyl ester. HPLC: Rₜ=9.88 (Method A). ¹H NMR (400 MHz, CDCl₃): 7.40 (dd, J=7.2, 2.7 Hz, 1H), 7.20-7.15 (m, 2H), 4.18 (dd, J=14.2, 7.0 Hz, 2H), 3.79 (s, 2H), 1.26 (t, J=6.8, Hz, 2H).

C. 2-(2,3-Dichloro-Phenyl)-3-[1-(3,4-dichloro-phenyl)-5-p-tolyl-1H-pyrazol-3-yl]-propionic acid. The title compound was prepared by Method 2 (Scheme A) from the product of Step B and the appropriate pyrazole bromide from Method 1: HPLC: Rₜ=3.89 (Method B). MS (ES+): mass calculated for C₂₅H₁₈Cl₄N₂O₂, 518.01; m/z found 519.0 [M+H]⁺. ¹H NMR (400 MHz, CDCl₃): 7.43 (d, J=2.3 Hz, 1H), 7.40 (dd, J=8.6, 1.5 Hz, 1H), 7.36 (dd, J=7.8, 1.2 Hz, 1H), 7.31 (d, J=8.1 Hz, 1H), 7.21 (t, J=8.1 Hz, 2H), 7.12 (d, J=8.8 Hz, 2H), 7.05-7.02 (m, 2H), 6.96 (dd, J=8.6, 2.5 Hz, 1H), 6.18 (s, 1H), 4.76 (dd, J=8.3, 6.6 Hz, 1H), 3.52 (dd, J=15.4, 8.1 Hz, 1H), 3.16 (dd, J=14.9, 7.3 Hz, 1H), 2.35 (s, 3H).

Method 3

### Synthesis of 4-Oxo-2-aryl-pentanoic Acids, such as

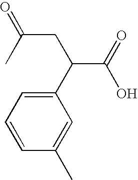

### 4-Oxo-2-m-tolyl-pentanoic acid

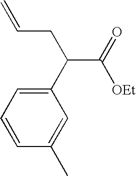

A. 2-m-Tolyl-pent-4-enoic acid ethyl ester. To a stirred solution 3-methylphenylacetic acid ethyl ester (50.0 g, 0.281 mol) in DMF (500 mL) at 0° C. under N₂ was added 60% NaH (12.3 g, 0.308 mol) in small portions. The mixture was allowed to warm to rt and stir for 1.5 h. In a second vessel, a stirred solution of allyl bromide (72.7 mL, 0.843 mol) in DMF (300 mL) was cooled to −42° C. (acetonitrile/CO₂) under N₂, and the enolate mixture was slowly added to this solution by cannula. After the addition was complete, the mixture was allowed to warm to rt and stir for 2 h. The mixture was then diluted with H₂O (100 mL) and the majority of the DMF was removed under reduced pressure. The mixture was then further diluted with H₂O (400 mL) and EtOAc (500 mL), and the layers were separated. The aqueous phase was extracted with EtOAc (3×150 mL) and the combined organic extracts were dried over Na₂SO₄ and filtered, and the solvent was removed under reduced pressure. Purification on silica gel (0-10% EtOAc in hexane) gave 57.4 g (93%) of desired ester as a light yellow oil. TLC (silica, 10% EtOAc/hexane): R_{f}=0.7. ¹H NMR (400 MHz, CDCl₃): 7.21 (t, J=7.8 Hz, 1H), 7.12 (s, 1H), 7.08 (t, J=7.8 Hz, 2H), 5.79-5.66 (m, 1H), 5.11-5.04 (m, 1H), 5.02-4.98 (m, 1H), 4.20-4.02 (m, 2H), 3.62-3.54 (m, 1H), 2.86-2.74 (m, 1H), 2.53-2.44 (m, 1H), 2.34 (s, 3H), 1.21 (t, J=7.1 Hz, 3H).

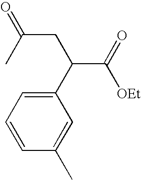

B. 4-Oxo-2-m-tolyl-pentanoic acid ethyl ester. A slow stream of O₂ was bubbled through a stirred suspension of 2-m-tolyl-pent-4-enoic acid ethyl ester (57.0 g, 0.261 mol), CuCl (25.7 g, 0.261 mol) and PdCl₂ (9.26 g, 0.052 mol) in 8:1 DMF/H₂O (130 mL) for 14 h. The mixture was diluted with CH₂Cl₂ (500 mL) and 9:1 saturated NH₄Cl/NH₄OH (500 mL). The mixture was stirred for 1 h and then filtered through a pad of celite. The layers were separated, and the organic phase was washed with 9:1 saturated NH₄Cl/NH₄OH (200 mL). The combined aqueous phases were extracted with CH₂Cl₂ (3×150 mL). The organics were then dried over Na₂SO₄ and filtered, and the solvent was removed under reduced pressure. Purification on silica gel (0-20% EtOAc in hexane) gave 34.4 g (56%) of desired ketone as a light yellow oil. TLC (silica, 10% EtOAc/hexane): R_{f}=0.3. ¹H NMR (400 MHz, CDCl₃): 7.20 (t, J=7.6 Hz, 1H), 7.10-7.03 (m, 3H), 4.20-4.00 (m, 3H), 3.37 (dd. J=10.4, 17.9 Hz, 1H), 2.69 (dd. J=4.3, 17.9 Hz, 1H), 2.33 (s, 3H), 2.17 (s, 3H), 1.20 (t, J=7.3 Hz, 3H).

C. 4-Oxo-2-m-tolyl-pentanoic acid. To a stirred solution of 4-oxo-2-m-tolyl-pentanoic acid ethyl ester (34.0 g, 145 mmol) in 3:1:1 THF/MeOH/H₂O (300 mL) was added LiOH.H₂O (30.5 g, 0.726 mol) and the mixture was stirred overnight at rt. The mixture was then heated to 65° C. for 2 h, cooled to rt, and was diluted with H₂O (250 mL) and 20% diethyl ether/hexane. The layers were separated, and the aqueous layer was adjusted to pH 1 with concd HCl at 0° C. The aqueous phase was then extracted with EtOAc (3×200 mL), dried over Na₂SO₄ and filtered, and then the solvent was removed under reduced pressure to afford 28.4 g (95%) of crude acid as a light yellow solid. TLC (silica, 10% EtOAc/hexane): R_{f=}0.3. ¹H NMR (400 MHz, CDCl₃): 7.21 (t, J=7.6 Hz, 1H), 7.11-7.05 (m, 3H), 4.08 (dd. J=4.0, 10.2 Hz, 1H), 3.35 (dd. J=10.2, 18.2 Hz, 1H), 2.70 (dd. J=4.0, 18.2 Hz, 1H), 2.34 (s, 3H), 2.17 (s, 3H).

Method 4

### Synthesis of 3-(1,5-Disubstituted-1H-pyrazol-3-yl)-2-aryl-propionic Acids and 3-(2,5-Disubstituted-4H-pyrazol-5-yl)-2-aryl-propionic Acids, such as

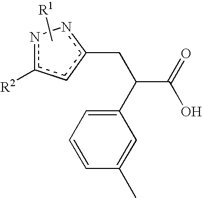

Scheme E. To a slurry of 10.0 g of 4-sulfamylbenzoyl AM resin (NovaBiochem, 1.21 mmol/g) in 1:1 THF/CH₂Cl₂ (70 mL) was added DMAP (0.201 g, 1.65 mmol), 4-oxo-2-m-tolyl-pentanoic acid (E1) (17.7 g, 86.0 mmol) prepared by Method 3, N,N-diisopropylethylamine (7.51 mL, 43.0 mmol), and diisopropylcarbodiimide (6.72 mL, 43.0 mmol). The mixture was shaken overnight, and the filtrate was drained under reduced pressure. The resin was then washed (3×5 mL) with 1:1 THF/CH₂Cl₂, MeOH, DMF, MeOH, and THF and then dried under vacuum overnight to give the coupled resin E2 (theoretical loading: 0.98 mmol/g). The resin was then loaded into a 48-position Bohdan miniblock (˜200 mg/well) along with the appropriate ester E5 (3.60 mmol, 18 equiv), and the inert atmosphere manifold was added (N₂). To each well was then added 1.0 M NaHMDS in THF (3.63 mmol, 18 equiv), and the block was heated to 50° C. overnight. The block was cooled, the solvent was removed under reduced pressure, and each well was washed (3×5 mL) with cold 4:1 AcOH/H₂O, THF, DMF, and MeOH. After the resin was dried under reduced pressure, the appropriate hydrazines E6 (2.40 mmol, 12 equiv) were then loaded into the wells of the block followed by MeOH (3.0 mL), providing a unique resin in each of the 48 wells of the block, and the reaction mixtures were heated to 65° C. and shaken overnight. The block was cooled, the solvent was removed under reduced pressure, and each well was washed (3×5 mL) with THF, MeOH, and THF. After the resin was dried under reduced pressure, THF (1.0 mL) was added to each well followed by 1.0 M (trimethylsilyl)diazomethane (TMSCHN₂) in hexane (1.0 mmol, 10 equiv), and the block was shaken for 1 h. The filtrates were drained under reduced pressure, and the TMSCHN₂ treatment was repeated. The resin was then diluted with 3:1:1 THF/MeOH/H₂O (2.5 mL/well), LiOH.H₂O (1.0 mmol, 10 equiv) was added to each well, and the block was heated to 50° C. overnight. The block was cooled and the reaction mixtures were drained into a 48-well Beckman plate. The resin was then washed with MeOH, DMF and THF (3.0 mL each), each wash being drained into a 48-well plate, and the solvent was removed under reduced pressure. The plated compounds were dissolved in DMF (1.5 mL total volume/well), and identical compounds were combined and purified on a Gilson 215 prep-HPLC system (Method G) giving the desired acids (A9) (0.5-7.0 mg, isolated as TFA salt) as well as, in some cases, the other regioisomer of the pyrazole. The 1,5-disubstituted and the 2,5-disubstituted pyrazole regioisomers were isolated and characterized, and the isomer structures were confirmed by assignment of COSY and NOESY spectra. For the 2,5-disubstituted pyrazole regioisomer, enhancement was observed between the N-aryl protons and the alkyl side-chain.

### Example 47

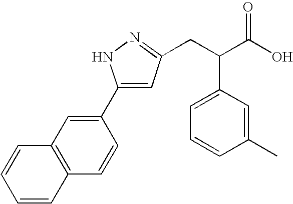

### 3-(5-Naphthalen-2-yl-1H-pyrazol-3-yl)-2-m-tolyl-propionic acid

The title compound was prepared by Method 4: HPLC: Rₜ=2.91 (Method B). MS (ES+): mass calculated for C₂₃H₂₀N₂O₂, 356.15; m/z found, 357.2 [M+H]⁺. ¹H NMR (400 MHz, CDCl₃): 8.08 (s, 1H), 7.87-7.70 (m, 4H), 7.49-7.41 (m, 2H), 7.36-7.23 (m, 4H), 7.19 (d, J=7.1 Hz, 1H), 6.58 (s, 1H), 3.95 (d, J=11.9 Hz, 1H), 3.66 (t, J=12.6 Hz, 1H), 3.05 (d, J=13.6 Hz, 1H), 2.42 (s, 3H).

### Example 48

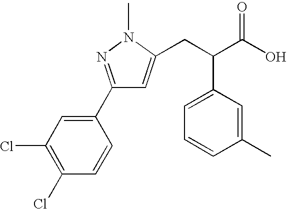

### 3-[5-(3,4-Dichloro-phenyl)-2-methyl-2H-pyrazol-3-yl]-2-m-tolyl-propionic acid

The title compound was prepared by Method 4: HPLC: Rₜ=3.30 (Method B). MS (ES+): mass calculated for C₂₀H₁₈Cl₂N₂O₂, 388.07; m/z found, 388.9 [M+H]⁺. ¹H NMR (400 MHz, CDCl₃): 7.81 (d, J=2.0 Hz, 1H), 7.54 (dd, J=8.3, 2.0 Hz, 1H) 7.42 (d, J=8.0 Hz, 1H), 7.16-7.10 (m, 4H), 6.30 (s, 1H), 3.92 (dd, J=8.9, 6.1 Hz, 1H), 3.74 (s, 3H), 3.45 (dd, J=15.4, 8.9 Hz, 1H), 3.00 (dd, J=15.4, 6.1 Hz, 1H), 2.35 (s, 3H).

### Example 49

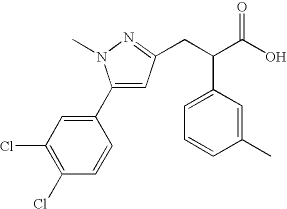

### 3-[5-(3,4-Dichloro-phenyl)-1-methyl-1H-pyrazol-3-yl]-2-m-tolyl-propionic acid

The title compound was prepared by Method 4: HPLC: Rₜ=3.18 (Method B). MS (ES+): mass calculated for C₂₀H₁₈Cl₂N₂O₂, 388.07; m/z found, 388.9 [M+H]⁺. ¹H NMR (400 MHz, CDCl₃): 7.50 (d, J=8.3 Hz, 1H), 7.45 (d, J=2.3 Hz, 1H), 7.24-7.14 (m, 4H), 7.10 (d, J=7.6 Hz, 1H), 6.03 (s, 1H), 4.03 (dd, J=9.7, 5.5 Hz, 1H), 3.79 (s, 3H), 3.46 (dd, J=14.9, 9.7 Hz, 1H), 3.03 (dd, J=14.9, 5.5 Hz, 1H), 2.34 (s, 3H).

### Example 50

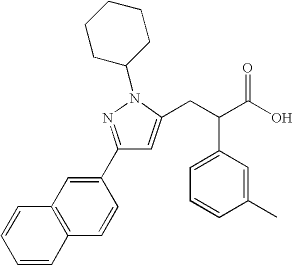

### 3-(2-Cyclohexyl-5-naphthalen-2-yl-2H-pyrazol-3-yl)-2-m-tolyl-propionic acid

The title compound was prepared by Method 4: HPLC: Rₜ=3.71 (Method B). MS (ES+): mass calculated for C₂₉H₃₀N₂O₂, 438.23; m/z found, 439.2 [M+H]⁺. ¹H NMR (400 MHz, CDCl₃): 8.20 (s, 1H), 7.88-7.78 (m, 4H), 7.51-7.44 (m, 2H), 7.28-7.22 (m, 1H), 7.18-7.11 (m, 3H), 6.48 (s, 1H), 4.08 (app tt, J=11.9, 3.5 Hz, 1H), 3.97 (dd, J=8.5, 6.8 Hz, 1H), 3.52 (dd, J=15.4, 8.5 Hz, 1H), 3.08 (dd, J=15.4, 6.8 Hz, 1H), 2.35 (s, 3H), 2.15-1.99 (m, 2H), 1.97-1.80 (m, 3H), 1.75-1.58 (m, 2H), 1.45-1.16 (m, 3H).

### Example 51

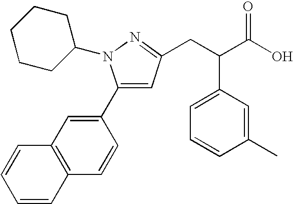

### 3-(1-Cyclohexyl-5-naphthalen-2-yl-1H-pyrazol-3-yl)-2-m-tolyl-propionic acid

The title compound was prepared by Method 4: HPLC: Rₜ=3.56 (Method B). MS (ES+): mass calculated for C₂₉H₃₀N₂O₂, 438.23; m/z found, 439.2 [M+H]⁺. ¹H NMR (400 MHz, CDCl₃): 7.95-7.85 (m, 3H), 7.79 (s, 1H), 7.60-7.55 (m, 2H), 7.38 (dd, J=8.3, 1.8 Hz, 1H), 7.24-7.12 (m, 3H), 7.08 (d, J=7.3 Hz, 1H), 6.10 (s, 1H), 4.18 (dd, J=9.5, 4.8 Hz, 1H), 4.14 (app tt, J=11.6, 3.8 Hz, 1H), 3.53 (dd, J=15.3, 9.5 Hz, 1H), 3.17 (dd, J=15.3, 4.8 Hz, 1H), 2.33 (s, 3H), 2.14-1.77 (m, 6H), 1.67-1.58 (m, 1H), 1.31-1.11 (m, 3H).

### Example 52

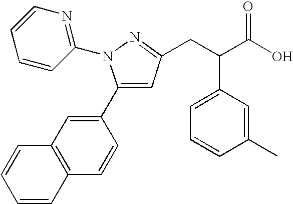

### 3-(5-Naphthalen-2-yl-1-pyridin-2-yl-1H-pyrazol-3-yl)-2-m-tolyl-propionic acid

The title compound was prepared by Method 4: HPLC: Rₜ=3.21 (Method B). MS (ES+): mass calculated for C₂₈H₂₃N₃O₂, 433.18; m/z found, 434.2 [M+H]⁺. ¹H NMR (400 MHz, CDCl₃): 8.34 (d, J=4.3 Hz, 1H), 7.83-7.62 (m, 5H), 7.52-7.45 (m, 2H), 7.33 (d, J=8.1 Hz, 1H), 7.29-7.14 (m, 5H), 7.13-7.03 (m, 1H), 6.34 (s, 1H), 4.17 (dd, J=9.6, 5.5 Hz, 1H), 3.60 (dd, J=14.9, 9.6 Hz, 1H), 3.16 (dd, J=14.9, 5.5 Hz, 1H), 2.35 (s, 3H).

### Example 53

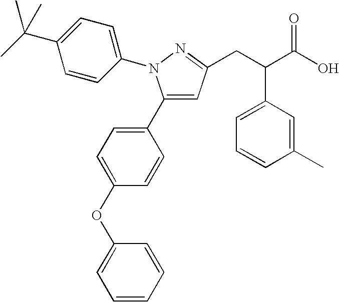

### 3-[1-(4-tert-Butyl-phenyl)-5-(4-phenoxy-phenyl)-1H-pyrazol-3-yl]-2-m-tolyl-propionic acid

The title compound was prepared by Method 4: HPLC: Rₜ=3.87 (Method B). MS (ES+): mass calculated for C₃₅H₃₄N₂O₃, 530.26; m/z found, 531.2 [M+H]⁺. ¹H NMR (400 MHz, CDCl₃): 7.40-7.05 (m, 13H), 7.02 (d, J=7.9 Hz, 2H), 6.87 (d, J=8.8 Hz, 2H), 6.20 (s, 1H), 4.10 (dd, J=9.5, 5.6 Hz, 1H), 3.54 (dd, J=14.9, 9.5 Hz, 1H), 3.12 (dd, J=14.9, 5.6 Hz, 1H), 2.34 (s, 3H), 1.29 (s, 9H).

### Example 54

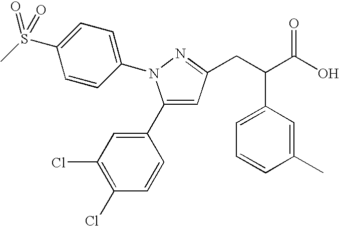

### 3-[5-(3,4-Dichloro-phenyl)-1-(4-methanesulfonyl-phenyl)-1H-pyrazol-3-yl]-2-m-tolyl-propionic acid

The title compound was prepared by Method 4: HPLC: Rₜ=3.24 (Method B). MS (ES+): mass calculated for C₂₆H₂₂Cl₂N₂O₄S, 528.07; m/z found, 529.1 [M+H]⁺. ¹H NMR (400 MHz, CDCl₃): 7.90 (d, J=8.6 Hz, 2H), 7.43 (d, J=8.6 Hz, 2H), 7.39 (d, J=8.5 Hz, 1H), 7.35 (d, J=2.0 Hz, 1H), 7.28-7.17 (m, 3H), 7.13 (d, J=7.4 Hz, 1H), 6.92 (dd, J=8.4, 2.0 Hz, 1H), 6.27 (s, 1H), 4.12 (dd, J=9.5, 5.8 Hz, 1H), 3.54 (dd, J=15.2, 9.5 Hz, 1H), 3.11 (dd, J=15.2, 5.8 Hz, 1H), 3.06 (s, 3H), 2.34 (s, 3H).

### Example 55

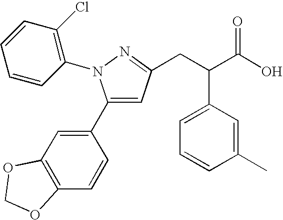

### 3-[5-Benzo[1,3]dioxol-5-yl-1-(2-chloro-phenyl)-1H-pyrazol-3-yl]-2-m-tolyl-propionic acid

The title compound was prepared by Method 4: HPLC: Rₜ=3.12 (Method B). MS (ES+): mass calculated for C₂₆H₂₁ClN₂O₄, 460.12; m/z found, 461.0 [M+H]⁺. ¹H NMR (400 MHz, CDCl₃): 7.44-7.14 (m, 7H), 7.09 (d, J=7.1 Hz, 1H), 6.66 (d, J=7.8 Hz, 1H), 6.61-6.55 (m, 2H), 6.18 (s, 1H), 5.92 (s, 2H), 4.09 (dd, J=8.9, 6.3 Hz, 1H), 3.52 (dd, J=14.9, 8.9 Hz, 1H), 3.14 (dd, J=14.9, 6.3 Hz, 1H), 2.33 (s, 3H).

### Example 56

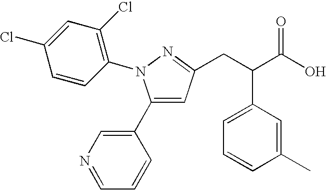

### 3-[1-(2,4-Dichloro-phenyl)-5-pyridin-3-yl-1H-pyrazol-3-yl]-2-m-tolyl-propionic acid

The title compound was prepared by Method 4: HPLC: Rₜ=2.50 (Method B). MS (ES+): mass calculated for C₂₄H₁₉Cl₂N₃O₂, 451.09; m/z found, 452.0 [M+H]⁺. ¹H NMR (400 MHz, CDCl₃): 8.60 (s, 1H), 8.58 (s, 1H), 7.56 (d, J=8.1 Hz, 1H), 7.44-7.30 (m, 4H), 7.24-7.15 (m, 3H), 7.10 (d, J=7.4 Hz, 1H), 6.44 (s, 1H), 4.09 (dd, J=9.3, 6.0 Hz, 1H), 3.55 (dd, J=14.9, 9.3 Hz, 1H), 3.15 (dd, J=14.9, 6.0 Hz, 1H), 2.34 (s, 3H).

### Example 57

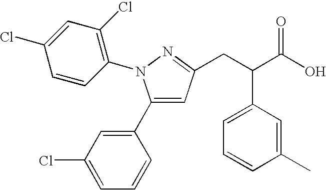

### 3-[5-(3-Chloro-phenyl)-1-(2,4-dichloro-phenyl)-1H-pyrazol-3-yl]-2-m-tolyl-propionic acid

The title compound was prepared by Method 4: HPLC: Rₜ₌3.53 (Method B). MS (ES+): mass calculated for C₂₅H₁₉Cl₃N₂O₂, 484.05; m/z found, 485.1 [M+H]⁺. ¹H NMR (400 MHz, CDCl₃): 7.42 (s, 1H), 7.32-7.13 (m, 8H), 7.10 (d, J=7.1 Hz, 1H), 6.90 (d, J=7.6 Hz, 1H), 6.26 (s, 1H), 4.10 (dd, J=9.1, 6.3 Hz, 1H), 3.52 (dd, J=14.9, 9.1 Hz, 1H), 3.13 (dd, J=14.9, 6.3 Hz, 1H), 2.34 (s, 3H).

Method 5

### Synthesis of 4-(4-Oxo-2-aryl-pentanoylsulfamoyl)-benzoic Acids, such as

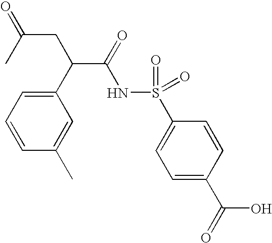

### 4-(4-Oxo-2-m-tolyl-pentanoylsulfamoyl)-benzoic acid

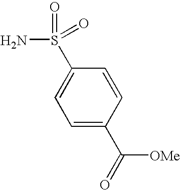

A. 4-Sulfamoyl-benzoic acid methyl ester. To a stirred suspension of 4-sulfamoyl-benzoic acid (25.0 g, 0.124 mol) in 4:1 CH₂Cl₂/MeOH at rt was added 1.0 M TMSCHN₂ in hexane (175 mL), and the reaction mixture was allowed to stir for 2 h. The mixture was diluted with 1 N NaOH (100 mL) and CH₂Cl₂ (150 mL), and the layers were separated. The organic layer was dried over Na₂SO₄, then filtered, and the solvent was removed under reduced pressure to afford the desired ester (25.2 g, 95%), which was used without further purification. ¹H NMR (400 MHz, DMSO-d₆): 8.14 (d, J=8.1 Hz, 2H), 7.96 (d, J=8.1 Hz, 2H), 7.58 (s, 2H), 3.90 (s, 3H).

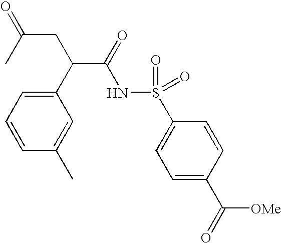

B. 4-(4-Oxo-2-m-tolyl-pentanoylsulfamoyl)-benzoic acid methyl ester. To a stirred solution of 4-sulfamoyl-benzoic acid methyl ester (6.01 g, 27.8 mmol), 4-oxo-2-m-tolyl-pentanoic acid (6.35 g, 30.7 mmol), N,N-diisopropylethylamine (12.2 mL, 69.5 mmol), and DMAP (5 mole %) in CH₂Cl₂ (275 mL) at rt under N₂ was added bromo-tripyrrolidino-phosphonium hexafluorophosphate (PyBroP) (18.1 g, 38.9 mmol), and the reaction mixture was allowed to stir overnight. The mixture was diluted with 1 M HCl (100 mL) and CH₂Cl₂ (150 mL), and the layers were separated. The organic phase was washed with 1 M HCl (1×100 mL), 1N NaOH (1×100 mL) and brine (1×100 mL). The organic layer was dried over Na₂SO₄, and then filtered, and the solvent was removed under reduced pressure. Purification on silica gel (0-15% EtOAc in hexane) gave. 12.0 g (99%) of desired ester as a white solid. ¹H NMR (400 MHz, CDCl₃): 8.15 (d, J=8.6 Hz, 2H), 7.99 (d, J=8.6 Hz, 2H), 7.18 (t, J=7.6 Hz, 1H), 7.10 (d, J=7.6 Hz, 1H), 6.87 (m, 2H), 3.97 (s, 3H), 3.93 (dd. J=4.3 and 9.5 Hz, 1H), 3.29 (dd. J=9.5 and 18.1 Hz, 1H), 2.60 (dd. J=4.3 and 18.1 Hz, 1H), 2.28 (s, 3H), 2.07 (s, 3H).

C. 4-(4-Oxo-2-m-tolyl-pentanoylsulfamoyl)-benzoic acid. To a stirred solution of 4-(4-oxo-2-m-tolyl-pentanoylsulfamoyl)-benzoic acid methyl ester (12.0 g, 27.7 mmol) in 3:1:1 THF/MeOH/H₂O (110 mL) was added LiOH.H₂O (5.84 g, 139 mmol), and the mixture was stirred overnight at rt. The mixture was then heated to 65° C. for 2 h, cooled to rt, and then was diluted with H₂O (100 mL) and 20% diethyl ether/hexane. The layers were separated, and the aqueous layer was adjusted to pH 1 with concd HCl at 0° C. The aqueous phase was then extracted with EtOAc (3×200 mL), dried over Na₂SO₄, and filtered, and the solvent was removed under reduced pressure to afford 10.6 g (96%) of crude acid as a white solid. TLC (silica, 5% MeOH—CH₂Cl₂): R_{f}=0.2. ¹H NMR (400 MHz, DMSO-d₆): 8.06 (d, J=8.1 Hz, 2H), 7.96 (d, J=8.1 Hz, 2H), 7.16 (t, J=7.6 Hz, 1H), 7.05 (d, J=7.6 Hz, 1H), 6.93 (d, J=7.6 Hz, 1H), 6.82 (s, 1H), 3.89 (dd. J=3.9, 10.6 Hz, 1H), 3.14 (dd. J=10.6, 18.3 Hz, 1H), 2.70 (dd. J=3.9, 18.3 Hz, 1H), 2.19 (s, 3H), 2.00 (s, 3H).

Method 6

### Synthesis of 3-(1,5-Disubstituted-1H-pyrazol-3-yl)-2-aryl-propionic Acids and 3-(2,5-Disubstituted-4H-pyrazol-5-yl)-2-aryl-propionic Acids, such as

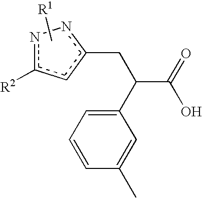

Scheme F. To a slurry of 5.0 g of 4-aminomethyl macroporous polystyrene resin (ArgoPore-NH₂-HL, 1.22 mmol/g) in THF (30 mL) was added HOBt (1.66 g, 12.2 mmol), 4-(4-oxo-2-m-tolyl-pentanoylsulfamoyl)-benzoic acid (E1) (4.81 g, 12.2 mmol) prepared by Method 5, and diisopropylcarbodiimide (1.91 mL, 12.2 mmol). The mixture was shaken overnight and the filtrate was drained under reduced pressure. The resin was then washed (3×5 mL) with THF, CH₂Cl₂, MeOH, DMF and THF and then dried under vacuum overnight to give the coupled resin F3 (−0.75 mmol/g based on elemental analysis of sulfur). The resin was then loaded into a 48-position Bohdan miniblock (−230 mg/well) along with the appropriate ester F6 (2.20 mmol, 12.0 equiv), and the inert atmosphere manifold was added (N₂). To each well was then added 1.0 M NaHMDS in THF (1.80 mmol, 12 equiv), and the block was heated to 50° C. overnight. The block was cooled, the solvent was removed under reduced pressure, and each well was washed (3×5 mL) with 5% TFA/THF, H₂O, THF, DMF, and MeOH. After the resin F4 was dried under reduced pressure, the appropriate hydrazines F7 (1.80 mmol, 10 equiv) were added to the wells followed by MeOH (3.0 mL) and N,N-diisopropylethylamine (0.32 mL, 1.8 mmol, for aryl hydrazines) or H₂SO₄ (2 drops, for alkyl hydrazines), creating a unique product in each well of the 48-well miniblock, and the reaction mixtures were heated to 65° C. overnight. The block was cooled, the solvent was removed under reduced pressure, and each well was washed (3×5 mL) with 5% TFA/THF, THF, MeOH, DMF and THF. After the resin F5 was dried under reduced pressure, THF (1.0 mL) was added to each well followed by 1.0 M TMSCHN₂ in hexane (1.0 mL, 14.0 equiv), and the block was shaken for 1 h. The filtrates were drained under reduced pressure and the TMSCHN₂ procedure was repeated. The resin was then diluted with 2:1 2N NaOH/THF (2.5 mL/well), and the block was heated to 50° C. overnight. The block was cooled, and the reaction mixtures were drained into a 48-well Beckman plate. The resin was then washed with MeOH, DMF and THF (3.0 mL each), each wash being drained into a 48-well plate, and the solvent was removed under reduced pressure. The plated compounds were dissolved in DMF (1.5 mL total volume/well), and identical compounds were combined and purified on a Gilson 215 prep-HPLC system (Method G) giving the desired acids (A9) (3.0-11.0 mg, isolated as TFA salt) as well as, in some cases, the other regioisomer of the pyrazole. The 1,5-disubstituted and the 2,5-disubstituted pyrazole regioisomers were isolated and characterized, and the isomer structures were confirmed by assignment of COSY and NOESY spectra. For the 2,5-disubstituted pyrazole regioisomer, enhancement was observed between the N-aryl protons and the alkyl side-chain.

### Example 58

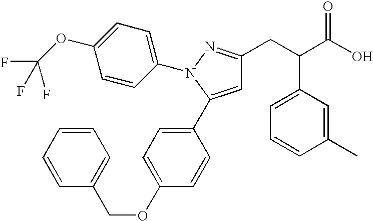

### 3-[5-(4-Benzyloxy-phenyl)-1-(4-trifluoromethoxy-phenyl)-1H-pyrazol-3-yl]-2-m-tolyl-propionic acid

The title compound was prepared by Method 6: HPLC: Rₜ=3.58 (Method B). MS (ES+): mass calculated for C₃₃H₂₇F₃N₂O₄, 572.19; m/z found, 573.5 [M+H]⁺. ¹H NMR (400 MHz, CDCl₃): 7.48-7.02 (m, 15H), 6.90 (d, J=8.6 Hz, 2H), 6.18 (s, 1H), 5.05 (s, 2H), 4.11 (dd, J=9.6, 5.6 Hz, 1H), 3.53 (dd, J=14.9, 9.6 Hz, 1H), 3.11 (dd, J=14.9, 5.6 Hz, 1H), 2.34 (s, 3H).

### Example 59

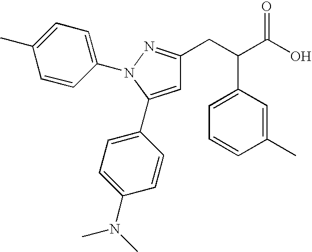

### 3-[5-(4-Dimethylamino-phenyl)-1-p-tolyl-1H-pyrazol-3-yl]-2-m-tolyl-propionic acid

The title compound was prepared by Method 6: HPLC: Rₜ=2.65 (Method B). MS (ES+): mass calculated for C₂₈H₂₉N₃O₂, 439.23; m/z found, 440.3 [M+H]⁺. ¹H NMR (400 MHz, CDCl₃): 7.24-7.03 (m, 12H), 6.24 (s, 1H), 4.15 (dd, J=9.9, 5.6 Hz, 1H), 3.54 (dd, J=14.9, 9.9 Hz, 1H), 3.30 (s, 3H), 3.14 (dd, J=14.9, 5.6 Hz, 1H), 2.37 (s, 3H), 2.36 (s, 6H).

### Example 60

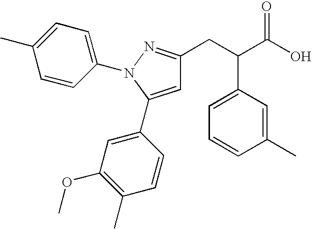

### 3-[5-(3-Methoxy-4-methyl-phenyl)-1-p-tolyl-1H-pyrazol-3-yl]-2-m-tolyl-propionic acid

The title compound was prepared by Method 6: HPLC: Rₜ=3.30 (Method B). MS (ES+): mass calculated for C₂₈H₂₈N₂O₃, 440.21; m/z found, 441.3 [M+H]⁺. ¹H NMR (400 MHz, CDCl₃): 7.24-7.08 (m, 8H), 7.02 (d, J=7.6 Hz, 1H), 6.69 (dd, J=7.6, 1.0 Hz, 1H), 6.54 (s, 1H), 6.21 (s, 1H), 4.14 (dd, J=9.4, 5.3 Hz, 1H), 3.58 (s, 3H), 3.54 (dd, J=15.0, 9.6 Hz, 1H), 3.14 (dd, J=15.0, 5.3 Hz, 1H), 2.35 (s, 3H), 2.34 (s, 3H), 2.18 (s, 3H).

### Example 61

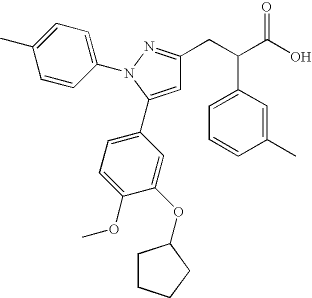

### 3-[5-(3-Cyclopentyloxy-4-methoxy-phenyl)-1-p-tolyl-1H-pyrazol-3-yl]-2-m-tolyl-propionic acid

The title compound was prepared by Method 6: HPLC: Rₜ=3.33 (Method B). MS (ES+): mass calculated for C₃₂H₃₄N₂O₄, 510.25; m/z found, 511.4 [M+H]⁺. ¹H NMR (400 MHz, CDCl₃): 7.25-7.05 (m, 9H), 6.82-6.79 (m, 1H), 6.50 (d, J=2.0 Hz, 1H), 6.20 (s, 1H), 4.39 (app tt, J=4.8, 4.8 Hz, 1H), 4.15 (dd, J=9.8, 5.4 Hz, 1H), 3.83 (s, 3H), 3.55 (dd, J=15.0, 9.8 Hz, 1H), 3.14 (dd, J=15.0, 5.4 Hz, 1H), 2.35 (s, 3H), 2.34 (s, 3H), 1.76-1.68 (m, 2H), 1.67-1.59 (m, 4H), 1.55-1.45 (m, 2H).

### Example 62

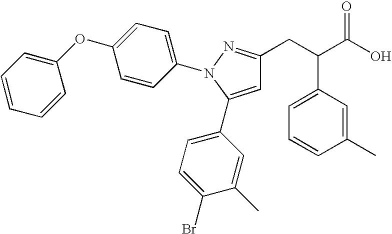

### 3-[5-(4-Bromo-3-methyl-phenyl)-1-(4-phenoxy-phenyl)-1H-pyrazol-3-yl]-2-m-tolyl-propionic acid

The title compound was prepared by Method 6: HPLC: Rₜ=3.69 (Method B). MS (ES+): mass calculated for C₃₂H₂₇BrN₂O₃, 566.12; m/z found, 567.4 [M+H]⁺. ¹H NMR (400 MHz, CDCl₃): 7.47-6.91 (m, 15H), 6.80 (dd, J=8.1, 2.0 Hz, 1H), 6.23 (s, 1H), 4.13 (dd, J=9.7, 5.5 Hz, 1H), 3.54 (dd, J=14.9, 9.7 Hz, 1H), 3.13 (dd, J=14.9, 5.5 Hz, 1H), 2.35 (s, 3H), 2.33 (s, 3H).

### Example 63

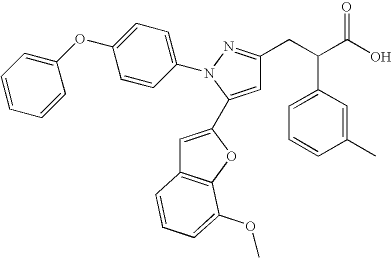

### 3-[5-(7-Methoxy-benzofuran-2-yl)-1-(4-phenoxy-phenyl)-1H-pyrazol-3-yl]-2-m-tolyl-propionic acid

The title compound was prepared by Method 6: HPLC: Rₜ=3.53 (Method B). MS (ES+): mass calculated for C₃₄H₂₈N₂O₅, 544.20; m/z found, 545.4 [M+H]⁺. ¹H NMR (400 MHz, CDCl₃): 7.43-7.35 (m, 3H), 7.31-7.01 (m, 12H), 6.80 (d, J=7.8 Hz, 1H), 6.68 (s, 1H), 6.23 (s, 1H), 4.14 (dd, J=9.2, 5.8 Hz, 1H), 3.98 (s, 3H), 3.54 (dd, J=14.9, 9.2 Hz, 1H), 3.14 (dd, J=14.9, 5.8 Hz, 1H), 2.35 (s, 3H), 2.34 (s, 3H).

### Example 64

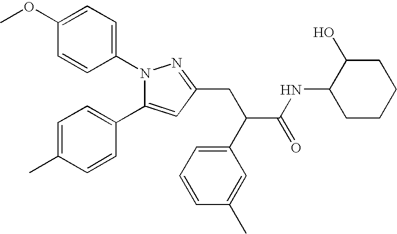

### N-(2-Hydroxy-cyclohexyl)-3-[1-(4-methoxy-phenyl)-5-p-tolyl-1H-pyrazol-3-yl]-2-m-tolyl-propionamide

To a solution of 3-[1-(4-methoxy-phenyl)-5-p-tolyl-1H-pyrazol-3-yl]-2-m-tolyl-propionic acid (product of Method 2) (100 mg, 0.23 mmol), EDC (65 mg, 0.35 mmol), and HOBT (46 mg, 0.34 mmol) in DMF (4.0 mL) was added trans-2-aminocyclohexanol hydrochloride (52 mg, 0.34 mmol) and DIEA (0.20 mL, 1.2 mmol). The reaction mixture was stirred for 24 h, diluted with EtOAc, and washed with 1.0 N NaOH (2×25 mL), water (1×25 mL), 5% formic acid (2×25 mL), water (1×25 mL) and brine (1×25 mL). The organic layer was dried (Na₂SO₄) and the solvent was removed under reduced pressure. Reversed-phase HPLC afforded 40 mg (33%) of N-(2-hydroxy-cyclohexyl)-3-[1-(4 -methoxy-phenyl)-5-p-tolyl-1H-pyrazol-3-yl]-2-m-tolyl-propionamide as a mixture of diastereomers. HPLC: Rₜ=3.17 (Method B). MS (ES+): mass calculated for C₃₃H₃₇N₃O₃, 523.28; m/z found 524.2 [M+H]⁺. ¹H NMR (400 MHz, CDCl₃): 7.92-7.85 (m, 1H), 7.26-7.10 (m, 6H), 7.05-7.01 (m, 3H), 6.94-6.91 (m, 2H), 6.32 (s, 0.5H), 6.29 (s, 0.5H), 4.42 (d, J=4.7 Hz, 0.5H), 4.34 (d, J=5.4 Hz, 0.5H), 3.90 (ddd, J=5.4, 9.4, 20.3 Hz, 1H), 3.76 (s, 3H), 3.24 (m, 0.5H), 3.17 (m, 0.5H), 2.85 (m, 1H), 2.30 (s, 1.5H), 2.28 (s, 1.5H), 2.27 (s, 3H), 1.75 (m, 1H), 1.55 (m, 2H), 1.13 (m, 4H), 0.97 (m, 1H).

### Example 65

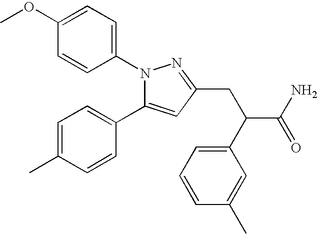

### 3-[1-(4-methoxy-phenyl)-5-p-tolyl-1H-pyrazol-3-yl]-2-m-tolyl-propionamide

A mixture of 3-[1-(4-methoxy-phenyl)-5-p-tolyl-1H-pyrazol-3-yl]-2-m-tolyl-propionic acid (product of Method 2) (0.10 g, 0.23 mmol) and CDI (85 mg, 0.52 mmol) in DMF (2.5 mL) was stirred at rt for 30 min. The solution was then cooled to 0° C., and ammonium carbonate (99 mg, 1.0 mmol) was added in portions. The reaction mixture was allowed to warm to rt and stirred for an additional 18 h. The reaction mixture was then diluted with water (25 mL) and extracted with EtOAc (3×25 mL). Organic layers were combined, washed with water (3×25 mL) and brine (1×25 mL) and dried with Na₂SO₄, and the solvent removed under reduced pressure giving 70 mg (71%) of the title compound. HPLC: Rₜ=9.38 (Method A). MS (ES+): mass calculated for C₂₇H₂₇N₃O₂, 425.21; m/z found 426.2 [M+H]⁺. ¹H NMR (400 MHz, DMSO-d₆): 7.50 (s, 1H), 7.22 (s, 1H), 7.20 (d, J=5.1 Hz, 2H), 7.14-7.10 (m, 3H), 7.04 (d, J=8.2 Hz, 2H), 6.93 (d, J=9.0 Hz, 2H), 6.82 (s, 1H), 6.27 (s, 1H), 3.89 (dd, J=5.5, 9.6 Hz, 1H), 3.76 (s, 3H), 3.34 (m, 1H), 2.82 (dd, J=5.5, 14.7 Hz, 1H), 2.29 (s, 3H), 2.27 (s, 3H).

### Example 66

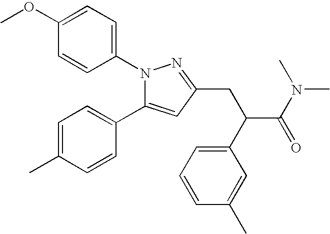

### 3-[1-(4-Methoxy-phenyl)-5-p-tolyl-1H-pyrazol-3-yl]-N,N-dimethyl-2-m-tolyl-propionamide

The title compound was prepared analogously to Example 64, where N,N-dimethylamine hydrochloride was substituted for trans-2-aminocyclohexanol hydrochloride. HPLC: Rₜ=10.13 (Method A). MS (ES+): mass calculated for C₂₉H₃₁N₃O₂, 453.24; m/z found 454.2 [M+H]⁺. ¹H NMR (400 MHz, DMSO-d₆): 7.22-7.08 (m, 7H), 7.06-7.03 (m, 3H), 6.93 (d, J=9.0 Hz, 2H), 6.25 (s, 1H), 4.39 (dd, J=5.6, 9.0 Hz, 1H), 3.76 (s, 3H), 3.35 (dd, J=8.8, 14.8 Hz, 1H), 2.95 (s, 3H), 2.81 (s, 3H), 2.80 (dd, J=5.6, 14.8 Hz, 1H), 2.28 (s, 3H), 2.27 (s, 3H).

### Example 67

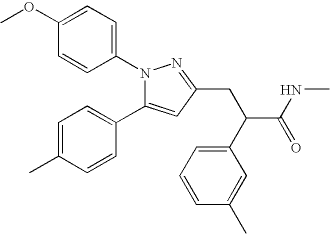

### 3-[1-(4-Methoxy-phenyl)-5-p-tolyl-1H-pyrazol-3-yl]-N-methyl-2-m-tolyl-propionamide

The title compound was prepared analogously to Example 64, where N-methylamine hydrochloride was substituted for trans-2-aminocyclohexanol hydrochloride. HPLC: Rₜ=9.62 (Method A). MS (ES+): mass calculated for C₂₈H₂₉N₃O₂, 439.23; m/z found 440.2 [M+H]⁺. ¹H NMR (400 MHz, DMSO-d₆): 7.99 (q, J=4.7 Hz, 1H), 7.20-7.18 (m, 3H), 7.14-7.09 (m, 4H), 7.04-7.01 (m, 3H), 6.93 (d, J=9.0 Hz, 2H), 6.22 (s, 1H), 3.85 (dd, J=5.8, 9.4 Hz, 1H), 3.76 (s, 3H), 3.35 (dd, J=9.4, 14.6 Hz, 1H), 2.86 (dd, J=5.7, 14.6 Hz, 1H), 2.54 (s, 1.5H), 2.53 (s, 1.5H), 2.329 (s, 3H), 2.27 (s, 3H).

### Example 68

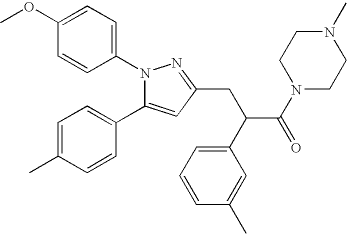

### 3-[1-(4-Methoxy-phenyl)-5-p-tolyl-1H-pyrazol-3-yl]-1-(4-methyl-piperazin-1-yl)-2-m-tolyl-propan-1-one

The title compound was prepared analogously to Example 64, where N-methyl piperazine was substituted for trans-2-aminocyclohexanol hydrochloride. HPLC: Rₜ=8.37 (Method A). MS (ES+): mass calculated for C₃₂H₃₆N₄O₂, 508.28; m/z found 509.2 [M+H]⁺. ¹H NMR (400 MHz, DMSO-d₆): 7.24-7.17 (m, 3H), 7.14-7.11 (m, 4H), 7.07 (d, J=7.6 Hz, 1H), 7.04 (d, J=8.2 Hz, 2H), 6.95 (d, J=9.0 Hz, 2H), 6.27 (s, 1H), 4.53 (dd, J=5.8, 8.8 Hz, 1H), 3.76 (s, 3H), 3.39 (dd, J=8.9, 15.0 Hz, 1H), 3.05 (br s, 4H), 2.90 (br s, 4H), 2.87 (dd, J=5.6, 15.0 Hz, 1H), 2.54 (s, 3H), 2.29 (s, 3H), 2.27 (s, 3H).

### Example 69

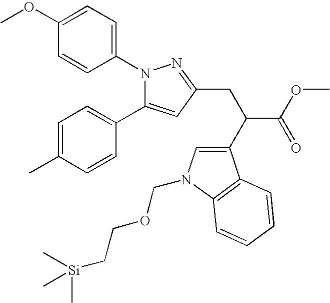

### 3-[1-(4-Methoxy-phenyl)-5-p-tolyl-1H-pyrazol-3-yl]-2-[1-(2-trimethylsilanyl-ethoxymethyl)-1H-indol-3-yl]-propionic acid methyl ester

A. [1-(2-Trimethylsilanyl-ethoxymethyl)-1H-indol-3-yl]-acetic acid methyl ester. To a suspension of sodium hydride (326 mg, 8.10 mmol) in DMF (13 mL) at 0° C. was added a solution of (1H-Indol-3-yl)-acetic acid methyl ester (1.0 g, 5.3 mmol) in DMSO (3 mL). The mixture was stirred at 0° C. for 30 min and then at rt for 1 h. The reaction mixture was cooled back down to 0° C., and SEMCI (1.35 mL, 8.41 mmol) was added neat. The reaction mixture was stirred at 0° C. for 15 min and then at rt for 1 h. The reaction mixture was then partitioned between water (200 mL) and diethyl ether (200 mL) followed by further extraction of the water layer with ether (2×200 mL) and drying of the combined organic layers with Na₂SO₄. After removal of the solvent under reduced pressure, the crude material was purified by flash chromatography (EtOAc/hexanes) giving 1.1 g (70%) of [1-(2-trimethylsilanyl-ethoxymethyl)-1H-indol-3yl]-acetic acid methyl ester. ¹H NMR (400 MHz, CDCl₃): 7.65 (d, J=7.8 Hz, 1H), 7.46 (d, J=8.1, 1H), 7.26 (m, 1H), 7.22 (m, 2H), 5.51 (s, 2H), 3.83 (s, 2H), 3.76 (s, 3H), 3.53 (t, J=7.9 Hz, 2H), 0.94 (t, J=7.9 Hz, 2H), 0.0 (s, 9H).

B. 3-[1-(4-Methoxy-phenyl)-5-p-tolyl-1H-pyrazol-3-yl]-2-[1-(2-trimethylsilanyl-ethoxymethyl)-1H-indol-3-yl]-propionic acid methyl ester. The title compound was synthesized via Method 2 from [1-(2-trimethylsilanyl-ethoxymethyl)-1H-indol-3yl]-acetic acid methyl ester (Step A, 0.17 g, 0.56 mmol), 3-bromoethyl-1-(4-methoxy-phenyl)-5-p-tolyl-1H-pyrazole (Method 1 pyrazole bromide, 0.10 g, 0.28 mmol), sodium hydride (22 mg, 0.56 mmol) and DMF (4.0 mL), yielding 140 mg (84%) of 3-[1-(4-methoxy-phenyl)-5-p-tolyl-1H-pyrazol-3-yl]-2-[1-(2-trimethylsilanyl-ethoxymethyl)-1H-indol-3-yl]-propionic acid methyl ester. HPLC: Rₜ=3.91 (Method B). MS (ES+): mass calculated for C₃₅H₄₁N₃O₄Si, 595.29; m/z found 596.27 [M+H]⁺. ¹H NMR (400 MHz, DMSO-d₆): 7.76 (d, J=7.8 Hz, 1H), 7.65 (d, J=8.2 Hz, 1H), 7.61 (s, 1H), 7.30 (t, J=7.6 Hz, 1H), 7.27-7.19 (m, 5H), 7.15 (d, J=8.1 Hz, 2H), 7.05 (d, J=9.0 Hz, 2H), 6.44 (s, 1H), 5.64 (s, 2H), 4.47 (t, J=7.6 Hz, 1H), 3.89 (s, 3H), 3.71 (s, 3H), 3.62-3.52 (m, 3H), 3.25 (dd, J=6.6, 14.9 Hz, 1H), 2.40 (s, 3H), 0.87 (t, J=8.0 Hz, 2H), 0.0 (s, 9H).

### Example 70

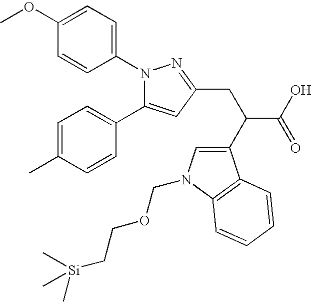

### 3-[1-(4-Methoxy-phenyl)-5-p-tolyl-1H-pyrazol-3-yl]-2-[1-(2-trimethylsilanyl-ethoxymethyl)-1H-indol-3-yl]-propionic acid

The title compound was synthesized by Method 2 from 3-[1-(4-methoxy-phenyl)-5-p-tolyl-1H-pyrazol-3-yl]-2-[1-(2-trimethylsilanyl-ethoxymethyl)-1H-indol-3-yl]-propionic acid methyl ester (Example 69, 0.19 g, 0.32 mmol), lithium hydroxide (40 mg, 0.96 mmol), THF (1.25 mL), water (0.43 mL) and MeOH (0.43 mL), giving 167 mg (89%) of 3-[1-(4-methoxy-phenyl)-5-p-tolyl-1H-pyrazol-3-yl]-2-[1-(2-trimethylsilanyl-ethoxymethyl)-1H-indol-3-yl]-propionic acid. HPLC: Rₜ=3.66 (Method B). MS (ES+): mass calculated for C₃₄H₃₉N₃O₄Si, 581.27; m/z found 582.3 [M+H]⁺. ¹H NMR (400 MHz, DMSO-d₆): 7.64 (d, J=8.2 Hz, 1H), 7.51 (d, J=8.2 Hz, 1H), 7.45 (s, 1H), 7.19-7.04 (m, 6H), 7.01 (d, J=8.2 Hz, 2H), 6.92 (d, J=9.0 Hz, 2H), 6.33 (s, 1H), 5.52 (s, 2H), 4.21 (m, 1H), 3.76 (s, 3H), 3.41 (m, 2H), 3.07 (dd, J=6.3, 14.3 Hz, 1H), 2.27 (s, 3H), 0.75 (t, J=8.0 Hz, 2H), 0.00 (s, 9H).

### Example 71

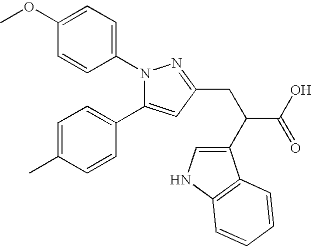

### 2-(1H-Indol-3-yl)-3-[1-(4-methoxy-phenyl)-5-p-tolyl-1H-pyrazol-3-yl]-propionic acid

A solution of 3-[1-(4-methoxy-phenyl)-5-p-tolyl-1H-pyrazol-3-yl]-2-[1-(2-trimethylsilanyl-ethoxymethyl)-1H-indol-3-yl]-propionic acid (Example 70, 0.17 g, 0.29 mmol) and 1.0 M TBAF (2.88 mL) in THF was heated to 60° C. for 24 h. The reaction mixture was cooled to rt, diluted with EtOAc (100 mL), and washed with water (3×30 mL) and brine (1×30 mL). The organic layer was dried with Na₂SO₄, and the solvent was removed under reduced pressure. The crude residue was purified by reversed-phase HPLC giving 111 mg (85%) of 2-(1H-indol-3-yl)-3-[1-(4-methoxy-phenyl)-5-p-tolyl-1H-pyrazol-3-yl]-propionic acid. HPLC: Rₜ=3.0 (Method B). MS (ES+): mass calculated for C₂₈H₂₅N₃O₃, 451.19; m/z found 452.2 [M+H]⁺. ¹H NMR (400 MHz, DMSO-d₆): 10.97 (s, 1H), 7.64 (d, J=6.3 Hz, 1H), 7.35 (d, J=8.1 Hz, 1H), 7.31 (d, J=2.4 Hz, 1H), 7.13-7.07 (m, 5H), 7.04 (d, J=8.1 Hz, 2H), 6.98 (t, J=8.0 Hz, 1H), 6.93 (d, J=9.0 Hz, 2H), 6.36 (s, 1H), 4.22 (dd, J=6.1, 9.0 Hz, 1H), 3.77 (s, 3H), 3.45 (dd, J=9.0, 14.7 Hz, 1H), 3.06 (dd, J=6.2, 14.7 Hz, 1H), 2.27 (s, 3H).

### Example 72

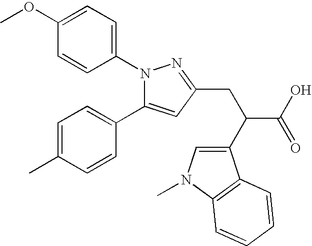

### 3-[1-(4-Methoxy-phenyl)-5-p-tolyl-1H-pyrazol-3-yl]-2-(1-methyl-1H-indol-3-yl)-propionic acid

A. (1-Methyl-1H-indol-3-yl)-acetic acid methyl ester. To a suspension of sodium hydride (104 mg, 7.61 mmol) in DMF (11 mL) was added a solution of 1H-indol-3-yl-acetic acid methyl ester (0.50 g, 2.6 mmol) in DMF (5.0 mL). The mixture was stirred for 1 h followed by addition of methyl iodide (1.1 g, 7.8 mmol). The reaction mixture was stirred for an additional 18 h, quenched, diluted with saturated ammonium chloride (200 mL), and then extracted with diethyl ether (3×100 mL). The combined organic layers were dried with Na₂SO₄, and the solvent was removed under reduced pressure. The crude residue was purified by flash chromatography (EtOAc/hexanes) giving 100 mg (19%) of (1-methyl-1H-indol-3-yl)-acetic acid methyl ester after purification. HPLC: Rₜ=8.91 (Method A). MS (ES+): mass calculated for Cl₂H₁₃NO₂, 203.09; m/z found 204.09 [M+H]⁺. ¹H NMR (400 MHz, CDCl₃): 7.60 (d, J=7.9 Hz, 1H), 7.30 (d, J=8.2 Hz, 1H), 7.23 (t, J=8.2 Hz, 1H), 7.13 (t, 7.4 Hz, 1H), 7.04 (s, 1H), 3.77 (s, 2H), 3.76 (s, 3H), 3.69 (s, 3H).

B. 3-[1-(4-Methoxy-phenyl)-5-p-tolyl-1H-pyrazol-3-yl]-2-(1-methyl-1H-indol-3-yl)-propionic acid. The title compound was prepared by Method 2 from (1-methyl-1H-indol-3-yl)-acetic acid methyl ester (0.10 g, 0.49 mmol), 3-bromoethyl-1-(4-methoxy-phenyl)-5-p-tolyl-1H-pyrazole (89 mg, 0.25 mmol), sodium hydride (19 mg, 0.49 mmol) and DMF (4.0 mL), giving 3-[1-(4-methoxy-phenyl)-5-p-tolyl-1H-pyrazol-3-yl]-2-(1-methyl-1H-indol-3-yl)-propionic acid methyl ester, which was not isolated. The ester was converted to the acid in situ by adding 2.5 mL (4.9 mmol) LiOH solution giving 57 mg (49%) of 3-[1-(4-methoxy-phenyl)-5-p-tolyl-1H-pyrazol-3-yl]-2-(1-methyl-1H-indol-3-yl)-propionic acid. HPLC: Rₜ=3.23 (Method B). MS (ES+): mass calculated for C₂₉H₂₇N₃O₃, 465.21; m/z found 466.2 [M+H]⁺. ¹H NMR (400 MHz, DMSO-d₆): 12.15 (br s, 1H), 7.64 (d, J=7.9 Hz, 1H), 7.40 (d, J=8.2 Hz, 1H), 7.32 (s, 1H), 7.17-7.10 (m, 5H), 7.05-7.03 (m, 3H), 6.93 (d, J=8.9 Hz, 2H), 6.38 (s, 1H), 4.22 (dd, J=9.1, 5.9 Hz, 1H), 3.76 (s, 6H), 3.44 (dd, J=14.7, 9.2 Hz, 1H), 3.04 (dd, J=5.9, 14.7 Hz, 1H), 2.27 (s, 3H).

### Example 73

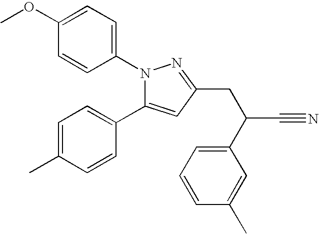

### 3-[1-(4-Methoxy-phenyl)-p-tolyl-1H-pyrazol-3-yl]-2-m-tolyl-propionitrile

To a solution of 3-[1-(4-methoxy-phenyl)-5-p-tolyl-1H-pyrazol-3-yl]-2-m-tolyl-propionamide (Example 65, 0.31 g, 0.73 mmol) in pyridine (0.115 mL, 1.46 mmol) and dioxane (2.0 mL) at 0° C. was added TFAA (0.11 mL, 0.80 mmol). The solution was stirred at 0° C. for 30 min, allowed to warm to rt and stirred for an additional 3 h. The solvent was removed under reduced pressure, and the residue was re-dissolved in EtOAc (100 mL). This solution was washed with water (1×50 mL) and brine (1×50 mL) and dried with Na₂SO₄, and then solvent was removed under reduced pressure giving 295 mg (>99%) of 3-[1-(4-methoxy-phenyl)-p-tolyl-1H-pyrazol-3-yl]-2-m-tolyl-propionitrile. HPLC: Rₜ=3.53 (Method B). MS (ES+): mass calculated for C₂₇H₂₅N₃O, 407.20; m/z found 408.2 [M+H]⁺. ¹H NMR (400 MHz, DMSO-d₆): 7.33-7.26 (m, 3H), 7.18-7.12 (m, 5H), 7.08 (d, J=8.2 Hz, 2H), 6.95 (d, J=8.9 Hz, 2H), 6.48 (s, 1H), 4.58 (dd, J=5.9, 9.6 Hz, 1H), 3.77 (s, 3H), 3.27 (dd, J=9.6, 14.6 Hz, 1H), 3.15 (dd, J=5.9, 14.6 Hz, 1H), 2.33 (s, 3H), 2.28 (s, 3H).

### Example 74

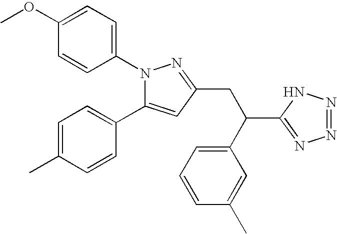

### 5-{2-[1-(4-Methoxy-phenyl)-5-p-tolyl-1H-pyrazol-3-yl]-1-m-tolyl-ethyl}-1H-tetrazole

3-[1-(4-Methoxy-phenyl)-p-tolyl-1H-pyrazol-3-yl]-2-m-tolyl-propionitrile (Example 73, 0.10 g, 0.24 mmol), sodium azide (32 mg, 0.50 mmol) and ammonium chloride (26 mg, 0.50 mmol) were mixed in DMF (3.0 mL) and heated at 100° C. for 4 days. The reaction mixture was cooled, diluted with water (25 mL) and extracted with EtOAc (3×25 mL). The combined organic layers were washed with brine (1×25 mL) and dried with Na₂SO₄, and the solvent was removed under reduced pressure yielding 21 mg (20%) of 5-{2-[1-(4-methoxy-phenyl)-5-p-tolyl-1H-pyrazol-3-yl]-1-m-tolyl-ethyl}-1H-tetrazole. HPLC: Rₜ=3.16 (Method B). MS (ES+): mass calculated for C₂₇H₂₆N₆O, 450.22; m/z found 451.2 [M+H]⁺. ¹H NMR (400 MHz, DMSO-d₆): 7.25-7.17 (m, 3H), 7.12 (d, J=7.9 Hz, 2H), 7.07 (d, J=7.4 Hz, 1H), 7.04 (d, J=9.0 Hz, 2H), 6.99 (d, J=8.1 Hz, 2H), 6.92 (d, J=9.0 Hz, 2H), 6.23 (s, 1H), 4.85 (dd, J=6.7, 9.2 Hz, 1H), 3.75 (s, 3H), 3.60 (dd, J=9.3, 14.8 Hz, 1H), 3.34 (dd, J=6.4, 14.4 Hz, 1H), 2.28 (s, 3H), 2.26 (s, 3H).

### Example 75

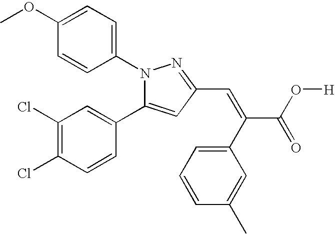

### (E)-3-[5-(3,4-Dichloro-phenyl)-1-(4-methoxy-phenyl)-1H-pyrazol-3-yl]-2-m-tolyl-acrylic acid

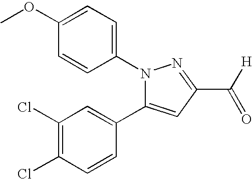

A. 5-(3,4-Dichloro-phenyl)-1-(4-methoxy-phenyl)-1H-pyrazole-3-carbaldehyde. To a stirred solution of [5-(3,4-dichlorophenyl)-1-(4-methoxyphenyl)-1H-pyrazol-3-yl]-methanol (Example 1 Step C, 1.0 g, 2.9 mmol) in CH₂Cl₂ (13 mL) under N₂ was added Dess-Martin periodinane (2.1 g, 4.9 mmol) at rt. After 3 h, Na₂S₂O₃ (5.0 g, 20 mmol) dissolved in saturated NaHCO₃ (25 mL) and EtOAc (25 mL) were added, and the mixture was stirred until the layers were clear. The layers were separated, and the aqueous phase was extracted with EtOAc (3×15 mL). The combined organic extracts were dried over Na₂SO₄ and filtered, and the solvent was removed under reduced pressure to afford 0.95 g (96%) of the crude aldehyde, which was used without further purification. HPLC: Rₜ=10.3 (Method A). ¹H NMR (400 MHz, CDCl₃): 9.98 (s, 1H), 7.32 (s, 1H), 7.30 (d, J=2.3 Hz, 1H), 7.19-7.16 (m, 2H), 6.95 (s, 1H), 6.91 (dd, J=8.1, 2.3 Hz, 1H), 6.88-6.84 (m, 2H), 3.78 (s, 3H).

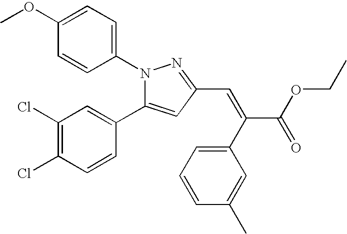

B. 3-[5-(3,4-Dichloro-phenyl)-1-(4-methoxy-phenyl)-1H-pyrazol-3-yl]-2-m-tolyl-acrylic acid ethyl ester. To a stirred solution containing sodium hydride (0.20 mg, 60% in mineral oil, 4.8 mmol) suspended in EtOH (5 mL) was added ethyl-m-tolyacetate (0.87 g, 4.9 mmol) at rt. After 30 min, 5-(3,4-dichloro-phenyl)-1-(4-methoxy-phenyl)-1H-pyrazole-3-carbaldehyde (Step A, 0.562 g, 1.63 mmol) in 2 mL DMF was added. The reaction mixture was stirred for 18 h at 70° C. The solvent was removed under reduced pressure, and the residue was purified by silica gel chromatography with 7:93 MeOH/CH₂Cl₂ to afford 220 mg (27.2%) of 3-[5-(3,4-dichloro-phenyl)-1-(4-methoxy-phenyl)-1H-pyrazol-3-yl]-2-m-tolyl-acrylic acid ethyl ester. HPLC: Rₜ=11.76 (Method A). MS (ES+): mass calculated for C₂₈H₂₄Cl₂N₂O3, 506.12; m/z found 507.0 [M+H]⁺. ¹H NMR (400 MHz, CDCl₃): 7.83-7.80 (m, 1H), 7.74-7.71 (m, 2H), 7.37-7.35 (m, 1H), 7.33-7.29 (m, 4H), 7.19 (d, J=4.5 Hz, 2H), 6.92-6.88 (m, 2H), 4.19 (dd, J=13.9, 7.2 Hz, 2H), 3.78 (s, 3H), 2.51 (s, 3H), 1.21 (t, J=6.8, Hz, 3H).

C. 3-[5-(3,4-Dichloro-Phenyl)-1-(4-methoxy-phenyl)-1H-pyrazol-3-yl]-2-m-tolyl-acrylic acid. To a stirred solution containing 3-[5-(3,4-dichloro-phenyl)-1-(4-methoxy-phenyl)-1-H-pyrazol-3-yl]-2-m-tolyl-acrylic acid ethyl ester (Step B, 50 mg, 0.10 mmol) was added 2 mL LiOH (2 M). After 4 h at 50° C., the solvent was removed under reduced pressure and the residue was purified by silica gel chromatography with 5:95 MeOH/CH₂Cl₂ to afford 34 mg (72.3%) of the title compound. HPLC: Rₜ=10.65 (Method A). MS (ES+): mass calculated for C₂₆H₂₀Cl₂N₂O₃, 478.09; m/z found 479.0 [M+H]⁺. ¹H NMR (400 MHz, CDCl₃): 7.35 (t, J=8.0 Hz, 1H), 7.28-7.23 (m, 3H), 7.15-7.11 (m, 3H), 7.09 (d, J=2.0 Hz, 1H), 6.88-6.86 (m, 2H), 6.77 (dd, J=8.3, 2.0 Hz, 1H), 5.45 (s, 1H), 3.82 (s, 3H), 2.39 (s, 3H).

### Example 76

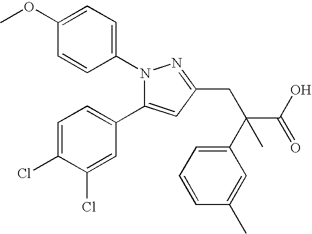

### 3-[5-(3,4-Dichloro-phenyl)-1-(4-methoxy-phenyl)-1H-pyrazol-3-yl]-2-methyl-2-m-tolyl-propionic acid

A. 3-[5-(3,4-Dichloro-phenyl)-1-(4-methoxy-phenyl)-1H-pyrazol-3-yl]-2-methyl-2-m-tolyl-propionic acid ethyl ester. To a solution of 3-[5-(3,4-dichloro-phenyl)-1-(4-methoxy-phenyl)-1H-pyrazol-3-yl]-2-m-tolyl-propionic acid ethyl ester (Method 2, product from alkylation step before hydrolysis) (50 mg, 0.10 mmol) in THF (1.0 mL) at 0° C. was added a 1.0 M solution of NaHMDS (0.15 mL, 0.15 mmol). The solution was stirred at 0° C. for 2 h, then iodomethane (41 mg, 0.29 mmol) was added neat. After stirring for 1 h the reaction was quenched with saturated ammonium chloride (50 mL), and the reaction mixture was extracted with EtOAc (3×50 mL). The combined organic layers were washed with brine (1×50 mL) and dried with Na₂SO₄, and the solvent was removed under reduced pressure. The crude material was purified by flash chromatography (EtOAc/hexanes) giving 31 mg (60%) of 3-[5-(3,4-dichloro-phenyl)-1-(4-methoxy-phenyl)-1H-pyrazol-3-yl]-2-methyl-2-m-tolyl-propionic acid ethyl ester. HPLC: Rₜ=3.79 (Method B). MS (ES+): mass calculated for C₂₉H₂₈Cl₂N₂O₃, 522.15; m/z found 523.1 [M+H]⁺. ¹H NMR (400 MHz, DMSO-d₆): 7.58 (d, J=8.4 Hz, 1H), 7.42 (d, J=2.0 Hz, 1H), 7.25 (t, J=7.6 Hz, 1H), 77.17-7.14 (m, 4H), 7.08 (d, J=7.4 Hz, 1H), 7.05 (dd, J=2.0 Hz, 8.3 Hz, 1H), 6.97 (d, J=8.9 Hz, 2H), 6.22 (s, 1H), 4.10 (m, 2H), 3.77 (s, 3H), 3.40 (d, J=13.9 Hz, 1H), 3.17 (d, J=13.9 Hz, 1H), 2.13 (s, 3H), 1.49 (s, 3H), 1.12 (t, J=7.1 Hz, 3H).

B. 3-[5-(3,4-Dichloro-phenyl)-1-(4-methoxy-phenyl)-1H-pyrazol-3-yl]-2-methyl-2-m-tolyl-propionic acid. The title compound was prepared by Method 2 from 3-[5-(3,4-dichloro-phenyl)-1-(4-methoxy-phenyl)-1H-pyrazol-3-yl]-2-methyl-2-m-tolyl-propionic acid ethyl ester (0.11 g, 0.21 mmol), lithium hydroxide (88 mg, 2.1 mmol), THF (2.3 mL), MeOH (0.87 mL) and water (0.87 mL) giving 93 mg (90%) of 3-[5-(3,4-dichloro-phenyl)-1-(4-methoxy-phenyl)-1H-pyrazol-3-yl]-2-methyl-2-m-tolyl-propionic acid. HPLC: Rₜ=3.42 (Method B). MS (ES+): mass calculated for C₂₇H₂₄Cl₂N₂O₃, 494.12; m/z found 495.0 [M+H]⁺. ¹H NMR (400 MHz, DMSO-d₆): 12.50 (s, 1H), 7.58 (d, J=8.4 Hz, 1H), 7.41 (d, J=2.0 Hz, 1H), 7.26-7.19 (m, 3H), 7.16 (d, J=9.0 Hz, 2H), 7.08 (d, J=7.1 Hz, 1H), 7.03 (dd, J=2.0 Hz, 8.4 Hz, 1H), 6.97 (d, J=9.0 Hz, 2H), 6.20 (s, 1H), 3.78 (s, 3H), 3.37 (d, J=14.0 Hz, 1H), 3.14 (d, J=14.0 Hz, 1H), 2.31 (s, 3H), 1.46 (s, 3H).

### Example 77

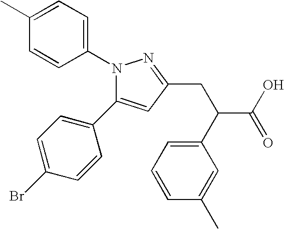

### 3-[5-(4-Bromo-phenyl)-1-p-tolyl-1H-pyrazol-3-yl]-2-m-tolyl-propionic acid

A. 2-m-Tolyl-5-trimethylsilanyl-pent-4-ynoic acid ethyl ester. To a −78° C. solution of m-tolyl-acetic acid ethyl ester (2.0 g, 11 mmol) in THF (37 mL), a 2.0 M solution of lithium diisopropylamine in THF (5.6 mL, 11 mmol) was added dropwise. The mixture was stirred at −78° C. for 1 h and then added to a −78° C. solution of propargyl bromide (5.6 mL, 11 mmol, 1 equiv) in THF (30 mL). The reaction mixture was allowed to warm to room temperature and stirred for 12 h. Diethyl ether (40 mL) and satd aq NH₄Cl (50 mL) were added, and the resulting aqueous layer was back-extracted with Et₂O (2×50 mL). The combined organic layers were washed with 1 N HCl (50 mL) then brine (50 mL), and dried (MgSO₄). The solvent was evaporated under reduced pressure, and the residue was purified by chromatography (silica gel, 20% ethyl acetate/hexanes) to afford the desired silanyl-pentynoic acid ester (2.90 g, 90% yield). TLC (silica gel, 1:9 EtOAc/hexanes): R_{f}=0.54. MS (ESI): mass calculated for C₁₇H₂₄O₂Si, 288.15; m/z found, 289.1 [M+H]⁺. ¹H NMR (400 MHz, CDCl₃): 7.17-6.96 (m, 4H), 4-13-3.99 (m, 2H), 3.65-3.62 (m, 1H), 2.82 (dd, J=16.8, 8.4 Hz, 1H), 2.54 (d, J=16.8, 7.0 Hz, 1H), 2.23 (s, 3H), 1.13 (t, J=10.0 Hz, 3H), 0.00 (s, 9H).

B. 6-(4-Bromo-phenyl)-6-oxo-2-m-tolyl-hex-4-ynoic acid ethyl ester. To a 0° C. solution of 2-m-tolyl-5-trimethylsilanyl-pent-4-ynoic acid ethyl ester (9.5 g, 33 mmol) and 4-bromobenzoyl chloride (9.4 g, 43 mmol, 1.3 equiv) in CH₂Cl₂ (550 mL) was added aluminum chloride (9.5 g, 50 mmol, 1.5 equiv) portionwise. The mixture was stirred at 0° C. for 2 h, then the reaction was quenched with satd aq potassium sodium tartrate (200 mL). The resulting mixture was stirred at room temperature for 2 h. The layers were separated, and the aqueous layer was back-extracted with CH₂Cl₂ (3×150 mL). The combined organic layers were washed with 1 N NaOH (70 mL) then brine (70 mL), and dried (MgSO₄). The solvent was evaporated under reduced pressure, and the residue was purified by chromatography (silica gel, 25% ethyl acetate/hexanes) to afford the desired benzoyl-pentynoic acid ester (9.2 g, 70%). TLC (silica gel, 1:9 EtOAc/hexanes): R_{f}=0.28. MS (ESI): mass calculated for C₂₁H₁₉BrO₃, 398.05; m/z found, 399/400 [M+H]⁺. ¹H NMR (500 MHz, CDCl₃): 7.14 (d, J=8.9 Hz, 2H), 7.14 (d, J=8.9 Hz, 2H), 7.29-7.14 (m, 3H), 4.23-4.12 (m, 2H), 3.88 (t, J=7.8 Hz, 1H), 3.09 (dAB syst., J=17.3, 7.8 Hz, 2H), 2.38 (s, 3H), 1.24 (t, J=9.2 Hz, 3H).

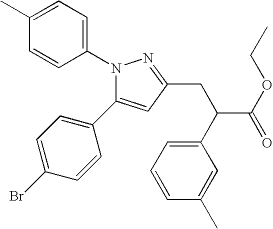

C. 3-[5-(4-Bromo-phenyl)-1-p-tolyl-1H-pyrazol-3-yl]-2-m-tolyl-propionic acid ethyl ester. To a solution of 6-(4-bromo-phenyl)-6-oxo-2-m-tolyl-hex-4-ynoic acid ethyl ester (7.5 g, 19 mmol) in THF (40 mL) was added hydrazine (4.5 g, 28 mmol, 1.5 equiv) and Cs₂CO₃ (9.0 g, 28 mmol, 1.5 equiv). The reaction mixture was stirred at room temperature for 12 h. The resulting mixture was diluted with ethyl acetate (30 mL), and a satd aq solution of cesium carbonate (50 mL) was added. The resulting aqueous layer was back-extracted with ethyl acetate (2×30 mL). The combined organic layers were washed with satd aq NaHCO₃ (50 mL) then brine (50 mL), and dried (MgSO₄). The solvent was evaporated under reduced pressure, and the residue was purified by chromatography (silica gel, 25% ethyl acetate/hexanes) to afford the desired compound (5.5 g, 58%). TLC (silica gel, 3:7 EtOAc/hexanes): R_{f}=0.35. MS (ESI): mass calculated for C₂₈H₂₇BrN₂O₂, 502.13; m/z found, 503/505 [M+H]⁺. ¹H NMR (400 MHz, CDCl₃): 7.39 (d, J=10.7 Hz, 2H), 7.25-7.01 (m, 10H), 6.17 (s, 1H), 4.19-4.03 (m, 3H), 3.52 (dd, J=14.7, 9.6 Hz, 1H), 3.09 (dd, J=14.7, 6.0, 1H), 2.35 (s, 6H), 1.19 (t, J=7.1 Hz, 3H).

D. 3-[5-(4-Bromo-phenyl)-1-p-tolyl-1H-pyrazol-3-yl]-2-m-tolyl-propionic acid. To a solution of 3-[5-(4-bromo-phenyl)-1-p-tolyl-1H-pyrazol-3-yl]-2-m-tolyl-propionic acid ethyl ester (100 mg, 0.2 mmol) was added LiOH (14 mg, 0.6 mmol, 3 equiv) in 2:1 THF/H₂O (1 mL). After 3 h at 45° C., the mixture was purified by preparative reversed-phase HPLC (acetonitrile/water) to afford the title compound (66 mg, 79%). HPLC: Rₜ=4.25 (Method A). MS (ESI): mass calculated for C₂₆H₂₃BrN₂O₂, 474.09; m/z found, 475/477 [M+H]⁺. ¹H NMR (500 MHz, CDCl₃): 7.40 (d, J=8.5 Hz, 2H), 7.22 (d, J=7.6 Hz, 2H), 7.19-7.05 (m, 7H), 7.01 (d, J=8.5 Hz, 2H), 6.23 (s, 1H), 4.10 (dd, J=9.6, 5.5 Hz, 1H), 3.53 (dd, J=14.8, 9.6 Hz, 1H), 3.13 (dd, J=14.8, 5.5 Hz, 1H), 2.36 (s, 3H), 2.34 (s, 3H).

The compounds of Examples 78-93 were made according to the synthetic methods outlined in Example 77 and Scheme L.

### Example 78

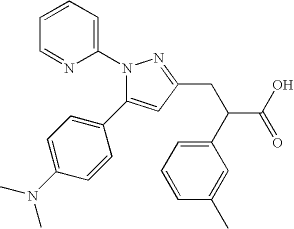

### 3-[5-(4-Dimethylamino-phenyl)-1-pyridin-2-yl-1H-pyrazol-3-yl]-2-m-tolyl-propionic acid

HPLC: Rₜ=3.90 (Method B). MS (ESI): mass calculated for C₂₆H₂₆N₄O₂, 426.21; m/z found, 427.2 [M+H]⁺. ¹H NMR (500 MHz, CDCl₃): 8.38 (d, J=6.3 Hz, 1H), 7.76 (td, J=7.4, 1.2 Hz, 1H), 7.40 (d, J=8.2 Hz, 1H), 7.24-7.18 (m, 4H), 7.11-7.07 (m, 3H), 6.22 (s, 1H), 4.14 (dd, J=9.6, 5.5 Hz, 1H), 3.56 (dd, J=15.0, 9.6 Hz, 1H), 3.12 (dd, J=15.0, 5.5 Hz, 1H), 3.08, (s, 6H), 2.34 (s, 3H).

### Example 79

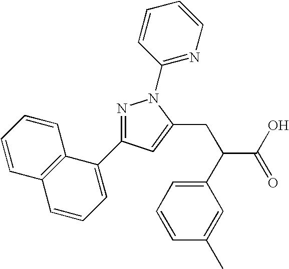

### 3-(5-Naphthalen-1-yl-2-pyridin-2-yl-2H-pyrazol-3-yl)-2-m-tolyl-propionic acid

HPLC: Rₜ=3.36 (Method B). MS (ESI): mass calculated for C₂₈H₂₃N₃O₂, 433.18; m/z found, 434.2 [M+H]⁺. ¹H NMR (500 MHz, CDCl₃): 8.44 (d, J=4.9 Hz, 1H), 8.25 (s, 1H), 8.09 (d, J=8.2 Hz, 1H), 8.03 (d, J=8.5 Hz, 1H), 7.89-7.82 (m, 4H), 7.50-7.46 (m, 2H), 7.28-7.18 (m, 4H), 7.09 (d, J=6.8 Hz, 1H), 6.64 (s, 1H), 4.34 (dd, J=9.0, 5.7 Hz, 1H), 3.94 (dd, J=14.8, 9.0 Hz, 1H), 3.66 (dd, J=14.8, 5.7 Hz, 1H), 2.34 (s, 3H).

### Example 80

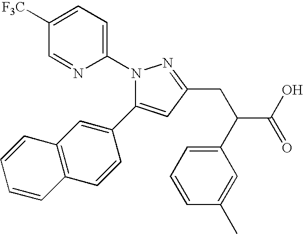

### 3-[5-Naphthalen-2-yl-1-(5-trifluoromethyl-pyridin-2-yl)-1H-pyrazol-3-yl]-2-m-tolyl-propionic acid

HPLC: Rₜ=3.41 (Method B). MS (ESI): mass calculated for C₂₉H₂₂F₃N₃O₂, 501.17; m/z found, 520/522 [M+H₃O]⁺. ¹H NMR (500 MHz, CDCl₃): 8.45 (s, 1H), 7.89-7.74 (m, 6H), 7.66 (d, J=8.5 Hz, 1H), 7.54-7.48 (m, 2H), 7.28-7.19 (m, 3H), 7.12-7.11 (m, 1H), 6.33 (s, 1H), 4.16 (dd, J=9.6, 5.7 Hz, 1H), 3.60 (dd, J=15.0, 9.6 Hz, 1H), 3.15 (dd, J=15.0, 5.7 Hz, 1H), 2.35 (s, 3H).

### Example 81

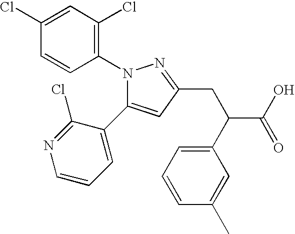

### 3-[5-(2-Chloro-pyridin-3-yl)-1-(2,4-dichloro-phenyl)-1H-pyrazol-3-yl]-2-m-tolyl-propionic acid

MS (ESI): mass calculated for C₂₄H₁₈Cl₃N₃O₂, 485.05; m/z found, 486/488 [M+H]⁺. ¹H NMR (500 MHz, CDCl₃): 8.38 (d, J=2.0 Hz, 1H), 7.70-7.67 (m, 2H), 7.59-7.53 (m, 2H), 7.25-7.19 (m, 2H), 7.13 (s, 1H), 7.04 (d, J=8.8 Hz, 1H), 6.88 (d, J=7.6 Hz, 1H), 6.04 (s, 1H), 3.95 (dd, J=7.0, 4.6 Hz, 1H), 3.62 (dd, J=17.0, 4.6 Hz, 1H), 3.00 (dd, J=17.0, 7.0 Hz, 1H), 2.34 (s, 1H).

### Example 82

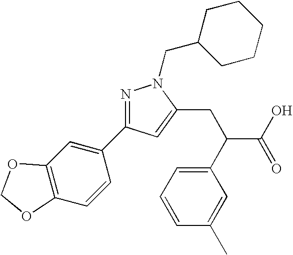

### 3-(5-Benzo[1,3]dioxol-5-yl-2-cyclohexylmethyl-2H-pyrazol-3-yl)-2-m-tolyl-propionic acid

MS (ESI): mass calculated for C₂₇H₃₀N₂O₄, 446.22; m/z found, 447.2 [M+H]⁺. ¹H NMR (500 MHz, CDCl₃): 7.30-7.25 (m, 2H), 7.21-7.20 (m, 2H), 7.16-7.15 (m, 2H), 6.82 (d, J=8.2 Hz, 1H), 6.22 (s, 1H), 3.96-3.86 (m, 3H), 3.43 (dd, J=16.0, 9.3 Hz, 1H), 2.99 (dd, J=16.0, 5.7 Hz, 1H), 2.36 (s, 3H), 1.72-1.53 (m, 5H), 1.21-1.12 (m, 3H), 0.98-0.92 (m, 2H).

### Example 83

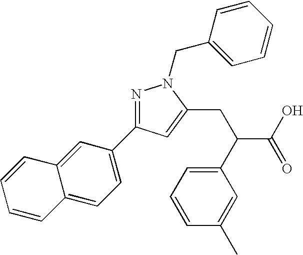

### 3-(2-Benzyl-5-naphthalen-2-yl-2H-pyrazol-3-yl)-2-m-tolyl-propionic acid

MS (ESI): mass calculated for C₃₀H₂₆N₂O₂, 446.20; m/z found, 447.8 [M+H]⁺. ¹H NMR (500 MHz, CDCl₃): 8.17 (s, 1H), 7.84-7.78 (m, 4H), 7.46-7.44 (m, 2H), 7.29-7.24 (m, 3H), 7.18 (t, J=7.6 Hz, 1H), 7.09-7.06 (m, 3H), 7.01-6.99 (m, 2H), 6.47 (s, 1H), 5.36 (AB syst., Jab=16 Hz, 2H), 3.74 (dd, J=8.7, 6.3 Hz, 1H), 3.39 (dd, J=15.0, 8.7 Hz, 1H), 2.92 (dd, J=15.0, 6.3 Hz, 1H), 2.29 (s, 3H).

### Example 84

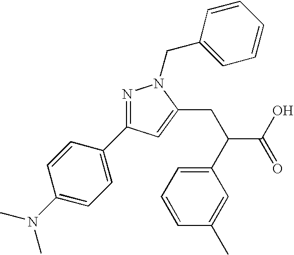

### 3-[2-Benzyl-5-(4-dimethylamino-phenyl)-2H-pyrazol-3-yl]-2-m-tolyl-propionic acid

MS (ESI): mass calculated for C₂₈H₂₉N₃O₂, 439.23; m/z found, 440.7 [M+H]⁺. ¹H NMR (500 MHz, CDCl₃): 7.38 (d, J=8.5 Hz, 2H), 7.31-7.25 (m, 5H), 7.20 (t, J=8.0 Hz, 1H), 7.10-7.06 (m, 3H), 7.01-7.00 (m, 2H), 6.37 (s, 1H), 5.33 (AB syst., Jab=16.0 Hz, 2H), 3.73 (dd, J=9.2, 5.7 Hz, 1H), 3.38 (dd, J=15.7, 9.2 Hz, 1H), 3.13 (s, 6H), 2.88 (dd, J=15.4, 5.7 Hz, 1H), 2.31 (s, 3H).

### Example 85

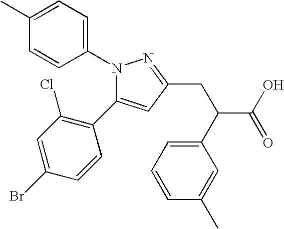

### 3-[5-(4-Bromo-2-chloro-phenyl)-1-p-tolyl-1H-pyrazol-3-yl]-2-m-tolyl-propionic acid

HPLC: Rₜ=4.30 (Method A). MS (ESI): mass calculated for C₂₆H₂₂BrClN₂O₂, 508.06; m/z found, 509/511 [M+H]⁺. ¹H NMR (500 MHz, CDCl₃): 7.53 (d, J=1.9 Hz, 1H), 7.32 (dd, J=8.2, 1.9 Hz, 1H), 7.22 (t, J=7.4 Hz, 1H), 7.17-7.15 (m, 2H), 7.11-7.06 (m, 3H), 7.03-6.98 (m, 3H), 6.20 (s, 1H), 4.08 (dd, J=9.0, 6.3 Hz, 1H), 3.55 (dd, J=14.8, 9.0 Hz, 1H), 3.18 (dd, J=14.8, 6.3 Hz, 1H), 2.34 (s, 3H), 2.31 (s, 3H).

### Example 86

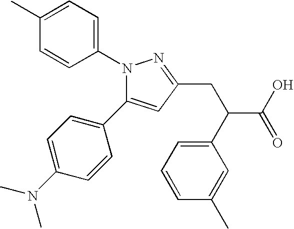

### 3-[5-(4-Dimethylamino-phenyl)-1-p-tolyl-1H-pyrazol-3-yl]-2-m-tolyl-propionic acid

HPLC: Rₜ=1.26 (Method H). MS (ESI): mass calculated for C₂₈H₂₉N₃O₂, 439.23; m/z found, 440.2 [M+H]⁺. ¹H NMR (400 MHz, CDCl₃): 7.30 (s, 3H), 7.24-7.20 (m, 3H), 7.13-7.07 (m, 2H), 6.97 (d, J=8.3 Hz, 2H), 6.67 (d, J=8.3 Hz, 2H), 6.13 (s, 1H), 4.01 (dd, J=9.3, 6.1 Hz, 1H), 3.50 (dd, J=14.9, 9.3 Hz, 1H), 3.07 (dd, J=14.9, 6.1 Hz, 1H), 2.36 (s, 3H), 2.34 (s, 3H).

### Example 87

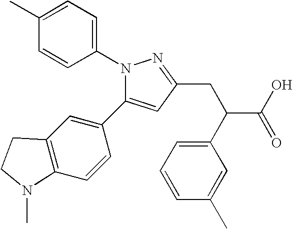

### 3-[5-(1-Methyl-2,3-dihydro-1H-indol-5-yl)-1-p-tolyl-1H-pyrazol-3-yl]-2-m-tolyl-propionic acid

HPLC: Rₜ=3.71 (Method A). MS (ESI): mass calculated for C₂₉H₂₉N₃O₂, 451.23; m/z found, 452.3 [M+H]⁺. ¹H NMR (500 MHz, CDCl₃): 7.26-7.10 (m, 8H), 6.94-6.89 (m, 2H), 6.56 (d, J=8.2 Hz, 1H), 6.20 (s, 1H), 4.13 (dd, J=9.6, 5.5 Hz, 1H), 3.54 (dd, J=14.8, 9.6 Hz, 1H), 3.48 (t, J=8.2 Hz, 2H), 3.13 (dd, J=14.8, 5.5 Hz, 1H), 2.96 (t, J=8.2 Hz, 2H), 2.85 (s, 3H), 2.34 (s, 3H).

### Example 88

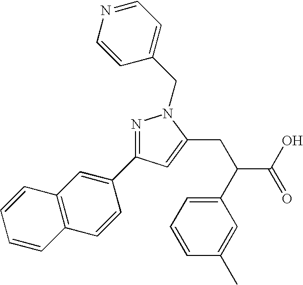

### 3-(5-Naphthalen-2-yl-2-pyridin-4-ylmethyl-2H-pyrazol-3-yl)-2-m-tolyl-propionic acid

MS (ESI): mass calculated for C₂₉H₂₅N₃O₂, 447.19; m/z found, 448.3 [M+H]⁺. ¹H NMR (400 MHz, CDCl₃): 8.56-8.55 (m, 2H), 8.17 (s, 1H), 7.86-7.78 (m, 4H), 7.48-7.44 (m, 2H), 7.32-7.31 (m, 2H), 7.17 (t, J=7.8 Hz, 1H), 7.07-7.04 (m, 3H), 6.70 (s, 1H), 5.52 (AB syst., Jab=17.9 Hz, 2H), 3.97 (dd, J=9.8, 4.8 Hz, 1H), 3.31 (dd, J=15.0, 9.8 Hz, 1H), 2.92 (dd, J=15.0, 4.8 Hz, 1H), 2.27 (s, 3H).

### Example 89

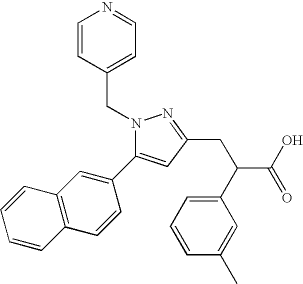

### 3-(5-Naphthalen-2-yl-1-pyridin-4-ylmethyl-1H-pyrazol-3-yl)-2-m-tolyl-propionic acid

MS (ESI): mass calculated for C₂₉H₂₅N₃O₂, 447.19; m/z found, 448.3 [M+H]⁺. ¹H NMR (400 MHz, CDCl₃): 8.65-8.64 (m, 2H), 7.89-7.86 (m, 2H), 7.80-7.70 (m, 1H), 7.70 (s, 1H), 7.56-7.52 (m, 2H), 7.30-7.19 (m, 6H), 7.13-7.11 (m, 2H), 6.36 (s, 1H), 5.51 (s, 1H), 4.13 (dd, J=10.1, 5.0 Hz, 1H), 3.55 (dd, J=14.6, 10.1 Hz, 1H), 3.38 (s, 1H), 3.10 (dd, J=14.6, 5.0 Hz, 1H), 2.33 (s, 3H).

### Example 90

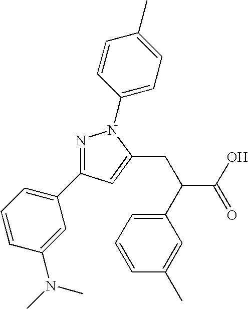

### 3-[5-(3-Dimethylamino-phenyl)-2-p-tolyl-2H-pyrazol-3-yl]-2-m-tolyl-propionic acid

HPLC: Rₜ=3.16 (Method A). MS (ESI): mass calculated for C₂₈H₂₉N₃O₂, 439.23; m/z found, 440.3 [M+H]⁺. ¹H NMR (400 MHz, CDCl₃): 7.64 (t, J=1.7 Hz, 1H), 7.50 (d, J=7.7 Hz, 1H), 7.39 (t, J=8.0 Hz, 1H), 7.28-7.24 (m, 4H), 7.19-7.12 (m, 2H), 7.07-7.05 (m, 1H), 7.01-7.00 (m, 2H), 3.83 (dd, J=9.0, 6.3 Hz, 1H), 3.43 (dd, J=15.5, 9.0 Hz, 1H), 3.11 (s, 3H), 2.99 (dd, J=15.5, 6.3 Hz, 1H), 2.42 (s, 3H), 2.29 (s, 3H).

### Example 91

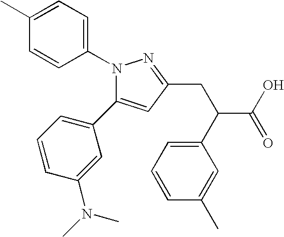

### 3-[5-(3-Dimethylamino-phenyl)-1-p-tolyl-1H-pyrazol-3-yl]-2-m-tolyl-propionic acid

HPLC: Rₜ=3.48 (Method A). MS (ESI): mass calculated for C₂₈H₂₉N₃O₂, 439.23; m/z found, 440.4 [M+H]⁺. ¹H NMR (400 MHz, CDCl₃): 7.36-7.33 (m, 2H), 7.23-7.19 (m, 3H), 7.15-7.09 (m, 7H), 6.36 (s, 1H), 4.10 (dd, J=9.9, 5.4 Hz, 1H), 3.54 (dd, J=14.7, 9.9 Hz, 1H), 3.11 (dd, J=14.9, 5.4 Hz, 1H), 2.97 (s, 6H), 2.34 (s, 6H).

### Example 92

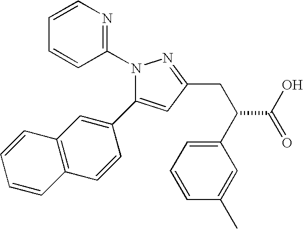

### (S)-3-(5-Naphthalen-2-yl-1-pyridin-2-yl-1H-pyrazol-3-yl)-2-m-tolyl-propionic acid

HPLC: Rₜ=5.95 (Method J). MS (ESI): mass calculated for C₂₈H₂₃N₃O₂, 433.18; m/z found, 434.1 [M+H]⁺. ¹H NMR (400 MHz, CDCl₃): 7.81-7.74 (m, 5H), 5.52-7.50 (m, 2H), 7.26-7.09 (m, 7H), 6.39 (s, 1H), 4.18 (dd, J=10.2, 4.9 Hz, 1H), 3.62 (dd, J=14.8, 10.2 Hz, 1H), 3.12 (dd, J=14.8, 4.9 Hz, 1H), 2.34 (s, 3H).

### Example 93

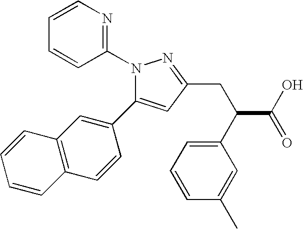

### (R)-3-(5-Naphthalen-2-yl-1-pyridin-2-yl-1H-pyrazol-3-yl)-2-m-tolyl-propionic acid

HPLC: Rₜ=3.95 (Method J). MS (ESI): mass calculated for C₂₈H₂₃N₃O₂, 433.18; m/z found, 434.1 [M+H]⁺. ¹H NMR (400 MHz, CDCl₃): 7.81-7.74 (m, 5H), 5.52-7.50 (m, 2H), 7.26-7.09 (m, 7H), 6.39 (s, 1H), 4.18 (dd, J=10.2, 4.9 Hz, 1H), 3.62 (dd, J=14.8, 10.2 Hz, 1H), 3.12 (dd, J=14.8, 4.9 Hz, 1H), 2.34 (s, 3H).

### Example 94

### (Amination)

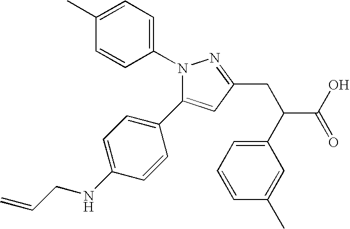

### 3-[5-(4-Allylamino-phenyl)-1-p-tolyl-1H-pyrazol-3-yl]-2-m-tolyl-propionic acid

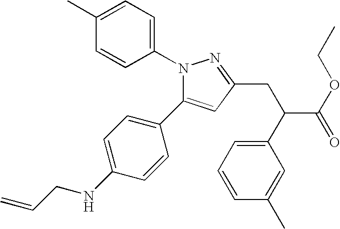

A. 3-[5-(4-Allylamino-phenyl)-1-p-tolyl-1H-pyrazol-3-tolyl-2-m-tolyl-propionic acid ethyl ester. To a mixture of Pd₂(dibenzylideneacetone)₃ (4 mg, 0.004 mmol, 1 mol %), 2-(di-tert-butylphosphino)biphenyl (6 mg, 0.02 mmol, 5 mol %) and K₃PO₄ (130 mg, 0.61 mmol, 1.5 equiv) was added a solution of 3-[5-(4-bromo-phenyl)-1-p-tolyl-1H-pyrazol-3-yl]-2-m-tolyl-propionic acid ethyl ester (Example 77, Step C; 200 mg, 0.4 mmol) in toluene (0.6 mL) followed by allylamine (0.030 mL, 0.48 mmol, 1.2 equiv). The resulting mixture was stirred at 110° C. for 12 h and then cooled to room temperature. Ethyl acetate (2 mL) and water (3 mL) were added, and the resulting aqueous layer was back-extracted with EtOAc (3×2 mL). The combined organic layers were washed with brine (3 mL), and then dried (MgSO₄). The solvent was evaporated under reduced pressure, and the residue was purified by chromatography (silica gel, 25% ethyl acetate/hexanes) to afford the desired compound (90 mg, 47%). HPLC: Rₜ=3.19 (Method B). MS (ESI): mass calculated for C₃₁H₃₃N₃O₂, 479.26; m/z found, 480.3 [M+H]⁺. ¹H NMR (500 MHz, CDCl₃): 7.29 (s, 1H), 7.27-7.04 (m, 7H), 6.96 (d, J=8.5 Hz, 2H), 6.49 (d, J=8.5 Hz, 2H), 6.07 (s, 1H), 5.96-5.89 (m, 1H), 5.29-5.25 (m, 1H), 5.18-5.16 (m, 1H), 4.20-4.14 (m, 1H), 4.10-4.02 (m, 2H), 3.76-3.75 (m, 2H), 3.52-3.45 (m, 1H), 3.08 (dd, J=14.5, 6.0 Hz, 1H), 2.34 (s, 6H), 1.19 (t, J=7.1 Hz, 1H).

B. 3-[5-(4-Allylamino-phenyl)-1-p-tolyl-1H-pyrazol-3-yl]-2-m-tolyl-propionic acid.

To a solution of 3-[5-(4-allylamino-phenyl)-1-p-tolyl-1H-pyrazol-3-yl]-2-m-tolyl-propionic acid ethyl ester (90 mg, 0.2 mmol) was added LiOH (14 mg, 0.58 mmol, 3 equiv) in 2:1 THF/H₂O (1 mL). After 3 h at 45° C., the mixture was purified by preparative reversed-phase HPLC (acetonitrile/water) to afford the desired compound (70 mg, 77%). MS (ESI): mass calculated for C₂₉H₂₉N₃O₂, 451.23; m/z found, 452.6 [M+H]⁺. ¹H NMR (500 MHz, CDCl₃): 7.21-7.03 (m, 8H), 6.93 (d, J=8.8, 2H), 6.26 (s, 1H), 5.88-5.83 (m, 1H), 5.29-5.24 (m, 2H), 4.06 (dd, J=10.4, 5.1 Hz, 1H), 3.79 (d, J=6.3 Hz, 2H), 3.54 (dd, J=15.0, 10.4 Hz, 1H), 3.09 (dd, J=15.05.1 Hz, 1H), 2.33 (s, 3H), 2.32 (s, 3H).

The compounds of Examples 95-101 were made according to the synthetic methods outlined in Example 94 and Scheme L.

### Example 95

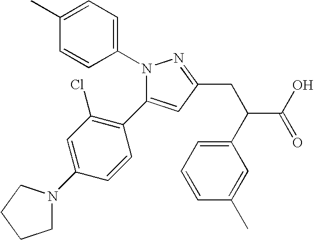

### 3-[5-(2-Chloro-4-pyrrolidin-1-yl-phenyl)-1-p-tolyl-1H-pyrazol-3-yl]-2-m-tolyl-propionic acid

HPLC: Rₜ=4.35 (Method A). MS (ESI): mass calculated for C₃₀H₃₀ClN₃O₂, 499.20; m/z found, 500.10 [M+H]⁺. ¹H NMR (500 MHz, CDCl₃): 7.23-7.15 (m, 3H), 7.10-7.05 (m 5H), 6.89 (d, J=8.8 Hz, 1), 6.49 (d, J=2.5 Hz, 1H), 6.32 (dd, J=8.8, 2.5 Hz, 1H), 6.15 (s, 1H), 4.12 (d, J=9.0, 6.0 Hz, 1H), 3.55 (dd, J=14.8, 9.0 Hz, 1H), 3.26-3.24 (m, 4H), 3.18 (dd, J=14.8, 6.0 Hz, 1H), 2.33 (s, 3H), 2.30 (s, 3H), 2.07-1.99 (m, 4H).

### Example 96

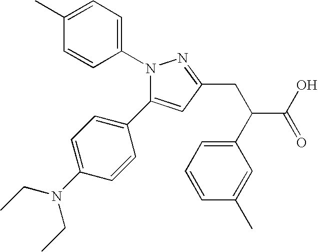

### 3-[5-(4-Diethylamino-phenyl)-1-p-tolyl-1H-pyrazol-3-yl]-2-m-tolyl-propionic acid

HPLC: Rₜ=3.21 (Method A). MS (ESI): mass calculated for C₃₀H₃₃N₃O₂, 467.26; m/z found, 468.3 [M+H]⁺. ¹H NMR (500 MHz, CDCl₃): 7.26-7.16 (m, 8H), 7.09-7.08 (m, 4H), 6.22 (s, 1H), 4.08 (dd, J=9.3, 6.0 Hz, 1H), 3.52 (dd, J=14.8, 9.3 Hz, 1H), 3.44 (q, J=7.1 Hz, 4H), 3.11 (dd, J=14.8 6.0 Hz, 1H), 2.34 (s, 3H), 2.32 (s, 3H), 1.09 (t, J=7.1 Hz).

### Example 97

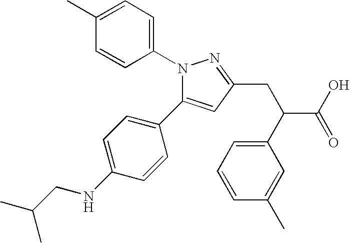

### 3-[5-(4-Isobutylamino-phenyl)-1-p-tolyl-1H-pyrazol-3-yl]-2-m-tolyl-propionic acid

HPLC: Rₜ=4.02 (Method A). MS (ESI): mass calculated for C₃₀H₃₃N₃O₂, 467.26; m/z found, 468.3 [M+H]⁺. ¹H NMR (500 MHz, CDCl₃): 7.20-6.99 (m, 8H), 6.98 (d, J=8.8 Hz, 2H), 6.81 (d, J=8.5 Hz, 2H), 6.17 (s, 1H), 4.07 (dd, J=9.9, 5.5 Hz, 1H), 3.52 (dd, J=14.8, 9.9 Hz, 1H), 3.08 (dd, J=14.8, 5.5 Hz, 1H), 2.96 (d, J=7.1 Hz, 2H), 2.32 (s, 3H), 2.31 (s, 3H), 1.95-1.92 (m, 1H), 0.96 (d J=6.5 Hz, 6H).

### Example 98

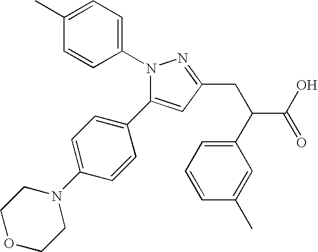

### 3-[5-(4-Morpholin-4-yl-phenyl)-1-p-tolyl-1H-pyrazol-3-yl]-2-m-tolyl-propionic acid

HPLC: Rₜ=3.86 (Method A). MS (ESI): mass calculated for C₃₀H₃₁N₃O₃, 481.24; m/z found, 482.2 [M+H]⁺. ¹H NMR (500 MHz, CDCl₃): 7.21-7.09 (m, 8H), 7.07 (d, J=8.8 Hz, 2H), 6.89 (d, J=8.8 Hz, 2H), 6.21 (s, 1H), 4.08 (dd, J=9.3, 5.8 Hz, 1H), 3.89-3.87 (m, 4H), 3.54 (dd, J=14.8, 9.3 Hz, 1H), 3.23-3.22 (m, 4H), 3.13 (dd, J=14.8, 5.8 Hz, 1H), 2.35 (s, 3H), 2.33 (s, 3H).

### Example 99

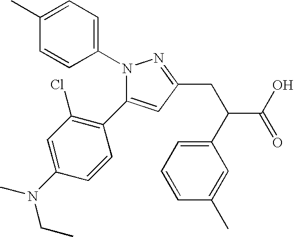

### 3-{5-[2-Chloro-4-(ethyl-methyl-amino)-phenyl]-1-p-tolyl-1H-pyrazol-3-yl}-2-m-tolyl-propionic acid

HPLC: Rₜ=4.1.3 (Method A). MS (ESI): mass calculated for C₂₉H₃₀ClN₃O₂, 487.20; m/z found, 488.1 [M+H]⁺. ¹H NMR (500 MHz, CDCl₃): 7.24-7.15 (m, 3H), 7.10-7.07 (m, 5H), 6.96 (d. J=8.7 Hz, 1H), 6.77 (d, J=2.4 Hz, 1H), 6.62 (dd, J=8.7, 2.4 Hz, 1H), 6.19 (s, 1H), 4.12 (dd, J=9.3, 6.0 Hz, 1H), 3.56 (dd, J=14.8, 9.3 Hz, 1H), 3.39 (q, J=7.1 Hz, 2H), 3.18 (dd, 14.8, 6.0 Hz, 1H), 2.94 (s, 3H), 2.34 (s, 3H), 2.31 (s, 3H), 1.13 (t, J=7.1 Hz, 3H).

### Example 100

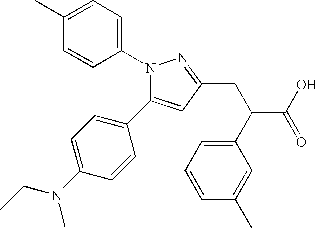

### 3-{5-[4-(Ethyl-methyl-amino)-phenyl]-1-p-tolyl-1H-pyrazol-3-yl}-2-m-tolyl-propionic acid

HPLC: Rₜ=3.29 (Method A). MS (ESI): mass calculated for C₂₉H₃₁N₃O₂, 453.24; m/z found, 454.3 [M+H]⁺. ¹H NMR (400 MHz, CDCl₃): 7.26-7.08 (m, 12H), 6.23 (s, 1H), 4.09-4.05 (m, 1H), 3.52 (dd, J=14.9, 9.3 Hz, 1H), 3.44 (q, J=7.1 Hz, 2H), 3.11 (dd, J=14.9, 6.1 Hz, 1H), 3.06 (s, 3H), 2.35 (s, 3H), 2.32 (s, 3H), 1.12 (t, J=7.1 Hz, 3H).

### Example 101

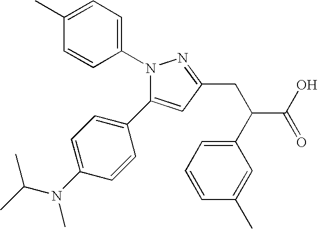

### 3-{5-[4-(Isopropyl-methyl-amino)-phenyl]-1-p-tolyl-1H-pyrazol-3-yl}-2-m-tolyl-propionic acid

HPLC: Rₜ=4.06 (Method A). MS (ESI): mass calculated for C₃₀H₃₃N₃O₂, 467.26; m/z found, 468.3 [M+H]⁺. ¹H NMR (400 MHz, CDCl₃): 7.34 (d, J=8.8 Hz, 2H), 7.26-7.06 (m, 10H), 6.26 (s, 1H), 4.09 (dd, J=9.6, 5.9 Hz, 1H), 3.81-3.78 (m, 1H), 3.53 (dd, J=14.9, 9.6 Hz, 1H), 3.12 (dd, J=14.9, 5.9 Hz, 1H), 3.11 (s, 3H), 2.36 (s, 3H), 2.33 (s, 3H), 1.28 (d, J=6.6 Hz, 6H).

### Example 102

### (Amidation)

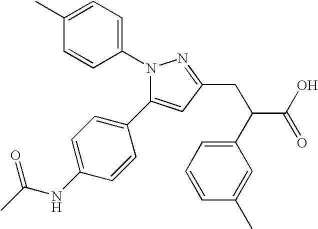

### 3-[5-(4-Acetylamino-phenyl)-1-p-tolyl-1H-pyrazol-3-yl]-2-m-tolyl-propionic acid

To a solution of 3-[5-(4-bromo-phenyl)-1-p-tolyl-1H-pyrazol-3-yl]-2-m-tolyl-propionic acid ethyl ester (Example 77, Step C; 100 mg, 0.2 mmol) in dioxane (0.6 mL) was added CuI (3 mg, 0.02 mmol, 10 mol %), (1R,2R)-N,N′-dimethyl-cyclohexane-1,2-diamine (0.003 mL, 0.02 mmol, 10 mol %), K₂CO₃ (55 mg, 0.40 mmol, 2.0 equiv) and N-methylformamide (15 mg, 0.26 mmol, 1.3 equiv). The mixture was stirred at 110° C. for 14 h, and then cooled to 45° C. prior to the addition of a solution of LiOH (28 mg, 1.2 mmol, 3 equiv) in 2:1 THF/H₂O (1 mL). After 3 h at 45° C., the reaction mixture was purified by preparative reversed-phase HPLC (acetonitrile/water) to afford the title compound (50 mg, 50%). HPLC: Rₜ=3.62 (Method A). MS (ESI): mass calculated for C₂₈H₂₇N₃O₃, 453.21; m/z found, 454.3 [M+H]⁺. ¹H NMR (500 MHz, CDCl₃): 7.43-7.39 (m, 3H), 7.25-7.17 (m, 3H), 7.10-7.06 (m, 6H), 6.24 (s, 1H), 4.09 (dd, J=10.0, 5.2 Hz, 1H), 3.53 (dd, J=15.0, 10.0 Hz, 1H), 3.13-3.09 (dd, J=15.0, 5.2 Hz, 1H), 2.34 (s, 6H), 2.16 (S, 3H).

The compounds of Examples 103 and 104 were made according to the synthetic methods outlined in Example 102 and Scheme L.

### Example 103

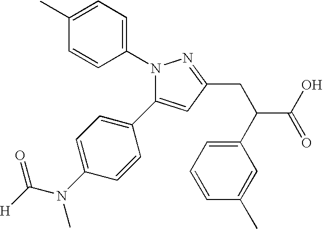

### 3-{5-[4-(Formyl-methyl-amino)-phenyl]-1-p-tolyl-1H-pyrazol-3-yl}-2-m-tolyl-propionic acid

HPLC: Rₜ=3.64 (Method A). MS (ESI): mass calculated for C₂₈H₂₇N₃O₃, 453.21; m/z found, 454.3 [M+H]⁺. ¹H NMR (500 MHz, CDCl₃): 8.50 (s, 1H), 7.25-7.08 (m, 8H), 7.19 (d, J=8.8 Hz, 2H), 7.07 (d, J=8.5 Hz, 2H), 6.24 (s, 1H), 4.11 (dd, J=9.6, 5.7 Hz, 1H), 3.55 (dd, J=15.0, 9.6 Hz, 1H), 3.30 (s, 3H), 3.14 (dd, J=15.0, 5.7 Hz, 1H), 2.36 (s, 3H), 2.24 (s, 3H).

### Example 104

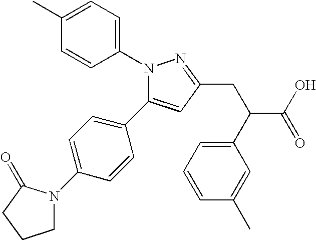

### 3-{5-[4-(2-Oxo-pyrrolidin-1-yl)-phenyl]-1-p-tolyl-1H-pyrazol-3-yl}-2-m-tolyl-propionic acid

HPLC: Rₜ₌3.75 (Method A). MS (ESI): mass calculated for C₃₀H₂₉N₃O₃, 479.22; m/z found, 480.3 [M+H]⁺. ¹H NMR (500 MHz, CDCl₃): 7.54 (d, J=8.8 Hz, 2H), 7.24-7.09 (m, 8H), 7.14 (d, J=8.8 Hz, 2H), 6.20 (s, 1H), 4.10 (dd, J=9.3, 5.7 Hz, 1H), 3.83 (t, J=7.0 Hz, 2H), 3.54 (dd, J=15.0, 9.3 Hz, 1H), 3.13 (dd, J=15.0, 5.7 Hz, 1H), 2.62 (t, J=8.2 Hz, 2H), 2.37 (s, 3H), 2.24 (s, 3H), 2.16 (quintet, J=8.0, 7.0 Hz, 2H).

### Example 105

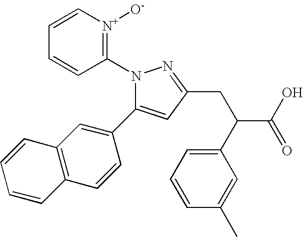

### 3-[5-Naphthalen-2-yl-1-(1-oxy-pyridin-2-yl)-1H-pyrazol-3-yl]-2-m-tolyl-propionic acid

To a solution of 3-(5-naphthalen-2-yl-1-pyridin-2-yl-1H-pyrazol-3-yl)-2-m-tolyl-propionic acid (Example 52; 10 mg, 0.02 mmol) in THF (0.6 mL) was added m-chloroperbenzoic acid (7 mg, 0.03 mmol, 1.5 equiv). The reaction mixture was stirred at room temperature for 3 h, and then diluted with CH₂Cl₂ (2 mL). A solution of 1 N NaOH (1 mL) was added, and the resulting aqueous layer was back-extracted with CH₂Cl₂ (2×2 mL). The combined organic layers were washed with brine (2 mL), dried (MgSO₄), and concentrated under reduced pressure. The residue was purified by preparative reversed-phase HPLC (acetonitrile/water) to afford the title compound (6 mg, 60%). HPLC: Rₜ=1.17 (Method H). MS (ESI): mass calculated for C₂₈H₂₃N₃O₃, 449.17; m/z found, 450.1 [M+H]⁺. ¹H NMR (500 MHz, CDCl₃): 8.25 (s, 1H), 7.78-7.69 (m, 5H), 7.48-7.39 (m, 4H), 7.35-7.30 (m, 1H), 7.30-7.20 (m, 3H), 7.10 (d, J=6.3 Hz, 1H), 4.14 (dd, J=10.0, 5.7 Hz, 1H), 3.59 (dd, J=15.0, 10.0, 1H), 3.12 (dd, J=15.0, 5.7 Hz, 1H), 2.34 (s, 3H).

### Example 106

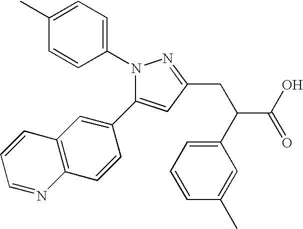

### 3-(5-Quinolin-6-yl-1-p-tolyl-1H-pyrazol-3-yl)-2-m-tolyl-propionic acid

To a solution of 3-[5-(4-allylamino-phenyl)-1-p-tolyl-1H-pyrazol-3-yl]-2-m-tolyl-propionic acid ethyl ester (Example 94, Step A; 70 mg, 0.15 mmol) in ethanol (1 mL) was added 10% Pd/C (26 mg) and methanesulfonic acid (0.01 mL, 0.15 mmol, 1 equiv). The mixture was stirred at 65° C. for 2 h. The catalyst was removed by filtering the reaction mixture through a CELITE® pad, and the pad was rinsed with EtOH (1.5 mL). The combined filtrates were concentrated under reduced pressure. The crude residue was dissolved in 1:1 THF/H₂O (1.5 mL), and LiOH was added (10 mg, 0.45 mmol, 3 equiv). After 3 h at 45° C., the mixture was purified by preparative reversed-phase HPLC (acetonitrile/water) to afford the title compound (26 mg, 35%) along with 3-[5-(4-amino-phenyl)-1-p-tolyl-1H-pyrazol-3-yl]-2-m-tolyl-propionic acid (20 mg, 35%). HPLC: Rₜ=3.18 (Method A). MS (ESI): mass calculated for C₂₉H₂₅N₃O₂, 447.19; m/z found, 448.2 [M+H]⁺. ¹H NMR (400 MHz, CDCl₃): 8.43 (d, J=8.5 Hz, 1H), 8.25 (d, J=8.8 Hz, 1H), 7.85 (d, J=1.7 Hz, 1H), 7.68 (dd, J=8.3, 4.8 Hz, 1H), 7.59 (dd, J=8.8, 1.7 Hz, 1H), 7.26-7.23 (m, 2H), 7.12 (s, 4H), 6.42 (s, 1H), 4.17 (dd, J=9.8, 5.3 Hz, 1H), 3.58 (dd, J=14.9, 9.8 Hz, 1H), 3.17 (dd, J=14.9, 5.3 Hz, 1H), 2.36 (s, 3H).

### Example 107

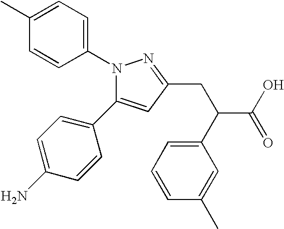

### 3-[5-(4-Amino-phenyl)-1-p-tolyl-1H-pyrazol-3-yl]-2-m-tolyl-propionic acid

Prepared according to the synthetic methods outlined in Example 106. HPLC: Rₜ=3.16 (Method A). MS (ESI): mass calculated for C₂₆H₂₅N₃O₂, 411.19; m/z found, 412.2 [M+H]⁺. ¹H NMR (400 MHz, CDCl₃): 7.30 (s, 2H), 7.24-7.21 (m, 2H), 7.13-7.07 (m, 4H), 6.97 (d, J=8.3 Hz, 2H), 6.67 (d, J=6.8 Hz, 2H), 6.13 (s, 1H), 4.01 (dd, J=9.3, 6.0 Hz, 1H), 3.49 (dd, J=14.6, 9.3 Hz, 1H), 3.07 (dd, J=14.6, 6.0 Hz, 1H), 2.34 (s, 6H).

### Example 108

(Preparation of Alkenes)

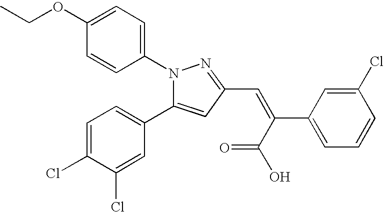

### (Z)-2-(3-Chloro-phenyl)-3-[5-(3,4-dichloro-phenyl)-1-(4-ethoxy-phenyl)-1H-pyrazol-3-yl]-acrylic acid

A. 5-(3,4-Dichloro-phenyl)-1-(4-ethoxy-phenyl)-1H-pyrazole-3-carbaldehyde. To a solution of Dess-Martin periodinane (2.0 g, 4.6 mmol, 2.0 equiv) in CH₂Cl₂ (10 mL) was added a solution of [5-(3,4-dichloro-phenyl)-1-(4-ethoxy-phenyl)-1H-pyrazol-3-yl]-methanol (prepared by the method of Example 1, Steps A-C; 0.84 g, 2.3 mmol) in CH₂Cl₂ (10 mL). The reaction mixture was stirred overnight at room temperature. Then the reaction was quenched with 1 M NaOH (10 mL), and the resulting mixture was stirred until the layers separated. The aqueous layer was back-extracted with CH₂Cl₂ (3×10 mL). The combined organic layers were washed with 1 M NaOH (20 mL) then H₂O (20 mL), dried (MgSO₄), and concentrated to provide the pure aldehyde as a dark brown oil (0.59 g, 1.6 mmol, 70%). TLC (silica gel, 1:1 EtOAc/hexanes): R_{f}=0.62. MS (ESI): mass calculated for C₁₈H₁₄Cl₂N₂O₂, 360.04; m/z found, 361 [M+H]⁺. ¹H NMR (400 mHz, CDCl₃): 10.05 (s, 1H), 7.38-7.36 (m, 2H), 7.25-7.21 (m, 2H), 7.0 (s, 1H), 7.0-6.97 (m, 1H), 6.93-6.91 (m, 2H), 4.06 (q, J=7.0 Hz), 1.44 (t, J=7.0 Hz, 3H).

B. 2-(3-Chloro-phenyl)-3-[5-(3,4-dichloro-phenyl)-1-(4-ethoxy-phenyl)-1H-Pyrazol-3-yl]-acrylic acid, E and Z stereoisomers. To a mixture of 5-(3,4-dichloro-phenyl)-1-(4-ethoxy-phenyl)-1H-pyrazole-3-carbaldehyde (0.33 g, 0.91 mmol) and 3-chlorophenyl acetic acid (0.23 g, 1.4 mmol) was added acetic anhydride (0.8 mL) and TEA (0.8 mL). The mixture was allowed to stir overnight at room temperature. The TEA was removed under reduced pressure, and the resulting mixture was purified on silica gel (MPLC, 0-5% MeOH/CH₂Cl₂) to provide exclusively the E acrylic acid as a brown foam (0.21 g, 46%). The foam was then dissolved in CHCl₃ (10 mL), and the solution was placed in quartz tubes and subjected to uv light overnight. The solvent was removed to provide a 1:1 mixture of E and Z stereoisomers. The stereoisomers were separated by preparative reversed-phase HPLC (acetonitrile/water) to afford the pure Z (0.033 g, 0.064 mmol, 15%) and E acrylic acids (0.043 g, 0.084 mmol, 20%). Z stereoisomer: TLC (silica gel, 9:1 CH₂Cl₂/MeOH): R_{f}=0.26. HPLC: Rₜ=7.35 (Method I). MS (ESI): mass calculated for C₂₆H₁₉Cl₃N₂O₃, 512.05; m/z found, 511/513 [M−H]. ¹H NMR (400 mHz, CDCl₃): 7.49-7.47 (m, 1H), 7.39-7.31 (m, 5H), 7.19-7.16 (m, 2H), 7.05 (s, 1H), 6.99-6.96 (m, 1H), 6.90-6.86 (m, 2H), 4.04 (q, J=7.0 Hz) 6.72 (s, 1H),): 1.44 (t, J=7.0 Hz, 3H).

### Example 109

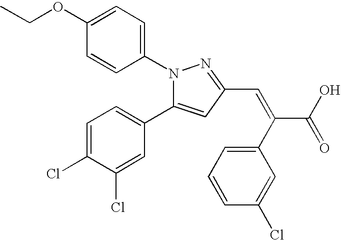

### (E)-2-(3-Chloro-phenyl)-3-[5-(3,4-dichloro-phenyl)-1-(4-ethoxy-phenyl)-1H-pyrazol-3-yl]-acrylic acid

HPLC: Rₜ=8.58. MS (ESI): mass calculated for C₂₆H₂₅N₃O₂, 512.0; m/z found, 513 [M+H]⁺. ¹H NMR (400 mHz, CDCl₃): 8.09 (s, 1H), 7.30 (m, 3H), 7.24 (m, 2H), 7.14 (m, 3H), 6.86 (m, 2H), 6.79 (m, 1H), 5.53 (s, 1H), 4.03 (q, J=7.0 Hz, 2H), 1.42 (t, J=7.0 Hz, 3H).

### Example 110

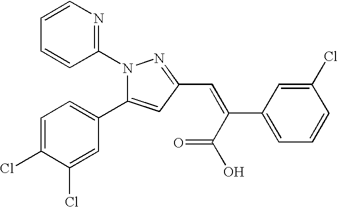

### (Z)-2-(3-Chloro-phenyl)-3-[5-(3,4-dichloro-phenyl)-1-pyridin-2-yl-1H-pyrazol-3-yl]-acrylic acid

This compound was prepared as described for the 4-ethoxyphenyl analog in EXAMPLE 108 substituting [5-(3,4-dichloro-phenyl)-1-pyridin-2-yl-1H-pyrazol-3-yl]-methanol (prepared by the method of Example 1, Steps A-C) for [5-(3,4-dichloro-phenyl)-1-(4-ethoxy-phenyl)-1H-pyrazol-3-yl]-methanol in Step A. TLC (silica gel, 9:1 CH₂Cl₂/MeOH): R_{f}=0.19. HPLC: Rₜ=5.63 (Method I). MS (ESI): mass calculated for C₂₃H₁₄Cl₃N₃O₂, 469.02; m/z found, 468/469 [M−H]⁻. ¹H NMR (400 mHz, CDCl₃): 8.26-8.25 (m, 1H), 7.79-7.77 (m, 1H), 7.58-7.56 (m, 1H), 7.47-7.46 (m, 1H), 7.37-7.22 (m, 6H), 7.02 (s, 1H), 7.00-6.98 (m, 1H), 6.74 (s, 1H).

### Example 111

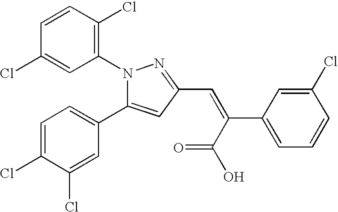

### (Z)-2-(3-Chloro-phenyl)-3-[5-(3,4-dichloro-phenyl)-1-(2,5-dichloro-phenyl)-1H-pyrazol-3-yl]-acrylic acid

This compound was prepared as described for the 4-ethoxyphenyl analog in EXAMPLE 108 substituting [5-(3,4-dichloro-phenyl)-1-(2,5-dichloro-phenyl)-1H-pyrazol-3-yl]-methanol for [5-(3,4-dichloro-phenyl)-1-(4-ethoxy-phenyl)-1H-pyrazol-3-yl]-methanol in Step A. TLC (silica gel, 9:1 CH₂Cl₂/MeOH): R_{f}=0.23. HPLC: Rₜ=7.95 (Method I). MS (ESI): mass calculated for C₂₄H₁₃Cl₅N₂O₂, 535.94; m/z found, 535/537 [M−H]⁻. ¹H NMR (400 mHz, CDCl₃): 7.51-7.49 (m, 2H), 7.45-7.32 (m, 7H), 7.07 (s, 1H), 6.97-6.94 (m, 1H), 6.82 (s, 1H).

### Example 112

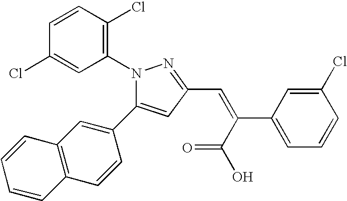

### (Z)-2-(3-Chloro-phenyl)-3-[1-(2,5-dichloro-phenyl)-5-naphthalen-2-yl-1H-pyrazol-3-yl]-acrylic acid

HPLC: Rₜ=5.28 (Method I). MS (ESI): mass calculated for C₂₈H₁₇Cl₃N₂O₂, 518.04; m/z found, 519/521 [M+H]⁺. ¹H NMR (500 MHz, CDCl₃): 7.83-7.72 (m, 4H), 7.54-7.51 (m, 4H), 7.42-7.38 (m, 4H), 7.35-7.33 (m, 2H), 7.11 (s, 1H), 6.87 (s, 1H).

### Example 113

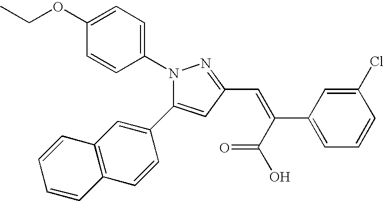

### (Z)-2-(3-Chloro-phenyl)-3-[1-(4-ethoxy-phenyl)-5-naphthalen-2-yl-1H-pyrazol-3-yl]-acrylic acid

HPLC: Rₜ=5.23 (Method I). MS (ESI): mass calculated for C₃₀H₂₃ClN₂O₃, 494.14; m/z found, 495.1 [M+H]⁺. ¹H NMR (500 MHz, CDCl₃): 7.84-7.83 (m, 2H), 7.80-7.77 (m, 2H), 7.56-7.52 (m, 2H), 7.49-7.48 (m, 1H), 7.39-7.37 (m, 1H), 7.33-7.32 (m, 2H), 7.26-7.24 (m, 3H), 7.08 (s, 1H), 6.86 (d, J=9.0 Hz, 2H), 6.77 (s, 1H), 4.03 (q, J=7.1 Hz, 2H), 1.41 (t, J=7.1 Hz, 1H).

### Example 114

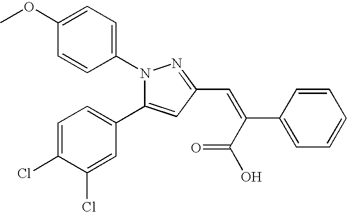

### (Z)-3-[5-(3,4-Dichloro-phenyl)-1-(4-methoxy-phenyl)-1H-pyrazol-3-yl]-2-phenyl-acrylic acid

HPLC: Rₜ=10.60 (Method A). MS (ESI): mass calculated for C₂₅H₁₈Cl₂N₂O₃, 464.07; m/z found, 465.1 [M+H]⁺. ¹H NMR (500 MHz, CDCl₃): 7.50-7.48 (m, 2H), 7.39-7.35 (m, 5H), 7.23 (d, J=9.0 Hz, 2H), 7.06 (s, 1H), 6.99 (dd, J=8.2, 1.9 Hz, 1H), 6.91 (d, J=9.0 Hz, 2H), 6.70 (s, 1H), 3.85 (s, 3H).

### Example 115

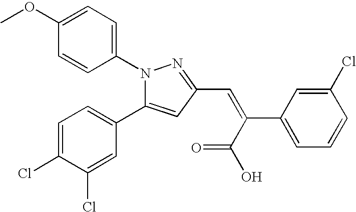

### (Z)-2-(3-Chloro-phenyl)-3-[5-(3,4-dichloro-phenyl)-1-(4-methoxy-phenyl)-1H-pyrazol-3-yl]-acrylic acid

HPLC: Rₜ=10.50 (Method A). MS (ESI): mass calculated for C₂₅H₁₇Cl₃N₂O₃, 498.03; m/z found, 499.0 [M+H]⁺. ¹H NMR (500 MHz, CDCl₃): 7.47 (br s, 1H), 7.41 (s, 2H), 7.39-7.37 (m, 1H), 7.35 (s, 2H), 7.22 (d, J=9.0 Hz, 2H), 7.04 (s, 1H), 7.00 (dd, J=8.2, 2.2 Hz, 1H), 6.92 (d, J=9.0 Hz, 2H), 6.70 (s, 1H), 3.85 (s, 3H).

### Example 116

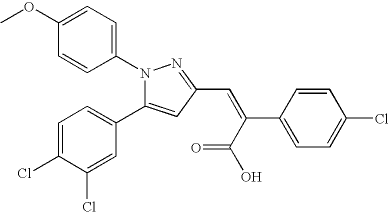

### (Z)-2-(4-Chloro-phenyl)-3-[5-(3,4-dichloro-phenyl)-1-(4-methoxy-phenyl)-1H-pyrazol-3-yl]-acrylic acid

HPLC: Rₜ=10.50 (Method A). MS (ESI): mass calculated for C₂₅H₁₇Cl₃N₂O₃, 498.03; m/z found, 499.0 [M+H]⁺. ¹H NMR (500 MHz, CDCl₃): 7.43-7.40 (m, 4H), 7.36 (d, J=8.8 Hz, 2H), 7.22 (d, J=9.0 Hz, 2H), 7.02 (s, 1H), 6.99 (dd, J=8.2, 2.2 Hz, 1H), 6.92 (d, J=9.0 Hz, 2H), 6.70 (s, 1H), 3.85 (s, 3H).

### Example 117

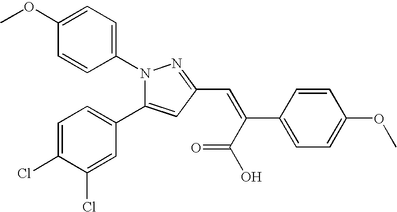

### (Z)-3-[5-(3,4-Dichloro-phenyl)-1-(4-methoxy-phenyl)-1H-pyrazol-3-yl]-2-(4-methoxy-phenyl)-acrylic acid

HPLC: Rₜ=5.60 (Method A). MS (ESI): mass calculated for C₂₆H₂₀Cl₂N₂O₄, 494.08; m/z found, 495.0 [M+H]⁺. ¹H NMR (500 MHz, CDCl₃): 7.44 (d, J=8.8 Hz, 2H), 7.40 (d, J=2.2 Hz, 1H), 7.38 (d, J=8.5 Hz, 1H), 7.21 (d, J=9.0 Hz, 2H), 7.00 (s, 1H), 6.96 (dd, J=8.5, 1.9 Hz, 1H), 6.92 (d, J=8.8 Hz, 2H), 6.91 (d, J=8.8 Hz, 2H), 6.68 (s, 1H), 3.85 (s, 3H), 3.84 (s, 3H).

### Example 118

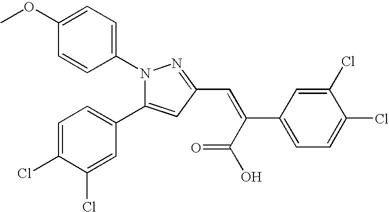

### (Z)-2-(3,4-Dichloro-phenyl)-3-[5-(3,4-dichloro-phenyl)-1-(4-methoxy-phenyl)-1H-pyrazol-3-yl]-acrylic acid

HPLC: Rₜ=6.20 (Method A). MS (ESI): mass calculated for C₂₅H₁₆Cl₄N₂O₃, 531.99; m/z found, 533.0 [M+H]⁺. ¹H NMR (500 MHz, CDCl₃): 7.58 (d, J=1.9 Hz, 1H), 7.45 (d, J=8.5 Hz, 1H), 7.41-7.39 (m, 2H), 7.32 (dd, J=8.5, 2.2 Hz, 1H), 7.22 (d, J=9.0 Hz, 2H), 7.03 (s, 1H), 6.99 (dd, J=8.2, 1.9 Hz, 1H), 6.93 (d, J=9.0 Hz, 2H), 6.71 (s, 1H), 3.86 (s, 3H).

### Example 119

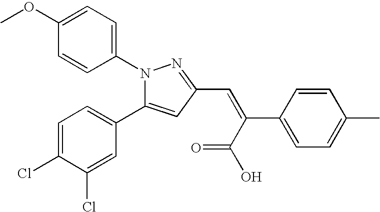

### (Z)-3-[5-(3,4-Dichloro-phenyl)-1-(4-methoxy-phenyl)-1H-pyrazol-3-yl]-2-p-tolyl-acrylic acid

HPLC: Rₜ=6.94 (Method A). MS (ESI): mass calculated for C₂₆H₂₀Cl₂N₂O₃, 478.09; m/z found, 479.1 [M+H]⁺. ¹H NMR (500 MHz, CDCl₃): 7.40-7.38 (m, 4H), 7.22-7.19 (m, 4H), 7.03 (s, 1H), 6.99 (dd, J=8.2, 1.9 Hz, 1H), 6.91 (d, J=9.0 Hz, 2H), 6.69 (s, 1H), 3.85 (s, 3H), 2.38 (s, 3H).

### Example 120

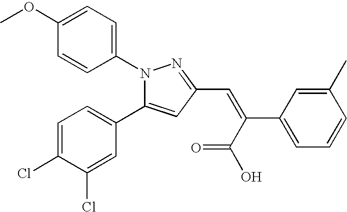

### (Z)-3-[5-(3,4-Dichloro-phenyl)-1-(4-methoxy-phenyl)-1H-pyrazol-3-yl]-2-m-tolyl-acrylic acid

HPLC: Rₜ=6.79 (Method A). MS (ESI): mass calculated for C₂₆H₂₀Cl₂N₂O₃, 478.09; m/z found, 479.1 [M+H]⁺. ¹H NMR (500 MHz, CDCl₃): 7.40 (d, J=2.2 Hz, 1H), 7.38 (d, J=8.2 Hz, 1H), 7.30-7.28 (m, 3H), 7.21 (d, J=9.0 Hz, 2H), 7.18-7.15 (m, 1H), 7.04 (s, 1H), 6.99 (dd, J=8.2, 1.9 Hz, 1H), 6.91 (d, J=9.0 Hz, 2H), 6.70 (s, 1H), 3.85 (s, 3H), 2.39 (s, 3H).

### Example 121

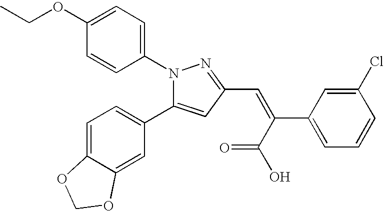

### (Z)-3-[5-Benzo[1,3]dioxol-5-yl-1-(4-ethoxy-phenyl)-1H-pyrazol-3-yl]-2-(3-chloro-phenyl)-acrylic acid

HPLC: Rₜ=6.38 (Method I). MS (ESI): mass calculated for C₂₇H₂₁ClN₂O₅, 488.11; m/z found, 489.1 [M+H]⁺. ¹H NMR (500 MHz, CDCl₃): 7.48 (br s, 1H), 7.36-7.35 (m, 1H), 7.31-7.30 (m, 2H), 7.23 (d, J=9.0 Hz, 2H), 7.02 (s, 1H), 6.89 (d, J=9.0 Hz, 2H), 6.79 (d, J=7.9 Hz, 1H), 6.75 (dd, J=8.2, 1.6 Hz, 1H), 6.67 (d, 1.6 Hz, 1H), 6.58 (s, 1H), 6.00 (s, 2H), 4.06 (q, J=6.9 Hz, 2H), 1.44 (t, 6.9 Hz, 3H).

### Example 122

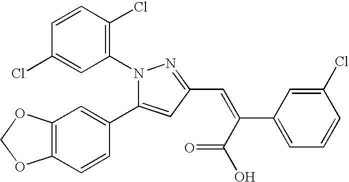

### (Z)-3-[5-Benzo[1,3]dioxol-5-yl-1-(2,5-dichloro-phenyl)-1H-pyrazol-3-yl]-2-(3-chloro-phenyl)-acrylic acid

A. 5-Benzo[1,3]dioxol-5-yl-1-(2,5-dichloro-phenyl)-1H-pyrazole-3-carbaldehyde. To a solution of Dess-Martin periodinane (2.3 g, 5.5 mmol, 2.0 equiv) in CH₂Cl₂ (10 mL) was added a solution of [5-benzo[1,3]dioxol-5-yl-1-(2,5-dichloro-phenyl)-1H-pyrazol-3-yl]-methanol (prepared by the method of Example 1, Steps A-C, 1.0 g, 2.8 mmol) in CH₂Cl₂ (10 mL). The reaction mixture was stirred overnight at room temperature. Then the reaction was quenched with 1 M NaOH (10 mL), and the resulting mixture was stirred until the layers separated. The aqueous layer was back-extracted with CH₂Cl₂ (3×10 mL). The combined organic layers were washed with 1 M NaOH (20 mL) then H₂O (20 mL), dried (MgSO₄), and concentrated to provide the pure aldehyde (1.04 g, 2.8 mmol, 99%). HPLC: Rₜ=5.35 (Method B). MS (ESI): mass calculated for C₁₇H₁₀Cl₂N₂O₃, 360.01; m/z found, 361 [M+H]⁺. ¹H NMR (400 mHz, CDCl₃): 10.05 (s, 1H), 7.50-7.43 (m, 1H), 7.25-7.21 (m, 2H), 7.7-7.26 (m, 1H), 6.96 (s, 1H), 6.74-6.72 (m, 1H), 6.68-6.65 (m, 2H), 5.97 (s, 2H).

B. 3-[5-Benzo[1,3]dioxol-5-yl-1-(2,5-dichloro-phenyl)-1H-pyrazol-3-yl]-2-(3-chloro-phenyl)-acrylic acid, E and Z stereoisomers. To a mixture of 5-benzo[1,3]dioxol-5-yl-1-(2,5-dichloro-phenyl)-1H-pyrazole-3-carbaldehyde (0.20 g, 0.55 mmol) and 3-chlorophenyl acetic acid (0.19 g, 0.82 mmol) was added acetic anhydride (1.0 mL) and TEA (1.0 mL). The mixture was allowed to stir overnight at room temperature. The TEA was removed under reduced pressure, and the resulting mixture was purified on silica gel (MPLC, 0-5% MeOH/CH₂Cl₂) to provide exclusively the E acrylic acid as a brown foam (0.14 g, 49%). The foam was then dissolved in CHCl₃ (10 mL), and the solution was placed in quartz tubes and subjected to uv/vis light overnight. The solvent was removed to provide a 1:1 mixture of E and Z stereoisomers. The stereoisomers were separated by preparative reversed-phase HPLC (acetonitrile/water) to afford the pure Z (0.02 g, 0.04 mmol, 15%) and E acrylic acids (0.03 g, 0.04 mmol, 20%). Z stereoisomer: HPLC: Rₜ=5.86 (Method I). MS (ESI): mass calculated for C₂₅H₁₅Cl₃N₂O₄, 512.01; m/z found, 513.0 [M+H]⁺. ¹H NMR (500 MHz, CDCl₃): 7.48 (br s, 1H), 7.45 (br s, 1H), 7.43 (s, 2H), 7.38-7.36 (m, 1H), 7.32-7.31 (m, 2H), 7.06 (s, 1H), 6.75 (d, J=8.5 Hz, 1H), 6.69 (s, 1H), 6.68 (d, J=8.2 Hz, 2H), 5.99 (s, 2H).

### Example 123

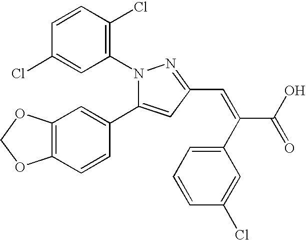

### (E)-3-[5-Benzo[1,3]dioxol-5-yl-1-(2,5-dichloro-phenyl)-1H-pyrazol-3-yl]-2-(3-chloro-phenyl)-acrylic acid

HPLC: Rₜ=4.82 (Method I). MS (ESI): mass calculated for C₂₅H₁₅Cl₃N₃O₂, 512.0; m/z found, 513 [M+H]⁺. ¹H NMR (500 mHz, CDCl₃): 8.05 (s, 1H), 7.43-7.34 (m, 3H), 7.26-7.24 (m, 4H), 6.65 (d, J=8.5 Hz, 1H), 6.45-6.43 (m, 2H), 5.93 (s, 2H), 5.49 (s, 1H).

### Example 124

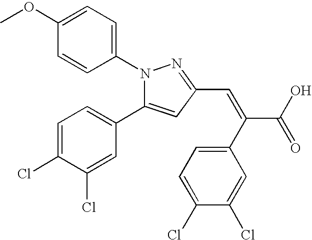

### (E)-2-(3,4-Dichloro-phenyl)-3-[5-(3,4-dichloro-phenyl)-1-(4-methoxy-phenyl)-1H-pyrazol-3-yl]-acrylic acid

HPLC: Rₜ=6.22 (Method I). MS (ESI): mass calculated for C₂₅H₁₆Cl₄N₂O₃, 531.99; m/z found, 532.9 [M+H]⁺. ¹H NMR (500 MHz, CDCl₃): 8.09 (s, 1H), 7.54 (d, J=8.2 Hz, 1H), 7.47 (d, J=1.9 Hz, 1H), 7.33 (d, J=8.2 Hz, 1H), 7.21 (dd, J=8.2, 1.9 Hz, 1H), 7.15 (s, 1H), 7.14 (d, J=9.0 Hz, 2H), 6.88 (d, J=9.0 Hz, 2H), 6.83 (dd, J=8.5, 2.2 Hz, 1H), 5.68 (s, 1H), 3.83 (s, 3H).

### Example 125

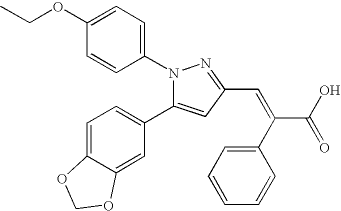

### (E)-3-[5-Benzo[1,3]dioxol-5-yl-1-(4-ethoxy-phenyl)-1H-pyrazol-3-yl]-2-(3-chloro-phenyl)-acrylic acid

HPLC: Rₜ=6.28 (Method I). MS (ESI): mass calculated for C₂₇H₂₁ClN₂O₅, 488.11; m/z found, 489.1 [M+H]⁺. ¹H NMR (500 MHz, CDCl₃): 8.09 (s, 1H), 7.40-7.38 (m, 3H), 7.26-7.23 (m, 1H), 7.16 (d, J=9.0 Hz, 2H), 6.85 (d, J=8.8 Hz, 2H), 6.68 (d, J=7.9 Hz, 1H), 6.50 (dd, J=7.9, 1.6 Hz, 1H), 6.45 (d, J=1.6 Hz, 1H), 5.93 (s, 2H), 5.46 (s, 1H), 4.03 (q, J=6.9 Hz, 2H), 1.42 (t, J=6.9 Hz, 3H).

### Example 126

(Reduction)

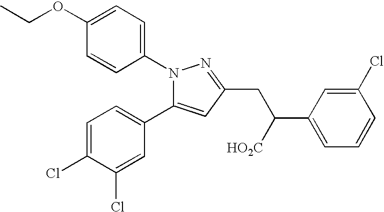

### 2-(3-Chloro-phenyl)-3-[5-(3,4-dichloro-phenyl)-1-(4-ethoxy-phenyl)-1H-pyrazol-3-yl]-propionic acid

To a solution of 2-(3-chloro-phenyl)-3-[5-(3,4-dichloro-phenyl)-1-(4-ethoxy-phenyl)-1H-pyrazol-3-yl]-acrylic acid (Example 108, Step B; 0.043 g, 0.084 mmol) in EtOH (5 mL) was added tosylhydrazine (0.22 g, 1.2 mmol). To the light yellow solution was added a mixture of NaOAc (0.098 g, 1.2 mmol) in H₂O (1 mL). The resulting mixture was heated to 100° C. overnight, then cooled to rt, diluted with H₂O (10 mL), and extracted with CH₂Cl₂ (3×10 mL). The combined organic layers were dried (MgSO₄) and then concentrated to provide a yellow oil. The oil was purified by preparative reversed-phase HPLC (acetonitrile/water) to afford the pure alkane as a colorless oil (10 mg, 23%). TLC (silica gel, 9:1 CH₂Cl₂/MeOH): R_{f}=0.43. HPLC: Rₜ=10.7 (Method A). MS (ESI): mass calculated for C₂₆H₂₁Cl₃N₂O₃, 514.06; m/z found, 513 [M−H]⁻. ¹H NMR (400 mHz, CDCl₃): 7.32-7.23 (m, 6H), 7.14-7.10 (m, 2H), 6.92-6.89 (m, 1H), 6.88-6.85 (m, 2H), 6.23 (s, 1H), 4.03 (q, J=6.9 Hz, 2H), 4.04-4.00 (m, 1H), 3.50 (dd, J=6.7, 14.7 Hz, 1H), 3.09 (dd, J=8.7, 14.7 Hz, 1H), (1.42 (t, J=7.0 Hz, 3H),

The compounds of Examples 127 and 128 were made according to the synthetic methods outlined in Example 126 and Scheme H.

### Example 127

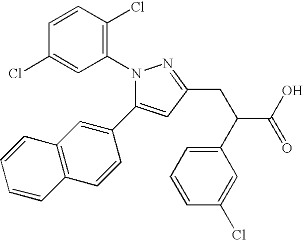

### 2-(3-Chloro-phenyl)-3-[1-(2,5-dichloro-phenyl)-5-naphthalen-2-yl-1H-pyrazol-3-yl]-propionic acid

HPLC: Rₜ=4.77 (Method B). MS (ESI): mass calculated for C₂₈H₁₉Cl₃N₂O₂, 520.05; m/z found, 521/523 [M+H]⁺. ¹H NMR (400 MHz, CDCl₃): 7.79-7.77 (m, 1H), 7.73-7.68 (m, 2H), 7.61-7.60 (m, 1H), 7.48-7.46 (m, 3H), 7.38-7.37 (m, 1H), 7.31-7.26 (m, 4H), 7.20 (dd, J=8.5, 1.8 Hz, 1H), 6.35 (s, 1H), 4.16 (dd, J=8.3, 7.0 Hz, 1H), 3.54 (dd, J=14.8, 8.3 Hz, 1H), 3.19 (dd, J=14.8, 7.0 Hz, 1H).

### Example 128

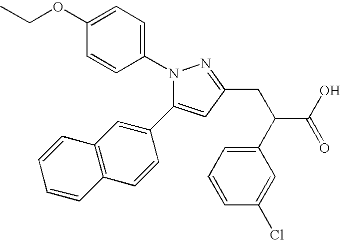

### 2-(3-Chloro-phenyl)-3-[1-(4-ethoxy-phenyl)-5-naphthalen-2-yl-1H-pyrazol-3-yl]-propionic acid

HPLC: Rₜ=5.07 (Method A). MS (ESI): mass calculated for C₃₀H₂₅ClN₂O₃, 497.0; m/z 497.1 [M+H]⁺. ¹H NMR (500 mHz, CDCl₃): 7.80-7.78 (m, 1H), 7.74-7.70 (m, 3H), 7.50-7.48 (m, 2H), 7.39 (s, 1H), 7.28-7.26 (m, 3H), 7.18-7.14 (m, 3H), 6.80 (d, J=8.8 Hz, 2H), 6.36 (s, 1H), 4.16 (dd, J=9.3, 6.0 Hz, 1H), 4.00 (q, J=6.8 Hz, 2H), 3.58 (dd, J=15.0, 9.3 Hz, 1H), 3.19 (dd, J=15.0, 6.0 Hz, 1H), 1.40 (t, J=6.8 Hz, 3H).

The compounds of Examples 129-132 were made according to the synthetic methods outlined in Scheme D.

### Example 129

(Preparation of Tetrazoles)

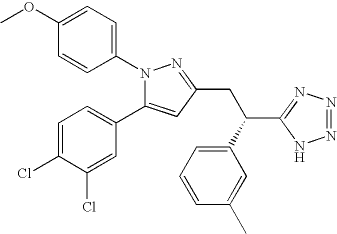

### 5-{(S)-2-[5-(3,4-Dichloro-phenyl)-1-(4-methoxy-phenyl)-1H-pyrazol-3-yl]-1-m-tolyl-ethyl}-1H-tetrazole

A. (S)-N-(2-Cyano-ethyl)-3-[5-(3,4-dichloro-phenyl)-1-(4-methoxy-Phenyl)-1H-pyrazol-3-yl]-2-m-tolyl-propionamide. To a 3-neck round-bottom flask was added (S)-3-[5-(3,4-dichloro-phenyl)-1-(4-methoxy-phenyl)-1H-pyrazol-3-yl]-2-m-tolyl-propionic acid (Example 1; 5.0 g, 9.9 mmol, 1.0 equiv), EDC (4.7 g, 24.7 mmol, 2.5 equiv) and HOBT (3.3 g, 24.7 mmol, 2.5 equiv) under nitrogen. N,N-Dimethylformamide (50 mL) was added, followed by 3-aminopropanenitrile (1.9 g, 24.7 mmol, 2.5 equiv) and diisopropylethylamine (6.8 mL, 39.6 mmol, 4.0 equiv). The reaction mixture was stirred overnight, then was diluted with ethyl acetate (200 mL), washed with 1 N HCl (100 mL), H₂O (100 mL), 10% sodium bicarbonate (100 mL), H₂O (100 mL) then brine (100 mL), and dried (sodium sulfate). The solvent was then removed under reduced pressure yielding the desired amide (5.35 g, 99%), which was used in the next step without purification. HPLC: Rₜ=7.89 (Method A). MS (ESI): mass calculated for C₂₉H₂₆Cl₂N₄O₂, 532.14; m/z found, 533.3 [M+H]⁺. ¹H NMR (500 MHz, CDCl₃): 7.31-7.30 (m, 2H), 7.23 (t, J=7.4 Hz, 1H), 7.19 (br s, 1H), 7.16-7.14 (m, 3H), 7.10 (d, J=7.4 Hz, 1H), 6.91 (dd, J=8.5, 2.2 Hz, 1H), 6.87 (d, J=9.0 Hz, 2H), 6.20 (s, 1H), 6.09 (t, J=6.0 Hz, 1H), 3.90 (dd, J=9.0, 6.0 Hz, 1H), 3.82 (s, 3H), 3.56-3.50 (m, 2H), 3.35-3.31 (m, 1H), 3.08 (dd, J=14.8, 6.0 Hz, 1H), 2.53-2.46 (m, 2H), 2.35 (s, 3H).

B. 3-(5-{(S)-2-[5-(3,4-Dichloro-Phenyl)-1-(4-methoxy-phenyl)-1H-pyrazol-3-yl]-1-m-tolyl-ethyl}-tetrazol-1-yl)-propionitrile. A 3-neck round-bottom flask was charged with (S)-N-(2-cyano-ethyl)-3-[5-(3,4-dichloro-phenyl)-1-(4-methoxy-phenyl)-1H-pyrazol-3-yl]-2-m-tolyl-propionamide (4.0 g, 7.5 mmol, 1.0 equiv) and triphenyl phosphine (4.91 g, 18.8 mmol, 2.5 equiv) under nitrogen. Acetonitrile was added, and the mixture was stirred at room temperature until all of the solids dissolved. The solution was then cooled to 0° C., and diisopropyl azodicarboxylate (3.79 mL, 18.8 mmol, 2.5 equiv) was added slowly via syringe. After the resulting mixture had stirred for 5 min, trimethylsilyl azide (3.0 mL, 22.5 mmol, 3 equiv) was added via syringe over 20 min. The reaction mixture was allowed to warm to room temperature and was stirred for 30 min, and then was stirred at 50° C. for 14 h. The mixture was cooled to room temperature, then to 0° C., and a solution of sodium nitrite (685 mg) in water (3.3 mL) was added. After 20 min a solution of cerric ammonium nitrate (5.5 g) in water (15.5 mL) was added, and the resulting mixture was stirred for 30 min. The mixture was then added to water (200 mL), and the resulting solution was extracted with dichloromethane (2×100 mL). The combined extracts were washed with brine (100 mL), dried (Na₂SO₄), and concentrated under reduced pressure. The crude residue was purified by flash chromatography (25% ethyl acetate/dichloromethane) yielding the desired protected tetrazole (2.1 g, 50%). HPLC: Rₜ=8.18 (Method A). MS (ESI): mass calculated for C₂₉H₂₅Cl₂N₇O, 557.15; m/z found, 558.3 [M+H]⁺. ¹H NMR (500 MHz, CDCl₃): 7.30 (d, J=8.2 Hz, 1H), 7.28-7.25 (m, 3H), 7.17-7.15 (m, 3H), 7.06 (d, J=9.0 Hz, 2H), 6.89-6.86 (m, 3H), 6.24 (s, 1H), 4.75 (dd, J=10.2, 5.3 Hz, 1H), 4.45-4.43 (m, 2H), 3.92 (dd, J=15.2, 10.2 Hz, 1H), 3.83 (s, 3H), 3.42 (dd, J=15.2, 5.3 Hz, 1H), 2.85-2.75 (m, 1H), 2.53-2.49 (m, 1H), 2.34 (s, 3H).

C. 5-{(S)-2-[5-(3,4-Dichloro-phenyl)-1-(4-methoxy-phenyl)-1H-pyrazol-3-yl]-1-m-tolyl-ethyl}-1H-tetrazole. To a solution of 3-(5-{(S)-2-[5-(3,4-dichloro-phenyl)-1-(4-methoxy-phenyl)-1H-pyrazol-3-yl]-1-m-tolyl-ethyl}-tetrazol-1-yl)-propionitrile (1.5 g, 2.7 mmol) in dichloromethane (25 mL) was added DBU (2.9 mL, 18.9 mmol, 7.0 equiv), and the mixture was stirred at room temperature for 48 h. Dichloromethane (200 mL) was added, and the resulting mixture was washed with 1 N HCl (2×100 mL) then water (100 mL), dried (Na₂SO₄), and concentrated under reduced pressure. The crude residue was purified by flash chromatography (50% dichloromethane/ethyl acetate) to afford the title compound (1.3 grams, 95%). HPLC: Rₜ=5.31 (Method A). MS (ESI): mass calculated for C₂₆H₂₂Cl₂N₆O, 504.12; m/z found, 505.3 [M+H]⁺. ¹H NMR (500 MHz, CDCl₃): 7.32 (d, J=8.2 Hz, 1H), 7.28-7.24 (m, 3H), 7.21 (t, J=7.7 Hz, 1H), 7.15 (d, J=8.8 Hz, 2H), 7.08 (d, J=7.7 Hz, 1H), 6.95-6.94 (m, 3H), 6.88 (dd, J=8.5, 2.2 Hz, 1H), 6.18 (s, 1H), 4.85 (dd, J=9.0, 3.6 Hz, 1H), 3.86 (s, 3H), 3.58 (dd, J=14.8, 8.5 Hz, 1H), 3.42 (dd, J=15.4, 3.6 Hz, 1H), 2.31 (s, 3H).

### Example 130

(Preparation of Tetrazoles)

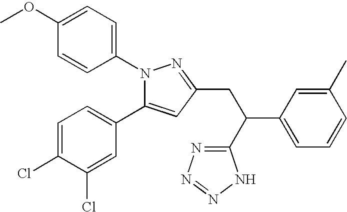

### 5-{2-[5-(3,4-Dichloro-phenyl)-1-(4-methoxy-phenyl)-1H-pyrazol-3-yl]-1-m-tolyl-ethyl}-1H-tetrazole

A. 3-[5-(3,4-Dichloro-phenyl)-1-(4-methoxy-phenyl)-1H-pyrazol-3-yl]-2-m-tolyl-propionitrile. To a solution of sodium bis(trimethylsilyl)amide (14.0 mL, 1.0 M solution in THF, 1.0 equiv) in tetrahydrofuran (56.0 mL) at 0° C. was added 3-methylbenzyl cyanide (1.84 g, 14.0 mmol, 1.0 equiv). This mixture was stirred at 0° C. for 30 min then was added to a solution of 3-bromomethyl-5-(3,4-dichloro-phenyl)-1-(4-methoxy-phenyl)-1H-pyrazole (prepared as in Method 1; 5.78 g, 14.0 mmol, 1.0 equiv) in tetrahydrofuran (56.0 mL) and allowed to stir for 2 h. The reaction was quenched with satd aq ammonium chloride (10.0 mL), and the resulting mixture was diluted with water (200 mL), and extracted with diethyl ether (2×100 mL). The combined extracts were dried (Na₂SO₄) and concentrated under reduced pressure. The crude material was purified by flash chromatography (25% ethyl acetate/hexanes) to yield the title intermediate (2.76 g, 43%). HPLC: Rₜ=13.44 (Method G). MS (ESI): mass calculated for C₂₆H₂₁Cl₂N₃O, 461.11; m/z found, 462.0 [M+H]⁺. ¹H NMR (500 MHz, CDCl₃): 7.36 (d, J=1.9 Hz, 1H), 7.33 (d, J=8.2 Hz, 1H), 7.28 (t, J=7.4 Hz, 1H), 7.24 (s, 1H), 7.23-7.21 (m, 1H), 7.18 (d, J=8.8 Hz, 2H), 7.19-7.16 (m, 1H), 6.95 (dd, J=8.5, 2.2 Hz, 1H), 6.89 (d, J=8.8 Hz, 2H), 6.42 (s, 1H), 4.22 (dd, J=9.6, 6.0 Hz, 1H), 3.83 (s, 3H), 3.30-3.21 (m, 2H), 2.38 (s, 3H).

B. 5-{2-[5-(3,4-Dichloro-phenyl)-1-(4-methoxy-phenyl)-1H-pyrazol-3-yl]-1-m-tolyl-ethyl}-1H-tetrazole. To a 48-mL pressure vessel (Chemglass) were added N,N-dimethylformamide (25.0 mL), 3-[5-(3,4-dichloro-phenyl)-1-(4-methoxy-phenyl)-1H-pyrazol-3-yl]-2-m-tolyl-propionitrile (2.76 g, 5.97 mmol, 1.0 equiv), ammonium chloride (1.58 g, 29.8 mmol, 5.0 equiv) and sodium azide (1.94 g, 29.8 mmol, 5.0 equiv). The screw-cap vessel was sealed and then placed in an oil bath heated to 90° C. for 48 h. The reaction mixture was cooled to room temperature, pH-adjusted with formic acid, diluted with water (100 mL), and extracted with ethyl acetate (3×50 mL). The combined extracts were washed with water (3×50 mL) then brine (50 mL), dried (Na₂SO₄), and concentrated under reduced pressure. The crude material was purified by flash chromatography (5% methanol/dichloromethane) to yield the title compound (1.9 g, 63%). HPLC: Rₜ=3.09 (Method A). MS (ESI): mass calculated for C₂₆H₂₂Cl₂N₆O, 504.12; m/z found, 505.1 [M+H]⁺. ¹H NMR (500 MHz, DMSO-d₆): 7.57 (d, J=8.5 Hz, 1H), 7.41 (d, J=2.2 Hz, 1H), 7.23-7.16 (m, 3H), 7.09-7.07 (m, 3H), 7.01 (dd, J=8.5, 2.2 Hz, 1H), 6.96 (d, J=9.0 Hz, 2H), 6.46 (s, 1H), 4.86 (dd, J=9.0, 6.6 Hz, 1H), 3.77 (s, 3H), 3.62 (dd, J=14.8, 9.3 Hz, 1H), 3.35 (dd, J=14.8, 6.6 Hz, 1H), 2.28 (s, 3H).

### Example 131

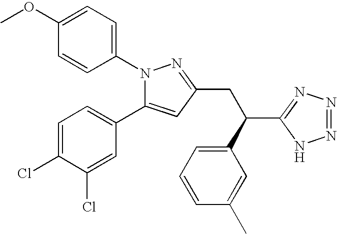

### 5-{(R)-2-[5-(3,4-Dichloro-phenyl)-1-(4-methoxy-phenyl)-1H-pyrazol-3-yl]-1-m-tolyl-ethyl}-1H-tetrazole

This compound was obtained by chiral-HPLC separation of the two enantiomers (Method C) from the racemic mixture prepared in Example 130. HPLC: Rₜ=5.31 (Method A). MS (ESI): mass calculated for C₂₆H₂₂Cl₂N₆O, 504.12; m/z found, 505.3 [M+H]⁺. ¹H NMR (500 MHz, CDCl₃): 7.32 (d, J=8.2 Hz, 1H), 7.28-7.26 (m, 3H), 7.21 (t, J=7.7 Hz, 1H), 7.15 (d, J=8.8 Hz, 2H), 7.08 (d, J=7.7 Hz, 1H), 6.94 (m, 3H), 6.88 (dd, J=8.5, 2.2 Hz, 1H), 6.18 (s, 1H), 4.85 (dd, J=9.0, 3.6 Hz, 1H), 3.86 (s, 3H), 3.58 (dd, J=14.8, 8.5 Hz, 1H), 3.42 (dd, J=15.4, 3.6 Hz, 1H), 2.31 (s, 3H).

### Example 132

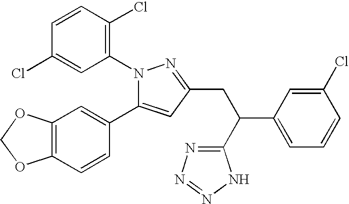

### 5-[2-[5-Benzo[1,3]dioxol-5-yl-1-(2,5-dichloro-phenyl)-1H-pyrazol-3-yl]-1-(3-chloro-phenyl)-ethyl]-1H-tetrazole

This compound was prepared by the procedure described in Example 130, substituting 5-benzo[1,3]dioxol-5-yl-3-bromomethyl-1-(2,5-dichloro-phenyl)-1H-pyrazole (prepared as in Method 1) for 3-bromomethyl-5-(3,4-dichloro-phenyl)-1-(4-methoxy-phenyl)-1H-pyrazole in step A. HPLC: Rₜ=5.21 (Method A). MS (ESI): mass calculated for C₂₅H₁₇Cl₃N₆O₂, 538.05; m/z found, 539.0 [M+H]⁺. ¹H NMR (500 MHz, CDCl₃): 7.46-7.41 (m, 2H), 7.32 (d, J=2.2 Hz, 1H), 7.26-7.23 (m, 2H), 7.14-7.04 (m, 2H), 6.70 (d, J=7.9 Hz, 1H), 6.57 (dd, J=8.2, 1.9 Hz, 1H), 6.54 (d, J=1.6 Hz, 1H), 6.17 (brs, 1H), 5.96 (s, 2H), 5.02 (dd, J=8.5, 4.4 Hz, 1H), 3.60 (dd, J=15.1, 8.8 Hz, 1H), 3.48 (dd, J=15.1, 40.4 Hz, 1H).

The compounds of Examples 133 and 134 were made according to the synthetic methods outlined in Scheme J.

### Example 133

(Ester-Arylation)

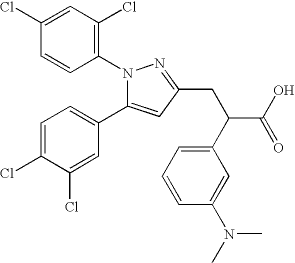

### 3-[5-(3,4-Dichloro-phenyl)-1-(2,4-dichloro-phenyl)-1H-pyrazol-3-yl]-2-(3-dimethylamino-phenyl)-propionic acid

A. 6-(3,4-Dichloro-phenyl)-6-hydroxy-4-oxo-hex-5-enoic acid bis-lithium salt. To a 3-neck flask was added diethyl ether (120 mL) and lithium bis(trimethylsilyl)amide (10.0 g, 59.9 mmol, 2.0 equiv) under nitrogen. The slurry was cooled to −78° C., then a solution of 1-(3,4-dichloro-phenyl)-ethanone (11.3 g, 59.9 mmol, 2.0 equiv) in diethyl ether (120 mL) was added dropwise. The mixture was stirred at −78° C. for 30 min, then a solution of succinic anhydride (3.0 g, 29.9 mmol, 1.0 equiv) in diethyl ether (60 mL) was added dropwise. The reaction mixture was stirred at −78° C. for 1 h then allowed to warm to room temperature and stirred 16 h. The resulting precipitate was filtered off, washed with diethyl ether (2×60 mL), and dried yielding a yellow powder (9.48 g, 99%), which was used in the next step without purification or characterization.

B. 3-[5-(3,4-Dichloro-phenyl)-1-(2,4-dichloro-phenyl)-1H-pyrazol-3-yl]-propionic acid. To a round-bottom flask was added 6-(3,4-dichloro-phenyl)-6-hydroxy-4-oxo-hex-5-enoic acid bis-lithium salt (9.48 g, 31.3 mmol, 1.0 equiv), 2,4 -dichloro-phenyl hydrazine hydrochloride (6.66 g, 31.3 mmol, 1.0 equiv) and EtOH (250 mL) under nitrogen. The mixture was stirred at room temperature for 24 h. The solvent was removed, and the crude residue was partitioned between 5% HCl and diethyl ether (200 mL each). The layers were separated, and the aqueous layer was extracted with diethyl ether (2×100 mL). The combined organic layers were washed with water (100 mL) then brine (100 mL), dried (Na₂SO₄), and concentrated under reduced pressure. Purification by flash chromatography (25% ethyl acetate/dichloromethane) afforded the title intermediate (4.5 g, 33%). HPLC: Rₜ=3.04 (Method A). MS (ESI): mass calculated for C₁₈H₁₂Cl₄N₂O₂, 427.97; m/z found, 429/431 [M+H]⁺. ¹H NMR (500 MHz, DMSO-d₆): 12.20 (br s, 1H), 7.82 (d, J=2.2 Hz, 1H), 7.68 (d, J=8.5 Hz, 1H), 7.61-7.59 (m, 2H), 7.50 (d, J=2.2 Hz, 1H), 7.05 (dd, J=8.2, 1.9 Hz, 1H), 6.73 (s, 3H), 2.88 (t, J=7.4 Hz, 2H), 2.64 (t, J=7.4 Hz, 2H).

C. 3-[5-(3,4-Dichloro-phenyl)-1-(2,4-dichloro-phenyl)-1H-pyrazol-3-yl]-propionic acid tert-butyl ester. To a 3-neck round bottom flask fitted with an air condenser was added 3-[5-(3,4-dichloro-phenyl)-1-(2,4-dichloro-phenyl)-1H-pyrazol-3-yl]-propionic acid (1.0 g, 2.3 mmol, 1.0 equiv) and toluene (23 mL) under nitrogen. The mixture was heated to 80° C. then N,N-dimethyl-di-tert-butylacetal (2.36 g, 11.6 mmol, 5.0 equiv) was added dropwise (neat). The reaction mixture was heated at 80° C. for 1 h then additional N,N-dimethyl-di-tert-butylacetal (2.36 g, 11.6 mmol, 5.0 equiv) was added. This mixture was stirred at 80° C. for 2 h then cooled to room temperature and partitioned between water (100 mL) and ether (100 mL). The organic layer was washed with 1 M sodium hydroxide (50 mL), water (50 mL) then brine (50 mL), dried (Na₂SO₄), and concentrated under reduced pressure. The crude material was then purified by flash chromatography (20% ethyl acetate/hexanes) to afford the desired ester (1.1 g, >99%). HPLC: Rₜ=3.59 (Method A). MS (ESI): mass calculated for C₂₂H₂₀Cl₄N₂O₂, 484.03; m/z found, 485.0 [M+H]⁺. ¹H NMR (500 MHz, DMSO-d₆): 7.81 (d, J=2.2 Hz, 1H), 7.65 (d, J=8.5 Hz, 1H), 7.61-7.59 (m, 2H), 7.48 (d, J=2.2 Hz, 1H), 7.05 (dd, J=8.2, 1.9 Hz, 1H), 6.71 (s, 1H), 2.87 (t, J=7.4 Hz, 2H), 2.61 (t, J=7.4 Hz, 2H), 1.38 (s, 9H).

D. 3-[5-(3,4-Dichloro-phenyl)-1-(2,4-dichloro-phenyl)-1H-pyrazol-3-yl]-2-(3-dimethylamino-phenyl)-propionic acid tert-butyl ester. To a mixture of palladium(II) acetate (3 mg, 5 mol %), 2-dicyclohexylphosphino-2′-(N,N-dimethylamino)biphenyl (10 mg, 5 mol %) and lithium bis(trimethylsilyl)amide (0.55 mL, 0.55 mmol, 1.1 equiv, 1.0 M solution in tetrahydrofuran) in toluene (0.5 mL) under nitrogen at −10° C., was added a solution of 3-[5-(3,4-dichloro-phenyl)-1-(2,4-dichloro-phenyl)-1H-pyrazol-3-yl]-propionic acid tert-butyl ester (243 mg, 0.50 mmol, 1.0 equiv) in toluene (1.0 mL). This mixture was stirred at −10° C. for 10 min, then (3-bromo-phenyl)-dimethyl-amine (42 mg, 0.21 mmol, 0.45 equiv) in toluene (0.5 mL) was added. The resulting solution was allowed to warm to room temperature then was heated to 80° C. for 3 h. The reaction mixture was cooled to room temperature, and the reaction was quenched with satd aq ammonium chloride (1.0 mL). Water (10.0 mL) was added, and the resulting mixture was extracted with diethyl ether (2×10 mL). The combined extracts were washed with brine (10 mL), dried (Na₂SO₄), and concentrated under reduced pressure. The crude material was purified by reversed-phase HPLC to afford the desired aryl acetic acid ester (20 mg, 16%). MS (ESI): mass calculated for C₃₀H₂₉Cl₄N₃O₂, 603.10; m/z found, 604.1 [M+H]⁺.

E. 3-[5-(3,4-Dichloro-phenyl)-1-(2,4-dichloro-phenyl)-1H-pyrazol-3-yl]-2-(3-dimethylamino-phenyl)-propionic acid. 3-[5-(3,4-Dichloro-phenyl)-1-(2,4-dichloro-phenyl)-1H-pyrazol-3-yl]-2-(3-dimethylamino-phenyl)-propionic acid tert-butyl ester (20 mg, 0.03 mmol) was dissolved in 1:1 trifluoroacetic acid/dichloromethane (1.0 mL) and stirred for 2 h. The reaction mixture was concentrated under reduced pressure, and the crude residue was dissolved in 1:1 acetonitrile/water (2.0 mL). The solution was lyopholized to afford the title compound (18 mg, >99%). HPLC: Rₜ=2.60 (Method B). MS (ESI): mass calculated for C₂₆H₂₁Cl₄N₃O₂, 547.04; m/z found, 548/550 [M+H]⁺. ¹H NMR (500 MHz, DMSO-d₆): 7.81 (d, J=1.9 Hz, 1H), 7.60-7.58 (m, 3H), 7.45 (d, J=2.2 Hz, 1H), 7.18 (t, J=7.9 Hz, 1H), 7.02 (dd, J=8.5, 2.2 Hz, 1H), 6.78 (m, 3H), 6.64 (s, 1H), 3.96 (dd, J=8.8, 6.6 Hz, 1H), 3.36 (dd, J=15.1, 9.0 Hz, 1H), 2.93 (dd, J=15.1, 6.6 Hz, 1H), 2.91 (s, 6H).

### Example 134

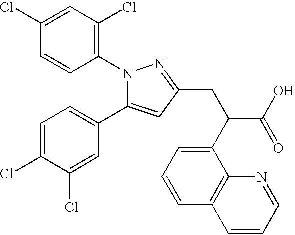

### 3-[5-(3,4-Dichloro-phenyl)-1-(2,4-dichloro-phenyl)-1H-pyrazol-3-yl]-2-quinolin-8-yl-propionic acid

The title compound was prepared as described in Example 133, substituting 8-bromo-quinoline for (3-bromo-phenyl)-dimethyl-amine in Step D. HPLC: Rₜ=2.99 (Method B). MS (ESI): mass calculated for C₂₇H₁₇Cl₄N₃O₂, 555.01; m/z found, 556.1 [M+H]⁺.

The compounds of Examples 135-138 were made according to the synthetic methods outlined in Scheme I.

### Example 135

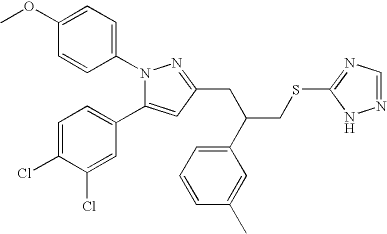

### 5-{3-[5-(3,4-Dichloro-phenyl)-1-(4-methoxy-phenyl)-1H-pyrazole-3-yl]-2-m-tolyl-propylsulfanyl}-1H-[1,2,4]-triazole

A. 3-[5-(3,4-Dichloro-phenyl)-1-(4-methoxy-phenyl)-1H-pyrazol-3-yl]-2-m-tolyl-propan-1-ol. To a 3-neck round-bottom flask charged with nitrogen was added 3-[5-(3,4-dichloro-phenyl)-1-(4-methoxy-phenyl)-1H-pyrazol-3-yl]-2-m-tolyl-propionic acid ethyl ester (see Method 2, product before hydrolysis; 798 mg, 1.57 mmol, 1.0 equiv) and tetrahydrofuran (6.0 mL). The mixture was cooled to −78° C., then diisobutyl aluminum hydride (4.7 mL, 1.0 M solution in tetrahydrofuran) was added dropwise. The reaction mixture was stirred at −78° C. for 30 min then allowed to warm to room temperature and stirred 1 h. The mixture was then poured slowly into a satd aq solution of Rochelle salt (50 mL). Diethyl ether (50 mL) was added, and the resulting mixture was stirred for 3 h. The organic layer was dried (Na₂SO₄) and concentrated under reduced pressure to afford 732 mg of the desired alcohol, which was used in the next step without purification.

B. 3-(3-Bromo-2-m-tolyl-propyl)-5-(3,4-dichloro-phenyl)-1-(4-methoxy-phenyl)-1H-pyrazole. To a 3-neck round-bottom flask was added phosphorus tribromide (599 mg, 2.77 mmol, 1.5 equiv) and dichloromethane (10 mL). The mixture was cooled to 0° C., then a solution of 3-[5-(3,4-dichloro-phenyl)-1-(4-methoxy-phenyl)-1H-pyrazol-3-yl]-2-m-tolyl-propan-1-ol (690 mg, 1.48 mmol, 1.0 equiv) in dichloromethane (3.0 mL) was added. The reaction mixture was allowed to warm to room temperature then was stirred for 16 h. The resulting mixture was loaded directly onto a silica gel column and purified by chromatography (25% ethyl acetate/hexanes) giving the desired bromide (480 mg, 61%). HPLC: Rₜ=3 80 (Method B). MS (ESI): mass calculated for C₂₆H₂₃BrCl₂N₂O, 528.04; m/z found, 529.0 [M+H]⁺.

C. 5-{3-[5-(3,4-Dichloro-phenyl)-1-(4-methoxy-phenyl)-1H-pyrazole-3-yl]-2-m-tolyl-propylsulfanyl}-1H-[1,2,4]-triazole. To a suspension of sodium hydride (4.0 mg, 60% dispersion in oil) in N,N-dimethylformamide (1.0 mL) at 0° C. was added a solution of 2H-[1,2,4]triazole-3-thiol (10.0 mg, 0.1 mmol, 1.1 equiv) in N,N-dimethylformamide (1.0 mL). The mixture was stirred at 0° C. for 30 min then a solution of 3-(3-bromo-2-m-tolyl-propyl)-5-(3,4-dichloro-phenyl)-1-(4-methoxy-phenyl)-1H-pyrazole (48 mg, 0.09 mmol, 1.0 equiv) in N,N-dimethylformamide (1.0 mL) was added. The reaction mixture was brought to room temperature then was stirred for 2 h. The reaction was quenched with satd aq ammonium chloride (1.0 mL), and the resulting mixture was diluted with water (10.0 mL), and extracted with ethyl acetate (3×10 mL). The combined organic layers were washed with water (10 mL) then brine (10 mL), dried (Na₂SO₄), and concentrated under reduced pressure. The crude residue was purified by reversed-phase HPLC to yield the title compound (39 mg, 80%). HPLC: Rₜ=3.26 (Method B). MS (ESI): mass calculated for C₂₈H₂₅Cl₂N₅OS, 549.12; m/z found, 550.1 [M+H]⁺. ¹H NMR (500 MHz, DMSO-d₆): 8.32 (br s, 1H), 7.50 (d, J=8.4 Hz, 1H), 7.35 (d, J=2.1 Hz, 1H), 7.07-7.04 (m, 5H), 6.95 (dd, J=8.4, 21. HZ, 2H), 6.89 (d, J=9.0 Hz, 2H), 6.31 (s, 1H), 3.70 (s, 3H), 3.48 (dd, J=12.9, 6.3 Hz, 1H), 3.36 (dd, J=12.7, 8.2 Hz, 1H), 3.26 (m, 1H), 3.07 (dd, J=14.9, 6.4 Hz, 1H), 2.91 (dd, J=14.9, 8.2 Hz, 1H), 2.21 (s, 3H).

### Example 136

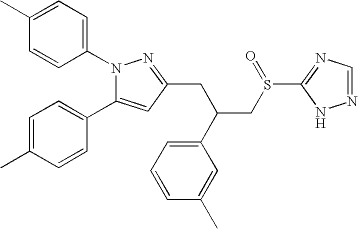

### 5-[3-(1,5-Di-p-tolyl-1H-pyrazol-3-yl)-2-m-tolyl-propane-1-sulfinyl]-1H-[1,2,4]triazole

To a cold (0° C., ice bath) solution of 5-[3-(1,5-d]-p-tolyl-1H-pyrazol-3-yl)-2-m-tolyl-propylsulfanyl]-1H-[1,2,4]triazole (177 mg, 0.37 mmol, 1.0 equiv) [prepared by substituting 3-(1,5-d]-p-tolyl-1H-pyrazol-3-yl)-2-m-tolyl-propionic acid ethyl ester (see Method 2, product before hydrolysis) for 3-[5-(3,4-dichloro-phenyl)-1-(4-methoxy-phenyl)-1H-pyrazol-3-yl]-2-m-tolyl-propionic acid ethyl ester in Step A of Example 135] in dichloromethane (2.0 mL) was added 3-chloroperoxy benzoic acid (90 mg, 0.41 mmol, 1.1 equiv). The reaction mixture was stirred at 0° C. for 15 min, stirred at 40° C. for 1 h, and then cooled to room temperature and stirred for 16 h. The solvent was evaporated under reduced pressure, and the crude material was purified by reversed-phase HPLC giving the desired sulfinyl triazole (165 mg, 90%). HPLC: Rₜ=2.88 (Method B). MS (ESI): mass calculated for C₂₉H₂₉N₅OS, 495.21; m/z found, 496.2 [M+H]⁺. ¹H NMR (500 MHz, DMSO-d₆): 8.79 (s, 1H), 7.00-7.23 (m, 12H), 6.30 (s, 0.5H), 6.14 (s, 0.5H), 3.81 (dd, J=12.5, 3.7 Hz, 0.5H) 3.72 (dd, J=12.9, 7.0 Hz, 0.5H), 3.37-3.60 (m, 1.5H), 3.28-3.25 (m, 0.5H), 2.97-3.15 (m, 2.0H), 2.31-2.27 (m, 9H).

### Example 137

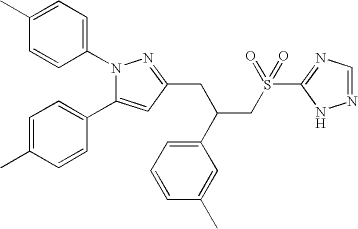

### 5-[3-(1,5-Di-p-tolyl-1H-pyrazol-3-yl)-2-m-tolyl-propane-1-sulfonyl]-1H-[1,2,4]triazole

To a flask was added 5-[3-(1,5-di-p-tolyl-1H-pyrazol-3-yl)-2-m-tolyl-propane-1-sulfinyl]-1H-[1,2,4]triazole (Example 136; 25 mg, 0.05 mmol), hydrogen peroxide (0.15 mL, 30% solution in water) and acetic acid (0.1 ml). The mixture was heated at 50° C. for 24 h and then cooled. Methanol (0.5 mL) and N,N-dimethylformamide (0.5 mL) were added to dissolve the resulting precipitate. This solution was then purified directly by reversed-phase chromatography yielding the title compound (24 mg, 95%). HPLC: Rₜ=2.97 (Method B). MS (ESI): mass calculated for C₂₉H₂₉N₅O₂S, 511.20; m/z found, 512.2 [M+H]⁺. ¹H NMR (500 MHz, DMSO-d₆): 14.87 (brs, 1H), 8.72 (s, 1H), 7.18 (d, J=8.2 Hz, 2H), 7.13 (d, J=8.0 Hz, 2H), 7.08 (d, J=7.0 Hz, 1H), 7.07-7.04 (m, 3H), 7.01-6.99 (m, 3H), 6.95 (d, J=7.4 Hz, 1H), 6.15 (s, 1H), 3.91 (d, J=6.6 Hz, 2H), 3.52-3.49 (m, 1H), 3.08 (dd, J=14.7, 7.6 Hz, 1H), 2.91 (dd, J=14.5, 7.4 Hz, 1H), 2.31 (s, 3H), 2.27 (s, 3H), 2.23 (s, 3H).

### Example 138

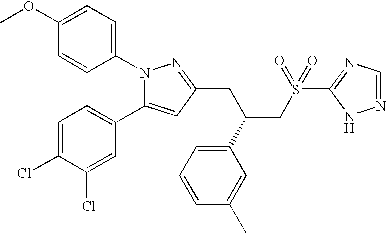

### 5-{(S)-3-[5-(3,4-Dichloro-phenyl)-1-(4-methoxy-phenyl)-1H-pyrazol-3-yl]-2-m-tolyl-propane-1-sulfonyl}-1H-[1,2,4]triazole

The title compound was prepared as outlined in Example 137, substituting the S enantiomer of 3-[5-(3,4-dichloro-phenyl)-1-(4-methoxy-phenyl)-1H-pyrazol-3-yl]-2-m-tolyl-propionic acid ethyl ester [available by chiral separation of ester prepared in Method 2] for the racemic 3-[5-(3,4-dichloro-phenyl)-1-(4-methoxy-phenyl)-1H-pyrazol-3-yl]-2-m-tolyl-propionic acid ethyl ester in Step A. HPLC: Rₜ=2.94 (Method B). MS (ESI): mass calculated for C₂₈H₂₅Cl₂N₅O₃S, 581.11; m/z found, 582.3 [M+H]⁺. ¹H NMR (500 MHz, DMSO-d₆): 14.87 (br s, 1H), 8.72 (s, 1H), 7.58 (d, J=8.5 Hz, 1H), 7.43 (d, J=2.2 Hz, 1H), 7.14 (d, J=9.0 Hz, 2H), 7.08 (d, J=7.4 Hz, 1H), 6.96-7.04 (m, 6H), 6.36 (s, 1H), 3.92 (d, J=6.3 Hz, 2H), 3.78 (s, 3H), 3.53-3.50 (m, 1H), 3.09 (dd, J=14.5, 7.4 Hz, 1H), 2.92 (dd, J=14.5, 7.7 Hz, 1H), 2.23 (s, 3H).

### Example 139

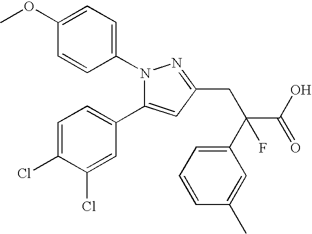

### 3-[5-(3,4-Dichloro-phenyl)-1-(4-methoxy-phenyl-1H-pyrazol-3-yl]-2-fluoro-2-m-tolyl-propionic acid

A. 3-[5-(3,4-Dichloro-phenyl)-1-(4-methoxy-phenyl)-1H-pyrazol-3-yl]-2-fluoro-2-m-tolyl-propionic acid ethyl ester. To a round-bottom flask containing lithium bis(trimethylsilyl)amide (0.47 mL, 1.0 M solution in tetrahydrofuran), and tetrahydrofuran (1.5 mL) at 0° C. under nitrogen, was added 3-[5-(3,4-dichloro-phenyl)-1-(4-methoxy-phenyl)-1H-pyrazol-3-yl]-2-m-tolyl-propionic acid ethyl ester (Method 2, product before hydrolysis; 200 mg, 0.39 mmol, 1.0 equiv) in tetrahydrofuran (1.5 mL). The mixture was allowed to stir at 0° C. for 1 h, then a solution of sultam-F (109 mg, 0.51 mmol, 1.5 equiv) in tetrahydrofuran (1.5 mL) was added, and the resulting solution was stirred at 0° C. for 2 h. The reaction was quenched with satd aq ammonium chloride (5 mL), and the resulting mixture was diluted with water (10 mL) and extracted with ethyl acetate (2×10 mL). The combined extracts were washed with water (10 mL) then brine (10 mL), dried (Na₂SO₄), and concentrated under reduced pressure. The crude residue was purified by reversed-phase HPLC giving the desired alpha-fluoro ester (164 mg, 80%). HPLC: Rₜ=3.66 (Method B). MS (ESI): mass calculated for C₂₈H₂₅Cl₂FN₂O₃, 526.12; m/z found, 527.2 [M+H]⁺.

B. 3-[5-(3,4-Dichloro-phenyl)-1-(4-methoxy-phenyl-1H-pyrazol-3-yl]-2-fluoro-2-m-tolyl-propionic acid. The title compound was made as outlined in Method 2 (Scheme A) by hydrolysis of the ester described in Step A. HPLC: Rₜ=3.34. MS (ESI): mass calculated for C₂₆H₂₁Cl₂FN₂O₃, 498.09; m/z found, 499.1 [M+H]⁺. ¹H NMR (500 MHz, DMSO-d₆): 7.59 (d, J=8.2 Hz, 1H), 7.45 (d, J=1.9 Hz, 1H), 7.38-7.36 (m, 2H), 7.33 (t, J=7.4 Hz, 1H), 7.21 (d, J=7.1 Hz, 1H), 7.17 (d, J=8.8 Hz, 2H), 7.07 (dd, J=8.2, 1.9 Hz, 1H), 6.98 (d, J=8.8 Hz, 2H), 6.48 (s, 1H), 3.77 (m, 1H), 3.78 (s, 3H), 3.42 (dd, J=17.0, 15.4 Hz, 1H), 2.35 (s, 3H).

### Example 140

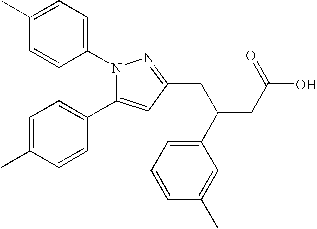

### 4-(1,5-Di-p-tolyl-1H-pyrazol-3-yl)-3-m-tolyl-butyric acid

A. 4-(1,5-Di-p-tolyl-1H-pyrazol-3-yl)-3-m-tolyl-butyronitrile. To a screw-cap vial were added 3-(3-bromo-2-m-tolyl-propyl)-1,5-di-p-tolyl-1H-pyrazole (prepared by the method of Example 67; 300 mg, 0.65 mmol, 1.0 equiv), sodium cyanide (160 mg, 3.3 mmol, 5.0 equiv) and N,N-dimethylformamide (3.0 mL). The sealed mixture was then heated at 100° C. for 48 h. The reaction mixture was cooled to room temperature, diluted with water (10 mL), and extracted with diethyl ether (3×10 mL). The combined extracts were washed with water (4×10 mL) then brine (10 mL), dried (Na₂SO₄), and concentrated under reduced pressure. The crude residue was purified by flash chromatography (25% ethyl acetate/hexanes) giving the desired nitrile (171 mg, 65%). MS (ESI): mass calculated for C₂₈H₂₇N₃, 405.22; m/z found, 406.2 [M+H]⁺.

B. 4-(1,5-Di-p-tolyl-1H-pyrazol-3-yl)-3-m-tolyl-butyric acid methyl ester. To a flask were added 4-(1,5-di-p-tolyl-1H-pyrazol-3-yl)-3-m-tolyl-butyronitrile (100 mg, 0.24 mmol), concd sulfuric acid (1.5 mL) and methanol (1.5 mL). The mixture was heated to reflux for 24 h. The reaction mixture was cooled to room temperature, poured into ice (20 g) and neutralized with satd sodium bicarbonate. The resulting solution was extracted with diethyl ether (3×10 mL), and the combined organic extracts were washed with water (10 mL) then brine (10 mL), dried (Na₂SO₄), and concentrated under reduced pressure. The crude residue was purified by reversed-phase HPLC yielding the desired ester (86 mg, 82%). HPLC: Rₜ=3.43 (Method B). MS (ESI): mass calculated for C₂₉H₃₀N₂O₂, 438.23; m/z found, 439.2 [M+H]⁺. ¹H NMR (500 MHz, CDCl₃): 7.19 (t, J=7.4 Hz, 1H), 7.01-7.13 (m, 11H), 6.15 (s, 1H), 3.56 (s, 3H), 3.54-3.52 (m, 1H), 3.11-3.08 (m, 2H), 2.77-2.75 (m, 2H), 2.36 (s, 3H), 2.32 (s, 6H).

C. 4-(1,5-Di-p-tolyl-1H-pyrazol-3-yl)-3-m-tolyl-butyric acid. The title compound was synthesized by Method 2 (Scheme A) by hydrolysis of the ester described in Step B. HPLC: Rₜ=3.14 (Method B). MS (ESI): mass calculated for C₂₈H₂₈N₂O₂, 424.22; m/z found, 425.8 [M+H]⁺. ¹H NMR (500 MHz, DMSO-d₆): 12.00 (br s, 1H), 6.98-7.19 (m, 12H), 6.23 (s, 1H), 3.39-3.37 (m, 1H), 3.00-2.87 (m, 2H), 2.71 (dd, J=15.5, 5.6 Hz, 1H), 2.56 (dd, J=15.6, 9.4 Hz, 1H), 2.31 (s, 3H), 2.27 (s, 6H).

### Example 141

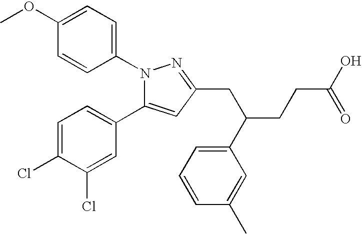

### 5-[5-(3,4-Dichloro-phenyl)-1-(4-methoxy-phenyl)-1H-pyrazol-3-yl]-4-m-tolyl-pentanoic acid

A. 3-[5-(3,4-Dichloro-phenyl)-1-(4-methoxy-phenyl)-1H-pyrazol-3-yl]-2-m-tolyl-propionaldehyde. To a flask containing 3-[5-(3,4-dichloro-phenyl)-1-(4-methoxy-phenyl)-1H-pyrazol-3-yl]-2-m-tolyl-propane-1-ol (prepared by the method of Example 67; 50 mg, 0.11 mmol, 1.0 equiv) and dichloromethane (2.0 mL) was added Dess-Martin reagent (89 mg, 0.21 mmol, 2.0 equiv) in one portion. The reaction mixture was stirred at room temperature for 30 min then poured into satd aq sodium bicarbonate (5.0 mL) containing sodium thiosulfate pentahydrate (5.0 equiv relative to Dess-Martin reagent). The resulting mixture was then diluted with dichloromethane (3.0 mL) and stirred vigorously for 2 h. The resulting organic layer was washed with water (5.0 mL) then brine (5.0 mL), dried (Na₂SO₄), and concentrated under reduced pressure, affording the desired aldehyde, which was used in the next step without purification. Rₜ=3.57 (Method B). MS (ESI): mass calculated for C₂₆H₂₂Cl₂N₂O₂, 464.11; m/z found, 465.0 [M+H]⁺.

B. 5-[5-(3,4-Dichloro-phenyl)-1-(4-methoxy-phenyl)-1H-pyrazol-3-yl]-4-m-tolyl-pent-2-enoic acid methyl ester. To a suspension of sodium hydride (30 mg, 60% dispersion in oil) in tetrahydrofuran (1.5 mL) at 0° C. was added methyl diethylphosphonoacetate (0.13 mL, 0.69 mmol, 1.0 equiv) neat. The mixture was stirred at 0° C. for 30 min, then a solution of 3-[5-(3,4-dichloro-phenyl)-1-(4-methoxy-phenyl)-1H-pyrazol-3-yl]-2-m-tolyl-propionaldehyde (320 mg, 0.69 mmol, 1.0 equiv) in tetrahydrofuran (1.5 mL) was added. The reaction mixture was allowed to warm to room temperature and was stirred 1 h. The reaction was quenched with 2 mL of water, and the resulting mixture was diluted with satd aq ammonium chloride (10 mL) then extracted with diethyl ether (3×20 mL). The combined extracts were washed with water (20 mL) then brine (20 mL), dried (Na₂SO₄), and concentrated under reduced pressure. The crude material was purified by flash chromatography (25% ethyl acetate/hexanes) giving the methyl ester (150 mg, 45%). HPLC: Rₜ=3.70 (Method B). MS (ESI): mass calculated for C₂₉H₂₆Cl₂N₂O₃, 520.13; m/z found, 521.2 [M+H]⁺.

C. 5-[5-(3,4-Dichloro-Phenyl)-1-(4-methoxy-Phenyl)-1H-pyrazol-3-yl]-4-m-tolyl-pentanoic acid methyl ester. To a flask containing ethyl acetate (1.0 mL), ethanol (1.0 mL) and a catalytic amount of Raney nickel was added 5-[5-(3,4-dichloro-phenyl)-1-(4-methoxy-phenyl)-1H-pyrazol-3-yl]-4-m-tolyl-pent-2-enoic acid methyl ester (92 mg, 0.17 mmol). The reaction mixture was stirred under H₂ (˜1 atm) for 2 h and then filtered through a CELITE® pad. The filtrate was concentrated under reduced pressure, and the crude residue was purified by reversed-phase HPLC giving the desired ester (81 mg, 91%). HPLC: Rₜ=3.68 (Method B). MS (ESI): mass calculated for C₂₉H₂₈Cl₂N₃O₃, 522.15; m/z found, 523.3 [M+H]⁺.

D. 5-[5-(3,4-Dichloro-phenyl)-1-(4-methoxy-phenyl)-1H-pyrazol-3-yl]-4-m-tolyl-pentanoic acid. The title compound was made by Method 2 (Scheme A) by hydrolysis of the ester of step C. HPLC: Rₜ=10.60 (Method A). MS (ESI): mass calculated for C₂₈H₂₆Cl₂N₂O₃, 508.13; m/z found, 509.0 [M+H]⁺. ¹H NMR (500 MHz, DMSO-d₆): 11.97 (br s, 1H), 7.57 (d, J=8.5 Hz, 1H), 7.44 (d, J=2.2 Hz, 1H), 7.19 (t, J=7.7 Hz, 1H), 7.15 (d, J=9.0 Hz, 2H), 7.07-7.02 (m, 4H), 6.96 (d, J=9.0 Hz, 2H), 6.42 (s, 1H), 3.77 (s, 3H), 2.92-2.89 (m, 3H), 2.29 (s, 3H), 2.00-1.99 (m, 3H), 1.80-1.77 (m, 1H).

### GENERAL EXPERIMENTAL DETAILS FOR 600 SERIES EXAMPLES

NMR spectra were obtained on a Bruker model DPX300 (300 MHz), DPX400 (400 MHz), or DPX500 (500 MHz) spectrometer. Chemical shifts are reported in ppm downfield of the tetramethylsilane reference. The format of the ¹H NMR data below is: chemical shift (multiplicity, coupling constant J in Hz, integration).

Mass spectra were obtained on an Agilent series 1100 MSD using electrospray ionization (ESI) in either positive or negative mode as indicated. The “mass calculated” for a molecular formula is the monoisotopic mass of the compound.

Thin Layer Chromatography (TLC) was performed using silica gel 60 F₂₅₄ pre-coated plates (size, 2.5×7.5 cm; thickness, 250 μm). The reaction products were detected by viewing the plates under a UV lamp (254 nm).

Melting points were determined on either an Electrothermal apparatus or on a Thomas-Hoover capillary melting point apparatus and are uncorrected.

Reverse Phase HPLC (Method V):

Column: Zorbax Eclipse XDB-C8, 5 mm, 4.6×150 mm;
Flow rate: 0.75 mL/min; %=220 & 254 nm;

Gradient (Acetonitrile/Water):

| | | | | |
|---|---|---|---|---|
| 1) | 0 to 8.0 | min | 1%-99% | Acetonitrile |
| 2) | 8.0 to 10.5 | min | 99% | Acetonitrile |
| 3) | after 10.5 | min | 1% | Acetonitrile |

### Example 600

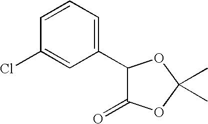

### 5-(3-Chloro-phenyl)-2,2-dimethyl-[1,3]dioxolan-4-one

To a 500-mL, one-necked, round-bottomed flask, equipped with a magnetic stir-bar was added, (R)-3-chloromandelic acid (24 g, 0.128 mol), 2,2-dimethoxypropane (16.1 g, 0.154 mol), and 250 mL of anhydrous benzene. The flask was then fitted with a Dean-Stark condenser and heated to reflux on a heating mantle. Heating was continued for about 18 h when TLC and HPLC analysis of a small aliquot removed from the reaction mixture indicated completion of reaction. The heating source was removed and after cooling to room temperature, the reaction mixture was concentrated under reduced pressure to yield a waxy solid (29.5 g, 100%). ¹H NMR and HPLC indicated the product to be of sufficient purity for use in the next step with out any further purification. ¹H NMR (500 MHz, CDCl₃): 7.47 (m, 1H), 7.35 (m, 3H), 5.36 (s, 1H), 1.73 (s, 3H), 1.68 (s, 3H).

### Example 601

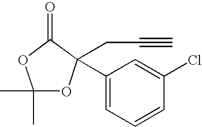

### 5-(3-Chloro-phenyl)-2,2-dimethyl-5-prop-2-ynyl-[1,3]dioxolan-4-one

A 1-L, three-necked, round-bottomed flask fitted with nitrogen inlet and magnetic stir-bar was charged with 5-(3-chloro-phenyl)-2,2-dimethyl-[1,3]dioxolan-4-one (22.5 g, 0.1 mol). Anhydrous THF (300 mL) was added through a cannula and the resulting solution was cooled to −78° C. in a dry ice-acetone bath. To this solution was added, through a cannula, LiHMDS (1 M solution in THF, 110 mL, 0.11 mol). After stirring for about 1 h, propargyl bromide (80% w/toluene, 11.8 mL, 16.25 g, 0.11 mol) was added dropwise using a syringe. The reaction mixture was allowed to stir at this temperature for about 1 h and the cooling bath was then removed allowing the reaction to warm to room temperature overnight. After quenching by adding satd. aq. NH₄Cl (100 mL) and EtOAc (200 mL), the mixture was transferred to a separatory funnel and the layers separated. The organic layer was washed with brine, dried over MgSO₄, and concentrated under reduced pressure. The pale-brown oil thus obtained was purified by pad-filtration through a silica-gel plug (10% EtOAc/hexanes) to obtain the title compound as pale yellow oil (23.8 g, 90%). HPLC (Method V): Rₜ=10.45 min. ¹H NMR (500 MHz, CDCl₃): 7.66 (m, 1H), 7.56 (m, 1H), 7.32 (m, 2H), 2.93 (dd, J=13.6, 2.0 Hz, 1H), 2.74 (dd, J=13.6, 2.0 Hz, 1H), 2.11 (t, J=2.0 Hz, 1H), 1.78 (s, 3H), 1.46 (s, 3H).

### Example 602

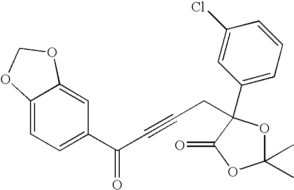

### 5-(4-Benzo[1,3]dioxol-5-yl-4-oxo-but-2-ynyl)-5-(3-chloro-phenyl)-2,2-dimethyl-[1,3]dioxolan-4-one

A 500-mL, three-necked, round-bottomed flask fitted with a magnetic stir-bar was charged with Pd(PPh₃)Cl₂ (0.38 g, 0.54 mmol), CuI (0.21 g, 1.1 mmol), and THF (150 mL) under N₂. The solution was degassed with a stream of N₂ for 5 min. Under N₂, N-methylmorpholine (7.26 mL, 66 mmol), benzo[1,3]dioxole-5-carbonyl chloride (10.1 g, 55.0 mmol) and a solution of 5-(3-chloro-phenyl)-2,2-dimethyl-5-prop-2-ynyl-[1,3]dioxolan-4-one (16.0 g, 60.5 mmol) in toluene (150 mL) were added sequentially. After the addition, the reaction mixture was degassed with a stream of N₂ for 5 min. The reaction mixture was then stirred under N₂ at room temperature for 16 h. The white precipitate that had formed was filtered off and washed with toluene (100 mL). The combined filtrates were washed with water (2×200 mL), brine (200 mL), dried over MgSO₄, and concentrated under reduced pressure. The crude product was obtained as a brown solid, which was used in the next reaction without further purification. HPLC (Method V): Rₜ=10.36 min. MS (ES+): exact mass calculated for C₂₂H₁₇ClO₆, 412.07; m/z found, 413.4 [M+H]⁺. ¹H NMR (500 MHz, CDCl₃): 7.74-7.70 (m, 1H), 7.69-7.64 (m, 1H), 7.63-7.58 (m, 1H), 7.50-7.45 (m, 1H), 7.40-7.35 (m, 2H), 6.86 (d, J=8.1 Hz, 1H), 6.07 (s, 2H), 3.19 (d, J=17.4 Hz, 1H), 3.10 (d, J=17.4 Hz, 1H), 1.77 (s, 3H), 1.48 (s, 3H).

### Example 603

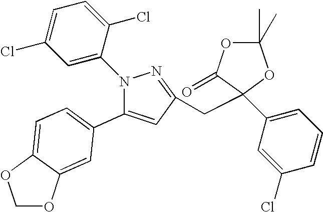

### 5-[5-Benzo[1, 3]dioxol-5-yl-1-(2,5-dichloro-phenyl)-1H-pyrazol-3-ylmethyl]-5-(3-chloro-phenyl)-2,2-dimethyl-[1,3]dioxolan-4-one

A 500-mL, one-necked, round-bottomed flask fitted with a reflux condenser and magnetic stir-bar was charged with 5-(4-benzo[1,3]dioxol-5-yl-4-oxo-but-2-ynyl)-5-(3-chloro-phenyl)-2,2-dimethyl-[1,3]dioxolan-4-one (obtained from Example 602) and EtOH (300 mL). To this suspension, 2,5-dichlorophenylhydrazine (10 g, 56.5 mmol) was added as a solid. The resulting suspension was heated to form a homogeneous solution and stirred at reflux temperature under an air atmosphere for 5 h. The solution was cooled to room temperature and the solvent was concentrated under reduced pressure. The resulting crude product was purified by silica-gel flash-column chromatography (EtOAc/hexanes) to afford the title compound as a white solid (24.2 g, 42.3 mmol, 77% for 2 steps). The product may also be purified by recrystallization from hot MeOH. HPLC (Method V): Rₜ=11.14 min. MS (ES+): exact mass calculated for C₂₈H₂₁Cl₃N₂O₅, 570.05; m/z found, 571.2 [M+H]⁺. ¹H NMR (500 MHz, CDCl₃): 7.12 (s, 1H), 7.68-7.62 (m, 1H), 7.38-7.30 (m, 5H), 6.70 (d, J=8.2 Hz, 1H), 6.66-6.60 (m, 2H), 6.36 (s, 1H), 5.95 (s, 2H), 3.44 (d, J=14.6 Hz, 1H), 3.26 (d, J=14.6 Hz, 1H), 1.42 (s, 6H).

### Example 604

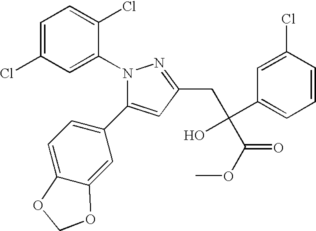

### 3-[5-Benzo[1,3]dioxol-5-yl-1-(2,5-dichloro-phenyl)-1H-pyrazol-3-yl]-2-(3-chloro-phenyl)-2-hydroxy-propionic acid methyl ester

To a 1-L, single-necked, round-bottomed flask was dissolved 5-[5-benzo[1,3]dioxol-5-yl-1-(2,5-dichloro-phenyl)-1H-pyrazol-3-ylmethyl]-5-(3-chloro-phenyl)-2,2-dimethyl-[1,3]dioxolan-4-one (20.17 g, 0.0353 mol) in anhydrous methanol (500 mL). To this stirring solution was added sodium methoxide (2.10 g, 0.0388 mol) in one portion. The solution was stirred at room temperature overnight. Upon completion, water (500 mL) was added to the flask. The reaction mixture was extracted with EtOAc (3×200 mL). All organic layers were combined, washed with brine, dried over MgSO₄, filtered and concentrated under vacuum. The crude alcohol was purified by silica-gel flash-column chromatography (25% EtOAc/hexanes) to afford the title compound (16.38 g, 85%) as a light yellow oil. HPLC (Method V): purity, 99%; Rₜ=10.85 min. MS (ES+): exact mass calculated for C₂₆H₁₉Cl₃N₂O₅, 544.04; m/z found, 545.2 [M+H]⁺. ¹H NMR (500 MHz, CDCl₃): 7.72 (m, 1H), 7.59 (dt, J=6.9, 1.7 Hz, 1H), 7.32 (m, 5H), 6.69 (m, 1H), 6.61 (m, 2H), 6.32 (s, 1H), 5.95 (s, 2H), 4.72 (s, 1H), 3.73 (s, 3H), 3.70 (d, J=14.7 Hz, 1H), 3.62 (d, J=14.7 Hz, 1H).

### Example 605

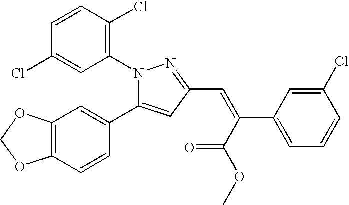

### (Z)-3-[5-Benzo[1,3]dioxol-5-yl-1-(2,5-dichloro-phenyl)-1H-pyrazol-3-yl]-2-(3-chloro-phenyl)-acrylic acid methyl ester

In a 500-mL, one-necked, round-bottomed flask was dissolved 3-[5-benzo[1,3]dioxol-5-yl-1-(2,5-dichloro-phenyl)-1H-pyrazol-3-yl]-2-(3-chloro-phenyl)-2-hydroxy-propionic acid methyl ester (16.38 g, 0.030 mol) in anhydrous CH₂Cl₂ (160 mL). The solution was cooled to 0° C. using an ice bath. At 0° C., triflic anhydride (16.92 g, 0.060 mol) was added dropwise with stirring. The resultant solution was stirred for 10 min. Anhydrous pyridine (11.82 g, 0.15 mol) was then added dropwise. The temperature of the mixture rose 8-10 degrees during this addition. The reaction flask was kept at 0° C. for an additional 1 h. The reaction was then allowed to warm to room temperature and was stirred for 48 h. The reaction was quenched by the addition of water (250 mL). The layers were separated and the aqueous layer was extracted with CH₂Cl₂ (2×100 mL). All organic layers were combined, washed with brine, dried over MgSO₄, filtered, and concentrated under reduced pressure to yield an oil. Purification by silica gel chromatography (10-25% EtOAc/hexanes) gave the title compound (14.0 g, 88%) with 30:1 Z to E selectivity (determined by ¹H NMR) as a light orange/tan foam. HPLC (Method V): purity, 99%; Rₜ=11.30 min. MS (ES+): exact mass calculated for C₂₆H₁₇Cl₃N₂O₄, 526.03; m/z found, 527.1 [M+H]⁺. ¹H NMR (500 MHz, CDCl₃): 7.49 (d, J=0.7 Hz, 1H), 7.47 (d, J=2.0 Hz, 1H), 7.33 (m, 5H), 7.04 (s, 1H), 6.72 (dd, J=8.3, 0.7 Hz, 1H), 6.66 (m, 2H), 6.59 (s, 1H), 5.96 (s, 2H), 3.91 (s, 3H).

### Example 606

### Alternative Preparation of Example 122

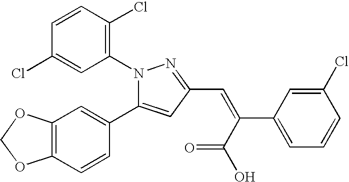

### (Z)-3-[5-Benzo[1,3]dioxol-5-yl-1-(2,5-dichloro-phenyl)-1H-pyrazol-3-yl]-2-(3-chloro-phenyl)-acrylic acid

In a 500-mL, single-necked, round-bottomed flask was dissolved (Z)-3-[5-benzo[1,3]dioxol-5-yl-1-(2,5-dichloro-phenyl)-1H-pyrazol-3-yl]-2-(3-chloro-phenyl)-acrylic acid methyl ester (14.0 g, 0.0265 mol) in 1,4-dioxane (140 mL). To this stirring solution was added deionized water (140 mL) followed by LiOH (3.17 g, 0.132 mol). The flask was then submerged into an oil bath that had been preheated to a temperature of 75-77° C. After 12 h, the heating bath was removed and the flask was cooled to 0° C. and the pH was adjusted with 6 N HCl to pH ˜4. The acidified solution was extracted with EtOAc (3×100 mL). All organic layers were combined, washed with brine, dried over MgSO₄, filtered, and concentrated under vacuum to yield the crude acid as a pale yellow, oily solid. The crude product was dissolved in anhydrous CH₂Cl₂ (100 mL) and 10% EtOAc in hexanes (100 mL) was slowly added with stirring. Precipitation began immediately. The stir bar was removed and the flask was left undisturbed at room temperature for several hours. The solids were collected and dried in a vacuum oven (house vacuum, 60° C., 3 days) to yield the title compound (11.1 g, 82% yield) as an off white crystalline powder. The product may also be purified by recrystallization from hot EtOH. Mp 174° C. HPLC (Method V): purity, 99%; Rₜ=10.49 min. MS (ES+): exact mass calculated for C₂₅H₁₅Cl₃N₂O₄, 512.01; m/z found, 513.1 [M+H]⁺. ¹H NMR (500 MHz, CDCl₃): 7.48 (m, 1H), 7.45 (m, 1H), 7.43 (m, 2H), 7.36 (m, 1H), 7.32 (m, 2H), 6.75 (dd, J=7.6, 0.9 Hz, 1H), 6.68 (m, 3H), 5.99 (s, 2H).

### Example 608

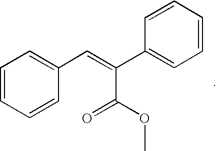

### (Z)-2,3-Diphenyl-acrylic acid methyl ester

In a 25-mL, one-necked, round-bottomed flask was dissolved 2-hydroxy-2,3-diphenyl-propionic acid methyl ester (0.523 g, 2.04 mmol) in anhydrous CH₂Cl₂ (5 mL). The solution was cooled to 0° C. using an ice bath. At 0° C., triflic anhydride (0.633 g, 2.24 mmol) was added dropwise with stirring. The resultant solution was stirred for 10 min. Anhydrous pyridine (0.403 g, 5.10 mmol) was then added dropwise. The reaction flask was kept at 0° C. for an additional 1 h. The reaction mixture was then allowed to warm to room temperature and was stirred for 48 h. The reaction was quenched by the addition of water (20 mL). The layers were separated and the aqueous layer was extracted with CH₂Cl₂ (2×15 mL). All organic layers were combined, washed with brine, dried over MgSO₄, filtered, and concentrated under reduced pressure to yield an oil. Purification by silica gel chromatography (10-25% EtOAc/hexanes) gave the title compound (0.427 g, 88%) with 44:1 Z to E selectivity (determined by ¹H NMR) as a light yellow oil. HPLC (Method V): purity, 99%; Rₜ=10.42 min. MS (ES+): exact mass calculated for C₁₆H₁₄O₂, 238.28; m/z found, 239.2 [M+H]⁺. ¹H NMR (500 MHz, CDCl₃): 7.47-7.45 (m, 2H), 7.40-7.29 (m, 8H), 7.04 (br s, 1H), 3.79 (d, J=2.6 Hz, 3H).

Examples 609-619 were prepared in a manner similar to that described for Example 608.

### Example 609

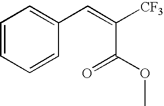

### (Z)-3-Phenyl-2-trifluoromethyl-acrylic acid methyl ester

The title compound was prepared from 2-benzyl-3,3,3-trifluoromethyl-2-hydroxypropionic acid methyl ester. HPLC (Method V): purity, 98%; Rₜ=10.17 min. MS (ES+): exact mass calculated for C₁₁H₉F₃O₂, 230.06; m/z not observed, did not ionize. ¹H NMR (500 MHz, CDCl₃): 7.41-7.38 (m, 1H), 7.35-7.33 (m, 3H), 7.19-7.18 (m, 2H), 3.80 (s, 3H).

### Example 610

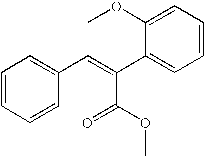

### (Z)-3-(2-Methoxyphenyl)-2-phenyl-acrylic acid methyl ester

The title compound was prepared from 2-hydroxy-3-(2-methoxyphenyl)-2-phenyl-propionic acid methyl ester. HPLC (Method V): purity, 99%; Rₜ=9.84 min. ¹H NMR (500 MHz, CDCl₃): 7.47-7.45 (m, 1H), 7.38-7.15 (m, 6H), 6.94-6.86 (m, 2H), 3.84 (s, 3H), 3.73 (s, 3H).

### Example 611

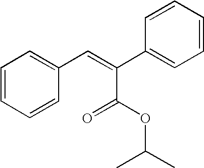

### (Z)-2,3-Diphenyl-acrylic acid isopropyl ester

The title compound was prepared from 2-hydroxy-2,3-diphenyl-propionic acid isopropyl ester. HPLC (Method V): purity, 94%; Rₜ=10.95 min. ¹H NMR (500 MHz, CD₃OD): 7.47-7.46 (m, 1H), 7.41-7.28 (m, 6H), 7.04 (brs, 1H), 5.14 (quint, J=6.3 Hz, 1H), 1.19 (d, J=6.3 Hz, 6H).

### Example 612

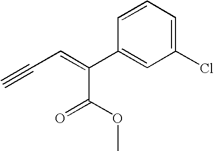

### (Z)-2-(3-Chlorophenyl)-pent-2-en-4-ynoic acid methyl ester

The title compound was prepared from 2-hydroxy-2-(3-chlorophenyl)-pent-4-ynoic acid methyl ester with a selectivity of 8:1 Z/E. HPLC (Method R): purity, 99%; Rₜ=9.61 min. MS (ES+): exact mass calculated for C₁₂H₉ClO₂, 220.03; m/z found, 221.2 [M+H]⁺. ¹H NMR: (500 MHz, CDCl₃): 7.38-7.24 (m, 4H), 6.17 (d, J=2.6 Hz, 1H), 3.88 (s, 3H); 3.48 (d, J=2.6 Hz, 1H).

### Example 613

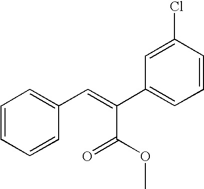

### (Z)-2-(3-Chloro-phenyl)-3-phenyl-acrylic acid methyl ester

The title compound was prepared from 2-(3-chloro-phenyl)-2-hydroxy-3-phenyl-propionic acid methyl ester.

### Example 614

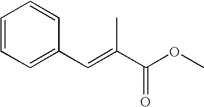

### (E)-2-Methyl-3-phenyl-acrylic acid methyl ester

The title compound was prepared from 2-hydroxy-2-methyl-3-phenyl-propionic acid methyl ester. The product was obtained as a mixture of the title compound and 2-benzyl-acrylic acid methyl ester.

### Example 615

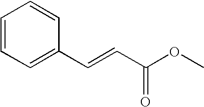

### (E)-3-Phenyl-acrylic acid methyl ester

The title compound was prepared from 2-hydroxy-3-phenyl-propionic acid methyl ester.

### Example 616

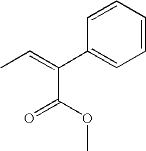

### (Z)-2-Phenyl-but-2-enoic acid methyl ester

The title compound was prepared from 2-hydroxy-2-phenyl-butyric acid methyl ester.

### Example 617

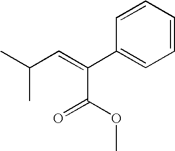

### (Z)-4-Methyl-2-phenyl-pent-2-enoic acid methyl ester

The title compound was prepared from 2-hydroxy-4-methyl-2-phenyl-pentanoic acid methyl ester.

### Example 618

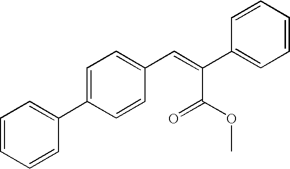

### (Z)-3-Biphenyl-4-yl-2-phenyl-acrylic acid methyl ester

The title compound was prepared from 3-biphenyl-4-yl-2-hydroxy-2-phenyl-propionic acid methyl ester.

### Example 619

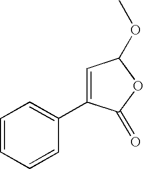

### 5-Methoxy-3-phenyl-5H-furan-2-one

The title compound was prepared from 3-[1,3]dioxolan-2-yl-2-hydroxy-2-phenyl-propionic acid methyl ester.

Examples 620-622 may be prepared using procedures, similar to that described in Example 608.

### Example 620

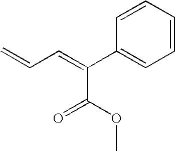

### (Z)-2-Phenyl-2,4-pentadienoic acid methyl ester

The title compound may be prepared from 2-hydroxy-2-phenyl-pent-4-enoic acid methyl ester.

### Example 621

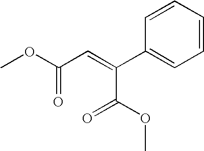

### (Z)-2-Phenyl-but-2-ene-dioic acid methyl ester

The title compound may be prepared from 2-hydroxy-2-phenylsuccinic acid methyl ester.

### Example 622

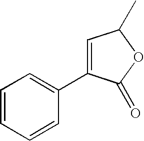

### 5-Methyl-3-phenyl-5H-furan-2-one

The title compound may be prepared from 2-hydroxy-2-phenyl-4-(tetrahydro-pyran-2-yloxy)-pentanoic acid methyl ester. One skilled in the art will recognize that any required treatment with acid, such as 1 N HCl, may be performed during the workup stage of the procedure.

Assay Method

### Cell Culture

CHO-K1 cells that had undergone stable transfection with the CCK-1 receptor were grown in DMEM supplemented with L-glutamine (2 mM), penicillin (50 units/mL) and streptomycin (50 μg/mL). Cells were cultured under continuous G418 selection (2 mM) and were harvested using a rubber cell scraper. CHO-K1 cells were sub-cultured a maximum of ten times before being reseeded from the original stocks.

### Membrane Preparation

Membranes were prepared from the stably transfected CHO-K1 cells. Frozen cell pellets (−40° C.) were thawed in 14 mL of buffer A (10 mM HEPES, 130 mM NaCl, 4.7 mM KCl, 5 mM MgCl, 1 mM EGTA and 15.4 mg/100 mL bacitracin at pH 7.2), adapted from Harper, E. A. et al. (Br. J. Pharmacol. 1996, 118, 1717-1726). The thawed pellets were homogenized using a Polytron PT-10 (7×1 s). The homogenates were centrifuged for 5 min at 1500 rpm (600×g), and the resulting pellets were discarded. The supernatants were re-centrifuged in order to collect the receptor-membrane pellets (25 min 15,000 rpm; 39,800×g), which were re-suspended in buffer A.

### Incubation Conditions

All assays were conducted in 96-well plates (GF/B millipore filter plates) using buffer A, with 0.3 μM PD-134,308, for the dilutions. The CCK-2 receptor ligand was included to eliminate the contribution of this receptor subtype to the binding. For the optimal cell number determination experiments 20 pM [125I]-BH-CCK-8S (50 μL 60 pM solution) was incubated with a range of cell concentrations (2.5×105 to 12.5×105 cells/well) in a total volume of 150 μL. Total binding of [125I]-BH-CCK-8S was determined in the presence of 15 μL of buffer A. Non-specific binding of [125I]-BH-CCK-8S was determined in the presence of 15 μL of 100 μM 2-naphthalenesulphonyl L-aspartyl-(2-phenethyl)amide (2-NAP: see Hull, R. A. et al. Br. J. Pharmacol. 1993, 108, 734-740), a CCK-1 receptor selective antagonist that is structurally unrelated to the radioligand [125I]-BH-CCK-8S. The assay preparation was incubated for 1 h at 21±3° C., and then the assay was terminated upon rapid filtration of the preparation under reduced pressure. The loaded filters were washed three times using undiluted PBS (100 μL), and then the residues were transferred to 5 mL scintillation tubes. Bound radioactivity was determined using a gamma counter (count time=1 min). From these experiments a cell concentration of 1 pellet in 40 mL of buffer (2.5×106 cells/mL) was chosen for use in other assays (below). To validate the radioligand concentration and incubation time for the assay, saturation and kinetic binding studies were also conducted (see M. F. Morton, The Pharmacological Characterization of Cholecystokinin Receptors in the Human Gastrointestinal Tract. PhD Thesis, University of London, 2000). The affinity of novel compounds was estimated by incubating membrane preparations with 15 μL of competing ligand (0.1 pM-1 mM) for 60 min at 21±3° C. The assay was then terminated according to the procedure outlined above.

### Data Analysis

The pKi values were determined using the equation of Cheng and Prusoff (Biochem. Pharmacol. (1973) 22, pp 3099-3108):

$${K}_{i} = \frac{I {C}_{50}}{1 + \frac{[ L ]}{{K}_{D}}}$$

To circumvent problems associated with computer-assisted data analysis of compounds with low affinity, the data obtained in the current study were weighted according to a method described by Morton (2000). In brief, 100% and 0% specific binding were defined independently using total binding and binding obtained in the presence of a high concentration of the reference antagonist, 2-NAP.

TABLE

| Example | pKi | Example | pKi | Example | pKi |
|---|---|---|---|---|---|
| 1 | 8.0 | 198 | 8.1 | 56 | 7.3 |
| 2 | 8.0 | 208 | 5.5 | 80 | 7.9 |
| 3 | 6.6 | 210 | 7.9 | 92 | 8.2 |
| 4 | 8.0 | 211 | 7.9 | 93 | 6.6 |
| 7 | 8.1 | 221 | 7.8 | 105 | 6.5 |
| 18 | 7.4 | 246 | 7.4 | 47 | 6.7 |
| 19 | 7.5 | 77 | 7.8 | 51 | 8.3 |
| 21 | 6.8 | 106 | 7.2 | 303 | 5.9 |
| 24 | 7.7 | 322 | 7.4 | 305 | 5.7 |
| 26 | 7.1 | 328 | 7.7 | 308 | 7.2 |
| 27 | 8.2 | 334 | 7.0 | 311 | 7.7 |
| 28 | 5.9 | 71 | 7.6 | 48 | 7.1 |
| 29 | 7.4 | 72 | 7.3 | 50 | 7.0 |
| 31 | 6.0 | 261 | 7.9 | 79 | 6.9 |
| 32 | 7.2 | 262 | 7.9 | 82 | 5.9 |
| 37 | 7.7 | 64 | 7.3 | 83 | 7.2 |
| 40 | 8.1 | 65 | 5.7 | 88 | 7.4 |
| 42 | 8.2 | 66 | 7.7 | 90 | 6.1 |
| 43 | 7.0 | 68 | 6.6 | 86 | 8.4 |
| 46 | 7.7 | 74 | 8.2 | 87 | 7.6 |
| 145 | 7.8 | 129 | 7.8 | 91 | 7.9 |
| 148 | 7.8 | 131 | 6.9 | 101 | 7.8 |
| 151 | 6.7 | 132 | 8.0 | 104 | 7.4 |
| 152 | 7.9 | 136 | 8.2 | 349 | 7.1 |
| 153 | 7.8 | 137 | 8.0 | 352 | 7.5 |
| 155 | 8.0 | 138 | 7.5 | 75 | 7.1 |
| 157 | 7.9 | 335 | 7.5 | 110 | 7.9 |
| 167 | 7.9 | 54 | 7.4 | 111 | 8.4 |
| 168 | 8.1 | 58 | 6.3 | 112 | 8.4 |
| 170 | 8.1 | 59 | 8.5 | 115 | 8.2 |
| 177 | 7.9 | 60 | 8.3 | 118 | 8.3 |
| 181 | 7.8 | 271 | 7.8 | 120 | 8.0 |
| 182 | 7.9 | 275 | 7.7 | 121 | 8.1 |
| 189 | 7.4 | 276 | 8.2 | 122 | 8.8 |
| 190 | 8.0 | 287 | 7.7 | 123 | 6.6 |
| 195 | 8.0 | 52 | 8.0 | 124 | 7.4 |
| | | | | 363 | 6.1 |

Having described the invention in specific detail and exemplified the manner in which it may be carried into practice, it will be apparent to those skilled in the art that innumerable variations, applications, modifications, and extensions of the basic principles involved may be made without departing from its sprit or scope. It is to be understood that the foregoing is merely exemplary and the present invention is not to be limited to the specific form or arrangements of parts herein described and shown.

What is claimed is:

1. A method of making a compound of formula (I′), 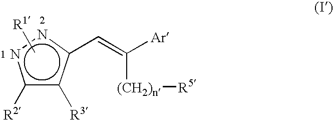
wherein,
R^{1′} is a 1- or 2-position substituent selected from the group consisting of —H,
a) phenyl, optionally mono-, di-, or tri-substituted with R^{p′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{p′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t′})COR^{t}, —(N—R^{t′})SO₂C₁₋₆alkyl (wherein R^{t′} is —H or —C₁₋₆alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH, and —COOC₁₋₆alkyl;
b) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{p′};
c) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{p′};
d) naphthyl, optionally mono-, di-, or tri-substituted with R^{p′};
e) a monocyclic aromatic hydrocarbon group having five ring atoms, having a carbon atom which is the point of attachment, having one carbon atom replaced by >O, >S, >NH, or >N(C₁₋₄alkyl), having up to two additional carbon atoms optionally replaced by N, optionally mono- or di-substituted with R^{p′} and optionally benzo or pyrido fused on the condition that two or fewer of said carbon ring atoms are replaced by a heteroatom, where the benzo or pyrido fused moiety is optionally mono-, di-, or tri-substituted with R^{p′};
f) a monocyclic aromatic hydrocarbon group having six ring atoms, having a carbon atom which is the point of attachment, having one or two carbon atoms replaced by N, optionally mono- or di-substituted with R^{p′} and optionally benzo or pyrido fused, where the benzo or pyrido fused moiety is optionally mono- or di-substituted with R^{p′};
g) adamantanyl or monocyclic C₅₋₇cycloalkyl, optionally having one or two carbon members optionally replaced with >O, >NH, or >N(C₁₋₄alkyl), optionally having one or two unsaturated bonds in the ring, and optionally having one of the ring atoms substituted with —OH, =0 or —CH₃;
h) a —C₁₋₈alkyl; and
i) —C₁₋₄alkyl, mono-substituted by a substituent selected from the group consisting of any one of a) to g);
R^{2′} is selected from the group consisting of:
i) phenyl, optionally mono-, di-, or tri-substituted with R^{q′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{q′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t′})COR^{t′}, —(N—R^{t′})SO₂C₁₋₆alkyl (wherein R^{t} is H or C₁₋₆-alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH and —COOC₁₋₆alkyl;
ii) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{q′};
iii) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{q′};
iv) naphthyl, optionally mono-, di-, or tri-substituted with R^{q′};
v) a monocyclic aromatic hydrocarbon group having five ring atoms, having a carbon atom which is the point of attachment, having one carbon atom replaced by >O, >S, >NH, or >N(C₁₋₆alkyl), having up to one additional carbon atoms optionally replaced by N, optionally mono- or di-substituted with R^{q′} and optionally benzo or pyrido fused on the condition that two or fewer of said carbon ring atoms are replaced by a heteroatom, where the benzo or pyrido fused moiety is optionally mono-, di-, or tri-substituted with R^{q′}; and
vi) a monocyclic aromatic hydrocarbon group having six ring atoms, having a carbon atom which is the point of attachment, having one or two carbon atoms replaced by N, optionally mono- or di-substituted with R^{p′} and optionally benzo or pyrido fused, where the benzo or pyrido fused moiety is optionally mono- or di-substituted with R^{q′};
R^{3′} is selected from the group consisting of —H, halo, and —C₁₋₆alkyl;
n′ is 0;
Ar′ is selected from the group consisting of:
A) phenyl, optionally mono-, di-, or tri-substituted with R^{r′} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{r′} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y′} and R^{z′} are independently selected from —H, —C₁₋₆alkyl, and —C₁₋₆alkenyl, or R^{y′} and R^{z′} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y′})R^{z′}, —(N—R^{t′})COR^{t′}, —(N—R^{t′})SO₂C₁₋₆alkyl (wherein R^{t′} is —H or —C₁₋₆alkyl or two R^{t′} in the same substituent may be taken together with the amide of attachment to form an otherwise aliphatic hydrocarbon ring, said ring having 4 to 6 members), —(C═O)C₁₋₆alkyl, —(S═(O)_{m′})—C₁₋₆alkyl (wherein m′ is selected from 0, 1, and 2), —SO₂N(R^{y′})R^{z′}, —SCF₃, halo, —CF₃, —OCF₃, —COOH, and —COOC₁₋₆alkyl;
B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r′};
C) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r′};
D) naphthyl, optionally mono-, di-, or tri-substituted with R^{r′};
E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r′} and optionally benzo or pyrido fused on the condition that two or fewer of said carbon ring atoms are replaced by a heteroatom, where the benzo or pyrido fused moiety is optionally mono-, di-, or tri-substituted with R^{r′};
F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r′} and optionally benzo or pyrido fused, where the benzo or pyrido fused moiety is optionally mono- or di-substituted with R^{r′};
G) adamantanyl or monocyclic C₅₋₇cycloalkyl, optionally having one or two carbon members optionally replaced with >O, >S, >NH, or >N(C₁₋₄alkyl), optionally having one or two unsaturated bonds in the ring, and optionally having one of the ring atoms substituted with —OH, =0 or —CH₃;
H) a —C₁₋₈alkyl wherein the carbon of attachment bears no hydrogen substituents, optionally mono-substituted by a substituent selected from the group consisting of any one of a) to g), and optionally mono-, di-, or tri-substituted by a R^{r′};
I) —C₂alkenyl or —C₂alkynyl, optionally mono-substituted by a substituent selected from the group consisting of any one of a) to h); and
R^{5′} is —COOR^{6′}, where R^{6′} is selected from the group consisting of —H and —C₁₋₄alkyl;
or an ester, enantiomer, diastereomer, racemic, or pharmaceutically acceptable salt thereof, comprising: providing an α-hydroxyester compound having
(a) an α-carbon member that is alkylated through an intervening methylene with a group that does not have a dehydration-removable hydrogen bonded to said methylene,
(b) an ester moiety with its carboxy group attached directly to said α-carbon member, and
(c) a substituent attached to said α-carbon member, wherein the volume of said substituent is greater than the volume of said ester moiety; and treating said α-hydroxyester compound with a dehydrating agent.

2. A method according to claim 1, wherein R^{1′}, optionally substituted with R^{p′}, is selected from the group consisting of hydrogen,
a) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
b) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
c) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
d) naphthyl,
e) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl,
f) pyridinyl, pyridinyl-N-oxide, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, 1-oxy-pyridin-2, 3, or 4-yl,
g) cyclopentyl, cyclohexyl, cycloheptyl, piperidin-2, 3 or 4-yl, 2-pyrrolin-2, 3, 4 or 5-yl, 3-pyrrolin-2 or 3-yl, 2-pyrazolin-3, 4 or 5-yl, morpholin-2, 3, 5 or 6-yl, thiomorpholin-2, 3, 5 or 6-yl, piperazin-2, 3, 5 or 6-yl, pyrrolidin-2 or 3-yl, homopiperidinyl, adamantanyl,
h) methyl, ethyl, n-propyl, i-propyl, n-butyl, i-butyl, t-butyl, n-pentyl, pent-2-yl, hexyl, hex-2-yl, and
i) —C₁₋₂alkyl mono-substituted with any one of the preferred substituents of a) to g).

3. A method according to claim 1, wherein R^{1′}, optionally substituted with R^{p′}, is selected from the group consisting of —H, methyl, phenyl, benzyl, cyclohexyl, cyclohexylmethyl, pyridinyl, pyridinylmethyl and pyridinyl-N-oxide.

4. A method according to claim 1, wherein R^{1′} is selected from the group consisting of phenyl, 2-methoxy-phenyl, 3-methoxy-phenyl, 4-methoxy-phenyl, 2,3-dimethoxy-phenyl, 3,4-dimethyoxy-phenyl, 2-chloro-phenyl, 3-chloro-phenyl, 4-chloro-phenyl, 2,4-dichloro-phenyl, 3,4-dichlorophenyl, 2,4-dichlorophenyl, 2,5-dichlorophenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 4-trifluoromethyl-phenyl, 3-trifluoromethoxy-phenyl, 4-trifluoromethoxy-phenyl, 4-t-butyl-phenyl, benzyl, cyclohexyl, pyridin-2-yl, pyridin-3-yl, pyridin-4-yl, 4-trifluoromethyl-2-pyridyl, 2-pyridyl-N-oxide, 4-methanesulfonyl-phenyl, 4-phenoxy-phenyl, 4-isopropyl-phenyl, 4-ethoxy-phenyl, 4-hydroxy-phenyl, 4-pyridinyl-methyl, benzo[1,3]diox-5-yl, 2,3-dihydro benzo[1,4]dioxin-6-yl, and cyclohexylmethyl.

5. A method according to claim 1, wherein R^{p′} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SOCH₃, —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

6. A method according to claim 1, wherein R^{p′} is selected from the group consisting of methyl, methoxy, ethoxy, chloro, fluoro, trifluoromethyl, trifluoromethoxy, t-butyl, methanesulfonyl, phenoxy, isopropyl, and hydroxy.

7. A method according to claim 1, wherein R², optionally substituted with R^{q′} is selected from the group consisting of:
i) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
ii) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
iii) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
iv) naphthyl,
v) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, and
vi) pyridinyl, pyridinyl-N-oxide, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl.

8. A method according to claim 1, wherein R^{2′}, optionally substituted with R^{q′} is selected from the group consisting of phenyl, naphthalenyl, pyridinyl, thiophenyl, benzothiophenyl, furanyl, benzofuranyl, indolyl, indolinyl, isoquinolinyl, and quinolinyl.

9. A method according to claim 1, wherein R^{2′} is selected from the group consisting of 4-methyl-phenyl, 2-chloro-phenyl, 3-chloro-phenyl, 4-chloro-phenyl, 3,4-dichloro-phenyl, benzo[1,3]dioxol-5-yl, 2,3-dihydro benzo[1,4]dioxin-6-yl, 4-methoxy-phenyl, phenyl, 4-phenoxy-phenyl, naphthalen-2-yl, pyridin-3-yl, 2-chloro-pyridin-3-yl, pyridin-4-ylmethyl, 4-benzyloxy-phenyl, 4-dimethylamino-phenyl, 4-bromo-3-methyl-phenyl, 3-methoxy-4-methyl-phenyl, 3-cyclopentyloxy-4-methoxy-phenyl, 4-bromo-2-chloro-phenyl, 4-bromo-phenyl, 3-dimethylamino-phenyl, 4-morpholin-1-yl-phenyl, 4-pyrrolidin-1-yl-phenyl, 4-(N-propylamino)-phenyl, 4-(N-isobutylamino)-phenyl, 4-diethylamino-phenyl, 4-(N-allylamino)-phenyl, 4-(N-isopropylamino)-phenyl, 4-(N-methyl-N-propylamino)-phenyl, 4-(N-methyl-N-isopropylamino)-phenyl, 4-(N-methyl-N-ethylamino)-phenyl, 4-amino-phenyl, 4-(N-methyl-N-propylamino)-2-chloro-phenyl, 4-(N-ethyl-N-methylamino)-2-chloro-phenyl, 4-(pyrrolidin-1-yl)-2-chloro-phenyl, 4-azetidinyl-phenyl, 4-(pyrrolidin-2-one-1-yl)-phenyl, 4-bromo-3-methyl-phenyl, 4-chloro-3-methyl-phenyl, 1-methyl-5-indolinyl, 5-indolinyl, 5-isoquinolinyl, 6-quinolinyl, benzo[1,3]diox-5-yl, and 7-methoxy-benzofuran-2-yl.

10. A method according to claim 1, wherein R^{q′} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SO(CH₃), —SO₂CH₃, —SO₂NH₂, —SO₂NHCH₃, —SO₂N(CH₃)₂, —SCF₃, —F, —Cl, —Br, —I, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

11. A method according to claim 1, wherein R^{q′} is selected from the group consisting of methyl, bromo, chloro, methoxy, cyclopentyloxy, phenoxy, benzyloxy, pyrrolidinyl, N-methyl-N-ethylamino and dimethylamino.

12. A method according to claim 1, wherein there are 0, 1, or 2 R^{q′} substituents.

13. A method according to claim 1, wherein R^{3′} is selected from the group consisting of —H, —F, —Cl, —Br, and —CH₃.

14. A method according to claim 1, wherein R^{3′} is —H.

15. A method according to claim 1, wherein Ar′, optionally substituted with R^{r′}, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl.

16. A method according to claim 1, wherein Ar′, optionally substituted with R^{r′}, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl.

17. A method according to claim 1, wherein Ar′ is selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃

18. A method according to claim 1, wherein there are 0, 1, or 2 R^{r′} substituents.

19. A method according to claim 1, wherein R^{r′} is selected from the group consisting of —OH, —CH₃, —CH₂CH₃, -propyl, -t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —CN, —NO₂, —C(O)NH₂, —C(O)N(CH₃)₂, —C(O)NH(CH₃), —NH(CO)H, —NHCOCH₃, —NCH₃(CO)H, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —Br, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

20. A method according to claim 1, wherein R^{r′} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl.

21. A method according to claim 1, wherein R^{5′} is —COOR^{6′}, where —COOR^{6′} is —COOH or a hydrolysable group.

22. A method according to claim 1, wherein R^{5′} is selected from the group consisting of —COOCH₃, —COOCH₂CH₃, and —COOCH(CH₃)₂.

23. A method of making a compound of formula (II), 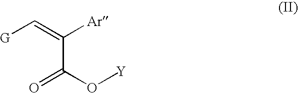
wherein,
G is selected from the group consisting of
a) phenyl, optionally mono-, di-, or tri-substituted with R^{p″} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{p″} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y′})R^{z′} (wherein R^{y″} and R^{z″} are independently selected from —H, —C₁₋₆alkyl, and —C₁₋₆alkenyl, or R^{y″} and R^{z″} may be taken together with the nitrogen of attachment to form an otherwise aliphatic hydrocarbon ring, said ring having 4 to 7 members, optionally having one carbon replaced with >0═N—, >NH, or >N(C₁₋₄alkyl), optionally having one carbon substituted with —OH, and optionally having one or two unsaturated bonds in the ring), —(C═O)N(R^{y″})R^{z′}, —(N—R^{t″})COR^{t″}, —(N—R^{t″})SO₂C₁₋₆alkyl (wherein R^{t″} is —H or —C₁₋₆alkyl, or two R^{t} in the same substituent may be taken together with the amide of attachment to form an otherwise aliphatic hydrocarbon ring, said ring having 4 to 6 members), —(C═O)C₁₋₆alkyl, —(S═(O)_{m″})—C₁₋₆alkyl (wherein m″ is selected from 0 and 2), —SO₂N(R^{y″})R^{z″}, —SCF₃, halo, —CF₃, —OCF₃, —COOH, and —COOC₁₋₆alkyl;
b) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{p″};
c) phenyl or pyridyl fused at two adjacent ring members to a four membered hydrocarbon moiety to form a fused six membered aromatic ring, which moiety has one or two carbon atoms replaced by N, the fused rings optionally mono-, di-, or tri-substituted with R^{p″};
d) naphthyl, optionally mono-, di-, or tri-substituted with R^{p″};
e) a monocyclic aromatic hydrocarbon group having five ring atoms, having a carbon atom which is the point of attachment, having one carbon atom replaced by >O, >S, >NH, or >N(C₁₋₄alkyl), having up to two additional carbon atoms optionally replaced by N, optionally mono- or di-substituted with R^{p″} and optionally benzo or pyrido fused on the condition that two or fewer of said carbon ring atoms are replaced by a heteroatom, where the benzo or pyrido fused moiety is optionally mono-, di-, or tri-substituted with R^{p″};
f) a monocyclic aromatic hydrocarbon group having six ring atoms, having a carbon atom which is the point of attachment, having one or two carbon atoms replaced by N, optionally mono- or di-substituted with R^{p″} and optionally benzo or pyrido fused, where the benzo or pyrido fused moiety is optionally mono- or di-substituted with R^{p″};
g) adamantanyl or monocyclic C₅₋₇cycloalkyl, optionally having one or two carbon members optionally replaced with >O, >S, >NH, or >N(C₁₋₄alkyl), and optionally having one or two unsaturated bonds in the ring and optionally having one of the ring atoms substituted with —OH, ═O, or —CH₃;
h) a —C₁₋₈alkyl, optionally mono-, di-, or tri-substituted with R^{p″} or a substituent selected from the group consisting of any one of a) to g);
i) —C₂alkenyl or —C₂alkynyl, optionally substituted with a substituent selected from the group consisting of any one of a) to h); and
j) —COOR^{7″} where R⁷″ is —C₁₋₈alkyl, aryl, heteroaryl, or C₄₋₈cycloalkyl;
Ar″ is selected from the group consisting of:
A) phenyl, optionally mono-, di-, or tri-substituted with R^{r″} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{r″} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y″})R^{z″} (wherein R^{y″} and R^{z″} are independently selected from —H, —C₁₋₆alkyl, and —C₁₋₆alkenyl, or R^{y″} and R^{z″} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y″})R^{z″}, —(N—R^{t″})COR^{t}, —(N—R^{t″})SO₂C₁₋₆alkyl (wherein R^{t″} is —H or —C₁₋₆alkyl, or two R^{t″} in the same substituent may be taken together with the amide of attachment to form an otherwise aliphatic hydrocarbon ring, said ring having 4 to 6 members), —(C═O)C₁₋₆alkyl, —(S═(O)_{m″})—C₁₋₆alkyl (wherein m″ is selected from 0 or 2), —SO₂N(R^{y″})R^{z″}, —SCF₃, halo, —CF₃, —OCF₃, —COOH, and —COOC₁₋₆alkyl;
B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r″};
C) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r″};
D) naphthyl, optionally mono-, di-, or tri-substituted with R^{r″};
E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r″} and optionally benzo or pyrido fused on the condition that two or fewer of said carbon ring atoms are replaced by a heteroatom, where the benzo or pyrido fused moiety is optionally mono-, di-, or tri-substituted with R^{r″}; and
F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r″} and optionally benzo or pyrido fused, where the benzo or pyrido fused moiety is optionally mono- or di-substituted with R^{r″};
G) adamantanyl or monocyclic C₅₋₇cycloalkyl, optionally having one or two carbon members optionally replaced with >O, >S, >NH or >N(C₁₋₄alkyl) and optionally having one or two unsaturated bonds in the ring and optionally having one of the ring atoms substituted with —OH, ═O, or —CH₃;
H) a —C₁₋₈alkyl wherein the carbon of attachment bears no hydrogen substituents, optionally mono-, di-, or tri-substituted by a R^{r″} or a substituent selected from the group consisting of any one of a) to g); and
I) —C₂alkenyl or —C₂alkynyl, optionally substituted with a substituent selected from the group consisting of any one of a) to h); and
Y is —H or —C₁₋₄alkyl;
or an ester, enantiomer, diastereomer, racemic, or pharmaceutically acceptable salt thereof, comprising: providing an α-hydroxyester compound having
(a) an α-carbon member that is alkylated through an intervening methylene with a group that does not have a dehydration-removable hydrogen bonded to said methylene,
(b) an ester moiety with its carboxy group attached directly to said α-carbon member, and
(c) a substituent attached to said α-carbon member, wherein the volume of said substituent is greater than the volume of said ester moiety; and treating said α-hydroxyester compound with a dehydrating agent.

24. A method according to claim 23, wherein G, optionally substituted with R^{p″}, is selected from the group consisting of
a) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
b) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
c) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
d) naphthyl,
e) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl,
f) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, 1-oxy-pyridin-2, 3, or 4-yl,
g) cyclopentyl, cyclohexyl, cycloheptyl, piperidin-2, 3 or 4-yl, 2-pyrrolin-2, 3, 4 or 5-yl, 3-pyrrolin-2 or 3-yl, 2-pyrazolin-3, 4 or 5-yl, morpholin-2, 3, 5 or 6-yl, thiomorpholin-2, 3, 5 or 6-yl, piperazin-2, 3, 5 or 6-yl, pyrrolidin-2 or 3-yl, homopiperidinyl, adamantanyl,
h) methyl, isopropyl, t-butyl, t-hexyl, —CF₃, —CF₂C₁₋₄alkyl,
i) ethenyl, ethynyl, cinnamyl, and
j) —COOmethyl, —COOphenyl, —COObenzyl, —COOcyclohexyl, —COOi-pentyl.

25. A method according to claim 23, wherein G, optionally substituted with R^{p″}, is selected from the group consisting of phenyl, cyclohexyl, pyridinyl, and pyrazolyl.

26. A method according to claim 23, wherein G is selected from the group consisting of phenyl, 2-methoxy-phenyl, 3-methoxy-phenyl, 4-methoxy-phenyl, 2,3-dimethoxy-phenyl, 3,4-dimethyoxy-phenyl, 2-chloro-phenyl, 3-chloro-phenyl, 4-chloro-phenyl, 2,4-dichloro-phenyl, 3,4-dichlorophenyl, 2,4-dichlorophenyl, 2,5-dichlorophenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 4-trifluoromethyl-phenyl, 3-trifluoromethoxy-phenyl, 4-trifluoromethoxy-phenyl, 4-t-butyl-phenyl, 4-trifluoromethyl-2-pyridyl, 4-methanesulfonyl-phenyl, 4-phenoxy-phenyl, 4-isopropyl-phenyl, 4-ethoxy-phenyl, 4-hydroxy-phenyl, benzo[1,3]diox-5-yl, 2,3-dihydro benzo[1,4]dioxin-6-yl, 3-pyrazolyl, pyridin-2-yl, pyridin-3-yl, pyridin-4-yl, cyclohexyl, morpholinyl, t-butyl, —CF₃, methyl, isopropyl, ethenyl, cinnamyl, —COOmethyl, —COOphenyl, and —COOcyclohexyl.

27. A method according to claim 23, wherein R^{p″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

28. A method according to claim 23, wherein R^{p″} is selected from the group consisting of methyl, methoxy, ethoxy, chloro, fluoro, trifluoromethyl, trifluoromethoxy, t-butyl, methanesulfonyl, phenoxy, isopropyl, and hydroxy.

29. A method according to claim 23, wherein Ar″, optionally substituted with R^{r″}, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl.

30. A method according to claim 23, wherein Ar″, optionally substituted with R^{r″}, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl.

31. A method according to claim 23, wherein Ar″ is selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5 -dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃.

32. A method according to claim 23, wherein there are 0, 1, or 2 R^{r″} substituents.

33. A method according to claim 23, wherein R^{r″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

34. A method according to claim 23, wherein R^{r″} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl.

35. A method according to claim 23, wherein Y is —H, methyl, ethyl, isopropyl, or propyl.

36. A method of making a compound of formula (III), 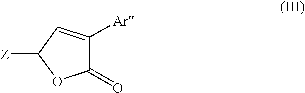
wherein,
Ar″ is selected from the group consisting of:
A) phenyl, optionally mono-, di-, or tri-substituted with R^{r″} or di-substituted on adjacent carbons with —OC₁₋₄alkyleneO—, —(CH₂)₂₋₃NH—, —(CH₂)₁₋₂NH(CH₂)—, —(CH₂)₂₋₃N(C₁₋₄alkyl)-, or —(CH₂)₁₋₂N(C₁₋₄alkyl)(CH₂)—;
R^{r″} is selected from the group consisting of —OH, —C₁₋₆alkyl, —OC₁₋₆alkyl, phenyl, —Ophenyl, benzyl, —Obenzyl, —C₃₋₆cycloalkyl, —OC₃₋₆cycloalkyl, —CN, —NO₂, —N(R^{y″})R^{z″} (wherein R^{y″} and R^{z″} are independently selected from —H, —C₁₋₆alkyl, and —C₁₋₆alkenyl, or R^{y″} and R^{z″} may be taken together with the nitrogen of attachment to form an otherwise aliphatic hydrocarbon ring, said ring having 4 to 7 members, optionally having one carbon replaced with >O, ═N—, >NH, or >N(C₁₋₄alkyl), optionally having one carbon substituted with —OH, and optionally having one or two unsaturated bonds in the ring), —(C═O)N(R^{y″})R^{z″}, —(N—R^{t″})COR^{t″}, —(N—R^{t′})SO₂C₁₋₆alkyl (wherein R^{t″} is —H or —C₁₋₆alkyl, or two R^{t″} in the same substituent may be taken together with the amide of attachment to form an otherwise aliphatic hydrocarbon ring, said ring having 4 to 6 members), —(C═O)C₁₋₆alkyl, —(S═(O)_{m″})—C₁₋₆alkyl (wherein m″ is selected from 0 or 2), —SO₂N(R^{y″})R^{z″}, —SCF₃, halo, —CF₃, —OCF₃, —COOH, and —COOC₁₋₆alkyl;
B) phenyl or pyridyl fused at two adjacent ring members to a three membered hydrocarbon moiety to form a fused five membered aromatic ring, which moiety has one carbon atom replaced by >O, >S, >NH, or >N(C₁₋₄alkyl), and which moiety has up to one additional carbon atom optionally replaced by N, the fused rings optionally mono-, di-, or tri-substituted with R^{r″};
C) phenyl fused at two adjacent ring members to a four membered hydrocarbon moiety to form a fused six membered aromatic ring, which moiety has one or two carbon atoms replaced by N, the fused rings optionally mono-, di-, or tri-substituted with R^{r″};
D) naphthyl, optionally mono-, di-, or tri-substituted with R^{r″};
E) a monocyclic aromatic hydrocarbon group having five ring atoms, having a carbon atom which is the point of attachment, having one carbon atom replaced by >O, >S, >NH, or >N(C₁₋₄alkyl), having up to one additional carbon atoms optionally replaced by N, optionally mono- or di-substituted with R^{r″} and optionally benzo or pyrido fused on the condition that two or fewer of said carbon ring atoms are replaced by a heteroatom, where the benzo or pyrido fused moiety is optionally mono-, di-, or tri-substituted with R^{r″}; and
F) a monocyclic aromatic hydrocarbon group having six ring atoms, having a carbon atom which is the point of attachment, having one or two carbon atoms replaced by N, having one N optionally oxidized to the N-oxide, optionally mono- or di-substituted with R^{r″} and optionally benzo or pyrido fused, where the benzo or pyrido fused moiety is optionally mono- or di-substituted with R^{r″};
G) adamantanyl or monocyclic C₅₋₇cycloalkyl, optionally having one or two carbon members optionally replaced with >O, >S, >NH or >N(C₁₋₄alkyl) and optionally having one or two unsaturated bonds in the ring and optionally having one of the ring atoms substituted with —OH, ═O, or —CH₃;
H) a —C₁₋₈alkyl wherein the carbon of attachment bears no hydrogen substituents, optionally mono-, di-, or tri-substituted by a R^{r″} or a substituent selected from the group consisting of any one of a) to g); and
I) —C₂alkenyl or —C₂alkynyl, optionally substituted with a substituent selected from the group consisting of any one of a) to h); and
Z is —C₁₋₈alkyl or —OC₁₋₈alkyl;
or an ester, enantiomer, diastereomer, racemic, or pharmaceutically acceptable salt thereof, comprising: providing an α-hydroxyester compound having
(a) an α-carbon member that is alkylated through an intervening methylene with a group that does not have a dehydration-removable hydrogen bonded to said methylene,
(b) an ester moiety with its carboxy group attached directly to said α-carbon member, and
(c) a substituent attached to said α-carbon member, wherein the volume of said substituent is greater than the volume of said ester moiety; and
treating said α-hydroxyester compound with a dehydrating agent.

37. A method according to claim 36, wherein Ar″, optionally substituted with Rr, is selected from the group consisting of:
A) phenyl, 5-, 6-, 7-, 8-benzo-1,4-dioxanyl, 4-, 5-, 6-, 7-benzo-1,3-dioxolyl, 4-, 5-, 6-, 7-indolinyl, 4-, 5-, 6-, 7-isoindolinyl, 1,2,3,4-tetrahydro-quinolin-4, 5, 6 or 7-yl, 1,2,3,4-tetrahydro-isoquinolin-4, 5, 6 or 7-yl,
B) 4-, 5-, 6- or 7-benzoxazolyl, 4-, 5-, 6- or 7-benzothiophenyl, 4-, 5-, 6- or 7-benzofuranyl, 4-, 5-, 6- or 7-indolyl, 4-, 5-, 6- or 7-benzthiazolyl, 4-, 5-, 6- or 7-benzimidazolyl, 4-, 5-, 6- or 7-indazolyl, imidazo[1,2-a]pyridin-5, 6, 7 or 8-yl, pyrazolo[1,5-a]pyridin-4, 5, 6 or 7-yl, 1H-pyrrolo[2,3-b]pyridin-4, 5 or 6-yl, 1H-pyrrolo[3,2-c]pyridin-4, 6 or 7-yl, 1H-pyrrolo[2,3-c]pyridin-4, 5 or 7-yl, 1H-pyrrolo[3,2-b]pyridin-5, 6 or 7-yl,
C) 5-, 6-, 7- or 8-isoquinolinyl, 5-, 6-, 7- or 8-quinolinyl, 5-, 6-, 7- or 8-quinoxalinyl, 5-, 6-, 7- or 8-quinazolinyl,
D) naphthyl,
E) furanyl, oxazolyl, isoxazolyl, 1,2,3-oxadiazolyl, 1,2,4-oxadiazolyl, 1,2,5-oxadiazolyl, 1,3,4-oxadiazolyl, thiophenyl, thiazolyl, isothiazolyl, pyrrolyl, imidazolyl, pyrazolyl, 1,2,3-triazolyl, 1,2,4-triazolyl, 3-indoxazinyl, 2-benzoxazolyl, 2- or 3-benzothiophenyl, 2- or 3-benzofuranyl, 2- or 3-indolyl, 2-benzthiazolyl, 2-benzimidazolyl, 3-indazolyl, oxazolopyridinyl,
F) pyridinyl, pyrazinyl, pyrimidinyl, pyridazinyl, 1-, 3- or 4-isoquinolinyl, 2-, 3- or 4-quinolinyl, 2- or 3-quinoxalinyl, 2- or 4-quinazolinyl, naphthyridinyl,
G) cyclopropyl, cyclobutyl, cyclopentyl, cyclohexyl, cycloheptyl, tertrahydrofuranyl, tetrahydropyranyl, pyrrolidinyl, piperidinyl, azepinyl, piperazinyl, N-methylpiperidinyl, 2-oxopiperidinyl, morpholinyl, thiomorpholinyl,
H) t-butyl, t-hexyl, —CF₃, —CF₂C₁₋₄alkyl, and
I) ethenyl, ethynyl, cinnamyl.

38. A method according to claim 36, wherein Ar″, optionally substituted with R^{r″}, is selected from the group consisting of phenyl, naphthalenyl, benzofuran-3-yl, 4, 5, 6 or 7-benzothiophenyl, 4, 5, 6 or 7-benzo[1,3]dioxolyl, 8-quinolinyl, 2-indolyl, 3-indolyl, pyridinyl, cyclopropyl, cyclopentyl, cyclohexyl, —CF₃, and t-butyl.

39. A method according to claim 36, wherein Ar″ is selected from the group consisting of phenyl, 2-methyl-phenyl, 3-methyl-phenyl, 4-methyl-phenyl, 2,5-dimethyl-phenyl, 2-trifluoromethyl-phenyl, 3-trifluoromethyl-phenyl, 2-fluoro-3-trifluoromethyl-phenyl, 2-fluoro-phenyl, 2,3-difluoro-phenyl, 2-chloro-phenyl, 3-chloro-phenyl, 4-chloro-phenyl, 2,3-dichloro-phenyl, 3,4-dichlorophenyl, 2,6-dichlorophenyl, 2-chloro-4-fluoro-phenyl, benzofuran-3-yl, 2-methoxy-phenyl, 3-methoxy-phenyl, 4-methoxy-phenyl, 2,3-dimethoxy-phenyl, 3-trifluoromethoxy-phenyl, 4-trifluoromethoxy-phenyl, 3-ethoxy-phenyl, naphthalen-1-yl, naphthalen-2-yl, benzo[b]thiophen-4-yl, 3-nitro-phenyl, benzo[1,3]dioxol-5-yl, pyridin-3-yl and pyridin-4-yl, 3-indolyl, 1-methyl-indol-3-yl, 4-biphenyl, 3,5-dimethyl-phenyl, 3-isopropoxy-phenyl, 3-dimethylamino-phenyl, 2-fluoro-5-methyl-phenyl, 2-methyl-3-trifluoromethyl-phenyl, t-butyl, and —CF₃.

40. A method according to claim 36, wherein there are 0, 1, or 2 R^{r″} substituents.

41. A method according to claim 36, wherein R^{r″} is selected from the group consisting of —OH, —CH₃, —CH₂CH₃, i-propyl, t-butyl, —OCH₃, —OCH₂CH₃, —OCH(CH₃)₂, cyclopropyl, cyclobutyl, cyclopentyl, cyclohexyl, —Ocyclopentyl, —Ocyclohexyl, phenyl, —Ophenyl, benzyl, —Obenzyl, —NO₂, —C(O)NH₂, —C(O)N(CH₃)₂, —C(O)NH(CH₃), —NHCOCH₃, —NCH₃COCH₃, —NHSO₂CH₃, —NCH₃SO₂CH₃, —C(O)CH₃, —SO₂CH₃, —SO₂NH₂, —SO₂NHCH₃, —SO₂N(CH₃)₂, —SCF₃, —F, —Cl, —CF₃, —OCF₃, —COOH, —COOCH₃, —COOCH₂CH₃, —NH₂, —NHCH₃, —NHCH₂CH₃, —NH(CH₂CH₂CH₃), —NH(CH(CH₃)CH₂CH₃), —NH(allyl), —NH(CH₂(CH₃)₂), —N(CH₃)₂, —N(CH₂CH₃)₂, —NCH₃(CH₂CH₂CH₃), —NCH₃(CH₂CH₃), —NCH₃(CH(CH₃)₂), pyrrolidin-2-one-1-yl, azetidinyl, piperidin-1-yl, 2- or 3-pyrrolin-1-yl, morpholin-4-yl, thiomorpholin-4-yl, piperazin-1-yl, pyrrolidin-1-yl, and homopiperidin-1-yl.

42. A method according to claim 36, wherein R^{r″} is selected from the group consisting of methyl, methoxy, ethoxy, isopropoxy, dimethylamino, fluoro, chloro, trifluoromethyl, trifluoromethoxy, nitro, phenyl, and trifluoromethylsulfanyl.

43. A method according to claim 36, wherein Z is selected from the group consisting of methyl, ethyl, propyl, isopropyl, butyl, t-butyl, isobutyl, hexyl, methoxy, ethoxy, propoxy, isopropoxy, butoxy, and hexyloxy.

44. A method according to claim 36, wherein Z is methyl or methoxy.

45. A method according to claim 23, wherein said α-hydroxyester compound is a compound of formula S5 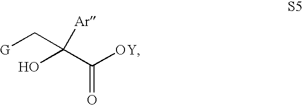
with G, Ar″ and Y defined as in claim 23.

46. A method according to claim 45, wherein said dehydrating agent is one of triflic an hydride, fluorosulfonic an hydride, methanesulfonyl chloride, and chemically compatible mixtures thereof.

47. A method according to claim 45, wherein said dehydrating agent is triflic anhydride in the presence of pyridine.

48. A method according to claim 45, further comprising a hydrolysis subsequent to said treating with said dehydrating agent.

49. A method according to claim 45, further comprising obtaining said α-hydroxyester compound of formula S5 by alkylating a mandelic acid analog of formula S1

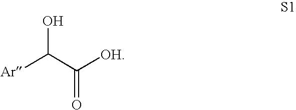

50. A method according to claim 49, further comprising protecting said mandelic acid analog prior to said alkylating to form a protected alkylated product, and deprotecting said protected alkylated product to form said compound of formula S5.

51. A method according to claim 49, wherein said alkylating comprises treating said mandelic acid analog of formula S2 with one of n-BuLi, LDA, LiHMDS, NaH, and chemically compatible mixtures thereof.

52. A method according to claim 36, wherein said α-hydroxyester compound is a compound of formula S23 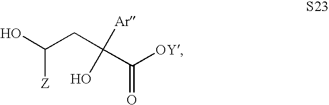
with Z, Ar″ as defined in claim 36, and Y′ being —C₁₋₄alkyl.

53. A method according to claim 52, wherein said dehydrating agent is one of triflic anhydride, fluorosulfonic anhydride, methanesulfonyl chloride, and chemically compatible mixtures thereof.

54. A method according to claim 52, wherein said dehydrating agent is triflic anhydride in the presence of pyridine.

55. A method according to claim 52, further comprising obtaining said α-hydroxyester compound of formula S23 by alkylating a mandelic acid analog of formula S1

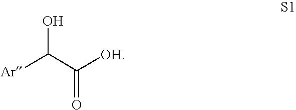

56. A method according to claim 55, further comprising protecting said mandelic acid analog prior to said alkylating to form a protected alkylated product, and deprotecting said protected alkylated product to form a compound of formula S23.

57. A method according to claim 56, wherein said alkylating comprises treating said mandelic acid analog with one of n-BuLi, LDA, LiHMDS, NaH, and chemically compatible mixtures thereof.

58. A method according to claim 1, wherein said α-hydroxyester compound is a compound of formula S13 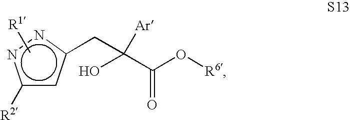
where R^{1′}, R^{2′}, Ar′, and R^{6′} are as defined in claim 1.

59. A method according to claim 58, wherein said dehydrating agent is one of triflic anhydride, fluorosulfonic anhydride, methanesulfonyl chloride, and chemically compatible mixtures thereof.

60. A method according to claim 58, wherein said dehydrating agent is triflic anhydride in the presence of pyridine.

61. A method according to claim 58, further comprising a hydrolysis subsequent to said treating with said dehydrating agent.

62. A method according to claim 58, further comprising obtaining said α-hydroxyester compound by alkylating a mandelic acid analog of formula S7 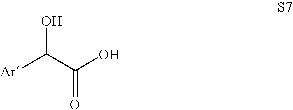
to form an acetylenic addition compound of formula S10

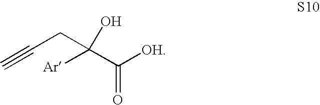

63. A method according to claim 62, further comprising forming an addition compound of formula S 11

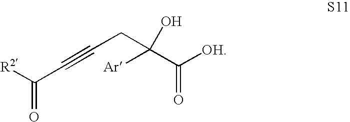

64. A method according to claim 63, further comprising condensing said compound of formula S11 with a suitably substituted hydrazine R^{1′}—NHNH₂ to form a pyrazole derivative.

65. A method according to claim 64, further comprising dehydrating said pyrazole derivative to form an ester of formula S14

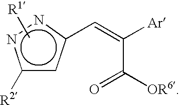

66. A method according to claim 65, further comprising hydrolyzing said ester of compound of formula S14 to obtain said compound of formula S14.

67. A method according to claim 58, wherein said α-hydroxyester compound is a compound of formula T6 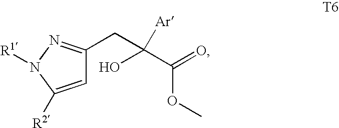
where R^{1′}, R^{2′}, and Ar′ are as defined in claim 58.

68. A method according to claim 67, wherein said dehydrating agent is one of triflic anhydride, fluorosulfonic anhydride, methanesulfonyl chloride, and chemically compatible mixtures thereof.

69. A method according to claim 67, wherein said dehydrating agent is triflic anhydride in the presence of pyridine.

70. A method according to claim 67, further comprising obtaining said α-hydroxyester compound by alkylating a mandelic acid analog of formula T2 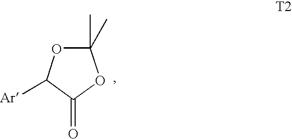
to form an acetylenic addition compound T3

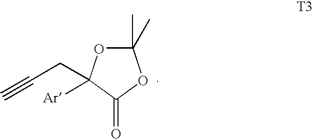

71. A method according to claim 70, further comprising a coupling step with said addition compound T3 to form an addition compound of formula T4

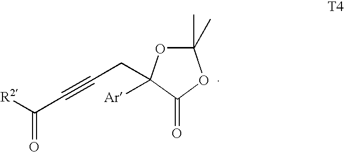

72. A method according to claim 71, further comprising condensing said compound of formula T4 with hydrazine R^{1′}—NHNH₂ to form a pyrazole derivative of formula T5

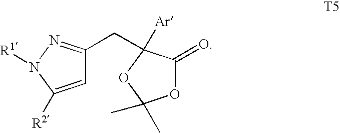

73. A method according to claim 72, further comprising deprotecting said pyrazole derivative of formula T5 to form a compound of formula T6

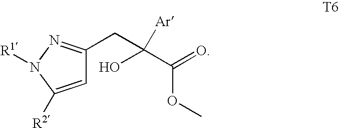

74. A method according to claim 73, further comprising dehydrating said pyrazole derivative of formula T6 to form an ester of compound of formula T7:

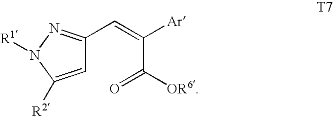

75. A method according to claim 74, further comprising hydrolyzing said ester of compound of formula T7 to obtain a compound of formula T7 in its acid form:

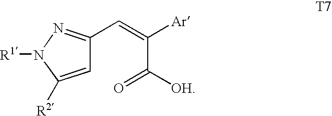

76. A method according to claim 58, wherein said compound of formula S13 is a compound of formula 604

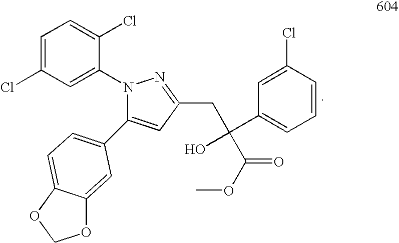

77. A method according to claim 76, wherein said dehydrating agent is one of triflic an hydride, fluorosulfonic an hydride, methanesulfonyl chloride, and chemically compatible mixtures thereof.

78. A method according to claim 76, wherein said dehydrating agent is triflic anhydride in the presence of pyridine.

79. A method according to claim 76, further comprising obtaining said α-hydroxyester compound of formula 604 by alkylating a mandelic acid analog of formula 600 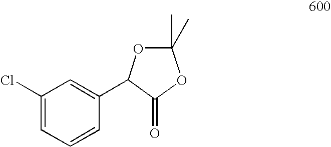
with propargyl bromide to form an acetylenic addition compound of formula 601

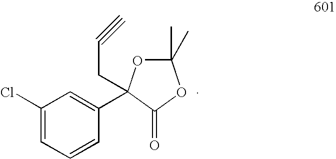

80. A method according to claim 77, further comprising forming a compound of formula 602 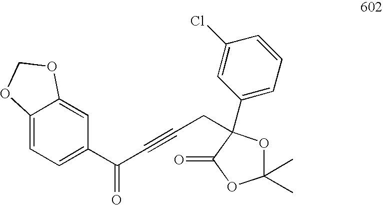
from said addition compound 601 in a coupling reaction.

81. A method according to claim 80, further comprising condensing said compound of formula 602 with a hydrazine to form a pyrazole derivative of formula 603

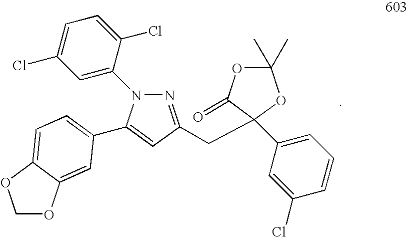

82. A method according to claim 81, further comprising deprotecting said pyrazole derivative of formula 603 to form a compound of formula 604

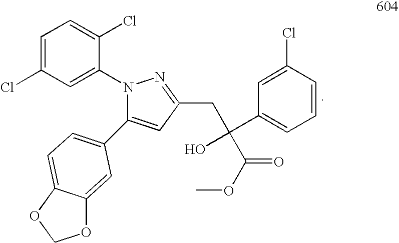

83. A method according to claim 82, further comprising dehydrating said pyrazole derivative of formula 604 to form a compound of formula 605

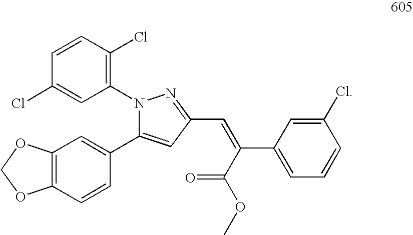

84. A method according to claim 83, further comprising hydrolyzing said ester of compound of formula 605 to obtain said compound of formula 606 [chemical structure]
